(19) United States
(12) Patent Application Publication
(10) Pub. No.: US 2024/0076624 A1
(43) Pub. Date: Mar. 7, 2024

(54) Methods and Tools for Studying Enteroendocrine Cells

(71) Applicant: Koninklijke Nederlandse Akademie Van Wetenschappen, Utrecht (NL)

(72) Inventors: Joep Beumer, Utrecht (NL); Jens Puschhof, Utrect (NL); Johannes Carolus Clevers, Utrecht (NL)

(73) Assignee: Koninklijke Nederlandse Akademie Van Wetenschappen, Utrecht (NL)

(21) Appl. No.: 17/915,771

(22) PCT Filed: Mar. 31, 2021
(86) PCT No.: PCT/EP2021/058527

§ 371 Date: Sep. 29, 2022

(30) Foreign Application Priority Data
Mar. 31, 2020 (GB) 2004706.4
Apr. 21, 2020 (GB) 2005794.9
Apr. 24, 2020 (GB) 2006092.7
May 13, 2020 (GB) 2007067.8

(51) Int. Cl. C12N 5/071; C12N 15/65; C40B 30/06

(52) U.S. Cl. CPC C12N 5/0679; C12N 15/65; C40B 30/06; C12N 2510/00; C12N 2513/00

(57) ABSTRACT

The disclosure relates to methods and tools for studying enteroendocrine cells, including intestinal reporter organoids, methods and media for making said organoids, uses of said organoids, and enteroendocrine cell targets and treatments.

### TECHNICAL FIELD

The invention is in the field of methods and tools for studying enteroendocrine cells (EECs).

### BACKGROUND

The principal function of the intestine is to digest food and absorb nutrients. A less studied aspect of intestinal function is its role as the largest hormone-producing organ, an activity performed by the enteroendocrine cells (EECs) (Gribble and Reimann, 2017). EECs are rare secretory cells, comprising <1% of the epithelial cells. Enteroendocrine cells (EECs) sense intestinal content and release hormones to regulate gastrointestinal activity, systemic metabolism and food intake. Apical EEC receptors are believed to sense chemicals in the intestinal lumen derived from food and microbiota. These receptors are predominantly GPCRs and include olfactory receptors (Furness et al., 2013). Hormones secreted by EECs can signal to the local enteric nervous system as well as to distant organs including the pancreas and the brain, e.g. through vagal neurons, thus controlling key physiological processes related to food intake, insulin release, secretion of digestive enzymes and bowel movement (Bellono et al. 2017; Kaelberer et al 2018; Raybould et al, 2007). Enteric hormones have also been proposed to orchestrate mucosal immunity (Worthington et al., 2018). Gut peptides have been found to be produced in the brain (Gardiner et al. 2010), which supports the findings that the brain can respond to the types of hormones secreted by the gut. EECs are emerging therapeutic targets for the management of metabolic diseases (i.e. obesity and diabetes), illustrated by recently introduced type 2 diabetes drugs that stabilize the hormone Glucagon-like peptide 1 (GLP-1) or activate its receptor, leading to release of insulin from pancreatic β-cells (Sharma et al., 2018).

EECs produce some 20 different hormones. GLP-1 and Glucose-dependent insulinotropic peptide (GIP) are the so-called incretin hormones that stimulate insulin secretion. The Enterochromaffin (EC) cells of the gut produce 90% of body serotonin and regulate bowel movement (Worthington et al., 2018). Motilin (MLN) is a human EEC hormone that is a pseudogene in the mouse genome, which controls gut contractions in the inter-digestive state (Worthington et al., 2018).

Cholecystokinin (CCK) regulates bile and pancreatic enzyme secretion. Multiple hormones control appetite, including the appetite-inducing (or orexigenic) Ghrelin (GHRL) and the appetite-reducing (anorexigenic) Peptide YY (PYY). Gastrin (GAST), while rare in the mouse intestine, is produced in the human duodenum and responds to luminal acid by regulating proton secretion of stomach parietal cells. Somatostatin (SST) is a peptide which inhibits secretion of most other intestinal hormones (Worthington et al., 2018).

Homeostatic renewal of the intestine is driven by Lgr5+ stem cells located at the bottom of the crypts of Lieberkuehn (Barker et al., 2007). Lgr5+ cells generate all differentiated intestinal cell types: the abundant absorptive enterocytes and the various secretory cell types. The murine EEC lineage consists of multiple subtypes, historically defined by their principle hormone product: L-cells (Glp-1, Pyy), I-cells (Cck), K-cells (Gastric inhibitory protein, Gip), N-cells (Neurotensin, Nts), S-cells (Secretin, Sct), enterochromaffin or EC-cells (Serotonin/5-HT), X-cells (Ghrl), G-cells (Gast) and D-cells (Sst) (Engelstoft et al., 2013a) (Gehart et al., 2019). While this nomenclature suggests that EEC phenotypes are hard-wired, the inventors have recently found that the crypt-villus BMP-signaling gradient induces hormone switching in individual murine EEC lineages (Beumer et al., 2018). Of note also, the abundance of individual EEC subtypes greatly differs in a region-specific fashion along the proximal-distal gastrointestinal axis. Studies on EECs have largely focused on murine models, exploiting a variety of reporter mice for subsets of EECs to monitor their responses to nutritional or genetic challenges (Goldspink et al., 2018). The inventors have recently described the developmental hierarchy of murine EEC subtypes using a mouse model in which endogenous Neurogenin-3 expression, the main determinant of EEC fate, was coupled to the production of two separate fluorescent proteins with different half-lives (Gehart et al., 2019). Single cell RNA sequencing of sorted EEC progenitors allowed for construction of a time-resolved development roadmap of the mouse EEC lineage. However, use of this Neurog3 tracer does not allow all different hormones and EEC subtypes to be distinguished.

The human diet and microbiome and that of rodents differ greatly (Nguyen et al., 2015). It is likely that the repertoire of sensory receptors and the secretory hormone responses differ significantly between these species.

Little is known about the molecular make-up of human EEC subtypes and the regulated secretion of individual hormones. The study of human EECs is challenging due to the paucity of these cells and the lack of physiologically relevant in vitro models, leaving many questions surrounding basic human EEC biology unanswered. Few human EEC immortalized cell lines exist, and these are known to differ substantially from their wildtype counterparts (Goldspink et al., 2018).

There is currently no atlas of human EEC subtypes. Although some inducers of hormone secretion have been described in mice, there has been no experimental model to systematically assess such secretagogues for human EECs.

### SUMMARY OF THE INVENTION

Human intestinal organoids represent a tractable system that could be used to address these challenges. The inventors have developed an organoid-based platform for functional and molecular studies of human EECs. In particular, the organoid-based platform described herein advantageously enables a detailed molecular and functional description of human EECs. A set of gut organoids has been engineered in which the major hormones are fluorescently tagged. A single-cell mRNA atlas has been generated for the different EEC subtypes, and their secreted products have been recorded by mass-spectrometry. A number of hormones, sensory receptors and transcription factors have been identified in human EECs for the first time and the practical applications of these are provided herein. Accordingly, the invention provides a rich resource to study human EEC development and function, for example, to identify regulators thereof.

Accordingly, the invention provides an intestinal reporter organoid comprising one or more EEC-specific genes tagged at its endogenous locus with a detectable marker and which comprises an inducible transcription factor for differentiating intestinal stem cells and/or intestinal cells with stem cell potential to EECs.

Accordingly, the invention also provides a human intestinal reporter organoid comprising one or more EEC-specific genes tagged at its endogenous locus with a detectable marker.

Also provided is a method for making a reporter organoid comprising making one or more intestinal organoids comprising stem cells and/or cells with stem cell potential in which one or more EEC-specific genes is tagged at its endogenous locus with a detectable marker, wherein, selecting for correct insertion of the detectable marker comprises differentiating stem cells and/or cells with stem cell potential in the intestinal organoid to EECs and selecting one or more organoids which express the detectable marker upon differentiating the cells to EECs.

Also provided is a method for making an intestinal organoid comprising EECs which comprises inhibiting Wnt signalling in an intestinal organoid comprising intestinal stem cells and/or intestinal cells with stem cell potential before differentiating the stem cells and/or cells with stem cell potential to EECs.

Also provided is a method for making an intestinal organoid comprising EECs, wherein the expansion medium and/or the differentiation medium in which the intestinal organoids comprising EECs are cultured comprises a BMP pathway activator.

Also provided is a method for making a clonal intestinal organoid as described herein.

Also provided is a clonal intestinal organoid which comprises stem cells and/or cells with stem cell potential which comprise an inducible transcription factor for differentiating intestinal stem cells and/or intestinal cells with stem cell potential to EECs.

Also provided is a method for enriching the population of EECs in an intestinal organoid, as described herein.

Also provided is an organoid obtained by or obtainable by a method according to the invention.

Also provided is a biobank comprising two or more different organoids described herein.

Also provided is the use of an organoid of the invention or a biobank of the invention in an assay.

Also provided is a culture medium or supernatant comprising secreted products from a human organoid of the invention, wherein the culture medium or supernatant is obtained by or is obtainable by culturing the organoid.

Also provided is a method for isolating one or more EECs or isolating one or more EECs of an EEC subtype of interest, as described herein.

Also provided is a method for studying secretion and/or expression and/or production of one or more EEC-specific proteins, as described herein.

Also provided is a method for identifying/screening/validating a compound for suitability for modulating secretion or production of one or more hormones and/or hormone precursors and/or modulating expression of one or more EEC-specific proteins, as described herein.

Also provided is a method for identifying/screening/validating a compound for suitability for controlling differentiation of one or more EEC subtypes, as described herein.

Also provided is a method for identifying/screening/validating a compound for suitability for treating or preventing a disease or disorder, as described herein.

Also provided is a method for determining whether a patient will respond to a compound for treating or preventing a disease or disorder, as described herein.

Also provided is a method for determining whether a compound affects secretion of one or more hormones and/or hormone precursors in EECs or in one or more subtypes thereof, as described herein.

Also provided is a method for testing the effect of one or more bacteria, a supernatant from a bacterial culture or a bacterially-derived compound on EECs or on one or more EEC subtypes, as described herein.

Also provided is a method for generating an EEC RNA expression atlas of EECs or of one or more subtypes of EEC, as described herein.

Also provided is a method for generating an EEC protein expression atlas, as described herein.

Also provided is a human EEC RNA expression or protein expression atlas.

Also provided is a method for isolating one or more human EECs or for isolating a population of human EECs, as described herein.

Also provided is an isolated population of human EECs.

Also provided is an isolated human EEC.

Also provided is a method for identifying one or more human EECs or for identifying a population of human EECs, as described herein.

Also provided is a pharmaceutical composition comprising a therapeutic compound which affects a target in a human EEC, wherein the pharmaceutical composition is formulated to be administered or delivered to the gut.

Also provided is a method for identifying/validating a drug target of interest in a human EEC, as described herein.

Also provided is a method for identifying/screening/validating a compound for targeting a target in a human EEC, as described herein.

Also provided is a method for identifying/screening/validating a compound for suitability for treating or preventing a disease or disorder, as described herein.

Also provided is a method for treating or preventing a disease or disorder comprising administering a compound that targets a target in a human EEC, wherein the target is implicated in the disease or disorder.

Also provided is a method for treating or preventing a disease or disorder comprising administering a compound found to affect secretion and/or production of one or more hormones and/or hormone precursors and/or expression of one or more hormones, hormone precursors and/or hormone synthesizing enzymes when used to target a target in a human EEC, wherein the one or more hormones, hormone precursors and/or hormone synthesizing enzymes is implicated in the disease or disorder.

Also provided is a method for modulating a physiological effect comprising modulating expression and/or secretion and/or production of, or agonising or antagonising one or more targets in a human EEC.

Also provided is a method for modulating expression and/or secretion and/or production of one or more hormones, hormone precursors and/or hormone synthesizing enzymes comprising targeting a cell surface receptor on human EECs.

Also provided is a method for treating or preventing a disease or disorder requiring appetite inhibition, diabetes or a neurodegenerative disease or disorder such as Parkinson's Disease or Alzheimer's Disease comprising administering a compound that targets the SCTR in human EECs to a patient.

Also provided is a method for treating or preventing diabetes, obesity or a neurodegenerative disease or disorder such as Parkinson's Disease or Alzheimer's Disease comprising administering a compound that targets the SCTR in human EECs to a patient.

Also provided is a method for treating or preventing a neuroendocrine tumour, comprising targeting the SCTR on small intestinal gastrin-producing G-cells.

Also provided is a method of treating or preventing a disease or disorder selected from an appetite-related disease or disorder (e.g. obesity, anorexia, cancer cachexia, obesity, diabetes (in particular type II diabetes), Prader-Willi syndrome), a gut motility disease or disorder (e.g. a bowel movement disorder, Parkinson's disease or gastroparesis), diabetes (in particular type II diabetes), a neurodegenerative disease or disorder (e.g. Parkinson's disease or Alzheimer's disease), and depression, comprising administering an HHEX knockout construct or an HHEX inhibitor to a patient, wherein the treating or preventing comprises inhibiting or knocking out HHEX in the patient's EECs.

Also provided is a method of treating or preventing type II diabetes comprising administering an HHEX knockout construct or an HHEX inhibitor to a patient, wherein the treating or preventing comprises inhibiting or knocking out HHEX in the patient's EECs.

Also provided is a method for treating IBS comprises administering an LMX1A knockout construct or an LMX1A inhibitor to a patient, wherein the treating or preventing comprises inhibiting or knocking out LMX1A in the patient's EECs.

Also provided is a method of regulating hormone secretion by an EEC comprising contacting the EEC with beta-ionone.

Also provided is a method for stimulating expression of one or more villus-restricted hormones and/or decreasing expression of one or more crypt-restricted hormones in human EECs, comprising activating BMP signalling in the human EECs, wherein the one or more villus-restricted hormones are MLN and/or SCT and/or wherein the one or more crypt-restricted hormones is GCG.

Also provided is a method for treating or preventing a gut motility disorder (e.g. a bowel movement disorder, Parkinson's disease or gastroparesis) or a disease or disorder requiring appetite inhibition comprising administering a BMP pathway activator to a patient, wherein the treating or preventing comprises activating BMP signalling in the patient's EECs.

Also provided is a method for treating or preventing a gut motility disorder (e.g. a bowel movement disorder, Parkinson's disease or gastroparesis) comprising administering a BMP pathway activator to a patient, wherein the treating or preventing comprises activating BMP signalling in the patient's EECs.

Also provided is a method for increasing Motilin expression and/or secretion and/or decreasing Ghrelin expression and/or secretion in human EECs which comprises activating BMP signalling in the human EECs.

Also provided is a method for modulating hormone expression in human EECs comprising contacting the human EECs with Midkine and/or modulating the endogenous expression of Midkine.

Also provided is a method for increasing the expression of one or more of GHRL, MLN, CCK, GCG, TPH1 and ChgA in one or more EECs comprising culturing the one or more EECs in the presence of Akkermansia muciniphila or in the presence of a supernatant from a culture of Akkermansia muciniphila.

Also provided is a method of treating or preventing a gut motility disorder comprising administering Akkermansia muciniphila to a patient.

### BRIEF DESCRIPTION OF THE FIGURES

FIG. 1: Production of region-specific human enteroendocrine cells in intestinal organoids

(A) Schematic representation of the generation of region-specific enteroendocrine cells (EECs). Organoids are established from different regions of the intestinal tract of different patients, after which doxycycline (dox)-induced overexpression of Neurogenin-3 (NEUROG3) can drive the production of EECs.

(B) qPCR analysis showing expression of hormones, control or differentiation condition after a pulse of dox. Expression levels are normalized to GADPH. The experiment was performed in n=2 independent experiments, and the mean expression and SEM are depicted.

(C) Transmission electron microscopy (TEM) of EECs in organoids showing polarized localization of hormone vesicles. Scale bar is 5 μm (left image) and 2 μm (right image).

(D) Concentration of supernatant GLP-1 determined by ELISA, in the absence (control) and presence of forskolin (FSK). Forskolin induces secretion of GLP-1, confirming functionality of EECs. The experiment was performed in n=3 independent experiments, and the mean concentration and SEM are depicted.

FIG. 2: Generation of a human enteroendocrine cell organoid toolbox

(A) Schematic workflow of generation of reporter organoids.

(B) Overview of EEC-TAG biobank comprising reporters for hormones across human duodenum, ileum and colon organoids. EECs are tagged with NHEJ (mNeon or dTomato). TPH1-positive ECs are tagged using HDR with mClover.

(C) Immunofluorescent staining confirms faithful reporter activity (knock-in left, stain middle and merge on right). Reporter expression always overlaps with the corresponding hormone. Scale bar is 50 μm.

(D) Subsequent rounds of hormone tagging allow the generation of multiple-hormone reporter organoids. Immunofluorescent staining of GCG/CHGA double reporter organoid. Scale bar is 50 μm.

(E) qPCR analysis showing expression of the olfactory receptor OR51E2 in different organoids enriched for EECs. Expressions levels are normalized to GADPH and relative to control organoids without EECs. The experiment was performed in n=2 independent experiments, and the mean expression and SEM are depicted.

(F) Fluorescent image of a TPH1^{mClover} NEUROG3^{dTomato} organoid that is transduced with the turquoise calcium sensor Tq-Ca-FLITS, containing a nuclear localization signal. Five examples of nuclei are highlighted of which the calcium response is followed after treatment with Beta-ionone, the agonist of OR51E2. EECs (top three curves) show increases in calcium flux (‘2-4’) with the exception of the TPH1⁺ cell (‘1’). The non-EEC (‘5’) does not show calcium increases upon Beta-ionone treatment.

FIG. 3: Single cell transcriptome atlas of human enteroendocrine cells

(A) Immunofluorescent staining on human intestinal section (ileum) confirms EEC-specific expression of NPW. Scale bar is 50 μm.

(B) t-SNE map displaying the human EEC atlas (n=2255 cells). Different circled areas/shades represent the 13 separate clusters, and BMP treated cells are highlighted. GAST- and GIP-positive cells (defined by a minimum of 25 unique transcripts per cell) show partly overlapping expression patterns (middle t-SNE map). GHRL- and MLN-positive cells (defined by a minimum of 25 unique transcripts per cell) also overlap partly (right t-SNE map).

(C) t-SNE maps displaying the specifically increased expression levels of hormone and marker gene expression grouped by the different human EEC subtypes from intestinal organoids. Bars display color-coded unique transcript expression (logarithmic scale; dark corresponds to low expression, light corresponds to high expression).

(D) t-SNE maps displaying the expression levels of hormone and marker gene expression in murine tissue EECs. Bars display color-coded unique transcript expression (logarithmic scale; dark corresponds to low expression, light corresponds to high expression).

(E) Immunofluorescent staining on duodenal sections confirms co-expression of GIP and GASTRIN. Scale bar is 50 μm.

FIG. 4: Novel human enteroendocrine cell markers

(A) t-SNE maps displaying the levels of hormone and marker gene expression in the different human EEC subtypes from intestinal organoids. Bars display color-coded unique transcript expression (logarithmic scale; dark corresponds to low expression, light corresponds to high expression).

(B) t-SNE maps displaying the levels of marker gene expression in the different human MX-cells from intestinal organoids. Bars display color-coded unique transcript expression (logarithmic scale; dark corresponds to low expression, light corresponds to high expression).

(C) Immunofluorescent staining on intestinal sections confirms co-expression of GHRL and the novel EEC peptide CBLN1 (left panel). Fluorescent in situ hybridization shows that GHRL⁺ cells express the cytokine receptor IL-20RA (right panel). All sections are from the human duodenum.

Scale bar is 50 μm.

FIG. 5: Transcriptional networks in human EECs

(A) t-SNE map displaying the expression level of EEC lineage transcription factors. Bars display color-coded unique transcript expression (logarithmic scale; dark corresponds to low expression, light corresponds to high expression). The murine and human patterns of expression of these genes among EEC subtypes are depicted (right table).

(B) qPCR analysis showing expression of hormones in wildtype organoids and LMX1A and HHEX knockout (KO) organoids. Expression is normalized to GADPH, and relative to wildtype. The experiment was performed as a technical duplicate, and the mean expression and SEM are depicted.

(C) Immunofluorescent staining on wildtype and HHEX and LMX1A knockout organoids. D-cells (marked by white arrow) are reduced after HHEX loss, while EC-cells do not decrease upon LMX1A knockout. Scale bar is 50 μm.

(D) Quantification of C. Number of hormone positive cells are counted on organoid sections and shown normalized to number of tdTomato+ cells and relative to WT.

FIG. 6: Enteroendocrine cells in human intestinal organoids display normal coexpression profiles

(A) qPCR analysis showing expression of hormones over the course of EEC differentiation.

Expressions levels are normalized to GADPH and relative to day 0. The experiment was performed in n=2 independent experiments, and the individual datapoints are depicted.

(B) qPCR analysis showing expression of hormones after different durations of doxycycline (Dox) challenge. Organoids were differentiated for 5 days, and treated without dox, for 6 hours, 24 hours, 48 hours or 120 hours (the full differentiation time) dox. Expressions levels are normalized to GADPH and relative to 48 h dox treatment. The experiment was performed in n=2 independent experiments, and the individual datapoints are depicted.

(C) Immunofluorescent staining of EEC-enriched organoids. Multiple hormones are expressed mutually exclusive. Scale bar is 50 μm.

FIG. 7: Manipulation of Wnt and BMP signaling allows controlling subspecification of EECs

(A) qPCR analysis showing expression of hormones after BMP treatment. Expression levels are normalized to GADPH, and relative to a non-treated control. The experiment was performed in n=2 independent experiments, and the mean expression and SEM are depicted.

(B) Immunofluorescent staining of BMP treated organoids. Scale bar is 50 μm.

(C) Schematic representation of morphogen gradients in the intestinal crypt related to sites of initiation of Neurogenin-3 (Ngn3) expression.

(D) Experimental paradigm. Different signalling pathways were modulated (BMP activation; BMPa, Wnt inhibition; Wnti, Notch inhibition; Notchi) either 24 hours before (pre) or at the start (post) of NEUROG3 expression mediated by doxycycline (dox) treatment. Control organoids were kept in standard expansion conditions (EXP) before dox treatment, and in standard differentiation conditions (ENR) after initiation of dox treatment.

(E) qPCR analysis showing expression of hormones after different treatments shown in. Expression levels are normalized to GADPH, and relative to a non-treated control. The experiment was performed in n=2 independent experiments, and the mean expression and SEM are depicted.

(F) Immunofluorescent staining of organoids differentiated towards EECs after a 24 hour inhibition of Wnt (pre Wnt i). Organoids are shown as a maximum projection. Scale bar is 50 μm.

(G) Quantification of (F). Number of positive cells were counted on n=5 organoid sections. Pre-inhibition of Wnt signalling caused a shift of L-cell to M-cell differentiation.

FIG. 8: Generation of a human single cell RNA sequencing atlas of the intestinal tract using organoids

(A) Fluorescence-activated cell sorting (FACS) gating parameters for sorting of different EEC subtypes from reporter organoids.

(B) Histogram displaying the total number of unique transcripts per cell (median number per cell is 7288 transcripts).

(C) A broad human intestinal organoid atlas (n=4281 cells) generated by single cell RNA sequencing and displayed using a t-distributed stochastic neighbour-embedding (t-SNE) map. Cells defined as EECs (see methods) are shown in black. Cells expressing NPW or VGF (>1 transcripts, respectively) are highlighted in black in the two right panels and are found exclusively in EEC clusters.

(D) t-SNE maps displaying lineage markers in the whole human intestinal organoid cell atlas (n=8448 cells). Bars display color-coded unique transcript expression (logarithmic scale).

FIG. 9: Single cell RNA sequencing of human EECs from organoids and mouse EECs from tissue

(A) t-SNE maps displaying the origin (left, tissue; right, reporter organoid) of cells from the human EEC atlas (n=2255 cells).

(B) The percentages of EECs corresponding to the different subtypes in proximal and distal SI organoids.

(C) t-SNE maps displaying the expression levels of hormones in the different murine EEC subtypes from intestinal tissue. Bars display color-coded unique transcript expression (logarithmic scale).

(D) t-SNE maps displaying conserved expression of different EC markers in human and mouse EECs. Bars display color-coded unique transcript expression (logarithmic scale).

(E) t-SNE maps displaying the levels of hormone and marker gene expression of human M/X cells in the different murine EEC subtypes from intestinal tissue. Bars display color-coded unique transcript expression (logarithmic scale).

(F) qPCR analysis showing expression of hormones after FGF-21 treatment during the 5 day EEC differentiation. Expressions levels are normalized to GADPH and relative to control organoids that are EEC differentiated without FGF-21 treatment. B-ACTIN is displayed as second housekeeping gene. The experiment was performed in n=2 independent experiments, and the mean expression and SEM are depicted.

FIG. 10: Subclustering of human EEC subtypes reveals heterogeneity including rare expression of PPY in L-cells

(A) Subclustering was performed on EECs sorted from different reporter organoids. t-SNE maps displaying the correlation between transcript levels and reporter intensity

(B) t-SNE maps displaying different hormones in EECs from the different reporter organoids. PPY-expressing cell form a distinct cluster of GCG⁺-cells.

(C) Immunohistochemistry of human ileal sections confirms PPY expression in vivo. Scale bar is 50 μm.

FIG. 11: BMP signaling regulates expression of hormones in individual enteroendocrine cells

(A) Fluorescent in situ hybridization on human ileal section shows crypt-restricted expression of GCG (dark arrowheads), whereas CHGA expression (light arrowheads) is expressed also in the villus. Scale bar is 50 μm.

(B) Violin plots depict the expression levels of selected hormones in single BMP-treated cells versus untreated cells in the EEC single cell RNA sequencing atlas. ID1 and ID2 are BMP target genes that confirm specific pathway activation of BMP agonist-treated cells.

(C) Snapshots are shown of GCG-neon reporter organoids that were treated with BMP after 2 days of dox treatment to induce NEUROG3-dTomato expression (=0 hr timepoint). BMP treatment blocks the appearance of GCG-positive cells, while pre-existing L-cells downregulate GCG expression. No cell death is observed. Scale bar is 50 μm.

FIG. 12: Bulk transcriptomic profiling of sorted enteroendocrine cell subtypes

(A) Experimental paradigm. Hormone reporter organoids are differentiated, after which subpopulations of EECs are sorted using FACS and processed for bulk RNA-sequencing or intracellular proteomics. In a separate experiment, the supernatant of organoids is collected after 24 hours forskolin stimulation and processed for proteomic analyses to determine the EEC secretome.

(B) Heatmaps showing the 20 most significant RNA markers enriched in purified reporter populations. In bold genes are highlighted that are also among the 20 most significant markers on protein level. Coloured bars represent Z-scores (dark colour=high Z-score).

(C) Heatmaps showing receptor expression most unique to TPH1⁺ or GCG⁺ cells. The receptor for EEC hormone Secretin (SCTR) is expressed highly in L-cells but not ECs, while ECs display unique expression of the PYY receptor (NPY1R).

FIG. 13: Secretin regulates release of GLP-1 from L-cells

(A) t-SNE map displaying the expression level of the Secretin receptor (SCTR) in the EEC single cell sequencing atlas. Bars display color-coded unique transcript expression (logarithmic scale).

(B) Fluorescent in situ hybridization on human ileal sections shows rare SCTR-expressing cells (dark arrowhead) that sometimes co-express CHGA (light arrowhead). Scale bar is 50 μm.

(C) Representative bright-field images of EEC-differentiated organoids after 24 hours Forskolin (FSK) or Secretin (SCT) treatment. Both FSK and SCT treatment causes swelling of organoids, indicative of cAMP activation. Scale bar is 1 mm.

(D) ELISA showing the fold increase in GLP-1 concentrations of EEC-enriched organoids after treatment with FSK or SCT. The experiment was performed in n=3 independent experiments, and the mean fold change and SEM are depicted.

(E) GCG-reporter organoids were differentiated towards EECs and treated with FSK or SCT. Intracellular levels of GCG-neon reduce over the course of FSK and SCT treatment. Scale bar is 2 mm.

FIG. 14: The Effect of bacteria and cytokines on EEC differentiation and function

(A) GCG and MLN reporter organoids were stimulated with a cytokine mix of IL-20RA agonists (Il-10, IL-19, IL-20, IL-24, IL-26) for 24 h. Loss of fluorescence (right panel) indicates secretion of motilin, but not glucagon.

(B) Akkermansia muciniphila supernatant was harvested after a 24 h incubation in ENR media (top). Organoids were induced for 24 h with doxycycline in ENR and then exposed for 96 h to the bacterial supernatant, bacterial lysate of live bacteria (bottom).

(C) qPCR measurements of relative hormone expression levels after Akkermansia muciniphila supernatant (second bar from left), lysate (right bar) or bacteria (second bar from right) exposure relative to unexposed organoids (left bar).

FIG. 15: Midkine treatment of EECs qPCR data showing expression of hormones upon treatment with Midkine.

### DETAILED DESCRIPTION

Methods for Making a Reporter Organoid

The inventors have developed a system in which EEC-specific genes, for example, hormones, hormone precursors and/or hormone synthesizing enzymes, are tagged with detectable markers in EECs in the organoids, for example, in organoids of the invention. Such organoids are described herein as “reporter organoids”. Since fusions of hormones and fluorescent proteins retain the ability to be secreted, the inventors envision the reporter organoids comprising tagged hormones and tagged hormone precursors to be used in studies aimed at identifying specific inducers of hormone secretion, or of controlling differentiation of individual EEC subtypes. Such methods are provided by the invention. Advantageously, the inventors have engineered a set of reporter organoids in which the major hormones, hormone precursors and/or hormone synthesizing enzymes are fluorescently tagged. Accordingly, in some embodiments, the major hormones, hormone precursors and/or hormone synthesizing enzymes are tagged. The major hormones and hormone precursors are CHGA, GCG, MLN, CCK, GAST, GIP, NTS, SST and GHRL and the major hormone synthesizing enzyme is TPH1. This in vitro model for studying hormone release from EEC subtypes and EEC differentiation advantageously does not rely on an animal model. It can also be used for the study of human EECs. The inventors' reporter system allows hormone secretion and EEC differentiation to be studied in live cells, for example, by direct visualisation of the fluorescence or through use of a sensor molecule. The ability to study hormone secretion and EEC differentiation also allows modulators of hormone secretion and EEC differentiation to be identified (e.g. agonists or antagonists).

Mouse models in which hormone expression is coupled to a fluorescent readout have previously been generated for several murine EEC hormones or hormone synthesizing enzymes: Glp-1, Chga, Gcg, Gip, Cck, Ghrl, Pyy, Sst and Tph1 (Engelstoft et al., 2013b, 2015; Gong et al., 2003; Parker et al., 2009; Reimann et al., 2008; Sommer and Mostoslavsky, 2014; Svendsen et al., 2016; Adriaenssens et al., 2015; Li et al., 2014; Beumer et al., 2018). These transgenic mouse models have been instrumental to study specific EEC subsets in mice. However, none of the mouse models utilise a secreted fusion protein comprising the hormone or hormone precursor tagged with a detectable marker. Use of such a system, as devised by the present inventors, advantageously allows live tracking of hormone production and secretion in organoids, which is not possible using the mouse models.

Thus, the invention provides a method for modifying an intestinal organoid comprising or consisting of tagging one or more EEC-specific genes in stem cells or cells with stem cell potential from the intestinal organoid with a detectable marker (e.g. a fluorescent tag) and generating one or more modified intestinal organoids. Similarly, there is provided a method for making a reporter organoid comprising or consisting of tagging one or more EEC-specific genes with a detectable marker (e.g. a fluorescent tag) in an intestinal stem cell and/or in an intestinal cell with stem cell potential and generating one or more intestinal organoids. In some embodiments, the one or more intestinal stem cells and/or intestinal cells with stem cell potential are obtained from or obtainable from an intestinal organoid. The EEC-specific gene is preferably tagged with the detectable marker at its endogenous locus.

An EEC-specific gene is a gene that is expressed in an EEC (for example, in all EECs or in one or more EEC subtypes) but is not expressed in other cells in the intestine. In some embodiments, an EEC-specific gene encodes an EEC-specific protein. An EEC-specific protein is a protein that is expressed in an EEC (for example, in all EECs or in one or more EEC subtypes) but is not expressed in any other cells in the intestine. Any gene that is specifically expressed in all EECs or in one or more EEC subtypes may be used. In some embodiments, the EEC-specific gene is a gene that is expressed in only one subtype of EEC, for example, an EEC subtype as described herein. For example, the EEC-specific gene may be selected from a gene encoding a hormone, hormone precursor, hormone synthesizing enzyme, transcription factor, receptor (e.g. a cell surface receptor, e.g. a GPCR), a protease, a kinase. In some embodiments, the EEC-specific gene encodes a target as described herein, for example, as identified using the EEC cell atlas. In some embodiments, the transcription factor is a transcription factor for differentiating intestinal stem cells and/or intestinal cells with stem cell potential to EECs. In some embodiments, the transcription factor is selected from NEUROG3, ATOH1/MATH1 and NEUROD1. In some embodiments, the receptor is a cell surface receptor, for example, a GPCR. In some embodiments, the EEC-specific gene is a hormone, hormone precursor, hormone synthesizing enzyme or a transcription factor for differentiating intestinal stem cells and/or intestinal cells with stem cell potential to EECs. In some embodiments, the EEC-specific gene is a transcription factor for differentiating intestinal stem cells and/or intestinal cells with stem cell potential to EECs. Preferably, the EEC-specific gene is a hormone, hormone precursor and/or a hormone synthesizing enzyme. More preferably, the EEC-specific gene is a hormone and/or a hormone precursor.

Accordingly, the invention provides a method for making a reporter organoid comprising or consisting of:
i. making one or more intestinal organoids comprising stem cells and/or cells with stem cell potential in which one or more EEC-specific genes is tagged at its endogenous locus with a detectable marker, and optionally
ii. differentiating stem cells and/or cells with stem cell potential in the intestinal organoid to EECs.

In some embodiments, the step of making one or more intestinal organoids comprising stem cells and/or cells with stem cell potential in which one or more EEC-specific genes is tagged at its endogenous locus with a detectable marker comprises or consists of:
i. contacting an intestinal stem cell and/or an intestinal cell with stem cell potential with a construct encoding the detectable marker, wherein the construct targets the endogenous locus of the EEC-specific gene; and
ii. generating one or more intestinal organoids by culturing the cells in an expansion medium.

In the various methods described herein, the intestinal stem cell or intestinal cell with stem cell potential may be present in a population of cells. Thus, in some embodiments, the methods comprise contacting a population of intestinal cells which comprises one or more intestinal stem cells and/or one or more intestinal cells with stem cell potential with the construct. In some embodiments, the population of intestinal cells comprises at least 2 (e.g. at least 2, 10, 50, 100, 500, 1000, 2000, 5000, 10000) intestinal stem cells and/or intestinal cells with stem cell potential.

The intestine contains various cells which are not stem cells but which have the potential to behave like a stem cell. An example of an intestinal cell with stem cell potential is a secretory progenitor cell marked by DII1. A further example of an intestinal cell with stem cell potential is an absorptive progenitor cell marked by Alpi, which can dedifferentiate upon receiving stem cell factors.

The step of differentiating stem cells and/or cells with stem cell potential to EECs may advantageously serve as a positive selection step for correct insertion of the detectable marker. The inventors have surprisingly found that use of differentiation of the cells to EECs as a positive selection step for cells comprising a correct insertion of the detectable marker advantageously allows reporter organoids to be constructed in an efficient manner. When intestinal stem cells are differentiated to EECs, they start expressing genes from the endogenous loci of their EEC-specific genes, for example, genes encoding transcription factors for differentiating intestinal stem cells and/or intestinal cells with stem cell potential to EECs, hormone encoding genes, genes encoding hormone precursors and genes encoding hormone synthesizing enzymes. If the detectable marker has integrated correctly into the endogenous locus, e.g. at the correct location and in the correct orientation, its expression will be initiated upon differentiation of the stem cells to EECs because it will depend on the genes at the EEC-specific gene locus being expressed. Thus, differentiating stem cells and/or cells with stem cell potential to EECs in order to detect expression of the detectable marker provides a simple, efficient screening step to select organoids comprising the desired tagged EEC-specific genes.

Accordingly, the invention provides a method for making a reporter organoid comprising or consisting of:
i. making one or more intestinal organoids comprising stem cells and/or cells with stem cell potential in which one or more EEC-specific genes is tagged at its endogenous locus with a detectable marker, wherein, selecting for correct insertion of the detectable marker comprises differentiating stem cells and/or cells with stem cell potential in the intestinal organoid to EECs and selecting one or more organoids which express the detectable marker upon differentiating the cells to EECs.

The inventors have also found that advantageously, a highly efficient selection step is achieved if the intestinal organoid comprises an inducible transcription factor for differentiating intestinal stem cells and/or intestinal cells with stem cell potential to EECs. For example, in some embodiments, the intestinal organoid comprises stem cells and/or cells with stem cell potential which comprise an inducible transcription factor for differentiating intestinal stem cells and/or intestinal cells with stem cell potential to EECs. Thus, the step of differentiating stem cells and/or cells with stem cell potential to EECs, may comprise overexpressing the transcription factor. However, the invention also encompasses differentiating stem cells and/or cells with stem cell potential in the intestinal organoid to EECs by other means.

Accordingly, in some embodiments, a method for making a reporter organoid comprises or consists of:
i. making one or more intestinal organoids comprising stem cells and/or cells with stem cell potential in which one or more EEC-specific genes is tagged at its endogenous locus with a detectable marker, and wherein the one or more organoids comprise stem cells and/or cells with stem cell potential comprising an inducible transcription factor for differentiating intestinal stem cells and/or intestinal cells with stem cell potential to EECs, wherein selecting for correct insertion of the detectable marker comprises overexpressing the transcription factor in order to differentiate stem cells and/or cells with stem cell potential in the intestinal organoid to EECs and selecting one or more organoids which express the detectable marker upon differentiating the cells to EECs.

In some embodiments, the method comprises generating the one or more cells which comprise the inducible transcription factor. Alternatively, in some embodiments, the method uses one or more cells with already comprise the inducible transcription factor.

Accordingly, in some embodiments, a method for making a reporter organoid, comprises or consists of:
i. Making or obtaining intestinal stem cells and/or cells with stem cell potential which comprise an inducible transcription factor for differentiating intestinal stem cells and/or intestinal cells with stem cell potential to EECs; and
ii. Using one or more stem cells and/or one or more cells with stem cell potential made or obtained in step i) to make one or more intestinal organoids comprising stem cells and/or cells with stem cell potential in which one or more EEC-specific genes is tagged at its endogenous locus with a detectable marker, wherein, selecting for correct insertion of the detectable marker comprises differentiating stem cells and/or cells with stem cell potential in the intestinal organoid to EECs, for example, by overexpressing the transcription factor, and selecting one or more organoids which express the detectable marker upon differentiating the cells to EECs.

The step of making or obtaining intestinal stem cells and/or cells with stem cell potential which comprise an inducible transcription factor for differentiating intestinal stem cells and/or intestinal cells with stem cell potential to EECs, preferably comprises establishing or obtaining a stable stem cell line of such cells, for example, in the form of an intestinal organoid. Thus, in some embodiments, the method comprises a further step after step i. and before step ii. of establishing a stable stem cell line of the intestinal stem cells and/or cells with stem cell potential comprising the inducible transcription factor. The stable cell line is preferably in the form of an intestinal organoid. The subsequent step of using one or more stem cells and/or cells with stem cell potential to make one or more intestinal organoids therefore preferably comprises using one or more intestinal stem cells and/or intestinal cells with stem cell potential from the stable stem cell line to make the one or more organoids. Thus, the invention similarly provides a method for making intestinal stem cells and/or cells with stem cell potential which comprise an inducible transcription factor for differentiating intestinal stem cells and/or intestinal cells with stem cell potential to EECs, comprising making such cells and establishing a stable stem cell line of such cells, for example, in the form of an intestinal organoid. Accordingly, the invention provides a stable stem cell line as described herein.

In some embodiments, the step of using the stem cells and/or cells with stem cell potential to make one or more intestinal organoids comprises or consists of the steps of:
i. Tagging one or more EEC-specific genes with a detectable marker at their endogenous locus in one or more stem cells and/or cells with stem cell potential obtained from a stable stem cell line of intestinal stem cells and/or cells with stem cell potential which comprise an inducible transcription factor for differentiating intestinal stem cells and/or intestinal cells with stem cell potential to EECs; and
ii. differentiating stem cells and/or cells with stem cell potential in the intestinal organoid to EECs, for example, by overexpressing the transcription factor, and selecting one or more organoids which express the detectable marker upon differentiating the cells to EECs.

The detectable marker is preferably a fluorescence marker. In some embodiments, it is known as a first detectable marker. In embodiments which describe one or more constructs encoding the first and/or second detectable markers, the one or more constructs preferably comprise a DNA sequence which encodes the first and/or second detectable marker. In embodiments which describe a construct targeting or tagging at the endogenous locus, the construct is preferably inserted into the endogenous locus. In preferred embodiments, a second detectable marker is introduced into the one or more intestinal stem cells and/or into the one or more intestinal cells with stem cell potential together with the first detectable marker. The second detectable marker may then be used to select cells which contain the first detectable marker. These selected cells may then be cultured to generate one or more intestinal organoids. In some embodiments, the second detectable marker is a fluorescent marker. In some embodiments, the second detectable marker is an antibiotic resistance marker. In some embodiments, the second detectable marker may optionally be called a selection marker.

For example, in some embodiments, the step of making one or more intestinal organoids comprising stem cells and/or cells with stem cell potential in which one or more EEC-specific genes is tagged at its endogenous locus with a first detectable marker comprises or consists of:
i. contacting an intestinal stem cell and/or an intestinal cell with stem cell potential with one or more constructs encoding the first detectable marker and a second detectable marker, wherein the construct encoding the first detectable marker targets the EEC-specific gene at its endogenous locus;
ii. selecting transfected cells that express the second detectable marker;
iii. generating one or more intestinal organoids by culturing the cells selected in step ii) in an expansion medium;
iv. differentiating stem cells and/or cells with stem cell potential in the one or more intestinal organoids to EECs, for example, by overexpressing the transcription factor; and
v. selecting one or more organoids which express the first detectable marker upon differentiating the cells to EECs.

The one or more intestinal stem cells and/or intestinal cells with stem cell potential used in a method of the invention (e.g. used in step i)) may be obtained from or obtainable from a stable intestinal stem cell line comprising stem cells and/or cells with stem cell potential that comprise an inducible transcription factor for differentiating intestinal stem cells and/or intestinal cells with stem cell potential to EECs. In some embodiments, such cells are obtained from or are obtainable from an organoid as described herein.

Accordingly, in some embodiments, a method for making a reporter organoid, comprises or consists of:
i. Making or obtaining intestinal stem cells and/or cells with stem cell potential which comprise an inducible transcription factor for differentiating intestinal stem cells and/or intestinal cells with stem cell potential to EECs;
ii. Establishing a stable stem cell line of the cells made in step i), for example, in the form of an intestinal organoid;
iii. contacting an intestinal stem cell and/or an intestinal cell with stem cell potential obtained from or obtainable from the stable stem cell line established in step ii) with one or more constructs encoding the first detectable marker and a second detectable marker, wherein the construct encoding the first detectable marker targets the EEC-specific gene at its endogenous locus;
iv. selecting transfected cells that express the second detectable marker;
v. generating one or more intestinal organoids by culturing the cells selected in step iv) in an expansion medium;
vi. differentiating stem cells and/or cells with stem cell potential in the intestinal organoid to EECs, for example, by overexpressing the transcription factor; and
vii. selecting one or more organoids which express the first detectable marker upon differentiating the cells to EECs.

In some embodiments, the one or more intestinal stem cells and/or intestinal cells with stem cell potential are obtained from an organoid by dissociating the organoid to generate a population of cells comprising the one or more stem cells and/or cells with stem cell potential or to generate one or more single cells. The organoid may be dissociated by any suitable method, e.g. by enzymatic digestion.

The inventors have found that intestinal organoids in which the stem cells have already been differentiated to EECs do not generally tend to expand efficiently in culture. Therefore, it is advantageous to make organoids in a form in which the intestinal organoids comprise the tagged EEC-specific gene(s) of interest and an inducible transcription factor for differentiating intestinal stem cells and/or intestinal cells with stem cell potential to EECs, but in which the stem cells have not yet been differentiated.

Thus, in some embodiments, the method advantageously further comprises dissociating the selected organoids and expanding the cells in an expansion medium to generate one or more organoids. These generated organoids comprise the required constructs but the stem cells and/or cells with stem cell potential in the organoids have not yet been differentiated to EECs. For example, in some embodiments, the method comprises a further step of dissociating the organoids selected in step vii and expanding the cells in an expansion medium to generate one or more organoids. The one or more organoids are preferably clonal organoids, for example, which have been established from a single cell. Reporter organoids generated in this way may optionally be known as a stable reporter organoid line. Accordingly, the invention provides a stable reporter organoid line as described herein. Reporter organoids can be stored in this form, for example, frozen, and when it is desired to use them, they can be thawed and cultured, e.g. in the presence of the inducer for the inducible promoter, to differentiate the stem cells to EECs.

In some embodiments, steps iv to vii comprise or consist of:
iv. culturing the cells obtained by step iii in an expansion medium, selecting transfected cells that express the second detectable marker gene and plating the selected cells as single cells;
v. generating one or more intestinal organoids by culturing the single cells obtained in step iv in an expansion medium;
vi. differentiating stem cells and/or cells with stem cell potential to EECs in one or more organoids generated in step v), e.g. by overexpressing the transcription factor, while culturing the one or more organoids in an expansion medium and/or a differentiation medium; and
vii. selecting one or more organoids which express the first detectable marker, dissociating the selected organoids and expanding the cells in an expansion medium to generate one or more organoids.

Accordingly, in some embodiments, a method for making a reporter organoid, comprises or consists of:
i. Making or obtaining intestinal stem cells and/or cells with stem cell potential which comprise an inducible transcription factor for differentiating intestinal stem cells and/or intestinal cells with stem cell potential to EECs;
ii. Establishing a stable stem cell line of the cells made in step i), for example, in the form of an intestinal organoid;
iii. contacting an intestinal stem cell and/or an intestinal cell with stem cell potential obtained from or obtainable from the stable stem cell line established in step ii) with one or more constructs encoding the first detectable marker and a second detectable marker, wherein the construct encoding the first detectable marker targets the EEC-specific gene at its endogenous locus;
iv. culturing the cells obtained by step iii in an expansion medium, selecting transfected cells that express the second detectable marker gene and plating the selected cells as single cells;
v. generating one or more intestinal organoids by culturing the single cells obtained in step iv in an expansion medium;
vi. differentiating stem cells and/or cells with stem cell potential to EECs in one or more organoids generated in step v), e.g. by overexpressing the transcription factor, while culturing the one or more organoids in an expansion medium and/or a differentiation medium; and
vii. selecting one or more organoids which express the first detectable marker, dissociating the selected organoids and expanding the cells in an expansion medium to generate one or more organoids.

In some embodiments of the various methods provided herein which describe the use of single cells, e.g. single cells obtained from organoids or culturing single cells, it is not necessary to use all the single cells that have been obtained. For example, in some embodiments, only a single cell is used and this would still fall within the scope of the invention. However, generally, more than one single cell will be used, e.g. in order to increase the efficiency of the methods.

In some embodiments, after the stable reporter organoid line has been made which comprises correct integration of the detectable marker, and optionally after the stable organoid line has been stored, e.g. by freezing and thawing, the method comprises a further step which comprises differentiating cells in the intestinal organoid to EECs. For example, in some embodiments, the method comprises a further step (e.g. step viii) of differentiating stem cells and/or cells with stem cell potential to EECs in one or more organoids (e.g. in one or more organoids generated in step (vii)) while culturing the one or more organoids in an expansion medium and/or a differentiation medium.

As mentioned above, the step of differentiating intestinal stem cells and/or cells with stem cell potential to EECs results in the one or more genes encoding the tagged EEC-specific genes (e.g. the tagged hormone, tagged hormone precursor and/or tagged hormone synthesizing enzyme) to be expressed. Thus, differentiating the cells to EECs generates a reporter organoid which comprises EECs that express tagged EEC-specific genes (e.g. which express and secrete tagged hormones or hormone precursors or which express tagged hormone synthesizing enzymes).

As mentioned above, the stable stem cell line of the cells made in step i) may be in the form of an intestinal organoid. Accordingly, in some embodiments, the intestinal stem cells and/or intestinal cells with stem cell potential in which the one or more EEC-specific genes are tagged are obtained from or are obtainable from an intestinal organoid. In some embodiments, the organoid is dissociated (e.g. by enzymatic digestion) to generate a population of cells (e.g. a stem cell culture) which comprises the stem cells and/or cells with stem cell potential, for example, using enzymatic digestion. For example, cells from an intestinal organoid may be dissociated into small clumps of stem cells which may then be transfected with the one or more constructs comprising the first detectable marker and preferably also the second detectable marker. Thus, in some embodiments, the one or more stem cells and/or cells with stem cell potential used in step (i) are present in a stem cell culture. For example, in some embodiments, they are a population of cells present in a stem cell culture.

Step i) of the method may further comprise the steps of establishing an intestinal organoid as described herein, and dissociating the organoid (e.g. by enzymatic digestion) to obtain the one or more intestinal stem cells and/or intestinal cells with stem cell potential, e.g. in the form of single cells or a population of intestinal cells. Alternatively, in some embodiments, the one or more intestinal stem cells and/or intestinal cells with stem cell potential are obtained from a pre-established intestinal organoid. The intestinal organoid may be established by the same party that carries out the remaining steps of the method or may be established by a different party.

Preferably, the organoid used to obtain the one or more stem cells and/or cells with stem cell potential comprises intestinal stem cells and/or intestinal cells with stem cell potential (preferably, stem cells) which comprise an inducible transcription factor for differentiating intestinal stem cells and/or intestinal cells with stem cell potential to EECs. Thus, the invention provides a method for making an intestinal organoid comprising stem cells and/or cells with stem cell potential which comprise an inducible transcription factor for differentiating intestinal stem cells and/or intestinal cells with stem cell potential to EECs, comprising or consisting of the steps of:
i) contacting an intestinal stem cell and/or an intestinal cell with stem cell potential with a construct comprising an inducible transcription factor for differentiating intestinal stem cells and/or intestinal cells with stem cell potential to EECs; and
ii) generating one or more intestinal organoids by culturing the cells in an expansion medium.

The invention also provides an intestinal organoid comprising stem cells and/or cells with stem cell potential which comprise an inducible transcription factor for differentiating intestinal stem cells and/or intestinal cells with stem cell potential to EECs. The organoid is preferably obtained by or obtainable by a method as provided herein.

In some embodiments of a method of making a reporter organoid, the organoid from which the one or more intestinal stem cells and/or intestinal cells with stem cell potential are obtained from or obtainable from is made by a method as described herein. Thus, in some embodiments, the one or more cells is obtained from or obtainable from an intestinal organoid comprising stem cells and/or cells with stem cell potential which comprise an inducible transcription factor for differentiating intestinal stem cells and/or intestinal cells with stem cell potential to EECs. The steps of making the intestinal organoid comprising stem cells and/or cells with stem cell potential which comprise an inducible transcription factor for differentiating intestinal stem cells and/or intestinal cells with stem cell potential to EECs may optionally be comprised in a method of the invention.

Preferably, the stem cells have not yet been differentiated to EECs (for example, by overexpression of the transcription factor) in the intestinal organoid used to obtain the one or more intestinal stem cells and/or intestinal cells with stem cell potential in which the one or more EEC-specific genes are tagged.

However, in some embodiments, the one or more intestinal stem cells and/or intestinal cells with stem cell potential are obtained from or are obtainable from an intestinal organoid that has been made using a method comprising or consisting of the following steps:
i) contacting an intestinal stem cell and/or an intestinal cell with stem cell potential with a construct comprising an inducible transcription factor for differentiating intestinal stem cells and/or intestinal cells with stem cell potential to EECs;
ii) generating one or more intestinal organoids by culturing the cells in an expansion medium; and
iii) differentiating stem cells and/or cells with stem cell potential to EECs in one or more intestinal organoids generated in step ii, e.g. by overexpressing the inducible transcription factor, while culturing the one or more intestinal organoids in an expansion medium and/or a differentiation medium.

In some embodiments, step iii) acts as a selection step to select organoids which comprise the inducible transcription factor for differentiating intestinal stem cells and/or intestinal cells with stem cell potential to EECs.

A clonal organoid line of a selected organoid is preferably established. These newly generated clonal organoids comprise stem cells and/or cells with stem cell potential because they themselves have not been differentiated to EECs. For example, a method as described herein may comprise a further step (e.g. step iv) which comprises selecting one or more organoids which express a detectable marker whose expression is linked to expression of the transcription factor, dissociating the selected organoids to single cells and expanding the single cells in an expansion medium to generate one or more clonal organoids. This can provide a stable cell line of intestinal stem cells and/or cells with stem cell potential which comprise an inducible transcription factor for differentiating intestinal stem cells and/or intestinal cells with stem cell potential to EECs. The intestinal stem cells and/or intestinal cells with stem cell potential for use in the step of tagging one or more EEC-specific genes with a detectable marker at its endogenous locus may therefore be obtained from the clonal organoid line if desired.

Previous attempts to create human EECs in vitro have relied on growth-factor based differentiation (Beumer et al., 2018) or overexpression of NEUROG3, the key transcription factor to instruct EEC fate (McCracken et al., 2014; Sinagoga et al., 2018). Both iPSC- (Zhang et al., 2019) and adult stem cell (ASC)-based (Chang-Graham et al., 2019) approaches have proven useful to understand aspects of human EEC biology, such as modelling of inherited NEUROG3 mutations and viral infection-mediated serotonin release (Chang-Graham et al., 2019). However, imperfect differentiation and regional restriction of the donor material have limited these studies to a subset of human EECs. In addition, maturity of the EECs was not demonstrated.

The inventors show that the percentage of EECs can be increased in intestinal organoids derived from different regions of the intestinal tract by overexpression of a transcription factor for differentiation of intestinal stem cells and/or intestinal cells with stem cell potential to EECs, such as Neurogenin 3 (NEUROG3), while culturing the intestinal organoids in a differentiation medium or an expansion medium. Accordingly, the invention provides a method for enriching the population of EECs in an intestinal organoid comprising overexpression of a transcription factor for differentiation of intestinal stem cells and/or intestinal cells with stem cell potential to EECs in the intestinal organoid. Preferably, the method comprises overexpression of NEUROG3 in stem cells and/or in cells with stem cell potential in the intestinal organoid. Thus, the invention provides a method for enriching the population of EECs in an intestinal organoid, comprising or consisting of the steps of:
i. Generating one or more intestinal stem cells and/or intestinal cells with stem cell potential which comprise an inducible transcription factor for differentiating intestinal stem cells and/or intestinal cells with stem cell potential to EECs;
ii. Generating one or more intestinal organoids from the intestinal stem cells and/or intestinal cells with stem cell potential; and
iii. Differentiating stem cells and/or cells with stem cell potential in the organoids to EECs, e.g. by overexpressing the inducible transcription factor.

For example, the invention provides a method for enriching the population of EECs in an intestinal organoid, comprising or consisting of the steps of:
i. contacting an intestinal stem cell and/or an intestinal cell with stem cell potential with a construct comprising an inducible transcription factor for differentiating stem cells and/or cells with stem cell potential to EECs;
ii. generating one or more intestinal organoids by culturing the cells in an expansion medium; and
iii. differentiating stem cells and/or cells with stem cell potential to EECs in one or more intestinal organoids generated in step ii, e.g. by overexpressing the inducible transcription factor, while culturing the one or more intestinal organoids in an expansion medium and/or a differentiation medium.

The invention also provides an organoid obtained by or obtainable by a method as described herein, for example, by a method for making a reporter organoid or by a method for enriching the population of EECs in an intestinal organoid.

In some embodiments, the one or more intestinal stem cells and/or intestinal cells with stem cell potential are obtained from an intestinal organoid, e.g. from a stable intestinal organoid line. Generally, the intestinal organoid will have initially been established from a tissue sample. In some embodiments, the cells are obtained directly from an intestinal tissue sample. In some embodiments, the intestinal tissue sample is derived from the small intestine. In some embodiments, the intestinal tissue sample is derived from the colon. In some embodiments, the intestinal tissue sample is derived from a region of the intestine selected from the proximal small intestine (duodenum), the distal small intestine (ileum), the ascending colon, the descending colon, the transverse colon, the sigmoid colon, the rectum and the jejunum. Thus, the use of intestinal organoids established from these regions of the intestine is encompassed by the invention.

In some embodiments of the various methods of the invention, the transcription factor for differentiating stem cells and/or cells with stem cell potential to EECs is selected from NEUROG3, ATOH1/MATH1 and NEUROD1. Preferably, the transcription factor is NEUROG3. For example, the one or more intestinal stem cells and/or intestinal cells with stem cell potential preferably comprise an inducible NEUROG3 construct. This may also be known herein as a NEUROG3 overexpression construct.

In embodiments in which the intestinal organoids are human intestinal organoids, the transcription factor gene is preferably the human transcription factor gene. In embodiments in which the intestinal organoids are established from tissue from a different organism, the transcription factor gene is preferably from that organism. For example, for human intestinal organoids, the NEUROG3 gene is preferably the human NEUROG3 gene. Similarly, for mouse intestinal organoids, the NEUROG3 gene is preferably the mouse NEUROG3 gene. The nucleic acid encoding the transcription factor may be obtained by any suitable method. In some embodiments, it is cloned from genomic DNA, for example, using the method described herein for the NEUROG3 gene. For example, in some embodiments, the NEUROG3 gene is obtained by or obtainable by cloning from human genomic DNA using the oligos described herein (SEQ ID Nos: 43 and 44). In some embodiments, the NEUROG3 gene comprises or consists of the sequence shown in SEQ ID NO:1. In some embodiments, the ATOH1/MATH1 gene comprises or consists of the sequence shown in SEQ ID NO:5. In some embodiments, the NEUROD1 gene comprises or consists of the sequence shown in SEQ ID NO:3. In some embodiments, a variant of the transcription factor gene may be used, for example a variant of the transcription factor gene having at least 75% (e.g. at least 80%, 85%, 90%, 95%, 98%, 99%) sequence identity with the cloned sequence or with the sequence shown in SEQ ID NO: 1, 3 or 5. In some embodiments, the NEUROG3 gene encodes a polypeptide comprising or consisting of the sequence shown in SEQ ID NO:2. In some embodiments, the ATOH1/MATH1 gene encodes a polypeptide comprising or consisting of the sequence shown in SEQ ID NO:6. In some embodiments, the NEUROD1 gene encodes a polypeptide comprising or consisting of the sequence shown in SEQ ID NO:4. In some embodiments, the transcription factor gene encodes the sequence of a variant of the transcription factor having at least 75% (e.g. at least 80%, 85%, 90%, 95%, 98%, 99%) sequence identity with the polypeptide shown in SEQ ID NO: 2, 4 or 6. Sequence identity between polypeptide sequences is preferably determined by pairwise alignment algorithm using the Needleman-Wunsch global alignment algorithm (Needleman& Wunsch (1970) J. Mol. Biol. 48, 443-453), using default parameters (e.g. with Gap opening penalty=10.0, and with Gap extension penalty=0.5, using the EBLOSUM62 scoring matrix). This algorithm is conveniently implemented in the needle tool in the EMBOSS package (Rice et al. (2000) Trends Genet 16:276-277). Sequence identity should be calculated over the entire length of the polypeptide sequence of the invention.

Similarly, a fragment of the transcription factor gene or a fragment of a variant thereof may be used. The variant or fragment is a variant or fragment which still has the function of differentiating intestinal stem cells and/or intestinal cells with stem cell potential to EECs. In some embodiments, the variant or fragment promotes expression of the broad EEC marker CHGA, for example, as described herein. In some embodiments, the variant decreases the Paneth cell product LYZ+, for example, as described herein. In some embodiments in which the reporter organoid comprises the transcription factor expressed in polypeptide form, the polypeptide may be a polypeptide described herein, e.g. comprising or consisting of a sequence as described herein, and may, for example, be a variant, fragment or fragment of a variant as described herein.

Methods for comparing the identity of two or more sequences are well known in the art. Thus for instance, programs available in the Wisconsin Sequence Analysis Package, version 9.1 (Devereux J et al, Nucleic Acids Res, 12,387-395, 1984, available from Genetics Computer Group, Madison, Wisconsin, USA), for example the program BESTFIT may be used to determine the % identity between two polynucleotides. BESTFIT uses the “local homology” algorithm of Smith and Waterman (J Mol Biol, 147,195-197, 1981, Advances in Applied Mathematics, 2,482-489, 1981) and finds the best single region of similarity between two sequences. Preferably, the parameters “Gap Weight” and “Length Weight” used are 50 and 3, for polynucleotide sequences.

Other programs for determining identity and/or similarity between sequences are also known in the art, for instance the BLAST family of programs (Altschul S F et al, J Mol Biol, 215,403-410, 1990, Altschul S F et al, Nucleic Acids Res., 25: 389-3402, 1997, available from the National Center for Biotechnology Information (NCBI), Bethesda, Maryland, USA and accessible through the home page of the NCBI at www.ncbi.nim.nih.gov) and FASTA (Pearson W R, Methods in Enzymology, 183, 63-99, 1990; Pearson W R and Lipman D J, Proc Nat Acad Sci USA, 85, 2444-2448, 1988, available as part of the Wisconsin Sequence Analysis Package).

In some embodiments, sequence identity (e.g. % sequence identity) is determined using a method as described herein.

In some preferred embodiments, the transcription factor is expressed from an overexpression construct comprising the gene encoding the transcription factor under control of an inducible promoter. In some alternative embodiments, the endogenous gene encoding the transcription factor is put under control of an inducible promoter. Methods which describe the use of a construct comprising the gene encoding the transcription factor under control of an inducible promoter may be adapted to instead use the endogenous gene encoding the transcription factor under control of an inducible promoter, if desired.

Any suitable inducible promoter may be used. In some embodiments, the inducible promoter is an antibiotic-inducible promoter, for example a doxycycline-inducible promoter, for example Tet-On. In preferred embodiments, the step of differentiating stem cells and/or cells with stem cell potential to EECs comprises culturing the one or more organoids in an expansion medium and/or in a differentiation medium in the presence of the inducer for the inducible promoter. Thus, in some embodiments, the expansion medium and/or the differentiation medium comprises the inducer for the inducible promoter. In some embodiments, the transcription factor is transiently overexpressed. For example, in some embodiments, the method comprises pulsed expression of the transcription factor. For example, in some embodiments, in order to achieve pulsed expression, the inducer for the inducible promoter (e.g. doxycycline) is present in the culture medium and then is removed and washed away after a limited period of time (for example, after about 48 hours). In some embodiments, the culture medium used to culture the intestinal organoids during the step of differentiating stem cells to EECs is a differentiation medium, for example, the basic medium ENR. In some embodiments, the method comprises pulsed expression of NEUROG3 in the basic medium ENR, for a suitable limited period of time, for example, for 48 hours. In some alternative embodiments, the transcription factor (e.g. NEUROG3) is constitutively expressed.

Overexpression of the inducible transcription factor (e.g. NEUROG3) preferably promotes expression of the broad EEC marker Chromogranin A (CHGA). In some embodiments, overexpression of the transcription factor (e.g. NEUROG3) also decreases the Paneth cell product LYZ+. In some embodiments, the decrease in the Paneth cell product LYZ+ is a decrease of 5% or more (e.g. 10%, 15%, 20%, 25%, 30% or more) compared to the Paneth cell product LYZ+ expression level before overexpression of the transcription factor. In some embodiments, expression of CHGA and/or expression of LYZ+ is measured using qPCR. Thus, in embodiments in which the transcription factor is transiently expressed, the duration of the transient expression may be chosen to ensure expression of the CHGA EEC marker and/or a decrease in the Paneth cell product LYZ+. In some embodiments, the transcription factor is transiently expressed for 48 hours.

For example, the inducer for the promoter (e.g. doxycycline) may be removed and washed away after 48 hours. However, the skilled person would be able to determine which other durations would be suitable for transient expression.

In some embodiments, the method of making one or more intestinal stem cells and/or one or more intestinal cells with stem cell potential for use in a method for making a reporter organoid comprises the step of contacting an intestinal stem cell and/or an intestinal cell with stem cell potential with a construct comprising the gene encoding the transcription factor (e.g. the NEUROG3 gene) under control of an inducible promoter.

Accordingly, the invention provides a method for making an intestinal organoid (e.g. a method for making (generating) an intestinal organoid comprising stem cells and/or cells with stem cell potential which comprise an inducible transcription factor for differentiating intestinal stem cells and/or intestinal cells with stem cell potential to EECs, e.g. for use in a method for making a reporter organoid), which comprises or consists of the steps of:
i. contacting an intestinal stem cell and/or an intestinal cell with stem cell potential with a construct comprising the transcription factor gene under control of an inducible promoter; and
ii. generating one or more intestinal organoids by culturing the cells in an expansion medium.

For example, in some embodiments, the method for making an organoid comprises or consists of the steps of:
i. contacting an intestinal stem cell and/or an intestinal cell with stem cell potential with a construct comprising the transcription factor gene under control of an inducible promoter;
ii. generating one or more intestinal organoids by culturing the cells in an expansion medium; and
iii. differentiating stem cells and/or cells with stem cell potential to EECs in one or more intestinal organoids generated in step ii., e.g. by overexpressing the transcription factor, while culturing the one or more intestinal organoids in an expansion medium and/or a differentiation medium.

In some embodiments, step iii) serves as a selection step for organoids which comprise the construct comprising the transcription factor gene under control of an inducible promoter. For example, step iii) may comprise selecting organoids which comprise the construct comprising the transcription factor by differentiating stem cells and/or cells with stem cell potential to EECs in one or more intestinal organoids generated in step ii by overexpressing the inducible transcription factor, while culturing the one or more intestinal organoids in an expansion medium and/or a differentiation medium. The method may then contain a further step of dissociating the selected organoids to single cells and expanding the single cells in an expansion medium to generate one or more clonal organoids. This further step may similarly be conducted in the method for enhancing the population of EECs in an intestinal organoid as described herein (also known herein as the method for enriching the population of EECs in an intestinal organoid). Intestinal organoids obtained by or obtainable by such methods are likewise provided by the invention. The one or more intestinal stem cells and/or intestinal cells with stem cell potential for use in making a reporter organoid are preferably obtained from this clonal organoid. Accordingly, a method for making an intestinal organoid comprising stem cells and/or cells with stem cell potential which comprise an inducible transcription factor for differentiating intestinal stem cells and/or intestinal cells with stem cell potential to EECs, e.g. for use in a method for making a reporter organoid, preferably comprises this further step of generating one or more clonal organoids.

Preferably, the step of differentiating stem cells and/or cells with stem cell potential to EECs comprises overexpressing a transcription factor for differentiation of intestinal stem cells to EECs (for example, NEUROG3) in the one or more intestinal organoids. The overexpression is preferably relative to the endogenous expression levels of the transcription factor (e.g. NEUROG3).

The step of contacting an intestinal stem cell and/or an intestinal cell with stem cell potential with the one or more constructs enables the construct to be introduced into one or more intestinal stem cells and/or into one or more intestinal cells with stem cell potential. The one or more constructs may be introduced into the cells by any suitable method.

For example, the construct comprising an inducible transcription factor for differentiating intestinal stem cells and/or intestinal cells with stem cell potential to EECs may be introduced into the intestinal stem cells or into the intestinal cells with stem cell potential using any suitable method.

In preferred embodiments, it is introduced using transduction, for example using a lentiviral vector.

In some embodiments, the lentiviral vector integrates (preferably stably integrates) into the genome of the stem cells or of the cells with stem cell potential. Thus, in some embodiments, the construct is a lentiviral vector construct. In some embodiments, the lentiviral vector has a backbone as described in Sachs et al. (2019)). Methods for transducing organoids with lentiviruses are known in the art (e.g. see Koo et al., 2012). In some alternative embodiments, an expression plasmid comprises the inducible transcription factor for differentiating intestinal stem cells and/or intestinal cells with stem cell potential to EECs and the method comprises transfecting the expression plasmid into the intestinal organoid, for example using electroporation. In some embodiments, the gene encoding the inducible transcription factor for differentiating intestinal stem cells and/or intestinal cells with stem cell potential to EECs does not integrate into the genome but is expressed from an expression plasmid in the stem cell. In some embodiments, the construct encoding the transcription factor under control of an inducible promoter integrates into the genome. As described elsewhere herein, a method of the invention preferably comprises establishing or obtaining a stable stem cell line of intestinal stem cells and/or cells with stem cell potential which comprise an inducible transcription factor for differentiating intestinal stem cells and/or intestinal cells with stem cell potential to EECs. Thus, in preferred embodiments, the intestinal stem cells and/or cells with stem cell potential stably comprise an inducible transcription factor for differentiating intestinal stem cells and/or intestinal cells with stem cell potential to EECs.

In some embodiments, the gene encoding the transcription factor is tagged with a detectable marker gene, for example, a fluorescent marker. In some embodiments, the detectable marker gene is separated from the reading frame of the transcription factor to avoid creating a fusion protein, for example, using a self-cleavable sequence (e.g. P2A). For example, in some embodiments, a fluorescent marker (e.g. dTomato) is inserted 3′ to the transcription factor reading frame and separated by a self-cleavable P2A sequence. In some embodiments, the transcription factor is the EEC-specific gene in a reporter organoid of the invention. In some embodiments, the transcription factor is the inducible transcription factor. Thus, in some embodiments, a detectable marker is present in the overexpression construct. Thus, in the methods of the invention, the constructs may comprise a means for separating the detectable marker from the transcription factor, for example, as described herein. In some embodiments, the step of selecting one or more organoids which comprise the construct comprising the transcription factor comprises selecting one or more organoids which express a detectable marker whose expression is linked to expression of the transcription factor. Differentiated cells which express the transcription factor are EECs. This, therefore, advantageously enables the detectable marker (e.g. the fluorescent marker) to be used to identify EECs. The invention therefore provides a method for identifying or isolating one or more EECs from an intestinal organoid of the invention comprising detecting expression of the transcription factor by detecting expression of a detectable marker linked to expression of the transcription factor.

Any suitable method for making an intestinal organoid may be used in the methods of the invention, for example, as described herein. For example, the intestinal organoids may be established using one or more intestinal epithelial stem cells, for example, using the methods described in any one of Sato, T. et al., (2011), WO 2010/090513, WO 2017/220586 and WO 2012/168930. The intestinal epithelial stem cells may in some embodiments be Lgr5+ epithelial stem cells. In some embodiments, the intestinal epithelial stem cells are healthy intestinal epithelial stem cells. However, the use of diseased intestinal epithelial stem cells is also envisaged. For example, in some embodiments, the diseased intestinal epithelial stem cells have been obtained from a tissue donor, for example, as described elsewhere herein. The use of diseased epithelial stem cells to establish organoids may be useful for studying disease processes, for example, how a diseased EEC compares with a healthy EEC, or investigating methods for treating a disease, for example, identifying suitable therapeutic compounds. In some embodiments, the intestinal organoid is established from adult tissue, e.g. healthy adult tissue or diseased adult tissue. In some embodiments, the intestinal organoid is established from fetal or infant tissue, e.g. healthy fetal or infant tissue or diseased fetal or infant tissue. The organoid may be dissociated (e.g. by digestion) to obtain the one or more intestinal stem cells and/or intestinal cells with stem cell potential, for example, to make a stem cell culture.

In some embodiments, the intestinal organoid is established from intestinal epithelial stem cells derived from the small intestine. In some embodiments, the intestinal organoid is established from intestinal epithelial stem cells derived from the colon. In some embodiments, the intestinal organoid is established from intestinal epithelial stem cells derived from the proximal small intestine (duodenum), the distal small intestine (ileum), the ascending colon, the descending colon, the transverse colon, the sigmoid colon, the rectum or the jejunum. Advantageously, all hormones produced by EECs can be studied with the use of organoids established from the duodenum, ileum and the ascending colon. Accordingly, in some embodiments, the intestinal organoid is established from intestinal epithelial stem cells derived from the proximal small intestine (duodenum, also referred to herein as a “duodenal organoid”). In some embodiments, the intestinal organoid is established from intestinal epithelial stem cells derived from the distal small intestine (ileum, also referred to herein as an “ileal organoid”). In some embodiments, the intestinal organoid is established from intestinal epithelial stem cells derived from the ascending colon (also referred to herein as a “colon organoid”). Thus, in some embodiments, the intestinal organoid is established from the proximal small intestine (duodenum), the distal small intestine (ileum) or the ascending colon.

In some embodiments, the one or more intestinal stem cells and/or one or more intestinal cells with stem cell potential is obtained from or is obtainable directly from intestinal tissue, for example, from an intestinal tissue sample from a donor. In some embodiments, the donor is a healthy donor. In some embodiments, the donor does not have a disease or disorder of the intestine. In some embodiments, the donor has a disease or disorder of the intestine. In some embodiments, the donor has a disease or disorder as described herein. In some embodiments, the donor is an adult donor. In some embodiments, the donor is a fetus or an infant. Techniques for processing the tissue sample in order to obtain the one or more intestinal stem cells and/or one or more intestinal cells with stem cell potential in a form suitable for use in the methods of the invention are known in the art.

In some embodiments, the steps for making an intestinal organoid comprising the inducible transcription factor for differentiating intestinal stem cells and/or intestinal cells with stem cell potential to EECs are encompassed within a method of the invention. In some embodiments, the step of dissociating the intestinal organoid prior to obtaining a stem cell culture comprising the one or more stem cells and/or cells with stem cell potential is similarly encompassed within a method of the invention. In some alternative embodiments, the methods of the invention are practised using pre-established organoids and/or pre-established stem cell cultures or single cells obtained from organoids.

The organoid is an intestinal organoid, for example, as described herein. Methods for making intestinal organoids are well known in the art and are described herein in detail.

The skilled person will understand which culture medium is suitable for use in each step of the method, for example, based on the teaching herein.

In some embodiments, the step of differentiating stem cells and/or cells with stem cell potential to EECs comprises culturing the cells in an expansion medium but not in a differentiation medium. In some embodiments, the step of differentiating stem cells and/or cells with stem cell potential to EECs comprises culturing the cells in a differentiation medium but not in an expansion medium. In some embodiments, the step of differentiating stem cells and/or cells with stem cell potential to EECs comprises culturing the cells in an expansion medium and then replacing it with a differentiation medium. In some embodiments, culturing in a differentiation medium of the invention is sufficient to differentiate stem cells and/or cells with stem cell potential to EECs. However, preferably, the step of differentiating stem cells and/or cells with stem cell potential to EECs comprises culturing the cells in the presence of the inducer for the inducible promoter of the transcription factor. This allows overexpression of the transcription factor.

In some embodiments, the expansion medium is removed prior to culturing the cells in the differentiation medium, e.g. by repeated washings or by splitting the cell culture.

Expansion Medium

Culturing intestinal stem cells in an expansion medium of the invention allows the intestinal stem cells and/or intestinal cells with stem cell potential to form intestinal organoids. An expansion medium is preferably a culture medium that establishes and expands intestinal organoids by allowing expansion of intestinal stem cells and/or intestinal cells with stem cell potential. The expansion medium may be any suitable expansion medium for intestinal epithelial stem cells (e.g. as described in WO 2012/168930, WO 2017/220586 or Sato, T. et al., Gastroenterology, 2011, 141: 1762-1772). For example, in some embodiments, the expansion medium is the HISC medium described in Sato, T. et al (supra).

In some embodiments, the intestinal organoids are established by culturing an epithelial stem cell and/or an epithelial cell with stem cell potential or a population of such cells derived from the intestine in contact with an extracellular matrix in the presence of an expansion medium.

In some embodiments, the expansion medium comprises (i) Wnt-3A or Wnt-3A conditioned medium or a Wnt surrogate molecule; (ii) a receptor tyrosine kinase ligand (e.g. EGF); (iii) a BMP inhibitor (e.g. Noggin); (iv) a Wnt agonist (e.g. R-spondin); (v) gastrin; (vi) nicotinamide; (vii) one or more TGF beta inhibitors (e.g. A83-01); and (viii) one or more p38 inhibitors (e.g. SB202190). For example, in some embodiments, the expansion medium comprises (i) Wnt-3A or Wnt-3A conditioned medium or a Wnt surrogate molecule; (ii) EGF; (iii) Noggin; (iv) R-spondin; (v) gastrin; (vi) nicotinamide; (vii) one or more TGF beta inhibitors (e.g. A83-01); and (viii) one or more p38 inhibitors (e.g. SB202190). This is known as the “HSi” medium.

In some embodiments, the expansion medium comprises (i) Wnt3a (e.g. at about 40 to 60% CM v/v, e.g. at about 50% CM v/v) or a Wnt surrogate molecule (e.g. a Wnt surrogate-Fc fusion protein for example, from U-Protein Express BV) (e.g. at about 0.1 to 0.7 nM or about 0.1 to 0.5 nM, e.g. at about 0.5 nM) or a Wnt-3A conditioned medium; (ii) EGF (e.g. at about 10 to 70 ng/ml, about 30 to 60 ng/ml, or about 50 ng/ml); (iii) Noggin (e.g. at about 50 to 150 ng/ml, about 75 to 125 ng/ml, or about 100 ng/ml or about 2-10% final volume, e.g. about 5% final volume); (iv) R-spondin (e.g. at about 200-300 μg/ml, 225-275 μg/ml, or about 250 μg/ml); (v) gastrin (e.g. at about 3 to 12 nM, or at about 5 nM or about 10 nM); (vi) Nicotinamide (e.g. at about 5 to 15 mM, e.g. about 10 mM); (vii) a TGFβ inhibitor (e.g. A83-01 at a concentration of about 400 to 600 nM, e.g. about 500 nM); and (viii) a p38 inhibitor (e.g. SB-203580 at a concentration of about 30 μM or SB-202190 at a concentration of about 10 μM).

In some embodiments, the expansion medium comprises (i) EGF (e.g. at about 10 to 50 ng/ml); (ii) Noggin conditioned medium (e.g. at about 50 to 100 ng/ml or about 5% final volume); (iii) R-spondin conditioned medium (e.g. at about 1 μg/ml or about 5% final volume); (iv) a p38 inhibitor (e.g. SB-203580 at a concentration of about 30 μM); (v) a TGF-β inhibitor (e.g. A83-01 at a concentration of about 500 nM); and (vi) Nicotinamide (e.g. at a concentration of about 10 mM).

In some embodiments, the expansion medium further comprises one or more of (e.g. 2 or more, 3 or more or all 4 of) (i) Primocin (Invivogen, cat. no. ant-pm1) (e.g. at about 25-75 μg/ml, e.g. about 50 μg/ml); (ii) n Acetylcysteine (e.g. at about 1 to 1.5 mM, for example, about 1 mM or about 1.25 mM); (iii) 1×B27; and (iv) PGE2 (e.g. at about 7-13 nM, e.g. about 10 nM).

In some embodiments, the expansion medium further comprises valproic acid (e.g. at about 1 mM) and/or a GSK 3 inhibitor (e.g. at about 3 μM, such as CHIR99021 at about 3 μM).

Advantageously, the inclusion of valproic acid and a GSK 3 inhibitor was found to result in a cell population that is enriched in stem cells.

In some embodiments, a medium that has been conditioned with the component can be used as a component in the expansion medium instead of adding the component itself. For example, in some embodiments, R-spondin conditioned medium is used instead of R-spondin and/or Noggin conditioned medium is used instead of Noggin. Similarly, in some embodiments, Wnt-3A conditioned medium is used instead of Wnt-3A.

Preferably, the cells are expanded to generate one or more (e.g. at least 2, 3, 4, 5, 6, 10, 15, 20, 50, 100, 250, 500 or 1000) organoids.

An example of a Wnt surrogate molecule for use in the expansion medium is the Wnt surrogate-Fc fusion protein for example, from U-Protein Express BV.

In some embodiments, the step of differentiating stem cells and/or cells with stem cell potential to EECs comprises culturing the organoids in a suitable expansion medium, for example, in an expansion medium as described herein. The expansion medium used in this step may be the same as the expansion medium used to establish the intestinal organoids or may be a different expansion medium. For example, in some embodiments, the step of differentiating stem cells to EECs comprises culturing the organoid in HSi medium. In some embodiments, a differentiation medium is not used in the step of differentiating stem cells to EECs.

Differentiation Medium

In some embodiments, the step of differentiating intestinal stem cells to EECs comprises culturing the organoid in a differentiation medium. A differentiation medium is preferably a culture medium which allows differentiation of the stem cells and/or cells with stem cell potential in the intestinal organoid to EECs, for example, in conjunction with overexpression of the inducible transcription factor described herein. In some embodiments, cells comprising the construct comprising the transcription factor under control of an inducible promoter or comprising an inducible promoter for the endogenous transcription factor are initially cultured in an expansion medium described herein and, once successful organoids have been established, the expansion medium is replaced with a differentiation medium described herein. Accordingly, in some embodiments after one or more (e.g. after two, three, four, five, six, seven, eight, nine, ten or more) passages, the expansion medium is replaced with a differentiation medium. In some embodiments, passaging is carried out weekly. In some embodiments, after two, three, four, five, six, seven, eight, nine, ten or more days, the expansion medium is replaced with a differentiation medium. For example, in some embodiments, the expansion medium is replaced with a differentiation medium after five or more days. In some embodiments, replacing one culture medium with another may comprise adding or withdrawing factors from the culture medium, as appropriate.

The inventors found that differentiation towards EECs was most efficient when withdrawing most of the growth factors essential for expansion of stem cells, including Nicotinamide, Wnt3A, the TGF-beta inhibitor A-83-01 and a p38-inhibitor. Accordingly, in some embodiments, the differentiation medium used in the step of differentiating stem cells to EECs does not comprise one or more of (e.g. 2, 3 or more or all 4 of) Nicotinamide, Wnt (e.g. Wnt3A), a TGF-beta inhibitor (e.g. A-83-01) and a p38-inhibitor.

In some preferred embodiments, the differentiation medium comprises a basic medium without stem cell factors, for example, the differentiation medium known as the ‘ENR’ medium which is described in Sato et al, 2009 (supra). Accordingly, in some preferred embodiments, the differentiation medium comprises EGF, Noggin and R-spondin. In some embodiments, the differentiation medium comprises EGF (e.g. 10-50 ng/ml, e.g. Peprotech), Noggin (100 ng/ml, e.g. Peprotech) and R-spondin (e.g. 500 ng/ml). This is known as the “ENR” medium. In some embodiments, the differentiation medium comprises a receptor tyrosine kinase ligand, a BMP inhibitor and a Wnt agonist.

In some embodiments, the differentiation medium does not comprise an EGFR pathway inhibitor. In some embodiments, the differentiation medium does not comprise a Notch inhibitor. In some embodiments, the differentiation medium does not comprise a Wnt inhibitor. In some embodiments, the differentiation medium does not comprise a BMP inhibitor. In some embodiments, the differentiation medium does not comprise a BMP pathway activator.

In some embodiments, a differentiation medium is a differentiation medium as described in WO 2017/220586.

In some embodiments, the step of differentiating stem cells and/or cells with stem cell potential to EECs comprises culturing the organoids in a basal medium. For example, the culture medium may comprise or consist of the basal medium without any stem cell factors. Stem cell factors are growth factors which support stem cell fate. Examples of stem cell factors include Wnt agonists such as R-spondin and BMP inhibitors such as Noggin. For example, in some embodiments, the basal medium is Advanced DMEM. Thus, in some embodiments, the basal medium does not comprise stem cell factors. The inventors have found that the presence of stem cell factors (which are typically present in an expansion medium) reduces EEC differentiation, but that many EECs are still obtained. Thus, in some embodiments, the basal medium comprises stem cell factors. The basal medium described herein is an example of a differentiation medium of the invention.

The expansion medium and/or the differentiation medium described herein, preferably comprise or consist of the components described herein in a basal medium.

Basal media for animal or human cell culture typically contain a large number of ingredients, which are necessary to support maintenance of the cultured cells. Suitable combinations of ingredients can readily be formulated by the skilled person, for example, taking into account the following disclosure. A basal medium for use in the invention will generally comprises a nutrient solution comprising standard cell culture ingredients, such as amino acids, vitamins, lipid supplements, inorganic salts, a carbon energy source, and a buffer, as described in more detail in the literature. In some embodiments, the culture medium is further supplemented with one or more standard cell culture ingredient, for example selected from amino acids, vitamins, lipid supplements, inorganic salts, a carbon energy source, and a buffer.

The skilled person will understand from common general knowledge the types of culture media that might be used as the basal medium for the step of expanding or differentiating stem cells and/or cells with stem cell potential to EECs. Potentially suitable cell culture media are available commercially, and include, but are not limited to, Dulbecco's Modified Eagle Media (DMEM), Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media.

For example, the basal medium may be selected from DMEM/F12 and RPMI 1640 supplemented with glutamine, insulin, penicillin/streptomycin and transferrin. In some embodiments, Advanced DMEM/F12 or Advanced RPMI is used, which is optimized for serum free culture and already includes insulin. In this case, said Advanced DMEM/F12 or Advanced RPMI medium may be supplemented with glutamine and penicillin/streptomycin. In some embodiments, AdDMEM/F12 (Invitrogen) supplemented with N2 and B27 is used. Preferably, the basal medium is DMEM, e.g. Advanced DMEM/F12. For example, the basal medium may comprise Advanced DMEM/F12, glutamine and B27.

In some embodiments, the basal medium comprises Advanced DMEM/F12, HEPES, penicillin/streptomycin, Glutamine, N-Acetylcysteine and B27.

In some embodiments, the basal culture medium comprises or consists of Advanced DMEM/F12 supplemented with penicillin/streptomycin, 10 mM HEPES, Glutamax, B27 (all from Life Technologies, Carlsbad, CA) and about 1 mM N-acetylcysteine (Sigma).

In some embodiments, said basal culture medium is supplemented with a purified, natural, semi-synthetic and/or synthetic growth factor and does not comprise an undefined component such as fetal bovine serum or fetal calf serum. Various different serum replacement formulations are commercially available and are known to the skilled person. Where a serum replacement is used, it may, for example, be used at between about 1% and about 30% by volume of the medium, according to conventional techniques.

The culture medium used in the invention may comprise serum, or may be serum-free and/or serum-replacement free, as described elsewhere herein. Culture media and cell preparations are preferably GMP processes in line with standards required by the FDA for biologics products and to ensure product consistency.

A culture medium of the invention will normally be formulated in deionized, distilled water. In some embodiments, a culture medium of the invention will advantageously be sterilized prior to use to prevent contamination, e.g. by ultraviolet light, heating, irradiation or filtration. The culture medium may be frozen (e.g. at −20° C. or −80° C.) for storage or transport. The medium may contain one or more antibiotics to prevent contamination. The medium may in some embodiments have an endotoxin content of less than 0.1 endotoxin units per ml, or may have an endotoxin content less than 0.05 endotoxin units per ml. Methods for determining the endotoxin content of culture media are known in the art.

A preferred basal culture medium is a defined synthetic medium that is buffered at a pH of 7.4 (preferably with a pH 7.2-7.6 or at least 7.2 and not higher than 7.6) with a carbonate-based buffer, while the cells are cultured in an atmosphere comprising between 5% and 10% CO₂, or at least 5% and not more than 10% CO₂, preferably 5% CO₂.

In embodiments in which the transcription factor is under control of an inducible promoter, organoids may be treated with an inducer of the inducible promoter. The inducer may be used at a suitable concentration. For example, where the promoter is a doxycycline-inducible promoter, organoids may be treated with doxycycline (e.g. Sigma) at a suitable concentration. An example of a suitable concentration is 1 μg/ml doxycycline (Sigma) in culture medium (e.g. in ENR medium). Thus, in some embodiments, the inducer is added at about 1 μg/ml to the culture medium. However, any suitable concentration may be used and the skilled person will be able to determine which concentrations are effective. Thus, the step of differentiating stem cells and/or cells with stem cell potential to EECs may comprise adding an inducer for the inducible promoter of the transcription factor (for example, doxycycline (Sigma) at 1 μg/ml) to the expansion medium and/or the differentiation medium. Thus, this step may comprise overexpressing the transcription factor (e.g. NEUROG3) in the intestinal organoid by culturing the organoid in the presence of the inducer of the inducible promoter. Similarly, this step may comprise culturing the one or more organoids established by culturing the cells in an expansion medium and/or a differentiation medium which comprises an inducer for the inducible promoter of the transcription factor (e.g. NEUROG3) (for example, doxycycline (Sigma) at 1 μg/ml). In some embodiments, the differentiation medium comprises a receptor tyrosine kinase ligand, a BMP inhibitor, a Wnt agonist and the inducer. In some embodiments, the differentiation medium comprises EGF, Noggin, R-spondin and the inducer (e.g. doxycycline), for example, the ENR medium and the inducer (e.g. doxycycline). Thus, in some embodiments, the differentiation medium comprises a basic medium without stem cell factors. Similarly, in some embodiments, the differentiation medium comprises the HSi medium and the inducer (e.g. doxycycline).

The inventors have found that hormone expression peaks after 5 days of initiation of NEUROG3 expression. Accordingly, in some embodiments, the step of differentiating stem cells and/or cells with stem cell potential to EECs comprises culturing the organoid for 5 days, for example, in the differentiation medium or the expansion medium. For example, in some embodiments, the differentiation step comprises culturing the organoid for 5 days after initiating differentiation, e.g. after contacting the organoid with the inducer. However, other lengths of time are also envisaged. Accordingly, in some embodiments, the step of differentiating stem cells and/or cells with stem cell potential to EECs comprises culturing the organoid for a period of time between 3-14 days, e.g. 3-12, 4-10, 4-8, 5-14, 5-10, 5-8, 3, 4, 5, 6, 7 or 8 days, after initiating differentiation, e.g. after contacting the organoid with the inducer. As mentioned above, in some embodiments the inducer is not present throughout the entire time that the cells are being differentiated. However, in some embodiments, the inducer is present throughout the entire time.

In some embodiments, after the stem cells have been differentiated to EECs, the organoids may then be used in an assay, for example, in a method as described herein. In some embodiments, the organoids are used to obtain an end-point measurement. For example, RNA or protein can be collected from the organoid for use in qPCR studies, for example, to detect the level of transcripts of an EEC-specific gene of interest, e.g. a hormone, hormone precursor and/or a hormone synthesizing enzyme.

The organoids of the invention can be frozen and thawed and put into culture without losing their genetic integrity or phenotypic characteristics and without loss of proliferative capacity. Thus the organoids can be easily stored and transported. Thus in some embodiments, the invention provides a frozen organoid. Preferably, an organoid of the invention is frozen before the stem cells and/or cells with stem cell potential have been differentiated to EECs. However, in some embodiments, an organoid comprising EECs is frozen.

In some embodiments, the method comprises the steps of generating one or more intestinal stem cells and/or intestinal cells with stem cell potential which comprise an inducible transcription factor for differentiating intestinal stem cells and/or intestinal cells with stem cell potential to EECs and generating one or more intestinal organoids from the intestinal stem cells and/or cells with stem cell potential, but does not comprise the step of differentiating stem cells in the one or more organoids to EECs. The organoids can be frozen after the step of generating the one or more intestinal organoids and before the stem cells are differentiated to EECs. Thus, in some embodiments, the invention provides a frozen organoid that has been obtained by or is obtainable by a method for enriching the population of EECs in an intestinal organoid as described herein wherein the method comprises steps i and ii of such method but omits step iii. Similarly, in some embodiments, the invention provides a frozen organoid that has been obtained by or is obtainable by a method for making a reporter organoid, wherein the stem cells and/or cells with stem cell potential in the reporter organoid have not been differentiated to EECs. Thus, in some embodiments, a frozen organoid of the invention does not comprise EECs. When the organoid is required for use, it can be thawed and cultured in an expansion medium and/or a differentiation medium in the presence of the inducer for the transcription factor. This allows differentiation of the stem cells to EECs. In some alternative embodiments, the organoid is frozen after the stem cells have been differentiated to EECs. In some embodiments, the organoid is frozen in expansion medium. In some embodiments, the organoid is frozen in differentiation medium. In some embodiments, the organoid is frozen in a basal medium.

In some embodiments, the method of enriching the population of EECs in an intestinal organoid further comprises the step of selecting an organoid in which expression of the transcription factor can be induced, making a clonal line of the selected organoid and then freezing the organoid, for example, in expansion medium. Thus, in some embodiments, the intestinal stem cells and/or cells with stem cell potential have not been differentiated to EECs. Similarly, the invention provides a frozen organoid that is a clonal organoid made by a method for making an intestinal organoid, as described herein.

The step of introducing a construct comprising a transcription factor under control of an inducible promoter (or comprising an inducible promoter for an endogenous transcription factor gene) and the step of tagging one or more EEC-specific genes may be carried out in any order or simultaneously. The inventors have found that tagging the endogenous EEC-specific gene (e.g. the endogenous hormone, hormone precursor or hormone synthesizing enzyme) with a detectable marker in stem cells which already comprise a construct for an inducible transcription factor is far more efficient than conducting the steps in the opposite order. Thus, in preferred embodiments, the endogenous EEC-specific gene is tagged in stem cells which already comprise a construct for an inducible transcription factor (e.g. an inducible NEUROG3 overexpression construct). However, in some alternative embodiments, the inducible transcription factor for differentiating intestinal stem cells and/or intestinal cells with stem cell potential to EECs is introduced into the stem cells after the first detectable marker has been introduced.

Tagging at the Endogenous Locus of the EEC-Specific Gene

The invention provides a method of making one or more intestinal organoids comprising stem cells and/or cells with stem cell potential in which one or more EEC-specific genes is tagged at its endogenous locus with a detectable marker, wherein the method comprises or consists of the steps of a) tagging one or more EEC-specific genes with a detectable marker at its endogenous locus in one or more intestinal stem cells and/or in one or more intestinal cells with stem cell potential; and b) generating one or more intestinal organoids by culturing the cells in an expansion medium. This method may be used to make a reporter organoid.

In some embodiments, the step of tagging one or more EEC-specific genes with a detectable marker at its endogenous locus in one or more intestinal stem cells and/or in one or more intestinal cells with stem cell potential comprises contacting an intestinal stem cell and/or an intestinal cell with stem cell potential with one or more constructs encoding the first detectable marker and preferably also a second detectable marker, wherein the construct encoding the first detectable marker targets the endogenous locus of the EEC-specific gene.

The method of making one or more intestinal organoids comprising stem cells and/or cells with stem cell potential in which one or more EEC-specific genes is tagged at its endogenous locus with a detectable marker may comprise the tagging step described herein and may further comprise one or more selection and expansion steps described herein.

For example, in some embodiments, the method comprises:
i. contacting an intestinal stem cell and/or an intestinal cell with stem cell potential with one or more constructs encoding a first detectable marker and a second detectable marker, wherein the construct encoding the first detectable marker targets the EEC-specific gene at its endogenous locus;
ii. culturing the cells in an expansion medium, selecting transfected cells that express the second detectable marker gene and plating the selected cells as single cells; and
iii. generating one or more intestinal organoids by culturing the single cells in an expansion medium.

In some embodiments, the method for making a reporter organoid comprises:
i. contacting an intestinal stem cell and/or an intestinal cell with stem cell potential with one or more constructs encoding a first detectable marker and a second detectable marker, wherein the construct encoding the first detectable marker targets the EEC-specific gene at its endogenous locus;
ii. culturing the cells in an expansion medium, selecting transfected cells that express the second detectable marker gene and plating the selected cells as single cells;
iii. generating one or more intestinal organoids by culturing the single cells in an expansion medium;
iv. differentiating stem cells and/or cells with stem cell potential to EECs in one or more organoids generated in step (iii), e.g. by overexpressing an inducible transcription factor for differentiating intestinal stem cells and/or intestinal cells with stem cell potential to EECs, while culturing the one or more organoids in an expansion medium and/or a differentiation medium; and
v. selecting one or more organoids which express the first detectable marker, dissociating the selected organoids and expanding the cells in an expansion medium to generate one or more organoids.

The one or more constructs may be any suitable constructs, e.g. circular vectors and/or linear vectors. In some embodiments, the one or more constructs comprise or consist of two or more circular vectors and/or linear nucleic acids. For example, in some embodiments, the one or more constructs comprise or consist of three circular vectors. In some embodiments, the one or more constructs comprise or consist of two circular vectors. In some embodiments, the one or more constructs comprise or consist of a circular vector and a linear nucleic acid. In some embodiments, the one or more constructs comprise one or more constructs in addition to the one or more constructs encoding the first detectable marker and/or the second detectable marker. For example, in some embodiments, the one or more additional constructs encode components of the CRISPR/Cas9 machinery, for example, Cas9 and/or one or more gRNAs. In some embodiments, components of the CRISPR/Cas9 machinery are comprised on the same constructs as encode the first detectable marker and/or the second detectable marker. In some embodiments, the first detectable marker and the second detectable marker are encoded by different constructs. In some embodiments, the first detectable marker and the second detectable marker are encoded by the same construct. In preferred embodiments, the one or more constructs are one or more DNA constructs. Any suitable vector backbone may be used in the one or more constructs. For example, the vector backbone may be a DNA backbone, for example, a pUC vector backbone such as pUC118. In some embodiments, the same vector backbone is used for at least two (e.g. 2 or 3) constructs. In some embodiments, different vector backbones are used for the different constructs.

In preferred embodiments, the EEC-specific gene is a gene encoding a hormone, hormone precursor or a hormone synthesizing enzyme. In further preferred embodiments, the EEC-specific gene is a hormone or a hormone precursor.

The cells in which the one or more EEC-specific genes are tagged are preferably intestinal stem cells and/or intestinal cells with stem cell potential. More preferably, they are intestinal stem cells.

The step of contacting an intestinal stem cell and/or intestinal cell with stem cell potential with one or more constructs in the various methods described herein is preferably conducted under conditions suitable for introducing the one or more constructs into the stem cell and/or into the cell with stem cell potential. Any suitable method for introducing the one or more constructs into the cell may be used. E.g. in some embodiments, the one or more constructs are introduced by transfection. The cells may be transfected using any suitable technique, for example, electroporation or transfection using Lipofectamine. In some embodiments, the one or more constructs are introduced by transduction.

In preferred embodiments, the one or more intestinal stem cells and/or intestinal cells with stem cell potential used in step (i) already comprise an inducible transcription factor as described herein for differentiating intestinal stem cells to EECs, for example, an inducible NEUROG3 construct. For example, in preferred embodiments, the cells in which the EEC-specific gene is tagged already comprise an inducible transcription factor as described herein for differentiating intestinal stem cells to EECs. However, in some alternative embodiments, the one or more intestinal stem cells and/or intestinal cells with stem cell potential used in step (i) do not comprise such an inducible transcription factor. For example, in some embodiments, the cells in which the EEC-specific gene is tagged do not comprise such an inducible transcription factor. Such inducible transcription factor may, if desired, then be introduced into the cells at a later stage in the method, preferably before the step of differentiating stem cells in the organoid to EECs. In some embodiments, the inducible transcription factor construct (e.g. the inducible NEUROG3 overexpression construct) is introduced into the cells after the step of generating one or more intestinal organoids that comprise the EEC-specific gene tagged with the detectable marker and before the step of differentiating stem cells and/or cells with stem cell potential in the intestinal organoid to EECs.

In some embodiments, only one EEC-specific gene is tagged in the reporter organoid. In some embodiments, more than one EEC-specific gene is tagged in the reporter organoid (e.g. 2 or more, e.g. 3, 4, 5 or more). In some embodiments, when more than one EEC-specific gene is being tagged, different first detectable markers may advantageously be used for the different EEC-specific genes, for example, fluorescent detectable markers of different colours. However, the same first detectable markers may be used, if desired.

In some embodiments, in order to tag a second EEC-specific gene in a reporter organoid, the steps for a method for making a reporter organoid are all repeated. Thus, the endogenous locus of the second EEC-specific gene may then be targeted. Further subsequent rounds may be repeated in order to tag further EEC-specific genes, for example, a third, fourth or fifth. Intestinal stem cells and/or intestinal cells with stem cell potential may be obtained from the reporter organoid as a starting point. For example, the reporter organoid may be dissociated to obtain the cells. It is not necessary to include the step of making or obtaining intestinal stem cells and/or cells with stem cell potential which comprise an inducible transcription factor for differentiating intestinal stem cells and/or intestinal cells with stem cell potential to EECs, because the intestinal stem cells and or intestinal cells with stem cell potential obtained from or obtainable from the reporter organoid made in the first round will already comprise the inducible transcription factor.

The first detectable marker gene advantageously acts as the tag which allows the EEC-specific gene to be detected. The second detectable marker may be used to detect successful transfection. Thus, in some embodiments, the second detectable marker acts as a selection marker. In some embodiments, the first detectable marker is selected from a fluorescent marker and an antibiotic resistance marker. Preferably, the first detectable marker gene is a fluorescent marker. The second detectable marker gene may be a fluorescent marker or any other suitable detectable marker, for example, an antibiotic resistance gene. In some embodiments, the second detectable marker is an antibiotic resistance gene. It may be advantageous to use an antibiotic resistance gene as the second detectable marker in some embodiments in which the gene of interest is expressed at a lower level than hormone protein/peptide-coding genes. For example, TPH1, the rate-limiting enzyme involved in the synthesis of serotonin, is expressed at a lower level than hormone peptide-coding genes, and so in some embodiments in which TPH1 is the gene of interest, antibiotic-mediated selection is preferably used to generate reporter organoid lines. Thus, in some embodiments, the second detectable marker is an antibiotic resistance gene when the EEC-specific gene is a hormone synthesizing enzyme. In some embodiments, the first and second detectable marker genes are fluorescent markers. In some embodiments, the first and second detectable markers are different fluorescent markers, for example, of different colours, for example, selected from mNeon, tdTomato and mCherry. In some embodiments, the first and second detectable marker are the same fluorescent marker. In some embodiments, the second detectable marker gene is constitutively expressed. For example, in some embodiments the second detectable marker gene is a constitutively expressed fluorescent marker. For example, in some embodiments, the first detectable marker gene is mNeon or td Tomato and the second detectable marker gene is constitutively expressed mCherry. In some embodiments, the antibiotic resistance gene is a Blasticidin resistance gene.

In some embodiments, the second detectable marker is comprised on the same construct as the first detectable marker. Thus, in some embodiments, the second detectable marker is integrated into the endogenous locus together with the first detectable marker. In some embodiments, the second detectable marker is not on the same construct as the first detectable marker.

Advantageously, the method may be used to generate a reporter organoid in which a hormone and/or a hormone precursor is tagged with the detectable marker as a fusion protein in one or more EECs. Thus, the first detectable marker preferably forms a fusion protein with the hormone or hormone precursor. The first detectable marker gene is preferably fused to the gene encoding the hormone or hormone precursor at a location which will not affect expression, function, post translational modification and/or secretion of the hormone or hormone precursor. In some embodiments, the first detectable marker is fused to the gene encoding the hormone or hormone precursor at a location which will not affect gene expression and/or secretion of the hormone or hormone precursor. Preferably, the first detectable marker is fused to the gene encoding the hormone or hormone precursor at a location which will minimise the loss of any amino acids from the encoded hormone or hormone precursor. In some embodiments, the first detectable marker is fused to the gene encoding the hormone or hormone precursor near the stop codon of the hormone or hormone precursor, for example, as close as possible to the stop codon of the gene to minimize amino acid loss. In some embodiments, fusion of the first detectable marker gene to the gene encoding the hormone or hormone precursor results in loss of five or fewer (e.g. 4, 3, 2, 1 or fewer or zero) amino acids from the C-terminus of the hormone or hormone precursor. However, in some embodiments, the first detectable marker does not form a fusion protein with the hormone or hormone precursor.

In embodiments in which the EEC-specific gene is not a hormone or a hormone precursor, the first detectable marker preferably does not form a fusion protein with the EEC-specific gene. For example, in preferred embodiments, the detectable marker does not form a fusion protein with a hormone synthesizing enzyme. In addition, as mentioned above, in some embodiments in which the EEC-specific gene is a hormone or hormone precursor, the first detectable marker does not form a fusion protein with the EEC-specific gene. However, in some embodiments, the first detectable marker does form a fusion protein with the EEC-specific gene. The first detectable marker is preferably inserted at the endogenous locus at a position that will not affect the expression and/or function of the EEC-specific gene. In some embodiments, the first detectable marker gene is separated from the reading frame of the EEC-specific gene by a self cleavable sequence (e.g. P2A). Use of a self cleavable sequence avoids creating a fusion protein of the endogenous EEC-specific gene of interest and the first detectable marker gene.

In some embodiments, the method comprises culturing the cells in an expansion medium, selecting transfected cells that express the second detectable marker gene and plating the selected cells as single cells. In some embodiments, the second detectable marker is constitutively expressed. In some embodiments, the cells are cultured for 3-7 days (for example, 4-6 days or 5 days) before cells are selected that express the second detectable marker gene. In embodiments in which the second detectable marker gene is a fluorescent molecule, FACS (e.g. FACS-ARIA (BD Biosciences)) can be used to select and sort cells.

In some embodiments, the method comprises generating one or more intestinal organoids by culturing the selected single cells in an expansion medium, for example, an expansion medium as described herein. The skilled person will know how to select a suitable expansion medium based on the teaching in the art in combination with the teaching provided herein. The skilled person will understand the appropriate length of time and conditions to culture the cells to obtain organoids and examples of suitable methods are described herein. In some embodiments, the cells are cultured for about two weeks in the expansion medium. In some embodiments, Rho kinase inhibitor (e.g. 10 μM, Calbiochem) and/or a Wnt-surrogate (e.g. 0.15 nM, U-Protein Expression) is added to the culture medium up to 1 week after selecting the transfected cells to enhance single cell outgrowth. Advantageously, this step allows clonal organoids to be generated.

The step of differentiating stem cells and/or cells with stem cell potential to EECs in one or more organoids while culturing the one or more organoids in an expansion medium and/or differentiation medium preferably comprises overexpressing the inducible transcription factor as described herein (e.g. NEUROG3). Methods for overexpressing the inducible transcription factor are described herein. For example, in some embodiments, the inducible transcription factor is overexpressed by culturing the one or more organoids in an expansion medium and/or a differentiation medium in the presence of the inducer for the inducible promoter. Differentiation of the stem cells in the organoid to EECs preferably results in one or more tagged EEC-specific genes (e.g. tagged hormones, tagged hormone precursors and/or tagged hormone synthesizing enzymes) being expressed which can then be detected using the first detectable marker gene. Thus, organoids which express the one or more tagged EEC-specific genes can be selected by detecting expression of the first detectable marker gene. Such organoids typically appear 2-3 days after activation of expression of the inducible transcription factor as described herein (e.g. NEUROG3). Hormones and hormone precursors, and therefore, the first detectable marker, typically have a vesicular localisation. Thus, in embodiments in which the first detectable marker gene is a fluorescent molecule and the EEC-specific gene is a hormone or hormone precursor, vesicular fluorescent signal typically appears after 2-3 days. Overexpression of the inducible transcription factor described herein is preferably achieved using an overexpression pulse. This can quickly identify organoids that have been correctly targeted and so allows the reporter organoids described herein to be made quickly. Methods for achieving an overexpression pulse are described herein. For example, in some embodiments, the inducer for the inducible promoter is present in the expansion medium and/or the differentiation medium for a limited period of time, e.g. for about 48 hours, and is then removed and washed away.

In some embodiments, the method comprises selecting one or more organoids which express the first detectable marker gene, dissociating the selected organoids and expanding the cells in an expansion medium to generate one or more organoids. Advantageously, this allows stable knock-in organoid lines to be developed. Any suitable method may be used to dissociate the organoids, for example, enzymatic digestion, for example, using TrypLE (e.g. TrypLE Express, Life Technologies).

In embodiments in which the first detectable marker is a fluorescent marker, the step of selecting one or more organoids which express the first detectable marker may involve selecting organoids which show visible fluorescence. In some embodiments, between 0.1 and 20% of the transfected cells have correct integration of the first detectable marker, e.g. the hormone reporter. In some embodiments, more than 20% of the transfected cells have correct integration of the first detectable marker, e.g. the hormone reporter.

Cells and/or organoids that express a fluorescent detectable marker gene can be identified by any suitable technique, for example, by FACs or by microscopy (e.g. fluorescence microscopy (e.g. using an EVOS microscope from Thermo Fisher Scientific) or confocal microscopy).

The one or more organoids generated by the step of selecting one or more organoids which express the first detectable marker, dissociating the selected organoids and expanding the cells in an expansion medium to generate one or more organoids, may optionally be called one or more stable reporter organoid lines. The various selection steps in the method ensure that the stable reporter organoid lines comprise stem cells and/or cells with stem cell potential comprising the endogenous EEC-specific genes tagged with the first detectable marker. Advantageously, in embodiments in which stem cells and/or cells with stem cell potential are used which comprise an inducible transcription factor for differentiating stem cells and/or cells with stem cell potential to EECs, the various selection steps in the method also ensure that stem cells and/or cells with stem cell potential in the stable reporter organoid line comprise the inducible transcription factor.

In some embodiments, the one or more organoids are frozen after making the stable organoid line, e.g. the stable reporter organoid line. They may subsequently be thawed and used in a method as described herein.

As mentioned above, in some embodiments, the method comprises an optional subsequent step which comprises differentiating stem cells to EECs, e.g. in the one or more stable reporter organoid lines, while culturing the one or more organoids in an expansion medium and/or a differentiation medium. For example, differentiation of the stem cells to EECs may be achieved by adding the inducer for the inducible promoter of a transcription factor as described herein (e.g. NEUROG3) to the culture medium in which the one or more organoids is cultured. Thus, in some embodiments, this subsequent step comprises culturing the one or more organoids in the presence of the inducer for the inducible promoter of a transcription factor as described herein (e.g. for NEUROG3).

In some embodiments, the method further comprises validating that a reporter organoid has been made. For example, in embodiments in which the EEC-specific gene is a hormone or hormone precursor gene, the method may further comprise stimulating secretion of the hormone or hormone precursor and visualising the effect on the detectable marker gene. For example, in some embodiments, upon stimulation of secretion, intracellular fluorescence decreases over time. For example, in some embodiments, intracellular fluorescence decreases after 12 hours. This is consistent with loss of stored, tagged hormones or tagged hormone precursors from the intracellular cytoplasmic vesicles upon secretion. In some embodiments, secretion is stimulated by treating the reporter organoids with a substance or composition that increases cAMP levels (such as a stimulator of adenylate cyclase, e.g. Forskolin). The method may further comprise monitoring for cell viability or cell death. For example, the method may comprise monitoring to ensure the cells in the reporter organoid are not dying or have not died.

Previously, introducing fluorescent molecules into organoids was difficult because the introduction of genetic material in untransformed cells such as adult stem cell derived organoids has proven to be challenging, as illustrated by the low number of genetically engineered organoids developed. The inventors have now developed a system based on CRISPR/Cas9. CRISPR/Cas9 allows the targeting of endogenous loci, mediated by the specificity of a guide RNA (gRNA). Subsequent repair through homology-directed-repair (HDR) or Non-homologous-end-joining (NHEJ) in turn allows for the introduction of exogenous genetic material (Bukhari and Müller, 2019; He et al., 2016; Schmid-Burgk et al., 2016). In some embodiments, the one or more EEC-specific genes is tagged with a detectable marker using non-homologous-end-joining (NHEJ). The inventors have optimized a strategy for site-specific introduction of DNA into organoids using NHEJ, a technique termed CRISPR-HOT (Homology-independent organoid transgenesis) (Artegiani, Hendriks et al., 2020). The inventors have found this method to be useful for introducing detectable markers such as fluorescent markers into organoids in order to tag EEC-specific genes such as hormones expressed by cells in the organoid (e.g. by the EECs in intestinal organoids). In the examples of the present application, the inventors have fluorescently labelled a series of secreted hormones and hormone precursors as fusion proteins using this method.

Accordingly, in some embodiments, the one or more constructs comprise or consist of (a) a first vector encoding a gRNA targeting the endogenous locus of the EEC-specific gene; (b) a second vector comprising a first detectable marker gene; and (c) a third vector encoding Cas9, a second detectable marker gene and a second gRNA that targets the vector backbone of the second vector promoting its linearization.

In some embodiments, the step of tagging one or more EEC-specific genes at its endogenous locus with a detectable marker in one or more intestinal stem cells and/or in one or more intestinal cells with stem cell potential comprises: contacting an intestinal stem cell and/or an intestinal cell with stem cell potential with (a) a first vector encoding a gRNA targeting the endogenous locus of the EEC-specific gene; (b) a second vector comprising a first detectable marker gene; and (c) a third vector encoding Cas9, a second detectable marker gene and a second gRNA that targets the vector backbone of the second vector promoting its linearization.

Thus, in some embodiments, the method for making a reporter organoid comprises:
(i) contacting an intestinal stem cell and/or an intestinal cell with stem cell potential with (a) a first vector encoding a gRNA targeting the endogenous locus of an EEC-specific gene; (b) a second vector comprising a first detectable marker gene; and (c) a third vector encoding Cas9, a second detectable marker gene and a second gRNA that targets the vector backbone of the second vector promoting its linearization;
(ii) culturing the cells in an expansion medium, selecting transfected cells that express the second detectable marker gene and plating the selected cells as single cells;
(iii) generating one or more intestinal organoids by culturing the single cells in an expansion medium;
(iv) differentiating stem cells and/or cells with stem cell potential to EECs in one or more organoids generated in step (iii), e.g. by overexpressing an inducible transcription factor for differentiating intestinal stem cells and/or intestinal cells with stem cell potential to EECs, while culturing the one or more organoids in an expansion medium and/or a differentiation medium; and
(v) selecting one or more organoids which express the first detectable marker, dissociating the selected organoids and expanding the cells in an expansion medium to generate one or more organoids.

The first gRNA targeting the endogenous locus of an EEC-specific gene is preferably designed to cut at a location which will not affect gene expression and/or post translational modification and/or cellular location and/or function. Preferably, the first gRNA is designed to cut at a location which will minimise the loss of any amino acids from the encoded EEC-specific protein. In some embodiments, the first gRNA targeting the endogenous locus of the EEC-specific gene is designed to cut at a location which will not affect gene expression and/or secretion (where relevant). In some embodiments, the first gRNA is designed to cut near the stop codon of the EEC-specific gene, for example, as close as possible to the stop codon of the EEC-specific gene to minimize amino acid loss. In some embodiments, cutting with the first gRNA results in loss of five or fewer (e.g. 4, 3, 2, 1 or fewer or zero) amino acids from the C-terminus of the protein encoded by the EEC-specific gene.

In some embodiments, the linearized second vector comprising the first detectable marker gene is inserted into the endogenous locus using non-homologous end joining (NHEJ).

In some embodiments, step (i) comprises using three different second gRNAs targeting the vector backbone of the second vector to cause the insertion of 0, 1 or 2 additional nucleotides in the endogenous locus. Advantageously, use of the three different second gRNAs targeting the vector backbone of the second vector which can cause the insertion of 0, 1 or 2 additional nucleotides in the endogenous locus increases the production of in-frame insertions of the first detectable marker gene. Thus, the different second gRNAs are preferably selected to increase the production of in-frame insertions of the first detectable marker gene. In some embodiments, the three different second gRNAs are termed gRNAs a, b and c: gRNA a causes the insertion of 0 additional nucleotides in the endogenous locus; gRNA b causes the insertion of 1 additional nucleotide in the endogenous locus; and gRNA c causes the insertion of 2 additional nucleotides in the endogenous locus. In some embodiments, more than three different second gRNAs are used, e.g. six different second gRNAs, to incorporate the combination of 0, 1, 2, 3, 4 or 5 additional nucleotides in the endogenous locus. In some less preferred alternative embodiments, instead of using three different second gRNAs, two different second gRNAs are used to cause the insertion of 0, 1 or 2 (e.g. 0 or 1; 0 or 2; or 1 or 2) additional nucleotides in the endogenous locus, or only a single second gRNAs is used. The skilled person would know how to design the sequence of the gRNAs using standard techniques known in the art.

In some embodiments, all of the different second gRNAs are encoded by a single vector. For example, in some embodiments, the three different second gRNAs are all encoded by a single vector. In some embodiments, each one of the different second gRNAs is encoded by a separate vector. For example, in some embodiments, each one of the three different second gRNAs is encoded by a separate vector. In some embodiments, a subset of the different second gRNAs is encoded by one vector and the other one or more different second gRNAs are encoded by one or more other vectors. For example, in some embodiments, two of the three different second gRNAs are encoded by one vector and the third gRNA is encoded by a different vector.

In some embodiments, the step of tagging one or more EEC-specific genes at its endogenous locus with a detectable marker in one or more intestinal stem cells and/or in one or more intestinal cells with stem cell potential comprises: contacting an intestinal stem cell and/or an intestinal cell with stem cell potential with (a) a construct encoding Cas9 and a gRNA targeting the endogenous locus of the EEC-specific gene; (b) a construct encoding the first detectable marker and the second detectable marker. The method for making a reporter organoid may therefore comprise this tagging step and the further and/or preceding steps described herein. In some embodiments, the construct of (a) is a circular vector, e.g. a circular DNA vector. In some embodiments, the construct of (a) is a linear DNA. In some embodiments, the construct of (b) is a linear DNA.

In some embodiments, the step of tagging one or more EEC-specific genes at its endogenous locus with a detectable marker in one or more intestinal stem cells and/or in one or more intestinal cells with stem cell potential comprises: contacting an intestinal stem cell and/or an intestinal cell with stem cell potential with (a) a construct encoding Cas9 and a gRNA targeting the endogenous locus of the EEC-specific gene; (b) a construct encoding the first detectable marker; and (c) a construct encoding the second detectable marker. The method for making a reporter organoid may therefore comprise this tagging step and the further and/or preceding steps described herein. In some embodiments, the construct of (a) is a circular vector, e.g. a circular DNA vector. In some embodiments, the construct of (a) is a linear DNA. In some embodiments, the construct of (b) and/or (c) is a linear DNA.

In some embodiments described herein, Cas9 and one or more gRNAs are encoded on separate constructs, and the methods described herein can be adapted accordingly.

In some embodiments, the one or more EEC-specific genes is tagged with a detectable marker using homology directed repair (HDR). HDR is a well-known technique in the art and accordingly the skilled person would understand how to carry it out. HDR requires large numbers of cells to be successful because the transfection efficiency is low. The intestinal organoids described herein, which comprise a high percentage of stem cells, advantageously allow HDR to be carried out on intestinal stem cells and/or intestinal cells with stem cell potential.

Advantageously, as described above, in some embodiments, the one or more cells used in step (i) are obtained from or are obtainable from an intestinal organoid, for example, as described herein, in which the stem cells and/or cells with stem cell potential already comprise an inducible transcription factor as described herein for differentiating stem cells to EECs. Thus, use of such an intestinal organoid to obtain the one or more cells for use in step (i) advantageously provides large numbers of stems cells for use in a method of making a reporter organoid as described herein.

In some embodiments, the nucleic acid construct comprises or consists of (a) a vector encoding a gRNA targeting an EEC-specific gene at its endogenous locus and Cas9; (b) a nucleic acid comprising a first detectable marker gene between a left homology arm (HA1) and a right homology arm (HA2), wherein HA1 and HA2 are homologous to regions 5′ and 3′ of the sequence cut by the gRNA, respectively, wherein a second detectable marker is encoded either by the vector encoding the gRNA and Cas9 or by the nucleic acid comprising the first detectable marker gene, wherein when the second detectable marker is encoded by the nucleic acid comprising the first detectable marker gene, the nucleic acid comprises the first detectable marker gene and the second detectable marker gene between the left homology arm and the right homology arm.

In some embodiments, the step of tagging one or more EEC-specific genes at its endogenous locus with a detectable marker in one or more intestinal stem cells and/or in one or more intestinal cells with stem cell potential comprises: contacting an intestinal stem cell and/or an intestinal cell with stem cell potential with (a) a vector encoding a gRNA targeting an EEC-specific gene at its endogenous locus and Cas9; (b) a nucleic acid comprising a first detectable marker gene between a left homology arm (HA1) and a right homology arm (HA2), wherein HA1 and HA2 are homologous to regions 5′ and 3′ of the sequence cut by the gRNA, respectively, wherein a second detectable marker is encoded either by the vector encoding the gRNA and Cas9 or by the nucleic acid comprising the first detectable marker gene, wherein when the second detectable marker is encoded by the nucleic acid comprising the first detectable marker gene, the nucleic acid comprises the first detectable marker gene and the second detectable marker gene between the left homology arm and the right homology arm.

In some embodiments, the invention provides a method for making a reporter organoid, wherein the method comprises:
(i) contacting an intestinal stem cell and/or an intestinal cell with stem cell potential with (a) a vector encoding a gRNA targeting an endogenous locus of an EEC-specific gene and Cas9; (b) a construct comprising a first detectable marker gene and a second detectable marker gene between a left homology arm (HA1) and a right homology arm (HA2), wherein HA1 and HA2 are homologous to regions 5′ and 3′ of the sequence cut by the gRNA, respectively;
(ii) culturing the cells in an expansion medium, selecting transfected cells that express the second detectable marker gene and plating the selected cells as single cells;
(iii) generating one or more intestinal organoids by culturing the single cells in an expansion medium;
(iv) differentiating stem cells to EECs in one or more organoids generated in step (iii), e.g. by overexpressing a transcription factor for differentiating intestinal stem cells and/or intestinal cells with stem cell potential to EECs, while culturing the one or more organoids in an expansion medium and/or a differentiation medium; and
(v) selecting one or more organoids which express the first detectable marker, dissociating the selected organoids and expanding the cells in an expansion medium to generate one or more organoids.

In some embodiments, the construct comprising the first detectable marker gene and the second detectable marker gene is a circular vector, e.g. a DNA vector such as a DNA plasmid. In some embodiments, the construct comprising the first detectable marker gene and the second detectable marker gene is a linear DNA. For example, in some embodiments, the cells are contacted with (a) a vector encoding a gRNA targeting an EEC-specific gene at its endogenous locus and Cas9; (b) a vector comprising a first detectable marker gene and a second detectable marker gene between a left homology arm (HA1) and a right homology arm (HA2), wherein HA1 and HA2 are homologous to regions 5′ and 3′ of the sequence cut by the gRNA, respectively.

The embodiments discussed above for the NHEJ method may be applied to the HDR method mutatis mutandis. For example, in some embodiments, the left homology arm (HA1) is just upstream of the stop codon and the right homology arm (HA2) is in the 3′UTR of the gene of interest. In some embodiments, HA1 and HA2 may be located immediately around the C-terminus of the gene of interest. Thus, in some embodiments, HA1 and HA2 are homologous to regions N-terminal and C-terminal of the stop codon of the endogenous gene of interest. This allows the gene to be tagged near its C-terminus whilst retaining functionality of the gene. In some embodiments, HA1 and HA2 are designed so that insertion of the first and second detectable marker genes results in loss of five or fewer (e.g. 4, 3, 2, 1 or fewer or zero) amino acids from the C-terminus of the protein encoded by the EEC-specific gene.

In some embodiments, the HDR method is used when an EEC-specific protein is of interest which is not expressed by a gene encoded by the cell but is instead synthesized by one or more encoded enzyme(s). For example, in such embodiments, the EEC-specific gene may encode an enzyme involved in production of a molecule, e.g. a hormone, e.g. an EEC-specific hormone. For example, in embodiments in which the EEC-specific protein of interest is a hormone that is not expressed by a gene but is instead synthesized by one or more encoded enzymes, for example, where the EEC-specific gene is a hormone synthesizing enzyme, the HDR method is particularly useful. For example, the neurotransmitter serotonin is a hormone that is secreted by Enterochromaffin cells (ECs). ECs are a subset of EECs. However, serotonin is not an encoded gene but is instead synthesized by other enzymes encoded by the ECs. Tryptophan hydroxylase 1 (TPH1) is the rate-limiting enzyme involved in the synthesis of serotonin. Accordingly, in some embodiments, the EEC-specific gene of interest is TPH1. Tagging TPH1 would be useful when the ability to study serotonin production is of interest, for example. In some alternative embodiments, dopa carboxylase (DDC) is the EEC-specific gene of interest and so is tagged. DDC is another enzyme involved in serotonin production. However, the HDR method may be used to tag any EEC-specific gene of interest, e.g. a hormone or hormone precursor expressed by the cell. Similarly, the NHEJ method may be used to tag any EEC-specific gene of interest. Thus, the methods of the invention can be used to tag a diverse range of secreted products or their production machinery.

In some embodiments, a hormone described herein is also a neurotransmitter. For example, serotonin is an example of a hormone which is also a neurotransmitter.

In some embodiments, step (i) of the method for making a reporter organoid comprises contacting an intestinal stem cell and/or an intestinal cell with stem cell potential with a construct comprising a detectable marker (e.g. a fluorescent marker) and a selectable marker (e.g. an antibiotic resistance gene) between a left homology arm (HA1) and a right homology arm (HA2), wherein the homology arms are homologous to regions 5′ and 3′ of the stop codon of the endogenous EEC-specific gene, respectively, and using HDR to introduce the construct into the stem cells and/or intestinal cells with stem cell potential.

In some embodiments, step (i) of the method for making a reporter organoid comprises contacting an intestinal stem cell and/or an intestinal cell with stem cell potential with (a) a first vector encoding a gRNA targeting the stop codon of an EEC-specific gene at its endogenous locus and Cas9; and (b) a nucleic acid comprising a first detectable marker gene which is a fluorescent marker gene and a second detectable marker gene which is an antibiotic resistance gene between a left homology arm (HA1) and a right homology arm (HA2), wherein HA1 and HA2 are homologous to regions 5′ and 3′ of the stop codon of the endogenous EEC-specific gene, respectively. In some embodiments, the EEC-specific gene is a hormone synthesizing enzyme.

Advantageously, the method (e.g the HDR method) may be used to generate a reporter organoid in which a hormone synthesizing enzyme gene of interest is tagged in one or more stem cells and/or cells with stem cell potential. In a differentiated organoid of the invention, the EEC-specific gene is advantageously tagged in one or more EECs.

In some embodiments, the term “one or more intestinal organoids” or “one or more organoids”, may be replaced with the term “intestinal organoids” or “organoids”, respectively.

Methods of the invention which comprise culturing stem cells in expansion medium generally make multiple organoids, generally in the region of 1000s of organoids. In some embodiments, a method of the invention generates one or more organoids, e.g. 1, or at least 2, 5, 10, 100, 500, 1000 organoids. The ability of generate 1000s of organoids in a few days or weeks is highly advantageous for high throughput screening embodiments, and is particularly advantageous for patient diagnostic embodiments, e.g. for use of the organoids in personalised medicine approaches in which the organoid is used to determine whether a particular patient of interest may be treatable using a certain compound (e.g. a drug).

In embodiments which describe a gene tagged with a detectable marker, the gene is tagged with a gene encoding the detectable marker.

The organoid used in a method of the invention or made by a method of the invention is preferably a human organoid. However, the use or production of organoids from other sources is also envisaged. For example, in some embodiments, the organoid is established from a mammal.

In some embodiments, the mammal is a small mammal, for example, a mouse, rat, hamster, guinea pig or rabbit.

Extracellular Matrix

In some embodiments, the steps of expanding and/or differentiating cells comprise culturing cells in contact with an extracellular matrix (ECM). Any suitable ECM may be used. Cells are preferably cultured in a microenvironment that mimics at least in part a cellular niche in which said cells naturally reside. A cellular niche is in part determined by the cells and by an ECM that is secreted by the cells in said niche. A cellular niche may be mimicked by culturing said cells in the presence of biomaterials or synthetic materials that provide interaction with cellular membrane proteins, such as integrins. An ECM as described herein is thus any biomaterial or synthetic material or combination thereof that mimics the in vivo cellular niche, e.g. by interacting with cellular membrane proteins, such as integrins.

In a preferred method of the invention, cells are cultured in contact with an ECM. “In contact” means a physical or mechanical or chemical contact, which means that for separating said resulting organoid or population of epithelial cells from said extracellular matrix a force needs to be used. In some embodiments, the ECM is a three-dimensional matrix. In some embodiment, the cells are embedded in the ECM. In some embodiments, the cells are attached to an ECM. A culture medium of the invention may be diffused into a three-dimensional ECM.

In another embodiments, the ECM is in suspension, i.e. the cells are in contact with the ECM in a suspension system. In some embodiments, the ECM is in the suspension at a concentration of at least 1%, at least 2% or at least 3%. In some embodiments, the ECM is in the suspension at a concentration of from 1% to about 10% or from 1% to about 5%. The suspension method may have advantages for upscale methods.

The culture medium and/or cells may be placed on, embedded in or mixed with the ECM.

Various ECM suitable for use in the invention are described in WO 2017/220586, which is hereby incorporated by reference. In some embodiments, the ECM is an ECM as described in WO 2017/220586.

In some embodiments, the organoids or cells are cultured in a medium comprising an integrin agonist with or without an ECM. In some embodiments, the integrin agonist is a stimulatory anti-integrin antibody. Examples of suitable anti-integrin antibodies include JBS2, HP1/3, SNAKA51, PTS25-2, PMI-1, MEM-83, NKI-L16, 496B, 12G10, 8A2, TS2/16, 15/7, HUTS-4, 8E3, N29, 9EG7, mAb 24, MEM-148, KIM127, CBR LFA-1/2, MEM-48, KIM185, AP3, AP5, LIBS6, LIBS2, 10F8, 2B8 and 2G3.

Components of the Culture Medium

In some embodiments, the culture medium comprises a receptor tyrosine kinase ligand. In the context of the invention, a receptor tyrosine kinase ligand is any ligand that activates a receptor tyrosine kinase (RTK). Many receptor tyrosine kinase ligands are mitogenic growth factors. Thus in some embodiments, the one or more receptor tyrosine kinase ligands comprises one or more mitogenic growth factor. There are approximately 20 different known classes of RTKs, including RTK class I (EGF receptor family) (ErbB family), RTK class II (Insulin receptor family), RTK class Ill (PDGF receptor family), RTK class IV (FGF receptor family), RTK class V (VEGF receptors family), RTK class VI (HGF receptor family), RTK class VII (Trk receptor family), RTK class VIII (Eph receptor family), RTK class IX (AXL receptor family), RTK class X (LTK receptor family), RTK class XI (TIE receptor family), RTK class XII (ROR receptor family), RTK class XIII (DDR receptor family), RTK class XIV (RET receptor family), RTK class XV (KLG receptor family), RTK class XVI (RYK receptor family), RTK class XVII (MuSK receptor family). In some embodiments, the one or more receptor tyrosine kinase ligands comprises ligands for one or more, or all of these 20 classes of RTKs. In some embodiments, the receptor tyrosine kinase ligand is EGF. For example, in some embodiments, EGF is used at about 1 ng/ml to 100 μg/ml, 10 ng/ml to 1 μg/ml, 20 ng/ml to 100 ng/ml, 30 ng/ml to 70 ng/ml, 40 ng/ml to 60 ng/ml, or 50 ng/ml.

In some embodiments, Wnt3A is recombinant human Wnt-3A. A soluble Wnt agonist, such as Wnt-3a, may be provided in the form of Wnt conditioned medium. For example, about 10% to about 70%, e.g. 40% to 60%, e.g. about 50% Wnt conditioned medium may be used.

In some embodiments, the culture medium comprises a BMP inhibitor. BMPs are small signalling molecules that bind to two classes of cell surface bone morphogenetic protein receptors (BMPR-I and BMPRII). The BMPR-I receptor class consists of three receptor types, activin receptor-like kinase-2 (ALK-2 or ActR-IA), ALK-3 (BMPR-IA) and ALK-6 (BMPR-IB). The BMPR-II receptor class is comprised of three receptor types, BMPR-II, ActR-IIA and ActR-IIB. Binding of BMPs results in the formation of heterotetrameric complexes containing two type I and two type II receptors. In addition to an extracellular binding domain, each BMP receptor contains an intracellular serine/threonine kinase domain. Following binding of BMPs, constitutively active type II receptor kinases phosphorylate type I receptor kinase domains that in turn phosphorylate BMP-responsive SMADs 1, 5, and 8, which can enter the cell nucleus and function as transcription factors. Phosphorylation of these specific SMADs results in various cellular effects, including growth regulation and differentiation. A BMP inhibitor is any inhibitor that results in a significant reduction in signaling via these pathways. For example, a BMP inhibitor may be able to disrupt the interaction of a BMP with a BMP receptor; bind to a BMP receptor and inhibit activation of downstream signalling; inhibit phosphorylation of Smad 1, Smad 5 or Smad 8; inhibit translocation of Smad 1, Smad 5 or Smad 8 to the nucleus; inhibit SMAD 1, SMAD 5 or SMAD 8 mediated transcription of target genes; or inhibit expression, folding or secretion of a BMP. In some embodiments, the BMP inhibitor reduces signaling via the BMPR-I receptor class. In some embodiments, the BMP inhibitor reduces signaling via BMPR-II receptor class. In some embodiments, the BMP inhibitor reduces signaling via SMAD 1/5/8. The inhibition may be direct or indirect.

Many BMP inhibitors are known in the art, e.g. as disclosed in Cuny, et al., (2008) Structure-activity relationship study of bone morphogenetic protein (BMP) signaling inhibitors. Bioorg Med Chem Lett 18: 4388-4392. Any of these BMP inhibitors are suitable for use in the methods of the invention. Methods for identifying suitable BMP inhibitors are known in the art. A suitable assay is described in Zilberberg et al., BMC Cell Biology 2007 8:41. Another suitable assay for a BMP inhibitor (in particular a BMP inhibitor that inhibits phosphorylation of Smad 1, 5 or 8 via ALK2 and ALK3) can be identified by a person skilled in the art using the cytobot cellular ELISA assay described in Cuny, et al., (2008) Structure-activity relationship study of bone morphogenetic protein (BMP) signaling inhibitors. Bioorg Med Chem Lett 18: 4388-4392.

In some embodiments the BMP inhibitor is selected from noggin, chordin, follistatin, gremlin, tsg (twisted gastrulation), sog (short gastrulation), dorsomorphin and LDN193189. In some embodiments, the BMP inhibitor is selected from:
a. noggin, sclerostin, chordin, CTGF, follistatin, gremlin, tsg, sog or an analog or variant thereof; and/or
b. dorsomorphin or LDN193189 or an analog or variant thereof.

In some preferred embodiments the BMP inhibitor is noggin. Noggin is particularly suitable for in vitro culture methods. In some embodiments, noggin is included in the culture medium at a final concentration of between 1 and 1000 ng/ml, between 10 and 1000 ng/ml, between 100 and 1000 ng/ml, between 1 and 500 ng/ml, between 1 and 200 ng/ml, between 1 and 100 ng/ml, between 10 and 500 ng/ml, between 20 and 500 ng/ml, between 10 and 200 ng/ml, between 20 and 200 ng/ml, between 50 and 500 ng/ml, or between 50 and 200 ng/ml. In some embodiments, noggin is included in the culture medium at a final concentration of about 100 ng/ml.

In some embodiments, the culture medium comprises a Wnt agonist. In some embodiments, the Wnt agonist is selected from any one of R-spondin 1-4 or a biologically active fragment or variant thereof. In some embodiments, the R-spondin is R-spondin 3. The Wnt agonist is preferably added to the media in an amount effective to stimulate a Wnt activity in a cell by at least 10%, more preferred at least 20%, more preferred at least 30%, more preferred at least 50%, more preferred at least 70%, more preferred at least 90%, more preferred at least 100%, relative to a level of said Wnt activity in the absence of said molecule, as assessed in the same cell type. As is known to a skilled person, Wnt activity can be determined by measuring the transcriptional activity of Wnt, for example by pTOPFLASH and pFOPFLASH Tcf luciferase reporter constructs (Korinek et al., 1997. Science 275:1784-1787).

R-spondin may be provided in the form of R-spondin conditioned medium or in the form of recombinant protein. Any suitable concentration of R-spondin may be used. For example, about 10 ng/ml to about 2.5 mg/ml, e.g. 50-1800 μg/ml, 180-1500 μg/ml, 10 ng/ml to about 1500 μg/ml, 50 μg/ml to about 1000 μg/ml, 150 μg/ml to about 750 μg/ml, 150 μg/ml to about 500 μg/ml, 200 μg/ml to about 350 μg/ml, or about 250 μg/ml. In some embodiments the R-spondin is used at a final concentration of at least 50 ng/ml, at least 100 ng/ml, at least 500 ng/ml, at least 1 μg/ml, at least 150 μg/ml. In some embodiments, R-spondin is used at a final concentration of about 250 μg/ml.

In some embodiments, the culture medium comprises a TGF-beta inhibitor. The TGF-beta inhibitor may be any agent that reduces the activity of the TGF-beta signalling pathway, preferably the signalling pathway that acts via Smad2 and/or Smad3, more preferably the signalling pathway that acts via ALK4, ALK5 or ALK7. In some embodiments the TGF-beta inhibitor is not a BMP inhibitor, i.e. the TGF-beta inhibitor is not Noggin.

There are many ways of disrupting the TGF-beta signalling pathway that are known in the art and that can be used in conjunction with this invention. For example, the TGF-beta signalling may be disrupted by: inhibition of TGF-beta expression by a small-interfering RNA strategy; inhibition of furin (a TGF-beta activating protease); inhibition of the pathway by physiological inhibitors; neutralisation of TGF-beta with a monoclonal antibody; inhibition with small-molecule inhibitors of TGF-beta receptor kinase 1 (also known as activin receptor-like kinase, ALK5), ALK4, ALK6, ALK7 or other TGF-beta-related receptor kinases; inhibition of Smad 2 and Smad 3 signalling e.g. by overexpression of their physiological inhibitor, Smad 7, or by using thioredoxin as an Smad anchor disabling Smad from activation (Fuchs, O. Inhibition of TGF-Signalling for the Treatment of Tumor Metastasis and Fibrotic Diseases. Current Signal Transduction Therapy, Volume 6, Number 1, January 2011, pp. 29-43(15)).

Various methods for determining if a substance is a TGF-beta inhibitor are known and might be used in conjunction with the invention. For example, a cellular assay may be used in which cells are stably transfected with a reporter construct comprising the human PAI-1 promoter or Smad binding sites, driving a luciferase reporter gene. Inhibition of luciferase activity relative to control groups can be used as a measure of compound activity (De Gouville et al. (2005) Br J Pharmacol. 145(2): 166-177). New TGF-beta inhibitors can therefore be easily identified by the skilled person in the art.

A TGF-beta inhibitor according to the present invention may, for example, be a protein, peptide, small-molecules, small-interfering RNA, antisense oligonucleotide, aptamer or antibody. The inhibitor may be naturally occurring or synthetic. In one embodiment, the TGF-beta inhibitor is an inhibitor of ALK4, ALK5 and/or ALK7. For example, the TGF-beta inhibitor may bind to and directly inhibit ALK4, ALK5 and/or ALK7. Examples of preferred small-molecule TGF-beta inhibitors that can be used in the context of this invention include but are not limited to the small molecule inhibitors listed in Table 1 below.

In some embodiments, the TGF-beta inhibitor is a small molecule inhibitor optionally selected from the group consisting of: A83-01, SB-431542, SB-505124, SB-525334, LY 364947, SD-208 and SJN 2511.

In some embodiments, no more than one TGF beta inhibitor is present in the culture medium. In other embodiments, more than one TGF beta inhibitor is present in the culture medium, e.g. 2, 3, 4 or more. In some embodiments, a culture medium of the invention comprises one or more of any of the inhibitors listed in Table 1. A culture medium may, for example, comprise any combination of one inhibitor with another inhibitor listed. For example, a medium may comprise SB-525334 or SD-208 or A83-01; or SD-208 and A83-01. The skilled person will appreciate that a number of other small-molecule inhibitors exist that are primarily designed to target other kinases, but at high concentrations may also inhibit TGF-beta receptor kinases. For example, SB-203580 is a p38 MAP kinase inhibitor that, at high concentrations (for example, approximate 10 μM or more) is thought to inhibit ALK5. Any such inhibitor that inhibits the TGF-beta signalling pathway can also be used in the context of this invention.

TABLE 1
Small-molecule TGF-beta inhibitors targeting receptor kinases

| | | IC50 | | | |
|---|---|---|---|---|---|
| Inhibitor | Targets | (nM) | Mol Wt | Name | Formula |
| A83-01 | ALK5 | 12 | 421.52 | 3-(6-Methyl-2-pyridinyl)-N- | C25H19N5S |
| | (TGF-βR1) | | | phenyl-4-(4-quinolinyl)-1H- | |
| | ALK4 | 45 | | pyrazole-1-carbothioamide | |
| | ALK7 | 7.5 | | | |
| SB-431542 | ALK5 | 94 | 384.39 | 4-[4-(1,3-benzodioxol-5-yl)-5-(2- | C22H16N4O3 |
| | ALK4 | | | pyridinyl)-1H-imidazol-2- | |
| | ALK7 | | | yl]benzamide | |
| SB-505124 | ALK5 | 47 | 335.4 | 2-(5-benzo[1,3]dioxol-5-yl-2-tert- | C20H21N3O2 |
| | ALK4 | 129 | | butyl-3Himidazol- | |
| | | | | 4-yl)-6-methylpyridine | |
| | | | | hydrochloride hydrate | |
| SB-525334 | ALK5 | 14.3 | 343.42 | 6-[2-(1,1-Dimethylethyl)-5-(6- | C21H21N5 |
| | | | | methyl-2-pyridinyl)-1H-imidazol- | |
| | | | | 4-yl]quinoxaline | |
| SD-208 | ALK5 | 49 | 352.75 | 2-(5-Chloro-2-fluorophenyl)-4- | C17H10ClFN6 |
| | | | | [(4-pyridyl)amino]pteridine | |
| LY-36494 | TGR-βRI | 59 | 272.31 | 4-[3-(2-Pyridinyl)-1H-pyrazol-4- | C17H12N4 |
| | TGF-βRII | 400 | | yl]-quinoline | |
| | MLK-7K | 1400 | | | |
| SJN-2511 | ALK5 | 23 | 287.32 | 2-(3-(6-Methylpyridine-2-yl)-1H- | C17H13N5 |
| | | | | pyrazol-4-yl)-1,5-naphthyridine | |

In some embodiments, the TGF-beta inhibitor (e.g. A83-01) is present in the culture medium at at least 1 nM for example, at least 5 nM, at least 50 nM, at least 100 nM, at least 300 nM, at least 450 nM or at least 475 nM. For example, the TGF-beta inhibitor (e.g. A83-01) is present in the culture medium at 1 nM-200 μM, 10 nM-200 μM, 100 nM-200 μM, 1 μM-200 μM, 10 nM-100 μM, 50 nM-100 μM, 50 nM-10 μM, 100 nM-1 μM, 200 nM-800 nM, 350-650 nM or at about 500 nM. Accordingly, in some embodiments, the culture medium comprises A83-01 at a concentration of about 500 nM.

In some embodiments, the culture medium comprises a p38 inhibitor. A p38 inhibitor is any inhibitor that, directly or indirectly, negatively regulates p38 signalling. In some embodiments, a p38 inhibitor according to the invention binds to and reduces the activity of p38 (GI number 1432). A p38 inhibitor is an agent that binds to and reduces the activity of at least one p38 isoform. Various methods for determining if a substance is a p38 inhibitor are known, and might be used in conjunction with the invention. Examples include: phospho-specific antibody detection of phosphorylation at Thr180/Tyr182, which provides a well-established measure of cellular p38 activation or inhibition; biochemical recombinant kinase assays; tumor necrosis factor alpha (TNFα) secretion assays; and DiscoverRx high throughput screening platform for p38 inhibitors (see http://www.discoverx.com/kinases/Iiterature/biochemical/collaterals/DRx_poster_p38%20KBA.pdf). Several p38 activity assay kits also exist (e.g. Millipore, Sigma-Aldrich).

Various p38 inhibitors are known in the art. In some embodiments, the inhibitor that directly or indirectly negatively regulates p38 signalling is selected from the group consisting of SB-202190, SB-203580, VX-702, VX-745, PD-169316, RO-4402257 and BIRB-796.

In some embodiments, the p38 inhibitor according to the invention binds to and reduces the activity of its target by more than 10%; more than 30%; more than 60%; more than 80%; more than 90%; more than 95%; or more than 99% compared to a control, as assessed by a cellular assay. Examples of cellular assays for measuring target inhibition are well known in the art as described above.

SB-203580 may, for example, be added to the culture medium at a concentration of between 50 nM and 100 μM, or between 100 nM and 50 μM, or between 1 μM and 50 μM. For example, SB-203580 may be added to the culture medium at approximately 30 μM.

In some embodiments, the culture medium of the invention further comprises gastrin. In some embodiments, the culture medium of the invention comprises gastrin at a concentration of 0.01-500 nM, 0.1-100 nM, 1-100 nM, 1-20 nM or 5-15 nM. For example, in some embodiments, the culture medium of the invention comprises gastrin at a concentration of about 10 nM.

In some embodiments, a method described herein comprises use of a Wnt inhibitor, e.g. in a culture medium. A Wnt inhibitor is defined as an agent that inhibits TCF/LEF-mediated transcription in a cell or in a population of cells. Accordingly, Wnt inhibitors suitable for use in the invention include:
(1) inhibitors of Wnt secretion (e.g. inhibitors of Porc, such as LGK974, IWP-1 or IWP-2),
(2) competitive and non-competitive inhibitors of the interaction between Wnt or R-spondin and their respective receptors (e.g. OMP-18R5, OMP54F28),
(3) factors that promote the degradation of components of the Wnt receptor complex, such as LRP (e.g. niclosamide) and factors that promote the degradation of R-spondin receptors, such as Znrf3 and/or Rnf43 or factors that activate Znrf3 and/or Rnf43,
(4) inhibitors of Dishevelled family proteins, such as inhibitors that reduce the binding of Dishevelled family proteins to Frizzled receptors and/or components of the destruction complex (e.g. Dapper family proteins, FJ9, sulindac, 3289-8625, J01-017a, NSC668036) or inhibitors that downregulate the expression of Dishevelled family proteins (e.g. niclosamide),
(5) factors that promote destruction complex activity, including (a) inhibitors of phosphatases (e.g. PP1, PP2A and/or PP2C) that dephosphorylate components of the destruction complex, such as axin and/or APC (e.g. okadaic acid or tautomycin) and (b) inhibitors of kinases (e.g. p38 MAPK, PKA, PKB, PKC, p90RSK or p70S6K) that phosphorylate GSK-3 (e.g. SB239063, SB203580 or Rp-8-Br-cAMP),
(6) inhibitors of the deoligomerisation of the destruction complex, such as inhibitors of Tankyrases 1 and/or 2 (e.g. XAV939, IWR1, JW74, JW55, 2-[4-(4-fluorophenyl)piperazin-1-yl]-6-methylpyrimidin-4(3H)-one or PJ34), and
(7) inhibitors of β-catenin target gene expression, including inhibitors of the β-catenin:TCF/Lef transcription complex, such as inhibitors that disrupt the β-catenin:TCF-4 complex (e.g. iCRT3, CGP049090, PKF118310, PKF115-584, ZTM000990, PNU-74654, BC21, iCRT5, iCRT14 or FH535) and inhibitors of the histone deacetylase SIRT1 (e.g. cambinol).

Any suitable Wnt inhibitor may be used as described in (1)-(7) above. For instance, in one preferred embodiment, the Wnt inhibitor is an inhibitor of Wnt secretion, such as a Porc inhibitor, e.g. selected from IWP-2, IWP-1 and LGK974. In another preferred embodiment, the Wnt inhibitor is an inhibitor of β-catenin target gene expression, for example, an inhibitor of the β-catenin:TCF/Lef transcription complex or an inhibitor of the histone deacetylase SIRT1 (e.g. cambinol). In some embodiments, the inhibitor of the β-catenin:TCF/Lef transcription complex is an inhibitor that disrupts the β-catenin:TCF-4 complex, for example an inhibitor selected from iCRT3, CGP049090, PKF118310, PKF115-584, ZTM000990, PNU-74654, BC21, iCRT5, iCRT14 and FH535.

The Wnt inhibitor is preferably added to the media in an amount effective to inhibit a Wnt activity in a cell by at least 10%, more preferred at least 20%, more preferred at least 30%, more preferred at least 50%, more preferred at least 70%, more preferred at least 90%, more preferred 100%, relative to a level of said Wnt activity in the absence of said molecule, as assessed in the same cell type. Wnt activity can be determined by measuring the transcriptional activity of Wnt, for example by pTOPFLASH and pFOPFLASH Tcf luciferase reporter constructs (Korinek et al. (1997) Science 275:1784-1787). Wnt inhibitors can therefore easily be identified by a skilled person using an assay known in the art.

In some embodiments, the expansion medium and/or the differentiation medium comprises a BMP pathway activator. Methods for identifying suitable BMP activators are known in the art. A suitable assay for measuring BMP activity is described in Zilberberg et al., BMC Cell Biology 2007 8:41.

In some embodiments, the BMP pathway activator is selected from BMP7, BMP4 and BMP2. BMP7 is preferred. BMP7 induces the phosphorylation of SMAD1 and SMAD5. Thus in some embodiments, the BMP pathway activator is any compound that is capable of inducing the phosphorylation of SMAD1 and SMAD5. In addition, where BMP7 is mentioned, any compound that induces the phosphorylation of SMAD1 or SMAD5 can be used instead of BMP7.

In some embodiments, the BMP pathway activator, such as BMP4 or BMP7 is present in the culture medium at at least 0.01 ng·ml, at least 0.1 ng/ml, at least 1 ng/ml, at least 10 ng/ml, at least 20 ng/ml, at least 25 ng/ml, at least 100 ng/ml, at least 500 ng/ml, at least 1 μg/ml, at least 10 μg/ml or at least 50 μg/ml. In some embodiments, the BMP pathway activator, such as BMP4 or BMP7 is present in the culture medium from about 0.01 ng/ml to about 500 ng/ml, from about 1 ng/ml to about 500 ng/ml, from about 10 ng/ml to about 500 ng/ml, from about 20 ng/ml to about 500 ng/ml. In some embodiments, the BMP pathway activator, such as BMP4 or BMP7, is present in the culture medium from about 0.01 ng/ml to about 200 ng/ml, from about 0.1 ng/ml to about 100 ng/ml, from about 1 ng/ml to about 100 ng/ml, from about 10 ng/ml to about 100 ng/ml, from about 10 ng/ml to about 50 ng/ml, from about 15 ng/ml to about 30 ng/ml. In some embodiments, the BMP pathway activator, such as BMP4 or BMP7 is present in the culture medium at about 25 ng/ml. In some embodiments, BMP4 is present in the culture medium from about 0.1 μg/ml to about 50 μg/ml, from about 1 to about 50 μg/ml, from about 5 μg/ml to about 25 μg/ml or from about 5 μg/ml to about 15 μg/ml. In some embodiments, BMP4 is present in the culture medium at about 10 μg/ml.

Methods for Affecting Differentiation of EECs

The inventors have found that the EEC cell types and/or the hormones expressed by the EECs can be altered by affecting various signalling pathways during the differentiation process. Such methods represent standalone aspects of the invention. They may also be combined with the methods described herein to make intestinal organoids and/or intestinal reporter organoids having altered subtypes and/or altered ratios of subtypes of EECs compared to EECs present in the region of the intestine from which the organoid was established. Similarly, such methods may be combined with the methods described herein to make intestinal organoids and/or intestinal reporter organoids which express different hormones and/or different levels of hormones compared to the types of hormones and/or levels of hormones produced by EECs present in the region of the intestine from which the organoid was established.

The inventors have surprisingly found that inhibiting Wnt signalling before the step of differentiating the stem cells and/or cells with stem cell potential to EECs (e.g. by overexpression of the inducible transcription factor) stimulates expression of Motilin (MLN). Wnt signalling may be inhibited by any suitable method, for example, by a Wnt inhibitor as described herein, for example, treatment with a Porcupine inhibitor such as IWP-2 (Stemgent, 5 μM). Further Wnt inhibitors that may be used in the invention are described in WO 2017/220586 and elsewhere herein. Thus, in some embodiments, the method comprises inhibiting Wnt signalling before differentiating stem cells and/or cells with stem cell potential to EECs (e.g. by overexpressing the inducible transcription factor, e.g. NEUROG3). The invention therefore further provides a method for stimulating expression of MLN in an intestinal organoid, which comprises inhibiting Wnt signalling in the organoid before differentiating the stem cells and/or cells with stem cell potential to EECs (e.g. by overexpressing the inducible transcription factor, e.g. NEUROG3). In some embodiments, the intestinal organoid is an organoid as described herein, for example, a reporter organoid. The methods for making intestinal organoids and reporter organoids described herein can be adapted accordingly to contain such a step, if desired.

For example, in some embodiments, the method for enriching the population of EECs in an intestinal organoid, comprises the steps of:
i. contacting an intestinal stem cell and/or an intestinal cell with stem cell potential with a construct comprising an inducible transcription factor for differentiating intestinal stem cells and/or intestinal cells with stem cell potential to EECs;
ii. generating one or more intestinal organoids by culturing the cells in an expansion medium;
iii. inhibiting Wnt signalling in the organoid; and
iv. culturing the one or more intestinal organoids in an expansion medium and/or a differentiation medium and overexpressing NEUROG3.

For example, in some embodiments, inhibiting Wnt signalling comprises adding a Wnt inhibitor to the expansion medium (e.g. used in step ii in the method above) after the one or more intestinal organoids have been generated. In some embodiments, the step of inhibiting Wnt signalling in an organoid comprises culturing the one or more organoids in an expansion medium comprising a Wnt inhibitor. In some embodiments, the expansion medium used in step iii is the same as the expansion medium used in step ii. In some embodiments, the expansion medium used in step iii is different from the expansion medium used in step ii. In some embodiments, step iii comprises culturing the one or more organoids for 2 to 3 days, for example, for 2 days. These methods can also apply to methods for making a reporter organoid, mutatis mutandis.

The invention also comprises methods in which Wnt signalling is not inhibited before the differentiation step.

In some embodiments, the methods described herein which involve inhibiting Wnt signalling before the differentiation step additionally or alternatively decrease expression of GCG. In some embodiments, the level of GCG is decreased relative to an organoid prepared by the same method but in which Wnt signalling has not been inhibited before the differentiation step. Accordingly, the invention provides a method for decreasing GCG expression in an intestinal organoid comprising inhibiting Wnt signalling before differentiating stem cells and/or cells with stem cell potential to EECs. In some embodiments, the intestinal organoid is an organoid as described herein, for example, a reporter organoid.

In some embodiments, the number of MLN-producing cells is increased relative to an organoid prepared by the same method but in which Wnt signalling has not been inhibited before the differentiation step. Accordingly, the invention provides a method for increasing the number of MLN-producing cells in an intestinal organoid comprising inhibiting Wnt signalling before differentiating stem cells and/or cells with stem cell potential to EECs.

In some embodiments, inhibiting Wnt signalling before the differentiation step results in a shift in the ratio between L-cells and M-X cells to decrease the number of L-cells and increase the number of M-X cells. L-cells secrete GLP-1. M-X cells secrete Ghrelin and Mln. GCG is known to be cleaved in vivo into a number of peptides including GLP-1. In some embodiments, inhibiting Wnt signalling before overexpressing the inducible transcription factor (e.g. NEUROG3) shifts the ratio of EECs from GLP1⁺L-cells to MLN⁺GHRL+M-X cells to decrease the number of GLP1⁺L-cells and increase the number of MLN⁺ GHRL+ M-X cells. Thus, the invention similarly provides a method for decreasing the number of GLP1⁺ L-cells and increasing the number of MLN⁺ GHRL+M-X cells comprising the steps described herein.

In some embodiments, the intestinal stem cells and/or intestinal cells with stem cell potential are present in a cell culture which is not an organoid. In other embodiments, they are present in an intestinal organoid. In some embodiments, the organoid is an intestinal organoid which is not an organoid of the invention. For example, in some embodiments, the intestinal organoid is not a reporter organoid. In some embodiments, the intestinal organoid does not comprise an inducible transcription factor for differentiating intestinal stem cells and/or intestinal cells with stem cell potential to EECs. The methods describing inhibiting Wnt signalling may be adapted for such embodiments mutatis mutandis.

The inventors have also found that withdrawal of Noggin from the ENR medium and addition of BMP induces villus-restricted hormones. Thus, in some embodiments, the step of differentiating stem cells and/or cells with stem cell potential to EECs is carried out in the presence of a BMP pathway activator and in the absence of Noggin. In some embodiments, the step is carried out in the presence of a BMP pathway activator and in the absence of a BMP inhibitor. In some embodiments, the BMP pathway activator is present throughout the differentiation step including when EECs are present. In some embodiments, the BMP pathway activator is added during the differentiation step and is present when EECs are present. In some embodiments, the BMP pathway activator is added after the intestinal stem cells and/or cells with stem cell potential have differentiated to EECs. Noggin (or in some embodiments a BMP inhibitor) is preferably not present when the BMP pathway activator is added.

Accordingly, the invention provides a method for stimulating expression of one or more villus-restricted hormones and/or decreasing expression of one or more crypt-restricted hormones in EECs, comprising activating BMP signalling in the EECs. For example, the step of activating BMP signalling in the EECs may comprise contacting EECs with a BMP pathway activator in the absence of Noggin (or in the absence of a BMP inhibitor). The step of contacting EECs with a BMP pathway activator in the absence of Noggin preferably comprises culturing the EECs in the presence of a BMP pathway activator and in the absence of Noggin (i.e. by using a culture medium comprising a BMP pathway activator which does not comprise Noggin). In some embodiments, the EECs are present in an intestinal organoid. In some embodiments, the EECs are present in a population of cells which is not an organoid. For example, in some embodiments, the EECs are obtained by or are obtainable by dissociating an organoid of the invention, and then the EECs are contacted with the BMP pathway activator. In some embodiments, the method for making an intestinal organoid and/or the method for making a reporter organoid as described herein may be adapted accordingly to include this step, if desired. For example, the method may comprise adding a BMP pathway activator to the culture medium during or after the differentiation step in order to contact EECs with the BMP pathway activator. In some embodiments, the method may comprise differentiating stem cells and/or cells with stem cell potential to EECs in the presence of a BMP pathway activator and in the absence of Noggin (or in the absence of a BMP inhibitor) so that BMP signalling is activated in the EECs.

As mentioned elsewhere herein, the cells (e.g. the stem cells, cells with stem cell potential and/or EECs) are preferably human cells.

In some embodiments, the one or more villus-restricted hormones comprise one or more of (e.g. 1, 2, 3 or more or all 4 of) PYY, NTS, SCT and MLN. In some embodiments, the one or more villus-restricted hormones are MLN and/or SCT. In some embodiments, the one or more crypt-restricted hormones is GCG.

For example, in some embodiments, the method for stimulating expression of one or more villus-restricted hormones and/or decreasing expression of one or more crypt-restricted hormones comprises differentiating stem cells and/or cells with stem cell potential to EECs in one or more intestinal organoids, e.g. by overexpressing an inducible transcription factor for differentiating intestinal stem cells and/or intestinal cells with stem cell potential to EEC, while culturing the one or more intestinal organoids in an expansion medium and/or a differentiation medium, wherein the expansion medium and/or differentiation medium comprises a BMP pathway activator but does not comprise Noggin. In some embodiments, the expansion medium and/or differentiation medium does not comprise a BMP inhibitor. The various expansion media and differentiation media described herein may be adapted accordingly.

In some embodiments, the differentiating comprises culturing the one or more intestinal organoids in an ENR differentiation medium which does not comprise Noggin but which comprises a BMP pathway activator.

Any suitable BMP pathway activator may be used in a method of the invention, for example, as described herein. In some embodiments, the BMP pathway activator is BMP-2 and/or BMP-4. In some embodiments the medium comprises BMP-2 (Peprotech, for example, at 30-70 ng/ml, 40-60 ng/ml, or about 50 ng/ml) and BMP-4 (Peprotech, for example, at 30-70 ng/ml, 40-60 ng/ml, or about 50 ng/ml).

In some embodiments, a method for making an intestinal organoid, e.g. a reporter organoid, comprises differentiating the stem cells and/or cells with stem cell potential to EECs in the absence of a BMP pathway activator. In some embodiments, a method of making an intestinal organoid (e.g. a reporter organoid) comprises differentiating the stem cells and/or cells with stem cell potential to EECs in the presence of a BMP pathway activator. For example, in some embodiments, the transcription factor is overexpressed in the absence of a BMP pathway activator. In some embodiments, the transcription factor is overexpressed in the presence of a BMP pathway activator. The presence or absence of a BMP pathway activator determines whether the crypt- or villus-versions of EECs are generated. BMP signals change the ratio of EEC hormones. Some hormones are only made in the presence of a BMP pathway activator, and some hormones are only made in the absence of a BMP pathway activator. Depending on which hormone is of interest, these signals can be modulated depending on whether a BMP pathway activator is present.

Similarly, the method may be used to enhance the expression of NTS in human L-cells while reducing production of GLP-1 and/or GCG. Again, this is achieved by activating BMP signalling in the human L-cells. In some embodiments, activating BMP signalling comprises contacting the human L-cells with a BMP pathway activator. In some embodiments, the contacting comprises contacting an intestinal organoid comprising L-cells with a BMP pathway activator. In some embodiments, the intestinal organoid is an organoid of the invention which comprises L-cells. In some embodiments, the intestinal organoid is a reporter organoid. For example, the BMP pathway activator may be present in the expansion medium and/or the differentiation medium described herein.

The inventors have shown previously that BMP signalling regulates differential hormone expression within the same murine EEC subtype, an observation that is extended here to human EECs for the hormone pair Ghrelin/Motilin for the first time. The inventors now show that BMP signalling regulates differential hormone expression within the same human EEC subtype. The inventors have surprisingly found that the ratio between Ghrelin and Motilin secretion in human M-X cells depends on BMP levels. The inventors have found that BMP represses Ghrelin secretion but enhances Motilin secretion in human EECs, in particular in human M-X cells. Thus, human MLN/GHRL-producing EECs appeared to undergo a BMP-controlled switch in hormone expression.

Thus, the invention provides a method for increasing Motilin expression and/or secretion in human EECs (preferably in M-X cells) which comprises activating BMP signalling in the human EECs. Similarly, the invention provides a method for decreasing Ghrelin expression and/or secretion in human EECs (preferably in M-X cells) which comprises activating BMP signalling in the human EECs. In addition, the invention provides a method for regulating differential hormone expression within a human EEC subtype comprising activating BMP signalling in the human EEC subtype. In particular, the invention provides a method for increasing Motilin expression and/or secretion and decreasing Ghrelin expression and/or secretion in human EECs (preferably in human M-X cells) comprising activating BMP signalling in the human EECs. In some embodiments, activating BMP signalling comprises contacting the human EECs with a BMP pathway activator. In some embodiments, the contacting comprises contacting an intestinal organoid comprising M-X cells with a BMP pathway activator. In some embodiments, the intestinal organoid is an organoid of the invention, for example, a reporter organoid. In some embodiments, the contacting comprises contacting human M-X cells with a BMP pathway activator. In some embodiments, the EECs are present in a population of cells which is not an organoid. In some embodiments, the human EECs are obtained by or are obtainable by dissociating an organoid of the invention, and then the EECs are contacted with the BMP pathway activator. For example, the contacting may comprise culturing the EECs or the organoid comprising EECs in the presence of a BMP pathway activator (i.e. by using a culture medium comprising a BMP pathway activator). In some embodiments, the contacting comprises administering a BMP pathway activator to a patient. In some embodiments, activating BMP signalling comprises contacting the human EECs with a BMP pathway activator. In some embodiments, the BMP pathway activator is BMP.

Motilin is associated with gut motility disorders, e.g. bowel movement disorders, Parkinson's disease and gastroparesis. A Motilin agonist (erythromycin) is clinically used to stimulate peristalsis. Thus, increasing Motilin expression would be advantageous for treatment of these conditions. Accordingly, the invention provides a method for treating a disease or disorder in which Motilin is implicated comprising administering a BMP pathway activator to a patient. For example, the invention provides a method for treating a gut motility disorder (e.g. a bowel movement disorder, Parkinson's disease or gastroparesis) comprising administering a BMP pathway activator to a patient. Similarly, there is provided a BMP pathway activator for use in treating a disease or disorder in which Motilin is implicated. For example, there is provided a BMP pathway activator for use in treating a gut motility disorder, e.g. as described herein. Motilin is also associated with hunger. Thus, also provided is a method for treating or preventing a disease or disorder requiring appetite stimulation comprising administering a BMP pathway activator to a patient. In some embodiments, the disease or disorder is anorexia. In some embodiments, the disease or disorder is cancer cachexia. In addition, Motilin is associated with diabetes, in particular type II diabetes. Thus, also provided is a method for treating or preventing diabetes comprising administering a BMP pathway activator to a patient. In some embodiments, the diabetes is type II diabetes. Motilin is also associated with increased gallbladder emptying. Thus, also provided is a method for treating or preventing a biliary movement disorder comprising administering a BMP pathway activator to a patient. For example, the invention provides a method for treating or preventing biliary diskinesia comprising administering a BMP pathway activator to a patient. Also provided is a method for treating or preventing a disease or disorder requiring appetite inhibition comprising administering a BMP pathway inhibitor to a patient. In some embodiments, the disease or disorder is obesity. In some embodiments, the disease or disorder is diabetes. In some embodiments, the disease or disorder is type II diabetes. In some embodiments, the disease or disorder is Prader-Willi syndrome. The BMP pathway activator may, for example, be formulated in a pharmaceutical composition, e.g. as described herein. For example, in some embodiments, the pharmaceutical composition is suitable for administration or delivery to the gut. Examples of BMP pathway activators and BMP pathway inhibitors are provided herein and may be used in the invention. In some embodiments, the BMP pathway activator is BMP. In some embodiments, the BMP pathway inhibitor is LDN193189 or an analogue or variant thereof.

The invention likewise provides a method for increasing Motilin expression and/or secretion and/or decreasing Ghrelin expression and/or secretion in human EECs (preferably in human M-X cells) comprising or consisting of activating BMP signalling in EECs by differentiating stem cells and/or cells with stem cell potential to EECs in one or more intestinal organoids, e.g. by overexpressing an inducible transcription factor for differentiating intestinal stem cells and/or intestinal cells with stem cell potential to EEC, while culturing the one or more intestinal organoids in an expansion medium and/or a differentiation medium, wherein the expansion medium and/or differentiation medium comprises a BMP pathway activator. In some embodiments, the BMP pathway activator is present throughout the differentiation step including when EECs are present in the organoid. In some embodiments, the BMP pathway activator is added during the differentiation step and is present when EECs are present in the organoid. In some embodiments, the BMP pathway activator is added after the intestinal stem cells and/or cells with stem cell potential have differentiated to EECs.

As mentioned above, any suitable BMP pathway activator may be used, for example, as described herein. In some embodiments, the BMP pathway activator is BMP.

Organoids

The invention provides a reporter organoid comprising one or more (e.g. 1, 2, 3, 4 or more, e.g. 2 or more) EEC-specific genes tagged at its endogenous locus with a detectable marker. The detectable marker is preferably a fluorescent marker, in particular a fluorescent protein. In some embodiments, the EEC-specific genes are tagged in stem cells and/or cells with stem cell potential in the reporter organoid. For example, in some embodiments, there is provided a reporter organoid which is a stable reporter organoid line comprising stem cells and/or cells with stem cell potential in which one or more EEC-specific genes is tagged at its endogenous locus with a detectable marker. In some embodiments, the EEC-specific genes are tagged in EECs in the reporter organoid. For example, there is provided a reporter organoid comprising EECs in which one or more EEC-specific genes is tagged at its endogenous locus with a detectable marker. For example, the reporter organoid may be a differentiated reporter organoid.

Also provided is a reporter organoid comprising one or more (e.g. 1, 2, 3, 4, or more, e.g. 2 or more) proteins encoded by EEC-specific genes tagged with a detectable marker as a fusion protein. In embodiments in which the protein encoded by the EEC-specific gene is tagged with a detectable marker as a fusion protein, the EEC-specific gene preferably encodes a hormone or hormone precursor. In some embodiments, the reporter organoid comprises one or more proteins encoded by EEC-specific genes and one or more detectable markers, wherein the protein encoded by the EEC-specific gene and the detectable marker do not form a fusion protein.

An EEC-specific gene tagged with a detectable marker is also referred to herein as a “tagged EEC-specific gene”. For example, a hormone tagged with a detectable marker is also referred to herein as a “tagged hormone”. A hormone synthesizing enzyme tagged with a detectable marker is also referred to herein as a “tagged hormone synthesizing enzyme”. A hormone precursor tagged with a detectable marker is also referred to herein as a “tagged hormone precursor”.

The EEC-specific gene may be any EEC-specific gene as described herein. For example, in some embodiments, a reporter organoid comprises one or more hormones, hormone precursors and/or hormone synthesizing enzymes tagged with a detectable marker. In some embodiments, a reporter organoid comprises one or more genes encoding a hormone, hormone precursor and/or hormone synthesizing enzyme tagged at its endogenous locus with a detectable marker gene. In some embodiments, a reporter organoid comprises one or more genes encoding a transcription factor for differentiating intestinal stem cells and/or intestinal cells with stem cell potential to EECs tagged at its endogenous locus with a detectable marker. In some embodiments, a reporter organoid comprises one or more transcription factors tagged with a detectable marker.

Embodiments which describe tagged proteins may also be applied to embodiments which describe tagging at the gene level, where appropriate, mutatis mutandis. Similarly, embodiments which describe tagging at the gene level may also be applied to embodiments which describe tagged proteins, where appropriate, mutatis mutandis.

Preferably, the reporter organoid is obtained by or is obtainable by a method of making a reporter organoid of the invention as described herein. Thus, the invention further provides a reporter organoid obtained by or obtainable by a method of making a reporter organoid of the invention as described herein. The description of the various features of the reporter organoids recited in the methods for making reporter organoids provided herein may be extrapolated to the reporter organoids provided by the invention, mutatis mutandis.

In some embodiments, a reporter organoid of the invention comprises a tagged hormone and a tagged hormone synthesizing enzyme; a tagged hormone and a tagged hormone precursor; or a tagged hormone synthesizing enzyme and a tagged hormone precursor. Tagging a hormone synthesizing enzyme or a hormone precursor may be of interest, for example, in embodiments in which there is a hormone that is not encoded by a gene but is instead synthesized using a hormone synthesizing enzyme or is derived from a hormone precursor, respectively.

In some embodiments, a reporter organoid comprises a tagged transcription factor (e.g. a transcription factor for differentiating intestinal stem cells and/or cells with stem cell potential to EECs) and a tagged hormone, tagged hormone precursor and/or tagged hormone synthesizing enzyme. Such a reporter organoid may be useful for studying the differential timing of expression of the tagged transcription factor in relation to expression of the tagged hormone, tagged hormone precursor and/or tagged hormone synthesizing enzyme.

In some embodiments, a reporter organoid of the invention comprises intestinal stem cells and/or intestinal cells with stem cell potential that have not been differentiated to EECs. For example, in some embodiments, the reporter organoid does not comprise EECs. In some embodiments, the intestinal stem cells and/or intestinal cells with stem cell potential in the reporter organoid have already been differentiated to EECs. In some embodiments, a reporter organoid of the invention is an organoid in which at least 10% of the cells are EECs. In some embodiments, a reporter organoid of the invention is an organoid in which at least 50% of the cells are EECs.

The reporter organoid of the invention is an intestinal organoid.

Also provided by the invention is an intestinal organoid as described herein. For example, there is provided an intestinal organoid which comprises an inducible transcription factor for differentiating intestinal stem cells and/or intestinal cells with stem cell potential to EECs. Likewise, also provided is an intestinal organoid obtained by or obtainable by a method of the invention. In some embodiments, the organoid has been obtained by or is obtainable by a method for enriching the population of EECs in an intestinal organoid, as described herein. In some embodiments, the intestinal organoid is a reporter organoid.

As mentioned above, in some embodiments, an intestinal organoid or a reporter organoid as provided herein comprises an inducible transcription factor for differentiating intestinal stem cells and/or intestinal cells with stem cell potential to EECs. In some embodiments, the inducible transcription factor is present in stem cells and/or in cells with stem cell potential in the organoid. In some embodiments, the inducible transcription factor is present in EECs in the organoid. For example, there is provided a reporter organoid or an intestinal organoid comprising stem cells and/or cells with stem cell potential which comprise an inducible transcription factor for differentiating intestinal stem cells and/or intestinal cells with stem cell potential to EECs. For example, in some embodiments, the reporter organoid comprises stem cells and/or cells with stem cell potential which comprise an inducible transcription factor for differentiating intestinal stem cells and/or intestinal cells with stem cell potential to EECs and in which one or more EEC-specific genes is tagged at its endogenous locus with a detectable marker in the stem cells and/or cells with stem cell potential. In some embodiments, the organoid is a clonal intestinal organoid which comprises stem cells and/or cells with stem cell potential which comprise an inducible transcription factor for differentiating intestinal stem cells and/or intestinal cells with stem cell potential to EECs. In some embodiments, the intestinal organoid is not enriched for EECs. For example, in some embodiments, an organoid of the invention does not comprise EECs. In some embodiments, the intestinal organoid is enriched for EECs. For example, in some embodiments, an organoid of the invention comprises EECs. In some embodiments, the intestinal organoid comprising the inducible transcription factor is a reporter organoid as described herein. In some embodiments, the reporter organoid comprises EECs which comprise an inducible transcription factor for differentiating intestinal stem cells and/or intestinal cells with stem cell potential to EECs and in which one or more EEC-specific genes is tagged at its endogenous locus with a detectable marker in the EECs. In some embodiments, the intestinal organoid comprises EECs which comprise the inducible transcription factor for differentiating intestinal stem cells and/or intestinal cells with stem cell potential to EECs. In some embodiments, the intestinal organoid does not comprise one or more (e.g. 1, 2, 3, 4 or more, e.g. 2 or more) EEC-specific genes tagged with a detectable marker. In some embodiments, the intestinal organoid does not comprise one or more (e.g. 1, 2, 3, 4 or more, e.g. 2 or more) hormones, hormone synthesizing enzymes and/or hormone precursors tagged with a detectable marker.

In some embodiments, the organoid is a clonal organoid. In some embodiments, the organoid is not a clonal organoid.

In some embodiments, the transcription factor for differentiating intestinal stem cells and/or intestinal cells with stem cell potential to EECs is selected from NEUROG3, ATOH1/MATH1 and NEUROD1, or a variant or fragment thereof or a fragment of a variant thereof. For example, in some embodiments, the transcription factor is NEUROG3 or a variant or fragment thereof or a fragment of a variant thereof. Preferably, the transcription factor is NEUROG3.

For example, in some preferred embodiments, the intestinal organoid comprises an inducible NEUROG3. In some preferred embodiments, the transcription factor for differentiating intestinal stem cells and/or intestinal cells with stem cell potential to EECs is expressed from an overexpression construct comprising the gene encoding the transcription factor under control of an inducible promoter. For example, in some embodiments, the intestinal organoid comprises an overexpression construct comprising NEUROG3 under control of an inducible promoter. In some embodiments, the overexpression construct comprising the gene encoding the transcription factor under control of an inducible promoter is integrated into the genome. For example, in some embodiments, the intestinal organoid comprises an overexpression construct comprising NEUROG3 under control of an inducible promoter, wherein the overexpression construct is integrated into the genome. In some embodiments, the overexpression construct is a lentiviral vector construct. In some preferred embodiments, the intestinal organoid stably comprises the transcription factor for differentiating intestinal stem cells and/or intestinal cells with stem cell potential to EECs. In some embodiments, the inducible promoter is a doxycycline inducible promoter, e.g. Tet-On. In some embodiments, the intestinal organoid is a reporter organoid as described herein. For example, in some embodiments, the intestinal organoid is a reporter organoid comprising a hormone, hormone precursor and/or hormone synthesizing enzyme tagged with a fluorescent marker. Accordingly, in some embodiments, a reporter organoid of the invention comprises a hormone, hormone precursor and/or hormone synthesizing enzyme tagged with a fluorescent marker, wherein the reporter organoid comprises an overexpression construct comprising NEUROG3 under control of an inducible promoter. The intestinal organoid is preferably a human intestinal organoid.

In some embodiments, the intestinal organoid has the organoid characteristics of an intestinal organoid obtained by the method as described in Sato, T. et al., (2011), WO 2010/090513, WO 2017/220586 and/or WO 2012/168930.

In some embodiments, the organoid is a differentiated intestinal organoid, e.g. in which the transcription factor (e.g. NEUROG3) has been overexpressed whilst culturing in differentiation medium and/or expansion medium as described herein.

Also provided is an intestinal organoid, wherein the organoid comprises more than 50% EECs and/or wherein the intestinal organoid comprises one or more EEC-specific genes (e.g. one or more hormones, hormone synthesizing enzymes, and/or hormone precursors) tagged with a detectable marker.

Preferably, the intestinal organoid is obtained by or is obtainable by a method of making a reporter organoid of the invention as described herein. In some embodiments, the organoid is obtained by or is obtainable by a method for enriching the population of EECs in an intestinal organoid, as described herein. The description of the various features of the organoids recited in the methods for making organoids provided herein may be extrapolated to the organoids provided by the invention, mutatis mutandis.

Embodiments described herein for intestinal organoids may also be applied to intestinal reporter organoids of the invention, mutatis mutandis.

The inventors have surprisingly found that the methods for enriching the population of EECs in an intestinal organoid described herein and/or differentiating intestinal stem cells and/or intestinal cells with stem cell potential to EECs by overexpressing a transcription factor as described herein can produce organoids in which more than 50% of the cells are EECs. The invention therefore provides an intestinal organoid in which more than 50% (e.g. more than 55%, 60%, 65%, 70%, 75%, 80%, 85%, 90%, 95% or 99%) of the cells are EECs. However, intestinal organoids comprising a lower percentage of EECs are also encompassed. For example, in some embodiments, there is provided an intestinal organoid in which at least 1% (e.g. at least 5%, 10%, 20%, 30%, 40%, 45%) of the cells are EECs. In some embodiments, at least 10% of the cells are EECs. Similarly, intestinal organoids comprising no EECs are also provided. For example, in some embodiments, the stem cells and/or intestinal cells with stem cell potential in an organoid of the invention have not been differentiated to EECs. In some embodiments, the organoid is a reporter organoid.

In some embodiments, more than 50%, more than 55%, more than 60%, more than 65%, more than 70%, more than 75%, more than 80%, more than 85%, more than 90%, more than 95%, or more than 99% of the cells in the organoid express one or more (e.g. at least 2, 3, 4, 5, 6, 7, 8, 9, 10, 11, 12, 13, 14 or 15) EEC markers. EEC markers include Chga, Chgb, Tac1, Tph1, Gip, Fabp5, Ghrl, Pyy, Nts, Neurod1, Sst, Sct, cholecystokinin, glucagon and/or pro-glucagon. Further EEC cell-types and their characteristics are described in Grun et al. (2015) Nature 525:251-255.

In some embodiments, the organoid of the invention is an intestinal organoid in which less than 50%, less than 40%, less than 30%, less than 20%, less than 10% or less than 1% of the cells express Goblet or Paneth cell markers (e.g. Lyz1, Defa6, Agr2, Gob5, Muc2, Ttf3 and/or Defa24).

In some embodiments, the organoid of the invention is an intestinal organoid in which less than 50%, less than 40%, less than 30%, less than 20%, less than 10% or less than 1% of the cells express enterocyte markers (e.g. Aldob, Apoa1 and/or Alpi).

In some embodiments, the organoid of the invention is an intestinal organoid in which less than 50%, less than 40%, less than 30%, less than 20%, less than 10% or less than 1% of the cells express Tuft cell markers (e.g. Dclk1 and/or Trpm5).

In some embodiments, marker expression is measured at the mRNA level. mRNA expression can be measured by single-cell RNA sequencing analysis, for example, as described herein.

As explained herein, not all organoids of the invention comprise at least 50% EECs. In particular, in some organoids of the invention (including some reporter organoids of the invention), the stem cells and/or cells with stem cell potential have not yet been differentiated to EECs. Similarly, organoids comprising a lower percentage of EECs are also encompassed, as described herein.

The inventors have surprisingly found that the methods of the invention which involve differentiating stem cells to EECs can produce organoids comprising EECs which retain their regional identity, including the endogenous repertoire of hormones. Accordingly, in some embodiments, the intestinal organoid comprises EECs of the same subtypes as are present in the region of the intestine from which the intestinal organoid was derived. In some embodiments, the organoid comprises EECs of the subtypes found in a region of the intestine selected from proximal small intestine (duodenum), the distal small intestine (ileum), the ascending colon, the descending colon, the transverse colon, the sigmoid colon, the rectum and the jejunum. In some embodiments, the organoid comprises EECs of the subtypes found in the proximal small intestine. In some embodiments, the organoid comprises EECs of the subtypes found in the distal small intestine. In some embodiments, the EEC subtypes found in the proximal or distal small intestine comprise one or more of (e.g. 1, 2, 3, 4, 5 or more or all 6 of) ECs, L-cells, K-cells (Gip+), G-cells (Gastrin+), M/X-cells (MIn+-Ghrl+) and D-cells (Sst+). In some embodiments, all of ECs, L-cells, K-cells (Gip+), G-cells (Gastrin+), M/X-cells (MIn+-Ghrl+) and D-cells (Sst+) are present. In some embodiments, the organoid comprises EECs selected from one or more of (e.g. 1, 2, 3, 4, 5 or more or all 6 of) Serotonin+ cells, GLP1+ cells, GIP+ cells, GAST+ cells, Motilin+ cells/Ghrelin+ cells and SST+ cells. In some embodiments, NTS+ cells are present instead of GLP-1+ cells. For example, GLP-1+ cells are always L-cells, but in the villus L-cells are NTS+ and not GLP-1+. In some embodiments, the organoid comprises EECs of the subtypes found in the ascending colon (for example, ECs (Tph1+) and/or L-cells (Gcg/Glp1+). In some embodiments, the organoid does not comprise EECs of subtypes which are not present in the region of the intestine from which the intestinal organoid was derived.

The inventors have also surprisingly found that the ratios of the different EEC subtypes in an organoid of the invention are the same as the ratios of the different EEC subtypes in the region of the intestine from which the organoid was established. Accordingly, in some embodiments, the intestinal organoid comprises EECs, wherein the ratios of the different EEC subtypes in the organoid is about the same as or is the same as the ratio of the different EEC subtypes in the region of the intestine from which the organoid was established. For example, in some embodiments, there is less than 10% (e.g. less than 7%, 5%, 3%, 1%, or 0%) variation of the ratios compared to the ratios in the region of the intestine from which the organoid was established. Thus, in some embodiments, the ratios are the same as the ratios in the region of the intestine from which the organoid was established. However, in some embodiments, the ratios of the different EEC subtypes in an organoid of the invention may be altered compared to the ratio of the different EEC subtypes in the region of the intestine from which the organoid was established, for example, by shifting EEC differentiation fate, for example, using a method described herein.

For example, in some embodiments, an organoid of the invention comprises an increased number of M-X cells and a decreased number of L-cells compared to the number of M-X cells and the number of L-cells in the region of the intestine from which the organoid was derived. For example, in some embodiments, an organoid of the invention comprises an increased number of MLN+ M-X cells and a decreased number of GLP1+ L-cells compared to the number of MLN+ M-X cells and the number of GLP1+ L-cells in the region of the intestine from which the organoid was derived. In some embodiments, an organoid of the invention generated by a method in which Wnt signalling has been inhibited before induction of transcription factor overexpression (e.g. NEUROG3 overexpression) in accordance with a method of the invention expresses an increased level of MLN and optionally a decreased level of GCG compared to the expression of MLN and optionally GCG in an organoid of the invention in which Wnt signalling has not been inhibited before induction of transcription factor overexpression.

In some embodiments, the organoid comprises EECs selected from one or more of (e.g. 2, 3, 4, 5, 6, 7, 8, 9, 10, 11, 12, 13 or more of or all 14 of) L-cells, N-cells, I-cells, K-cells, M-cells, S-cells, enterochromaffin (or EC-) cells, D-cells, enterochromaffin-like-cells, X-cells, M-X cells, G-cells, K-G-cells and P-cells.

In some embodiments, the organoid comprises EECs selected from one or more of (e.g. 1, 2, 3, 4, 5, 6, 7, 8, 9, 10, 11 or more or all 12 of) GLP1+ cells, PYY+ cells, CCK+ cells, Serotonin+ cells, PPY+ cells, NTS+ cells, GIP+ cells, Motilin+ cells, Ghrelin+ cells, Motilin+ and Ghrelin+ cells, Angiotensin+ cells, SST+ cells, GAST+ cells, and GIP+ and GAST+ cells.

Secretion of GCG-derived products (GLP-1, GLP-2), Peptide YY (PYY), Pancreatic Polypeptide Y (PPY) and/or Neurotensin (NTS) identifies an EEC as an L cell. In some embodiments, secretion of GCG-derived products (GLP-1, GLP-2) identifies an EEC as an L cell. For example, GLP-1+ cells are always L-cells, but in the villus L-cells are NTS+ and not GLP-1+. Secretion of PYY, identifies an EEC as an L cell. Secretion of PPY, identifies an EEC as an L cell. Secretion of CCK, identifies an EEC as an I cell. Secretion of serotonin, CHGA and/or CHGB identifies an EEC as an EC cell. In some embodiments, secretion of serotonin identifies an EEC as an EC cell. Secretion of NTS, identifies an EEC as an N cell. Secretion of GIP, identifies an EEC as a K cell. Secretion of motilin, identifies an EEC as an M cell. Secretion of Ghrelin, identifies an EEC as an X cell. Secretion of motilin, ghrelin, angiotensin and cerebellin 1 identifies an EEC as an M-X cell. In some embodiments, secretion of motilin and ghrelin identifies an EEC as an M-X cell. Secretion of angiotensin, identifies an EEC as an M/X cell. Secretion of SST, identifies an EEC as a D cell. Secretion of GAST, identifies an EEC as a G cell. Secretion of GIP and GAST identifies an EEC as a K-G cell.

In some embodiments, the one or more EEC subtypes are one or more EEC subtypes as described herein.

The skilled person will be able to determine which EEC cell types are present in an organoid of the invention, and their ratios, using for example, a technique selected from fluorescence assays (e.g. fluorescent plate reader or fluorescence microscopy), qPCR, RNA sequencing, ELISA, immunohistochemistry and proteomic analysis. For example, the EEC type present in a reporter organoid of the invention may be determined by determining if a tagged hormone that corresponds to a cell type is expressed, e.g. by visualisation of the fluorescent marker.

In some embodiments, the intestinal organoid comprises EECs which produce the same hormones, hormone synthesizing enzymes and/or hormone precursors as are produced endogenously by EECs in the region of the intestine from which the intestinal organoid was derived. In some embodiments, the hormones and/or hormone precursors produced by the EECs comprise one or more of (e.g. 2, 3, 4, 5, 6, 7, 8, 9, 10, 11, 12, 13, 14, 15, 16, 17, 18, 19, 20, 21, 22, 23, 24, 25 or more or all 26 of) CCK, CHGA, CHGB, GAST, GCG, GHRL, GIP, MLN, NTS, PYY, REG4, SCG2, SCG3, SCGN, SST, UCN3, MDK, PAM, REG3A, NPW, NUCB2 and VGF and optionally TAC3, PPY, NPW and/or CBLN1. In some embodiments, the hormones and/or hormone precursors produced by the EECs comprise one or more of (e.g. 2, 3, 4, 5, 6, 7, 8, 9, 10, 11, 12, 13, 14, 15 or more, or all 16 of) CCK, CHGA, CHGB, GAST, GCG, GHRL, GIP, MLN, NTS, PYY, REG4, SCG2, SCG3, SCGN, SST, UCN3. In some embodiments, the hormones produced by the EECs comprise one or more of (e.g. 2, 3, 4, 5 or more or all 6 of) MDK, PAM, REG3A, NPW, NUCB2, VGF. In some embodiments, the hormones and/or hormone precursors produced by the EECs comprise one or more of (e.g. 2, 3, 4, 5, 6, 7, 8, 9 or more of, or all 10 of) CHGA, MLN, GAST, GIP, CCK, serotonin, GCG, GHRL, SST, NTS. In some preferred embodiments, the intestinal organoid comprises EECs which alternatively or additionally secrete serotonin and/or somatostatin.

In some embodiments, a duodenal organoid of the invention expresses proximal SI hormones. In some embodiments, the duodenal organoid secretes one or more of (e.g. 1, 2, 3, 4, 5, 6, 7, 8, 9, 10, 11, 12, 13, 14, 15, 16 or more of all 17 of) CCK, CHGA, CHGB, GAST, GHRL, GIP, MLN, NTS, SCG2, SCG3, SST, MDK, PAM, REG3A, NPW, NUCB2, VGF. In some embodiments, the duodenal organoid secretes one or more of (e.g. 1, 2, 3, 4, 5, 6, 7 or more of all 8 of) CCK, CHGA, CHGB, GAST, MLN, SCG3 and SST. The inventors have shown all of these to be present in the secretome of duodenal organoids as full length proteins. In some embodiments, the duodenal organoid secretes one or more of (e.g. 1, 2, 3, 4, 5, 6, 7, 8, 9, 10 or more of all 11 of) CCK, CHGA, CHGB, GAST, GHRL, GIP, MLN, NTS, SCG2, SCG3 and SST. The inventors have shown all of these to be present in the secretome of duodenal organoids as processed peptides. In some embodiments, the duodenal organoid secretes one or more of (e.g. 1, 2 or more or all 3 of) MDK, PAM and REG3A. These are novel proteins which the inventors have shown to be present in the secretome of duodenal organoids as full length proteins. In some embodiments, the duodenal organoid secretes one or more of (e.g. 1, 2, 3, 4 or more or all 5 of) NPW, NUCB2, PAM, REG3A, VGF. These are novel proteins which the inventors have shown to be present in the secretome of duodenal organoids as processed peptides. In some embodiments, a duodenal organoid expresses one or more of (e.g. 2, 3, 4, 5 or more or all 6 of) GAST, GIP, CCK, MLN, GHRL and/or SST. In some embodiments, proximal SI hormones, for example, GAST, GIP, CCK, GHRL and/or MLN, are enriched in a duodenal organoid compared to in an ileal organoid. In some embodiments, a duodenal organoid of the invention comprises EECs which express MLN but which do not express GAST and/or comprises EECs which express GAST but which do not express MLN. In some embodiments, a duodenal organoid of the invention comprises one or more EECs which produce both GIP and CCK. The duodenal organoid of the invention preferably comprises one or more EECs which additionally secrete serotonin and/or somatostatin.

In some embodiments, an ileal organoid of the invention expresses distal SI hormones. In some embodiments, the ileal organoid secretes one or more of (e.g. 1, 2, 3, 4, 5, 6, 7, 8, 9, 10, 11, 12, 13, 14, 15, 16, 17, 18, 19 or more of all 20 of) CHGA, CHGB, GAST, GCG, GHRL, GIP, MLN, NTS, PYY, REG4, SCG2, SCG3, SCGN, SST, UCN3, MDK, NUCB2, PAM, VGF, NPW. In some embodiments, the ileal organoid secretes one or more of (e.g. 1, 2, 3, 4, 5, 6, 7, 8, 9, 10 or more of all 11 of) CHGA, CHGB, GCG, MLN, PYY, REG4, SCG2, SCG3, SCGN, SST. The inventors have shown all of these to be present in the secretome of ileal organoids as full length proteins. In some embodiments, the ileal organoid secretes one or more of (e.g. 1, 2, 3, 4, 5, 6, 7, 8, 9, 10, 11 or more of all 12 of) CHGA, GAST, GCG, GHRL, GIP, MLN, NTS, PYY, SCG2, SCG3, SST, UCN3. The inventors have shown all of these to be present in the secretome of ileal organoids as processed peptides. In some embodiments, the ileal organoid secretes one or more of (e.g. 1, 2, 3 or more or all 4 of) MDK, NUCB2, PAM and VGF. These are novel proteins which the inventors have shown to be present in the secretome of ileal organoids as full length proteins. In some embodiments, the ileal organoid secretes one or both of NPW and VGF. These are novel proteins which the inventors have shown to be present in the secretome of ileal organoids as processed peptides. In some embodiments, an ileal organoid expresses one or more of (e.g. 2, 3 or more of or all 4 of) NTS, PYY, GCG and/or SST. In some embodiments, distal SI hormones, for example, NTS, PYY and GCG, are enriched in an ileal organoid compared to a duodenal organoid. GCG encodes the preproglucagon prehormone, a protein precursor to a set of hormones including GLP-1. In some embodiments, an ileal organoid of the invention comprises EECs which express GHRL but which do not express CHGA and/or comprises EECs which express CHGA but which do not express GHRL. In some embodiments, an ileal organoid of the invention comprises EECs which produce serotonin but which do not produce GLP-1 and/or comprises EECs which produce GLP-1 but which do not produce serotonin. The ileal organoid of the invention preferably comprises one or more EECs which additionally secrete serotonin and/or somatostatin.

In some embodiments, an ileal organoid of the invention expresses SST at a comparable level to SST expression in a duodenal organoid of the invention. By a “comparable level” is meant less than 20% (e.g. less than 10%) variation. In some embodiments, SST expression is determined at the mRNA level. In some embodiments, SST expression is determined at the protein level. In some embodiments, an ileal organoid of the invention expresses less than 20% (e.g. less than 10%) more SST than a duodenal organoid of the invention. In some embodiments, SST expression is determined at the mRNA level. In some embodiments, SST expression is determined at the protein level.

The human colon produces only a small repertoire of hormones: serotonin-producing ECs and L-cells positive for GCG and PYY. This was shown in a recent single cell RNA sequencing study of inflammatory bowel disease patients and healthy controls which generated the profile of 83 colonic EECs (Parikh et al., 2019). Accordingly, in some embodiments, a colon organoid of the invention contains serotonin-producing ECs and L-cells positive for GCG and PYY. In some embodiments, a colon organoid of the invention contains serotonin-producing ECs and GCG-expressing EECs. In some embodiments, a colon organoid of the invention contains serotonin-producing ECs and GCG-expressing EECs but does not contain any other hormone-producing EECs and/or does not contain any other hormones produced by EECs. In some embodiments, the GCG-expressing EECs are L-cells positive for GCG and PYY.

Whether or not an organoid expresses (produces) a hormone can be detected at the mRNA level using mRNA transcripts, for example, by single cell RNA sequencing analysis. Alternatively, hormone expression can be detected by immunofluorescent staining for the majority of hormones, or for example, by mass spectrometry-based evidence. These methods are made possible using the large numbers of EECs available from the organoids of the invention.

In some embodiments, the hormones are full length proteins. In some embodiments, the hormones are processed peptides. In some embodiments, they are a mixture of full length proteins and processed peptides.

In some embodiments, a reporter duodenal organoid comprises one or more of (e.g. 2, 3, 4, 5 or more of, or all 6 of) the following: EECs comprising tagged CHGA; M cells comprising tagged MLN; G cells comprising tagged GAST; K cells comprising tagged GIP; I cells comprising tagged CCK; and EC cells comprising tagged TPH1.

In some embodiments, a reporter ileal organoid comprises one or more of (e.g. 2, 3, 4, 5, 6 or more of, or all 7 of) the following: EECs comprising tagged CHGA; L cells comprising tagged GCG; M cells comprising tagged MLN; X cells comprising tagged GHRL; D cells comprising tagged SST; N cells comprising tagged NTS; and EC cells comprising tagged TPH1.

In some embodiments, a reporter colon organoid comprises one or both of: L cells comprising tagged GCG; and EC cells comprising tagged TPH1.

In some embodiments, the hormone, hormone synthesizing enzyme and/or hormone precursor that is tagged in a reporter organoid of the invention is a hormone, hormone synthesizing enzyme and/or hormone precursor that is described herein as being expressed and/or secreted by human EECs, by human EECs of one or more specific subtypes, and/or by human EECs derived from a particular region of the intestine.

In some embodiments, a reporter duodenal organoid of the invention comprises a tagged hormone selected from one or more of (e.g. 1, 2, 3, 4, 5, 6, 7, 8, 9, 10, 11, 12, 13, 14, 15, 16 or more or all 17 of) CCK, CHGA, CHGB, GAST, GHRL, GIP, MLN, NTS, SCG2, SCG3, SST, MDK, PAM, REG3A, NPW, NUCB2, VGF.

In some embodiments, a reporter ileal organoid of the invention comprises a tagged hormone and/or hormone precursor selected from one or more of (e.g. 1, 2, 3, 4, 5, 6, 7, 8, 9, 10, 11, 12, 13, 14, 15, 16, 17, 18, 19 or more or all 20 of) CHGA, CHGB, GAST, GCG, GHRL, GIP, MLN, NTS, PYY, REG4, SCG2, SCG3, SCGN, SST, UCN3, MDK, NUCB2, PAM, VGF, NPW.

In some embodiments, only one EEC-specific gene (e.g. only one hormone or one hormone synthesizing enzyme or one hormone precursor) is tagged in each reporter organoid. In some embodiments, a reporter organoid of the invention comprises only one or more tagged hormones and does not comprise a tagged hormone synthesizing enzyme or a tagged hormone precursor. For example, in some embodiments, a reporter organoid of the invention comprises only one or more tagged hormone synthesizing enzymes and does not comprise a tagged hormone or a tagged hormone precursor. For example, in some embodiments, a reporter organoid of the invention comprises only one or more tagged hormone precursors and does not comprise a tagged hormone or a tagged hormone synthesizing enzyme.

In some embodiments, two or more (e.g. 2, 3, 4, or more) different EEC-specific genes are tagged in the same reporter organoid. In some embodiments, two or more (e.g. 2, 3, 4, or more) different EEC-specific genes are tagged in the same EEC within the reporter organoid. In some, embodiments, two or more (e.g. 2, 3, 4 or more) different EEC-specific genes are tagged in different EECs within the reporter organoid. Having multiple EEC-specific genes tagged in the same reporter organoid, e.g. within the same EEC, may be advantageous for methods which involve comparative analysis of the effects on the different EEC-specific genes, for example, the effects of a compound of interest. Such embodiments may be particularly useful in embodiments in which the EEC-specific genes are one or more hormones, hormone precursors and/or hormone synthesizing enzymes. It is similarly useful for studying the effect of a compound on a tagged transcription factor and a tagged hormone, hormone precursor and/or tagged hormone synthesizing enzyme. In some embodiments, the two or more EEC-specific genes comprise or consist of a tagged hormone and a tagged hormone precursor.

Many of the hormones tagged in the present invention have not been endogenously tagged before, for example due to the absence of the murine counterpart (e.g. Motilin). Somatostatin has been reported only indirectly in mice using hormone-driven Cre recombinase expression (Somatostatin). Thus, a reporter organoid comprising tagged Motilin is specifically provided. Similarly, a reporter organoid comprising tagged Somatostatin is specifically provided.

The features of the organoids of the invention can be extrapolated to the reporter organoids of the invention mutatis mutandis.

The organoid is an intestinal organoid. In some embodiments, the organoid is a mammalian organoid. The organoid is preferably a human organoid. However, in some alternative embodiments, it is from a mouse, rabbit, rat, hamster, guinea pig or other non-human mammal.

In some embodiments, the organoid has a cystic structure with a central lumen. In some embodiments the central lumen is surrounded by an epithelial monolayer.

The EECs in an organoid of the invention preferably display a normal morphology as visualized by transmission electron microscopy.

Also provided is an organoid or a population of EECs of the invention in a culture medium, e.g. an expansion medium or a differentiation medium, for example, as described herein.

In an embodiment, an organoid of the invention is an organoid which is still being cultured using a method of the invention and is therefore in contact with an extracellular matrix. Preferably, in such an embodiment, an organoid of the invention is embedded in a non-mesenchymal extracellular matrix.

In some embodiments, the organoid is a differentiated organoid. In some embodiments, a differentiated organoid is a three-dimensional structure comprising differentiated epithelial cell types. A differentiated organoid is typically self-organising, meaning that the three-dimensional arrangement of the cells in the organoid occurs spontaneously as the cells differentiate. In some embodiments, a differentiated organoid is derived from epithelial stem cells, optionally expressing Lgr5. In some embodiments, an intestinal organoid described herein is a differentiated organoid. In some embodiments, a differentiated organoid as described herein is an organoid in which more than 1% (e.g. more than 5%, 10%, 20%, 30%, 40%, 50%, 60%, 70%, 80%) of the cells are EECs. In some embodiments, a differentiated organoid may be an organoid in which more than 50% of the cells are EECs.

In some embodiments, the invention provides a differentiated organoid or a population of one or more differentiated cells obtainable or obtained by a method of the invention.

A ‘population’ of cells is any number of cells greater than 1, but is preferably at least 10 cells, at least 50 cells, at least 100 cells, at least 500 cells, at least 1×10³ cells, at least 1×10⁴ cells, at least 1×10⁵ cells, at least 1×10⁶ cells, at least 1×10⁷ cells, at least 1×10⁸ cells, or at least 1×10⁹ cells.

An organoid according to the present invention may comprise a population of cells of at least 10 cells, at least 50 cells, at least 100 cells, at least 500 cells, at least 1×10³ cells, at least 1×10⁴ cells, at least 1×10⁵ cells, at least 1×10⁶ cells, at least 1×10⁷ cells or more. In some embodiments, each organoid comprises between approximately 1×10³ cells and 5×10³ cells; generally, 10-20 organoids may be grown together in one well, for example of a 24 well plate.

It is clear to the skilled person that an organoid of the invention is not a naturally occurring tissue fragment and/or does not comprise a blood vessel.

Organoids of the invention are, for example, distinguished from naturally occurring tissue because they comprise only epithelial cell types (and not mesenchymal cells or other structural cell types). Thus in some embodiments, the organoid of the invention comprises only epithelial cells. In some embodiments, the organoids do not comprise non-epithelial cells. For example, in a particular embodiment, the organoids do not comprise mesenchymal cells.

The differentiation medium described herein preferably induces or promotes a specific differentiation of cells during at least three days of culture. In some embodiments, the cells are cultured in the differentiation medium for at least five days. Differentiation may be measured by detecting the presence of a specific marker associated with the enteroendocrine lineage, as defined herein. In some embodiments, depending on the identity of the marker, the expression of said marker may be assessed by RTPCR or immuno-histochemistry after at least 3, 4, 5, 7, 8, 9, 10, 11, 12, 13, 14 or more days of culture in a differentiation medium as defined herein.

The organoid of the invention or the population of cells of the invention preferably comprises at least 50% viable cells, more preferred at least 60% viable cells, more preferred at least 70% viable cells, more preferred at least 80% viable cells, more preferred at least 90% viable cells. Viability of cells may be assessed using Hoechst staining or Propidium Iodide staining in FACS. The viable cells preferably possess corresponding in vivo functions or characteristics. For example, viable EECs preferably possess enteroendocrine functions or characteristics of EECs.

A definitive hallmark of a mature EEC is its ability to secrete hormones. The invention provides a cell culture comprising one or more organoids of the invention and one or more secreted hormones.

Biobanks and Other Uses

Collections of organoids of the invention representative of different regions of the intestine are advantageous for use as biobanks. For example, when the organoids are in their differentiated form, the biobanks may comprise organoids comprising different types of EECs representative of different regions of the intestine. Such biobanks are provided by the invention. For example, a biobank of the invention may comprise or consist of a first organoid of the invention established from a first region of the intestine and a second organoid of the invention established from a second region of the intestine. Similarly, the biobank may comprise or consist of one or more (e.g. 1, 2, 3, 4, 5, 6 or more) additional organoids of the invention each established from a different region of the intestine. For example, the invention provides a biobank that comprises or consists of two or more (e.g. 2, 3, 4, 5, 6, 7 or more) organoids of the invention which are each established from different regions of the intestine. In some embodiments, the regions of the intestine are selected from the proximal small intestine (duodenum), the distal small intestine (ileum), the ascending colon, the descending colon, the transverse colon, the sigmoid colon, the rectum and the jejunum. In some embodiments, the biobank comprises or consists of organoids which together comprise or consist of organoids established from all the regions of the intestinal tract. In some embodiments, the biobank comprises or consists of organoids established from the proximal small intestine, the distal small intestine and/or the ascending colon. In some embodiments, the biobank comprises or consists of organoids which together comprise or consist of all the EEC subtypes from the intestinal tract.

In some embodiments, the organoids in the biobank are present in their undifferentiated form, for example, as described herein. For example, in some embodiments, the organoids do not comprise EECs. In some embodiments, the organoids of the invention are present in their differentiated form, for example, as described herein. For example, in some embodiments, the organoids comprise EECs. In some embodiments, a mixture of organoids in their undifferentiated form and organoids in their differentiated form are present.

The organoids described herein, for example comprising EECs or those comprising stem cells and/or cells with stem cell potential that have not yet been differentiated to EECs, and EECs isolated from the organoids, can be used in cellular assays, drug screening, and toxicity assays.

In some embodiments, there is provided a biobank of reporter organoids of the invention. In some embodiments, the biobank of reporter organoids comprises two or more (e.g. 2, 3, 4, 5, 6, 7, 8, 9, 10, 11, 12, 15, 20 or more) different reporter organoids. In some embodiments, the biobank consists of or consists essentially of reporter organoids established from the intestine. For example, in some embodiments, all of the reporter organoids in the biobank are intestinal organoids. In some embodiments, the two or more reporter organoids are established from the same region of the intestine and comprise different tagged EEC-specific genes (e.g. tagged hormones, tagged hormone synthesizing enzymes and/or tagged hormone precursors). In some embodiments, the two or more reporter organoids are established from different regions of the intestine and comprise the same or different tagged EEC-specific genes (e.g. tagged hormones, tagged hormone synthesizing enzymes and/or tagged hormone precursors). In some embodiments, the biobank of reporter organoids comprises two or more (e.g. 2, 3, 4, 5, 6, 7, 8, 9, 10, 12, 15 or more) different tagged EEC-specific genes (e.g. tagged hormones, tagged hormone synthesizing enzymes and/or tagged hormone precursors). Any tagged EEC-specific genes may be present in the biobank, for example, EEC-specific genes as described herein.

In some embodiments, the biobank comprises tagged hormones but does not comprise any tagged hormone synthesizing enzymes or tagged hormone precursors. In some embodiments, the biobank comprises tagged hormone synthesizing enzymes but does not comprise any tagged hormones or tagged hormone precursors. In some embodiments, the biobank comprises tagged hormone precursors but does not comprise any tagged hormones or tagged hormone synthesizing enzymes. In some embodiments, the biobank comprises tagged hormones and tagged hormone synthesizing enzymes. In some embodiments, the biobank comprises tagged hormones and tagged hormone precursors. In some embodiments, the biobank comprises tagged hormone synthesizing enzymes and tagged hormone precursors. In some embodiments, the biobank comprises tagged hormones, tagged hormone synthesizing enzymes and tagged hormone precursors. In some embodiments, the biobank comprises tagged transcription factors. In some embodiments, the biobank comprises tagged hormones, hormone precursors, hormone synthesizing enzymes and/or transcription factors. In some embodiments, the biobank does not comprise any other tagged EEC-specific genes.

In some embodiments, the tagged hormones and/or hormone precursors in the biobank of reporter organoids comprise or consist of or consist essentially of all of the hormones and/or hormone precursors secreted by EECs. In some embodiments, the tagged hormones and/or hormone precursors in the biobank of reporter organoids comprise or consist of or consist essentially of all the hormones and/or hormone precursors secreted by a subset of EECs. In some embodiments, the tagged hormones in the biobank of reporter organoids comprise or consist of at least 1, 2, 3, 4, 5, 6, 7, 8, 9, 10, 11, 12, 13, 14 or 15 hormones and/or hormone precursors secreted by EECs or a subset of EECs. In some embodiments, the subset of EECs are the EECs present in an organoid established from a particular region of the intestine, for example, from a region selected from the proximal small intestine (duodenum), the distal small intestine (ileum), the ascending colon, the descending colon, the transverse colon, the sigmoid colon, the rectum and the jejunum. In some embodiments, the biobank of reporter organoids comprises one or more (e.g. 1, 2, 3, 4, 5, 6, 7, 8, 9, 10, 11, 12, 13, 14, 15 or more) tagged hormones and/or hormone precursors secreted by duodenal, ileal and/or colon organoids. In some embodiments, the subset of EECs is an EEC subtype, for example, selected from one of L-cells, N-cells, I-cells, K-cells, M-cells, S-cells, enterochromaffin (or EC-) cells, D-cells, enterochromaffin-like-cells, X-cells, M-X cells, G-cells, K-G-cells, P-cells, GLP1+ cells, PYY+ cells, CCK+ cells, Serotonin+ cells, PPY+ cells, NTS+ cells, GIP+ cells, Motilin+ cells, Ghrelin+ cells, Angiotensin+ cells, SST+ cells and GAST+ cells.

Advantageously, the tagged hormones in the biobank of reporter organoids may consist of or consist essentially of hormones secreted by EECs or a subset of EECs, for example, by EECs from a particular region of the intestine or by one or more types of EEC. The presence of other organoids in the biobank which comprise other tagged proteins (e.g. other tagged hormones, e.g. in organoids established from a tissue other than the intestine or in organoids established from a different region of the intestine) or no tagged proteins (e.g. no tagged hormones) would not cause the biobank to fall outside the scope of the invention, for example, if the purpose of the biobank is for use in studying hormones produced by EECs from the intestine, or another aspect associated with EEC hormones or EEC biology. Thus, biobanks additionally comprising organoids comprising other tagged proteins or no tagged proteins are also envisaged. For example, in some embodiments, the biobank comprises organoids comprising no more than 10 (e.g. no more than 8, 5, 3, 2, 1) tagged proteins other than tagged EEC-specific genes (e.g. other than tagged hormones produced by EECs). In some embodiments, the biobank comprises organoids comprising no more than 10 (e.g. no more than 8, 5, 3, 2, 1) tagged proteins other than tagged hormones, tagged hormone synthesizing enzymes and/or tagged hormone precursors produced by EECs.

In some embodiments, the tagged hormone synthesizing enzymes in the biobank of reporter organoids comprise or consist of or consist essentially of all of the hormone synthesizing enzymes encoded by EECs or at least one hormone synthesizing enzyme for each hormone that is synthesized by EECs. In some embodiments, the tagged hormone synthesizing enzymes in the biobank of reporter organoids comprise or consist of or consist essentially of all the hormone synthesizing enzymes encoded by a subset of EECs. For example, in some embodiments, the subset of EECs is the EECs that are present in an organoid derived from a particular region of the intestine. In some embodiments, the tagged hormone synthesizing enzymes in the biobank of reporter organoids comprise or consist of or consist essentially of at least one hormone synthesizing enzyme for each hormone that is synthesized by a subset of EECs. In some embodiments, the tagged hormone synthesizing enzymes in the biobank of reporter organoids comprise or consist of at least 1, 2, 3, 4, 5, 6, 7, 8, 9, 10, 11, 12, 13, 14 or 15 hormone synthesizing enzymes encoded by EECs or by a subset of EECs. In some embodiments, the subset of EECs are the EECs present in an organoid established from a particular region of the intestine, for example, from a region selected from the proximal small intestine (duodenum), the distal small intestine (ileum), the ascending colon, the descending colon, the transverse colon, the sigmoid colon, the rectum and the jejunum. In some embodiments, the biobank of reporter organoids comprises one or more (e.g. 1, 2, 3, 4, 5, 6, 7, 8, 9, 10, 11, 12, 13, 14, 15 or more) tagged hormone synthesizing enzymes encoded by duodenal, ileal and/or colon organoids. In some embodiments, the subset of EECs is an EEC subtype, for example, selected from one of L-cells, N-cells, I-cells, K-cells, M-cells, S-cells, enterochromaffin (or EC-) cells, D-cells, enterochromaffin-like-cells, X-cells, M-X cells, G-cells, K-G-cells, P-cells, GLP1+ cells, PYY+ cells, CCK+ cells, Serotonin+ cells, PPY+ cells, NTS+ cells, GIP+ cells, Motilin+ cells, Ghrelin+ cells, Angiotensin+ cells, SST+ cells and GAST+ cells.

Advantageously, the tagged hormone synthesizing enzymes in the biobank of reporter organoids may consist of or consist essentially of hormone synthesizing enzymes encoded by EECs or a subset of EECs. However, the presence of other organoids in the biobank which comprise other tagged proteins (e.g. other hormone synthesizing enzymes) or no tagged proteins (e.g. no tagged hormone synthesizing enzymes) would not cause the biobank to fall outside the scope of the invention, for example, if the purpose of the biobank is for use in studying hormones (or hormone synthesizing enzymes) produced/encoded by EECs, or another aspect associated with EEC hormones or EEC biology. Thus, biobanks additionally comprising organoids comprising other tagged proteins or no tagged proteins are also envisaged. For example, in some embodiments, the biobank comprises organoids comprising no more than 10 (e.g. no more than 8, 5, 3, 2, 1) tagged proteins other than tagged hormone synthesizing enzymes produced by EECs.

In some embodiments, the tagged hormone precursors in the biobank of reporter organoids comprise or consist of or consist essentially of all of the hormone precursors encoded by EECs. In some embodiments, the tagged hormone precursors in the biobank of reporter organoids comprise or consist of or consist essentially of all the hormone precursors encoded by a subset of EECs. In some embodiments, the tagged hormone precursors in the biobank of reporter organoids comprise or consist of or consist essentially of at least 1, 2, 3, 4, 5 or 10 hormone precursors encoded by EECs or by a subset of EECs. In some embodiments, the subset of EECs are the EECs present in an organoid established from a particular region of the intestine, for example, from a region selected from the proximal small intestine (duodenum), the distal small intestine (ileum), the ascending colon, the descending colon, the transverse colon, the sigmoid colon, the rectum and the jejunum. In some embodiments, the biobank of reporter organoids comprises one or more (e.g. 1, 2, 3, 4 or more) tagged hormone precursors encoded by duodenal, ileal and/or colon organoids. In some embodiments, the subset of EECs is an EEC subtype, for example, selected from one of L-cells, N-cells, I-cells, K-cells, M-cells, S-cells, enterochromaffin (or EC-) cells, D-cells, enterochromaffin-like-cells, X-cells, M-X cells, G-cells, K-G-cells, P-cells, GLP1+ cells, PYY+ cells, CCK+ cells, Serotonin+ cells, PPY+ cells, NTS+ cells, GIP+ cells, Motilin+ cells, Ghrelin+ cells, Angiotensin+ cells, SST+ cells and GAST+ cells.

Advantageously, the tagged hormone precursors in the biobank of reporter organoids may consist of or consist essentially of hormone precursors encoded by EECs or a subset of EECs. However, the presence of other organoids in the biobank which comprise other tagged proteins (e.g. other tagged hormone precursors, e.g. in organoids derived from a different organ) or no tagged proteins (e.g. no tagged hormone precursors) would not cause the biobank to fall outside the scope of the invention, for example, if the purpose of the biobank is for use in studying hormones produced by EECs, or another aspect associated with EEC hormones or EEC biology. Thus, biobanks additionally comprising organoids comprising other tagged proteins or no tagged proteins are also envisaged. For example, in some embodiments, the biobank comprises organoids comprising no more than 10 (e.g. no more than 8, 5, 3, 2, 1) tagged proteins other than tagged hormone precursors encoded by EECs.

As mentioned above, the EECs used and/or recited in all aspects of the invention are preferably human EECs. Similarly, the organoids used and/or recited in all aspects of the invention are preferably human organoids.

In some embodiments, the biobank of reporter organoids comprises two or more (e.g. 2, 3, 4, 5, 6, 7, 8, 9 or more) organoids which are established from two or more (e.g. 2, 3, 4, 5, 6, 7, 8, 9 or more) different regions of the intestine, e.g. as described herein. In some embodiments, each reporter organoid member of the biobank is established from a different region of the intestine, e.g. as described herein.

In some embodiments, the biobank of reporter organoids comprises a first organoid established from a first region of the intestine which comprises a particular EEC type and one or more additional organoids established from one or more additional regions of the intestine which comprise the same EEC type. Such a biobank may be of interest for comparing hormone production by the particular EEC type in different regions of the intestine. For example, such a biobank may be useful for determining whether the same or different hormones are produced by the same EEC type in different regions of the intestine and/or determining whether the level of a particular hormone produced by the particular EEC type is the same or different in different regions of the intestine. The term “EEC type” is used herein interchangeably with “EEC subtype”.

In some embodiments, the biobank of reporter organoids comprises duodenal, ileal and/or colon organoids comprising EEC-specific genes (e.g. hormones) tagged with one or more detectable markers. In some embodiments, the biobank of reporter organoids comprises reporter organoids established from the proximal small intestine (duodenum), the distal small intestine (ileum), the ascending colon, the descending colon, the transverse colon, the sigmoid colon, the rectum and/or the jejunum, in which EEC-specific genes (e.g. hormones) are tagged with one or more detectable markers. In some embodiments, the biobank of reporter organoids comprises one or more duodenal, ileal and/or colon organoids in which one or more hormones is tagged with one or more detectable markers. In some embodiments, the biobank of reporter organoids comprising one or more tagged hormones additionally or alternatively comprises one or more reporter organoids in which a hormone synthesizing enzyme is tagged with a detectable marker and/or one or more reporter organoids in which a hormone precursor is tagged with a detectable marker. In some embodiments, the biobank of reporter organoids comprises duodenal, ileal and/or colon organoids comprising one or more hormone synthesizing enzymes tagged with one or more detectable markers and/or one or more hormone precursors tagged with one or more detectable markers.

In some embodiments, the biobank of reporter organoids comprises duodenal organoids in which one or more EEC-specific genes (e.g. one or more hormones, hormone synthesizing enzymes and/or hormone precursors) is tagged with one or more detectable markers. In some embodiments, the biobank of reporter duodenal organoids does not comprise reporter organoids established from other regions of the intestine. For example, in some embodiments, a biobank of reporter duodenal organoids does not comprise reporter ileal organoids and/or reporter colon organoids. In some embodiments, the biobank consists of or consists essentially of reporter duodenal organoids. In some embodiments, the reporter duodenal organoids comprise one or more of (e.g. 2, 3, 4, 5 or more of, or all 6 of) the following: EECs comprising tagged CHGA; M cells comprising tagged MLN; G cells comprising tagged GAST; K cells comprising tagged GIP; I cells comprising tagged CCK; and EC cells comprising tagged TPH1.

Similarly, in some embodiments, the biobank of reporter organoids comprises ileal organoids in which one or more EEC-specific genes (e.g. one or more hormones, hormone synthesizing enzymes and/or hormone precursors) is tagged with one or more detectable markers. In some embodiments, the biobank of reporter ileal organoids does not comprise reporter organoids established from other regions of the intestine. For example, in some embodiments, a biobank of reporter ileal organoids does not comprise reporter duodenal organoids and/or reporter colon organoids. In some embodiments, the biobank consists of or consists essentially of reporter ileal organoids. In some embodiments, the reporter ileal organoids comprise one or more of (e.g. 2, 3, 4, 5, 6 or more of, or all 7 of) the following: EECs comprising tagged CHGA; L cells comprising tagged GCG; M cells comprising tagged MLN; X cells comprising tagged GHRL; D cells comprising tagged SST; N cells comprising tagged NTS; and EC cells comprising tagged TPH1.

Similarly, in some embodiments, the biobank of reporter organoids comprises colon organoids in which one or more EEC-specific genes (e.g. one or more hormones, hormone synthesizing enzymes and/or hormone precursors) are tagged with one or more detectable markers. In some embodiments, the biobank of reporter colon organoids does not comprise other reporter organoids established from other regions of the intestine. For example, in some embodiments, a biobank of reporter colon organoids does not comprise reporter duodenal organoids and/or reporter ileal organoids. In some embodiments, the biobank consists of or consists essentially of reporter colon organoids. In some embodiments, the reporter colon organoids comprise one or both of: L cells comprising tagged GCG; and EC cells comprising tagged TPH1.

In some embodiments, the biobank of reporter organoids comprises duodenal and ileal organoids as described herein. In some embodiments, the biobank of reporter organoids comprises duodenal and colon organoids as described herein. In some embodiments, the biobank of reporter organoids comprises ileal and colon organoids as described herein. In some embodiments, the biobank of reporter organoids comprises duodenal, ileal and colon organoids as described herein.

In some embodiments, the biobank of reporter organoids comprises or consists of two or more of reporter duodenal organoids comprising one or more of (e.g. 2, 3, 4, 5 or more of, or all 6 of) the following: EECs comprising tagged CHGA; M cells comprising tagged MLN; G cells comprising tagged GAST; K cells comprising tagged GIP; I cells comprising tagged CCK; and EC cells comprising tagged TPH1; reporter ileal organoids comprising one or more of (e.g. 2, 3, 4, 5, 6 or more of, or all 7 of) the following: EECs comprising tagged CHGA; L cells comprising tagged GCG; M cells comprising tagged MLN; X cells comprising tagged GHRL; D cells comprising tagged SST; N cells comprising tagged NTS; and EC cells comprising tagged TPH1; and reporter colon organoids comprising one or both of: L cells comprising tagged GCG; and EC cells comprising tagged TPH1.

For example, in some embodiments, the biobank of reporter organoids comprises or consists of reporter duodenal organoids comprising EECs comprising tagged CHGA; M cells comprising tagged MLN; G cells comprising tagged GAST; K cells comprising tagged GIP; I cells comprising tagged CCK; and EC cells comprising tagged TPH1; reporter ileal organoids comprising EECs comprising tagged CHGA; L cells comprising tagged GCG; M cells comprising tagged MLN; X cells comprising tagged GHRL; D cells comprising tagged SST; N cells comprising tagged NTS; and EC cells comprising tagged TPH1; and reporter colon organoids comprising L cells comprising tagged GCG; and EC cells comprising tagged TPH1.

In some embodiments, the tagged hormones and/or hormone precursors in the biobank comprise one or more of (e.g. 2, 3, 4, 5, 6, 7, 8 or more of, or all 9 of) CHGA, MLN, GAST, GIP, CCK, GCG, GHRL, SST and NTS. These hormones and hormone precursors are tagged in the EEC-TAG biobank made by the inventors which is described in the examples. In some embodiments, the tagged hormones and/or hormone precursors in the biobank comprise one or more of (e.g. 2, 3, 4, 5, 6, 7, 8, 9, 10, 11, 12, 13, 14, 15, 16, 17, 18, 19, 20, 21, 22, 23, 24, 25 or more or all 26 of) CCK, CHGA, CHGB, GAST, GCG, GHRL, GIP, MLN, NTS, PYY, REG4, SCG2, SCG3, SCGN, SST, UCN3, MDK, PAM, REG3A, NPW, NUCB2 and VGF, and optionally TAC3, PPY, NPW and/or CBLN1. In some embodiments, the tagged hormones and/or hormone precursors in the biobank comprise one or more of (e.g. 2, 3, 4, 5, 6, 7, 8, 9, 10, 11, 12, 13, 14, 15 or more, or all 16 of) CCK, CHGA, CHGB, GAST, GCG, GHRL, GIP, MLN, NTS, PYY, REG4, SCG2, SCG3, SCGN, SST, UCN3. In some embodiments, the tagged hormones in the biobank comprise one or more of (e.g. 2, 3, 4, 5, 6 or more or all 6 of) MDK, PAM, REG3A, NPW, NUCB2, VGF. In some embodiments, the hormone precursor GCG is removed from these lists.

In some embodiments of the biobank, the hormone synthesizing enzyme that is tagged with a detectable marker is TPH1 or DDC. In some embodiments, the hormone synthesizing enzyme that is tagged with a detectable marker is TPH1. TPH1 is the rate-limiting enzyme involved in the synthesis of the hormone serotonin. Thus, in some embodiments, biobank additionally comprises a tagged hormone synthesizing enzyme, e.g. TPH1. TPH1 is also tagged in the EEC-TAG biobank made by the inventors which is described in the examples.

In some embodiments of the biobank, the hormone precursor that is tagged with a detectable marker is GCG. GCG is the precursor of the hormone GLP1.

In some embodiments, a biobank of the invention comprises one or more of (2, 3, 4, 5, 6, 7, 8, 9 or more or all of) CHGA, MLN, GAST, GIP, CCK, GCG, GHRL, SST, NTS and TPH1 each tagged with a detectable marker. These are all tagged in the EEC-TAG biobank described in the examples.

Preferably each organoid member of the biobank of reporter organoids comprises one or more tagged EEC-specific genes. However, in some embodiments, some organoid members of the biobank (e.g. less than 30%, less than 20%, less than 10%, less than 5%, less than 2%, less than 1%) do not comprise a tagged EEC-specific gene. In some embodiments, each reporter organoid member of the biobank comprises no more than four (e.g. no more than three, no more than two, no more than one) tagged EEC-specific genes. In some embodiments, different reporter organoid members of the biobank comprise different numbers of tagged EEC-specific genes.

In some embodiments, each of the different tagged EEC-specific genes (e.g. tagged hormones, tagged hormone synthesizing enzymes and/or tagged hormone precursors) in the biobank of reporter organoids is tagged with a different detectable marker. In some embodiments, each of the different tagged EEC-specific genes in the biobank of reporter organoids is tagged with the same detectable marker. In some embodiments, two or more different EEC-specific genes in the biobank of reporter organoids are tagged with the same detectable marker. In some embodiments, the identity of the detectable marker is determined according to the origin of the organoid or the type of cell being labelled. For example, in some embodiments, a first detectable marker is used to tag EEC-specific genes in duodenal organoids, a second detectable marker is used to tag EEC-specific genes in ileal organoids and/or a third detectable marker is used to tag EEC-specific genes in ascending colon organoids. In some embodiments, a first detectable marker is used to tag EEC-specific genes in a first EEC type and a second detectable marker is used to tag EEC-specific genes in a second EEC type.

In some embodiments, the detectable marker is a fluorescent molecule. In some embodiments, the different detectable markers are different fluorescent molecules. In some embodiments, the different detectable markers are fluorescent molecules having different colours, e.g. selected from mNeon, mClover, dTomato, and mCherry.

The biobank of intestinal reporter organoids comprising tagged hormones, tagged hormone synthesizing enzymes and/or tagged hormone precursors described herein in the examples is optionally termed the “EEC-TAG” biobank.

The description of the reporter organoid members of the biobank as provided herein can also be applied to the reporter organoids of the invention as described herein mutatis mutandis, for example, when they are not necessarily part of a biobank. Similarly, the description of the reporter organoids provided herein can also be applied to the reporter organoids members of the biobank described herein, mutatis mutandis. Similarly, the description of the organoids provided herein can also be applied to the reporter organoids described herein and reporter organoid members of the biobank as described herein, mutatis mutandis. Similarly, the description of the reporter organoids provided herein and reporter organoid members of the biobank provided herein can also be applied to the organoids of the invention as described herein, mutatis mutandis.

In some embodiments, there is provided a culture medium comprising a reporter organoid of the invention. In some embodiments, there is provided a culture medium comprising a biobank of reporter organoids of the invention. In some embodiments, there is provided a culture medium comprising a reporter organoid of the invention and one or more tagged hormones and/or tagged hormone precursors. In some embodiments, there is provided a culture medium comprising a biobank of reporter organoids of the invention and one or more tagged hormones and/or tagged hormone precursors. In some embodiments, there is provided a culture medium comprising an intestinal organoid of the invention. In some embodiments, there is provided a culture medium comprising an intestinal organoid of the invention and one or more hormones and/or hormone precursors. Thus, the culture medium may comprise both the one or more reporter organoids (or intestinal organoids) and the one or more (tagged) hormones and/or (tagged) hormone precursors.

Examples of suitable culture media are well known in the art and are also described herein. In some embodiments, the culture medium is an expansion medium. In some embodiments, the culture medium is a differentiation medium. The hormones and/or hormone precursors are preferably hormones and/or hormone precursors that are secreted by EECs, for example, by one or more EEC subtypes. In some embodiments, the hormones and/or hormone precursors are one or more hormones as described herein.

Also provided are the tagged proteins themselves, for example, one or more tagged proteins encoded by EEC-specific genes (e.g. one or more tagged hormones, tagged hormone synthesizing enzymes and/or tagged hormone precursors) as described herein. In some embodiments, there is provided one or more tagged proteins encoded by one or more EEC-specific genes (e.g. one or more tagged hormones, tagged hormone synthesizing enzymes and/or tagged hormone precursors) obtainable by or obtained by a method of the invention. In some embodiments, there is provided a library of tagged proteins encoded by EEC-specific genes (e.g. tagged hormones, tagged hormone synthesizing enzymes and/or tagged hormone precursors). For example, the library may comprise two or more (e.g. 2, 3, 4, 5, 6, 7, 8, 9, 10 or more) different tagged proteins encoded by EEC-specific genes (e.g. two or more different tagged hormones, tagged hormone synthesizing enzymes and/or tagged hormone precursors). The description of the tagged EEC-specific genes (e.g. the tagged hormones, tagged hormone synthesizing enzymes and/or tagged hormone precursors) provided herein in relation to the reporter organoids and the reporter organoid biobank can also be applied to the tagged proteins of the invention, mutatis mutandis.

Also provided is a culture medium comprising one or more tagged hormones and/or tagged hormone precursors or comprising a library of tagged hormones and/or tagged hormone precursors. Also provided is a supernatant comprising one or more tagged hormones and/or tagged hormone precursors or comprising a library of tagged hormones and/or tagged hormone precursors. Similarly, there is provided a library of two or more (e.g. 2, 3, 4, 5, 6, 7, 8, 9, 10 or more) culture media which each comprise a different tagged hormone and/or tagged hormone precursor or which each comprise a different combination of tagged hormones and/or tagged hormone precursors. Similarly, there is provided a library of two or more (e.g. 2, 3, 4, 5, 6, 7, 8, 9, 10 or more) supernatants which each comprise a different tagged hormone and/or tagged hormone precursor or which each comprise a different combination of tagged hormones and/or tagged hormone precursors.

In some embodiments, the tagged hormones, tagged hormone synthesizing enzymes and/or tagged hormone precursors are isolated from the organoid culture medium and/or from the organoid, for example, from the EECs. Thus, the invention provides one or more isolated tagged proteins, for example, isolated tagged hormones, isolated tagged hormone synthesizing enzymes and/or isolated tagged hormone precursors and libraries thereof, as described herein.

Also provided is a library comprising two or more (e.g. 2, 3, 4, 5, 6, 7, 8, 9, 10 or more) isolated hormones, hormone synthesizing enzymes and/or hormone precursors as described herein. In some embodiments, the hormones, hormone synthesizing enzymes and/or hormone precursors comprise a tag. In some embodiments, the hormones, hormone synthesizing enzymes and/or hormone precursors do not comprise a tag. In some embodiments, none of the hormones, hormone synthesizing enzymes and/or hormone precursors in the library comprises a tag. In some embodiments, the library comprises a combination of hormones, hormone synthesizing enzymes and/or hormone precursors which do not comprise a tag and tagged hormones, tagged hormone synthesizing enzymes and/or tagged hormone precursors. In some embodiments, at least 40% (e.g. at least 50%, 60%, 70%, 80%, 90%, 95%) of the hormones, hormone synthesizing enzymes and/or hormone precursors do not comprise a tag. In some embodiments, the hormones, hormone synthesizing enzymes and/or hormone precursors are obtained by a method of the present invention and the tag is then removed from the hormone, hormone synthesizing enzyme and/or hormone precursor. Methods for removing the tags are well known in the art. Alternatively, the hormones, hormone synthesizing enzymes and/or hormone precursors may be obtained by any other suitable method. For example, they may be obtained either individually or in groups from other sources (e.g. from the supernatants of other cells or synthesized de novo) and collected together to make the library of the invention.

Identifying/Studying/Isolating EEC Subtypes and EECS Using the Organoids of the Invention

Prior to the present invention, it was not possible to isolate live human EECs. The invention therefore opens up the possibility of studying the biology and function of individual human EECs and their secreted products for the first time.

Advantageously, the tagged EEC-specific genes, e.g. the tagged proteins encoded by the EEC-specific genes, in the reporter organoids or biobank of reporter organoids of the invention allow EECs and subtypes of EECs from the intestinal organoids to be studied. Accordingly, the invention provides a method for studying EECs or one or more EEC subtypes comprising detecting expression of a detectable marker (e.g. a fluorescent marker) linked to an EEC-specific gene in a reporter organoid of the invention. Advantageously the reporter organoids allow EEC subtypes to be identified and studied, for example, by monitoring expression of the tagged EEC-specific gene and/or monitoring secretion of the tagged hormone and/or tagged hormone precursor and/or monitoring production of a hormone from a hormone precursor and/or constructing a cell atlas as described herein. In embodiments in which the tagged EEC-specific gene is a hormone or a hormone precursor, expression and/or secretion may be monitored. Moreover, rare subtypes of EECs may be identified using the reporter organoids and optionally studied, e.g. using a cell atlas as described herein. Likewise, the intestinal organoids of the invention which are not reporter organoids similarly allow EECs from the intestinal organoids to be studied.

For example, the invention provides a method for studying secretion of one or more hormones and/or hormone precursors by EECs wherein the method comprises detecting hormones and/or hormone precursors secreted by EECs in an intestinal organoid of the invention. In some embodiments, the detecting uses a technique selected from monitoring for a decay or a change in location of a fluorescently tagged hormone and/or hormone precursor; a calcium reporter in EECs in the organoid as a proxy for induction of secretion; ELISA to measure secreted hormones and/or hormone precursors in the organoid supernatant; or mass spectrometry of the supernatant to detect and quantify secreted hormones and/or hormone precursors (secretomics), for example, as described herein. In some embodiments, whether or not a hormone and/or hormone precursor is secreted is detected. In some embodiments, the level of secreted hormone and/or hormone precursor is detected.

For example, the invention provides a method for studying expression of one or more EEC-specific genes (e.g. hormones, hormone precursors and/or hormone synthesizing enzymes) in EECs, wherein the method comprises detecting the expression of the one or more EEC-specific genes (e.g. hormones, hormone precursors and/or hormone synthesizing enzymes) by EECs in an intestinal organoid of the invention. In some embodiments, the detecting uses a technique selected from detecting expression of a fluorescent marker with which the hormone, hormone precursor and/or hormone synthesizing enzyme is tagged; and qPCR to assess the presence of transcripts of the one or more EEC-specific genes (e.g. hormones, hormone precursors and/or hormone synthesizing enzymes). In some embodiments, the method comprises studying whether or not an EEC-specific gene is expressed. In some embodiments, the method comprises studying the level of expression of the one or more EEC-specific genes. Thus, the detecting may comprise assessing the level of expression of a fluorescent marker or assessing the transcript levels by qPCR. For example, in some embodiments which use a reporter organoid of the invention, the detecting comprises detecting expression of the detectable marker (e.g. the fluorescent marker) with which the EEC-specific gene is tagged.

For example, the invention provides a method for studying production of one or more hormones that are produced from one or more hormone precursors secreted by EECs wherein the method comprises detecting one or more hormones produced from one or more hormone precursors secreted by EECs in an intestinal organoid of the invention. Thus, in some embodiments, the hormone is produced extracellularly by processing of a hormone precursor. For example, the hormone GLP-1 is produced extracellularly by processing of the hormone precursor GCG. In some embodiments, the detecting uses a technique selected from ELISA to measure the produced hormone in the organoid supernatant; or mass spectrometry of the organoid supernatant to detect and quantify the produced hormones (secretomics), for example, as described herein. Preferably, ELISA is used. In some embodiments, whether or not a hormone is produced is detected. In some embodiments, the level of the produced hormone is detected.

The technique used for detecting the detectable marker will depend on the type of detectable marker that is used. For example, fluorescence detection is a suitable technique when the detectable marker is a fluorescence marker. However, other techniques are suitable for other types of detectable marker. The skilled person will understand which types of technique are suitable depending on the type of detectable marker being used. For example, in embodiments in which the detectable marker is an antibiotic resistance marker, detecting antibiotic resistance is a suitable technique. For example, in some embodiments, detecting the expression of the one or more EEC-specific genes (e.g. hormones, hormone precursors and/or hormone synthesizing enzymes) tagged with an antibiotic resistance marker by EECs in an intestinal organoid of the invention comprises culturing the intestinal organoid in the presence of the antibiotic (e.g. blasticidin). Thus, in some embodiments, cell survival indicates expression of the EEC-specific gene.

The description above of the methods for studying secretion and/or expression and/or production, including the various techniques that may be used, may be extrapolated to the other methods described herein, for example, methods which involve contacting an organoid of the invention with a compound, mutatis mutandis.

In some embodiments of the various methods provided herein, the intestinal organoid of the invention is an intestinal organoid which comprises an inducible transcription factor for differentiating intestinal stem cells and/or intestinal cells with stem cell potential to EECs. In some embodiments, at least 10% of the cells in the intestinal organoid are EECs. For example, in some embodiments, the intestinal organoid comprises EECs that have been generated by overexpression of a transcription factor for differentiating intestinal stem cells and/or intestinal cells with stem cell potential to EECs. In some embodiments, the intestinal organoid does not comprise EECs. For example, in some embodiments, the intestinal organoid comprises stem cells and/or cells with stem cell potential which comprise the inducible transcription factor. In some embodiments, the organoid is a clonal organoid as described herein. In some embodiments, the organoid is a reporter organoid, as described herein. In some embodiments, the organoid is an intestinal organoid as described herein, which is obtained by or is obtainable by a method for enriching the population of EECs in an intestinal organoid. Thus, in some embodiments, the organoid is not a reporter organoid.

In some embodiments of the various methods provided herein which relate to studying secretion, expression and/or production of an EEC-specific protein (e.g. a hormone, hormone precursor and/or hormone synthesizing enzyme) the organoid comprises EECs. In embodiments which relate to contacting an organoid of the invention with a compound to determine the effects of the compound, the organoid may in some embodiments be an organoid which comprises EECs or in some alternative embodiments, it may be an organoid which does not comprise EECs. For example, in embodiments which relate to determining whether a compound affects differentiation of EECs in an organoid of the invention, the organoid that is contacted with the compound is preferably an organoid which does not comprise EECs. The differentiation of the stem cells and/or cells with stem cell potential to EECs can then be monitored.

In embodiments in which the organoid is a reporter organoid, the studying secretion of one or more hormones and/or hormone precursors by EECs preferably comprises detecting secretion of one or more tagged hormones and/or tagged hormone precursors by EECs in an intestinal organoid of the invention. Similarly, in embodiments in which the organoid is a reporter organoid, the studying expression of one or more EEC-specific genes (e.g. hormones, hormone precursors and/or hormone synthesizing enzymes) by EECs preferably comprises detecting the expression of one or more tagged EEC-specific genes (e.g. tagged hormones, tagged hormone precursors and/or tagged hormone synthesizing enzymes) by EECs in an intestinal organoid of the invention.

However, in some embodiments in which a reporter organoid is used, an alternative technique may be used, e.g. to study expression and/or production, such as qPCR, ELISA or secretomics. Use of a reporter organoid of the invention is useful when using an alternative technique to study expression and/or production because expression and/or production of the one or more EEC-specific genes may additionally be studied using the detectable marker, e.g. the fluorescent tag, alongside the alternative technique, if desired, for example, to confirm that the respective EEC-specific gene is being made. For example, in embodiments in which production of a hormone from a hormone precursor is being studied, e.g. using ELISA of the supernatant, it may be useful to monitor expression and/or secretion of the hormone precursor as well, for example, by monitoring expression and/or secretion of the tagged hormone precursor in a reporter organoid of the invention. In some embodiments, use of a reporter organoid enables FACS to be used to enrich for a cell subtype population based on a tagged EEC-specific gene (e.g. a tagged hormone, tagged hormone precursor or tagged hormone synthesizing enzyme) and then qPCR may be used to determine the expression of specific genes within the population.

The use of a reporter organoid is beneficial for high throughput screens compared to ELISAs. For example, it allows many conditions to be measured rapidly and instantly. In some embodiments which describe determining the levels of RNA and/or intracellular proteins, use of a reporter organoid advantageously allows purification of a subpopulation of EECs to pinpoint an effect to this population.

In some embodiments, the level of secretion, expression and/or production is a relative level of secretion, expression and/or production, e.g. compared to a different hormone, hormone precursor and/or hormone synthesizing enzyme, or compared to an EEC in a different state, for example, compared to an EEC that has not/has been contacted with a compound, or compared to an EEC from a different region of the intestine.

The invention also provides a method for studying EECs and/or one or more EEC subtypes comprising (i) contacting an intestinal organoid of the invention with one or more compounds; and (ii) determining whether secretion and/or expression and/or production of one or more EEC-specific genes (e.g. one or more hormones, hormone precursors and/or hormone synthesizing enzymes) is affected. Various methods for studying EECs and/or one or more EEC subtypes are described further herein.

The invention provides a method for visualising the EECs or one or more EEC subtypes in situ in the organoid of the invention, e.g. by visualising the location of a fluorescent detectable marker linked to an EEC-specific gene produced by the EECs or by the one or more EEC subtypes, e.g. a fluorescent detectable marker linked to a hormone, hormone synthesizing enzyme or hormone precursor. Optionally, in embodiments which involve visualising the location of one or more EEC subtypes, the method may further comprise visualising the location of all EECs in the organoid using a detectable marker, e.g. a fluorescent marker. For example, the EEC-specific gene that is tagged with the detectable marker may be a transcription factor as described herein or CHGA. Thus, in some embodiments, the EEC-specific gene is selected from NEUROG3, ATOH1/MATH1, NEUROD1 or CHGA. In some embodiments, the EEC-specific gene is NEUROG3. In some embodiments, the organoid is a reporter organoid. In some embodiments, the organoid is not a reporter organoid and for example, the detectable marker is a marker whose expression is linked to expression of the inducible transcription factor.

In some embodiments, the gene encoding the detectable marker is separated from the EEC-specific gene, for example, by a self-cleavable site, for example, by a P2A site. In some embodiments, the detectable marker is a fluorescent protein. Thus, in some embodiments, the method is for determining the location of EECs and/or EEC subtypes in an intestinal organoid of the invention. The results can be extrapolated to determine the location of the cells in vivo in the region of the organ from which the organoid was established. Accordingly, in some embodiments, the method for studying EECs or one or more EEC subtypes is for determining the location of EECs and/or one or more EEC subtypes in the region of the intestine from which the organoid was established. In some embodiments, the method allows determination of the EEC subtypes present in an organoid established from a particular region of the intestine. For example, in some embodiments, the method for studying EECs or one or more EEC subtypes allows determination of the EEC subtypes present in an organoid established from a particular region of the intestine. This information can be extrapolated to the intestine in vivo. Accordingly, in some embodiments, the method of the invention is for determining the EEC subtypes present in an organoid of the invention established from a particular region of the intestine. For example, the method of the invention may allow determination of the EEC subtypes present in vivo in the region of the intestine from which the organoid was established. For example, in some embodiments, the method comprises determining which hormones, hormone synthesizing enzymes and/or hormone precursors are expressed by EECs in one or more reporter organoids of the invention established from a particular region of the intestine by detecting expression of one or more detectable markers (e.g. fluorescent markers) linked to hormones, hormone synthesizing enzymes and/or hormone precursors in the reporter organoids, and determining which EECs are present based on which hormones, hormone synthesizing enzymes and/or hormone precursors are expressed.

For example, if GCG is expressed, then an L-cell is present; if TPH1 is expressed, then an EC-cell is present; if GHRL and MLN is expressed, then an M-X cell is present; if MLN but no GHRL is expressed then an M-cell is present; if GHRL but no MLN is expressed than an X-cell is present; if CCK is expressed, then an I-cell is present; if GIP and GAST are expressed, then a K-G cell is present; if GIP but no GAST is expressed then a K-cell is present; if GAST but no GIP is expressed then a G-cell is present; if NTS is expressed then an N-cell is present and/or if SST is expressed, then a D-cell is present.

Accordingly, in some embodiments, there is provided a method for determining whether one or more EEC subtypes is present, wherein the EEC subtypes are selected from L-cells, EC-cells, M-X cells, M-cells, X-cells, I-cells, K-G cells, K-cells, G-cells, N-cells and D-cells. Alternatively, in some embodiments, the EEC subtype may be named according to the hormone expressed. The method may comprise the steps described herein.

Advantageously, the ability to visualise EECs and/or one or more EEC subtypes enables the EECs and/or the one or more EEC subtypes to be isolated. In some embodiments, the EECs and/or EEC subtypes are isolated from the organoids before being studied. In some embodiments, the EEC subtypes are isolated from each other before being studied.

For example, the detectable marker linked to the EEC-specific gene (e.g. the hormone, hormone synthesizing enzyme or hormone precursor) advantageously allows EECs and subtypes of EECs to be isolated from organoids. The invention therefore provides a method for isolating one or more EECs or isolating one or more EECs of an EEC subtype of interest, wherein the method comprises identifying cells in a reporter organoid of the invention that express a detectable marker linked to an EEC-specific gene (e.g. a hormone, hormone synthesizing enzyme or hormone precursor) that identifies the EECs or the subtype of EECs of interest, respectively, and isolating said cell or cells. Examples of EEC-specific genes and hormones, hormone synthesizing enzymes and hormone precursors that can be used to identify EECs and EEC subtypes are described herein. The cell or cells isolated by a method of the invention are also provided. Similarly, a cell or cells obtained by or obtainable by a method of the invention are provided.

The invention therefore further provides the use of a tagged EEC-specific gene (e.g. a tagged hormone, tagged hormone synthesizing enzyme or tagged hormone precursor) to isolate one or more EECs or to isolate one or more EECs of an EEC subtype. For example, in the case of EECs, tagged GCG may be used to isolate one or more L-cells; tagged TPH1 may be used to isolate one or more EC-cells; tagged GHRL and/or tagged MLN may be used to isolate one or more M-X cells, M-cells or X-cells, respectively; tagged CCK may be used to isolate one or more I-cells; tagged GIP and/or tagged GAST may be used to isolate one or more K-G cells, K-cells or G-cells, respectively; tagged NTS may be used to isolated one or more N-cells; and/or tagged SST may be used to isolate one or more D-cells.

In some embodiments, the method comprises isolating a population of cells comprising or consisting of all subtypes of EECs from the organoid. In some embodiments, the method comprises isolating a population of cells comprising or consisting of one or more (e.g. 1, 2, 3, 4, 5 or more or all) subtypes of EECs from the organoid. In some embodiments, the detectable marker (e.g. fluorescent marker) is linked to an EEC-specific gene (e.g. a transcription factor or a hormone, hormone synthesizing enzyme or hormone precursor) that is expressed in one or more (e.g. 1, 2, 3, 4, 5 or more or all) subtypes of EECs in the organoid. For example, in some embodiments, the detectable marker is linked to a transcription factor for differentiating intestinal stem cells and/or intestinal cells with stem cell potential to EECs, e.g. to NEUROG3, MATH1/ATOH1 or NEUROD1. In some embodiments, the detectable marker is linked to the hormone CHGA. CHGA is a broad marker for all EECs. In some embodiments, cells expressing detectable markers (e.g. fluorescent markers) linked to different EEC-specific genes are selected and grouped together. In some embodiments, cells expressing a detectable marker (e.g. a fluorescent marker) linked to an EEC-specific gene (e.g. a hormone, hormone synthesizing enzyme or hormone precursor) specific for that EEC subtype are selected and grouped together.

The cells that are isolated are preferably live cells. Accordingly, the invention provides a method for isolating one or more live EECs or isolating one or more live cells of an EEC subtype of interest, for example, as described herein. Similarly, in some embodiments, the method comprises isolating a population of live cells comprising or consisting of one or more (e.g. 1, 2, 3, 4, 5 or more or all) subtypes of EECs. Preferably, the live EECs are live human EECs.

Isolated single cells or populations of cells obtained by or obtainable by the methods or uses described herein are also provided by the invention. Similarly provided are isolated single cells or populations of cells described herein. As mentioned above, the cells that are isolated are preferably live cells. Thus, the invention provides one or more isolated live EECs, e.g. of one or more EEC subtypes, for example, as described herein. Similarly, the invention provides an isolated population of cells which comprises or consists of live EECs or of one or more live EEC subtypes. For example, the invention provides an isolated population of cells which comprises or consists of a single live EEC subtype. Preferably, the live EECs are live human EECs. In some embodiments, the isolated population of cells comprises at least 2 EECs, for example, at least 10, 20, 50, 100, 200, 1000, 5000 or 10,000 EECs. Subtypes of EEC are described herein.

Also provided are isolated single cells or populations of cells, as described herein, for use in therapy. Similarly provided are isolated single cells or populations of cells, as described herein, for use in a method of treating or preventing a disease or disorder as described herein. The skilled person will understand which disease or disorder can be treated or prevented depending on the cell type, for example, using the teaching provided herein.

For example, the invention provides an isolated EEC selected from the group consisting of: an L-cell, I-cell, K-cell, N-cell, enterochromaffin or EC-cell, M-cell, X-cell, M-X cell, G-cell, K-G cell, and D-cell. Similarly, the invention provides an isolated EEC selected from the group consisting of: an GLP1+ cell, PYY+ cell, CCK+ cell, Serotonin+ cell, PPY+ cell, NTS+ cell, GIP+ cell, Motilin+ cell, Ghrelin+ cell, Motilin+ and Ghrelin+ cell, Angiotensin+ cell, SST+ cell, GAST+ cell, and GIP+ and GAST+ cell. Preferably, the isolated cell is a human cell. An isolated population of such cells is also provided. In embodiments of the various aspects of the invention which describe the use of one or more EEC subtypes, the subtypes are preferably selected from these lists.

An isolated population of cells which comprises or consists of EECs or of one or more EEC subtypes is also provided herein. In some embodiments, a population comprises more than 50%, 60%, 70%, 80%, 90%, 95%, 98%, 99% EECs or comprises more than 50%, 60%, 70%, 80%, 90%, 95%, 98%, 99% cells of one or more EEC subtypes.

Any suitable method in the art for sorting and/or isolating the EECs or the subtypes of EECs based on the detectable marker, e.g. the fluorescent marker, may be used. In some embodiments, FACS is used to sort and isolate cells based on expression of a fluorescent marker. For example, FACS may be used to separate and isolate the EECs or the subtypes thereof from other cells, based on expression of the fluorescent marker linked to the EEC-specific gene (e.g. the hormone, hormone synthesizing enzyme or hormone precursor). In some alternative embodiments, the location of the EECs or of the different subtypes thereof is assessed by visualising the fluorescent marker, for example using confocal microscopy. The cells expressing the fluorescent marker may then be isolated, if desired.

Other Methods Using the Organoids and Biobank

The organoids (e.g. the reporter organoids) described herein may be of use in a method as described herein, for example, for generating a cell atlas, for performing differentiation screens using a tagged EEC-specific gene such as a hormone to detect which cell population is made in response to a particular stimulus, for secretion screens, for example, determining how release of a specific hormone is triggered, or for drug discovery screens or in any other method described herein. The intestinal organoids described herein are all provided by the present invention. Thus, the invention provides the use of an organoid of the invention, e.g. a reporter organoid, in an assay.

In some embodiments, a method of the invention allows a determination to be made as to whether EECs are biased towards a subtype which results in more or less production of one or more specific hormones by modulating a target. This may optionally be called a differentiation screen.

In some embodiments, a method of the invention allows a determination to be made as to whether one or more specific hormones is released upon engagement of a target. This may optionally be called a secretion screen.

For the differentiation screens, the organoid is preferably contacted with the one or more compounds before or during the step of differentiating stem cells and/or cells with stem cell potential in the organoid to EECs. For example, the organoid may be contacted with the one or more compounds before, after or simultaneously with the inducer for the inducer promoter of the transcription factor for differentiating intestinal stem cells to EECs. Thus, the organoid used for a differentiation screen is preferably a stable organoid line (e.g. a clonal organoid). For secretion screens, the organoid is preferably contacted with the one or more compounds after the step of differentiating stem cells in the organoid to EECs.

In some embodiments, a method of the invention comprises performing an assay selected from a fluorescence assay, a qPCR assay, a calcium reporter assay, an ELISA assay or a secretomics assay. For example, a fluorescence assay may be used to detect fluorescent tagged hormones and/or to measure fluorescent tagged hormone levels, for example, using a fluorescent plate reader or a fluorescence microscope. In some embodiments, fluorescence decay and/or the location of the tagged hormones and/or tagged hormone precursors is visualised and/or quantified. In some embodiments, a qPCR assay is used to assess hormone transcript levels. In some embodiments, a calcium reporter is used as a proxy for induction of secretion. In some embodiments, an ELISA is used to measure secreted hormones and/or hormone precursors and/or hormones produced from hormone precursors in the organoid supernatant. In some embodiments, secretomics is used to detect and quantify secreted products (e.g. upon stimulation) or to detect and quantify a hormone produced from a hormone precursor by performing mass spectrometry on the organoid culture supernatant.

In preferred embodiments, the one or more EEC-specific genes (e.g. one or more hormones, hormone precursors and/or hormone synthesizing enzymes) are tagged with a fluorescent marker. The fluorescence assay may comprise assessing fluorescent reporter levels. In some embodiments, these are assessed using a fluorescent plate reader or fluorescence microscope.

Expression of transcription factors is an intermediate step to stem cells and/or cells with stem cell potential becoming mature EECs. For example, upregulation of a specific transcription factor may predispose a cell to become an EEC of a particular subtype. In some embodiments, the invention provides a method for determining whether modulating expression of a target transcription factor controls differentiation of EECs, wherein the method comprises (i) contacting an intestinal organoid of the invention with a compound that modulates expression of the transcription factor; (ii) determining whether the expression level of the transcription factor is affected; (iii) determining whether the transcript levels or production of one or more hormones, hormone precursors and/or hormone synthesizing enzymes is affected; (iv) extrapolating an effect on transcript levels of one or more hormones, hormone precursors and/or hormone synthesizing enzymes to a change in the number of cells of one or more EEC subtypes present; and (v) extrapolating an effect on the number of cells of one or more EEC subtypes present to the change in expression levels of the transcription factor. For example, in some embodiments, step (iv) extrapolates an effect on transcript levels to a change in the ratio of one or more EEC subtypes compared to one or more other EEC subtypes. In some embodiments, step (ii) is conducted using a fluorescence assay. In some embodiments, step (iii) is conducted using a qPCR assay. The qPCR assay is preferably conducted after a suitable period of time from induction of differentiation of the stem cells to EECs. For example, in some embodiments, the qPCR assay is conducted 3-7, 4-6 or 5 days after induction of differentiation. Step (i) is preferably conducted in a differentiation medium or an expansion medium of the invention. In some embodiments, a reporter organoid is used which comprises more than one tagged transcription factor and the method may be used to detect the cascade of transcription factor activation that leads to differentiation of EECs. In some embodiments, a reporter organoid is used which comprises the tagged transcription factor and also comprises one or more tagged hormones, hormone precursors and/or hormone synthesizing enzymes. In some further alternative embodiments, step (iii) of the method instead comprises contacting a reporter organoid comprising one or more tagged hormones, hormone precursors and/or hormone synthesizing enzymes with the compound and determining expression levels of the one or more tagged hormones, hormone precursors and/or hormone synthesizing enzymes, e.g. using fluorescence detection, e.g. using a method as described herein, instead of using qPCR to determine transcript levels. Step (iv) may be adapted accordingly. In some embodiments, the organoid used in step (i) is a reporter organoid which comprises the transcription factor tagged at its endogenous locus with a detectable marker, wherein the transcription factor is under control of an inducible promoter. In some embodiments, the organoid used in step (i) is an intestinal organoid of the invention which comprises an inducible transcription factor for differentiating intestinal stem cells and/or cells with stem cell potential to EECs. Thus, in some embodiments, the compound used in step (i) is the inducer for the inducible promoter of the transcription factor. In some embodiments, the organoid used in step (i) is a stable organoid line, as described herein.

In some embodiments, the EEC-specific transcription factor is selected from ARX, ASCL1, HHEX, LMX1A, MNX1, NKX2-2, PAX4, PAX6, SOX4 and RFX6.

Also provided is a method for identifying/validating a drug target of interest in EECs or in one or more EEC subtypes, wherein the method comprises (i) contacting one or more organoids of the invention with a compound specific for the target; and (ii) determining whether secretion and/or expression and/or production of one or more hormones, hormone precursors and/or hormone synthesizing enzymes is affected. In embodiments in which secretion and/or expression and/or production of one or more hormones, hormone precursors and/or hormone synthesizing enzymes is affected, the method may further comprise identifying/screening/validating the compound for suitability for treating or preventing a disease or disorder using a method as described herein, wherein the compound is known to target the drug target of interest.

The term “for identifying/validating” means “for identifying and/or validating”.

The target used in the various methods of the invention is a target expressed by EECs, e.g. preferably by human EECs. For example, in some embodiments, the target is expressed on the surface of EECs (for example, in all EECs or in one or more subtypes thereof). In some embodiments, the target is expressed inside EECs. In embodiments which describe the target being “in an EEC”, e.g. “in a human EEC”, this encompasses a target on the surface of the EEC and a target inside the EEC. The target is targeted in the EEC, e.g. in the human EEC. The target may be any RNA or protein expressed by an EEC. The gene encoding an RNA or protein is also encompassed as a target of the invention. The target is preferably a protein. The skilled person will understand what types of compound are suitable for targeting the target depending upon whether it is a protein, RNA or DNA. In some embodiments, expression of the target is determined at the protein level. In some embodiments, expression of the target is determined at the RNA level. In some embodiments, the RNA or protein is expressed specifically in EECs or in one or more EEC subtypes. Preferably, the one or more targets is a target identified herein (for example, in the description, the claims, the examples or the figures) as being expressed in EECs or in a particular EEC subtype. For example, in preferred embodiments, the one or more targets is an RNA or protein identified herein (for example, in the description, the claims, the examples or the figures) as being expressed in EECs or in a particular EEC subtype. In some embodiments, the target is expressed in one EEC subtype but is not expressed in any other EEC subtypes. In some embodiments, the target is identified as being expressed in EECs or in one or more EEC subtypes using a human EEC atlas, for example, a human EEC atlas as described herein. Thus, in some embodiments, the target is a target described herein. In some embodiments, the one or more targets has been identified as being expressed in human EECs or in one or more human EEC subtypes for the first time in the present invention. In some embodiments, the one or more targets are expressed in one or more human EECs but are not expressed in murine EECs. In some embodiments, the one or more targets are expressed in one or more human EECs but are not expressed in EECs from any other organism.

In some embodiments, the target is a cell surface receptor, for example, selected from the various cell surface receptors described herein. In some embodiments, the target is a transcription factor, for example, selected from the various transcription factors described herein. In some embodiments, the target is a hormone, for example, selected from the various hormone described herein. In some embodiments, the target is an EEC-specific gene (e.g. a protein encoded by an EEC-specific gene). In some embodiments, the target is a target of interest. In some embodiments, the target is unknown.

In some embodiments of the various methods described herein, the one or more tagged hormones, tagged hormone precursors and/or tagged hormone synthesizing enzymes are two or more (e.g. 2, 3, 4, 5, 6, 7, 8 or more) tagged hormones, tagged hormone precursors and/or tagged hormone synthesizing enzymes.

The skilled person will be able to select a suitable culture medium for the intestinal organoid for use in each of the steps of a method of the present invention. For example, for method steps which involve differentiating stem cells and/or cells with stem cell potential to EECs, the culture medium may be an expansion medium and/or a differentiation medium, for example, as described herein. A preferred culture medium for differentiating stem cells and/or cells with stem cell potential to EECs is the ENR medium, as described herein. For methods steps which comprise the use of an organoid in which the stem cells have already been differentiated to EECs, the culture medium may in some embodiments be a culture medium suitable for maintaining the intestinal organoid which does not comprise components that would affect the readout of the assay. In some embodiments, a basal medium is used, for example, as described herein. In some embodiments, the basal medium does not comprise any cell specific factors. For example, in some embodiments, DMEM is used. In some embodiments which comprise the use of an organoid in which the stem cells have already been differentiated to EECs, the culture medium is a differentiation medium, for example, the ENR medium. For example, for method steps which involve introducing one or more constructs into an intestinal stem cell and/or into an intestinal cell with stem cell potential, the culture medium may be a culture medium suitable for stem cell culture.

In some embodiments, the step of contacting an organoid of the invention with the compound comprises culturing the organoid in the presence of the compound, e.g. by adding the compound to the culture medium. In some embodiments, the step of contacting an organoid of the invention with the compound comprises injecting the compound into the lumen of the organoid, for example, as described in Bartfeld, S. et al., 2015. In some embodiments, the method is repeated so that the compound is added to the culture medium in one experiment, and so the compound is injected into the lumen of the organoid in the other experiment. A change in secretion and/or expression and/or production of the EEC-specific gene in either of the experiments may then be detected.

The invention provides a method for identifying/screening/validating a compound for suitability for modulating secretion and/or production of one or more hormones and/or hormone precursors and/or modulating expression of one or more EEC-specific genes, wherein the method comprises (i) contacting an organoid of the invention with the compound; and (ii) determining whether secretion or production of one or more hormones and/or hormone precursors is affected and/or whether expression of one or more EEC-specific genes is affected.

In preferred embodiments of the various methods described herein, the EEC-specific gene is a hormone, hormone precursor or a hormone synthesizing enzyme. In more preferred embodiments, the EEC-specific gene is a hormone or a hormone precursor. For example, these may be tagged in a reporter organoid of the invention.

In embodiments which describe secretion, expression, production, etc. of EEC-specific genes, it will be understood that this encompasses detecting secretion, expression, production, etc. at the protein level. The term “EEC-specific gene” may therefore be replaced with “EEC-specific protein” where appropriate.

In some embodiments, the method is for identifying/screening/validating a compound for suitability for modulating, increasing, inducing, reducing or ceasing secretion and/or expression and/or production of one or more EEC-specific genes (e.g. one or more hormones, hormone precursors and/or hormone synthesizing enzymes), wherein the method comprises (i) contacting an organoid of the invention with the compound; and (ii) determining whether secretion and/or expression and/or production of the one or more EEC-specific genes (e.g. hormones, hormone precursors and/or hormone synthesizing enzymes) is modulated, increased, induced, reduced or ceased, respectively. Accordingly, such a method may be used to identify/screen/validate an inducer of gene expression or of hormone or hormone precursor secretion.

In some embodiments, the organoid is a reporter organoid and the method further comprises comparing the results to the results obtained from contacting one or more further reporter organoids of the invention which the same compound, wherein said one or more further reporter organoids comprise one or more different tagged EEC-specific genes (e.g. different tagged hormones, tagged hormone precursors and/or tagged hormone synthesizing enzymes). A finding that the compound modulates, increases, induces, ceases or decreases secretion and/or expression and/or production of the first EEC-specific protein (e.g. hormone, hormone precursor or hormone synthesizing enzyme), but does not modulate, increase, induce, cease or decrease secretion and/or expression and/or production of the one or more further different EEC-specific proteins (e.g. hormones, hormone precursors and/or hormone synthesizing enzymes) suggests that the compound is suitable for modulating, increasing, inducing, ceasing or reducing, respectively, secretion and/or expression and/or production, respectively, of the specific EEC-specific protein. The EEC-specific gene is preferably a hormone, hormone precursor or a hormone synthesizing enzyme.

As a further example, in embodiments in which step (i) of a method for identifying/screening/validating a compound uses a reporter organoid, the reporter organoid may comprise more than one (e.g. 2, 3 or more) tagged EEC-specific genes (e.g. tagged hormones, tagged hormone precursors and/or tagged hormone synthesizing enzymes). Relative changes of the different tagged EEC-specific proteins (e.g. of the different tagged hormones, tagged hormone precursors and/or tagged hormone synthesizing enzymes) to the same stimulus can then be compared. Thus, the method may be used to determine if a compound modulates, increases, induces, ceases or reduces secretion and/or expression and/or production of a specific EEC-specific protein in the EEC (e.g. a specific hormone, hormone precursor or hormone synthesizing enzyme in the EEC) but does not modulate, increase, induce, cease or reduce secretion and/or expression and/or production of one or more other EEC-specific proteins in the EEC (e.g. other hormones, hormone precursors and/or hormone synthesizing enzymes).

The term “for identifying/screening/validating” means “for identifying, screening and/or validating”.

Also provided is a method for identifying/screening/validating a compound for suitability for treating or preventing a disease or disorder, wherein the method comprises (i) contacting an organoid of the invention with the compound; and (ii) determining whether secretion and/or expression and/or production of an EEC-specific protein (e.g. a hormone, hormone precursor and/or hormone synthesizing enzyme) is affected. In some embodiments, the compound is a candidate compound.

It is known in the art that a number of the hormones, for example, those that are produced by EECs, are implicated in a number of diseases and disorders (for example, see the introduction and the description herein). Accordingly, in some embodiments, a reporter organoid is obtained/made in which one or more (e.g. 1, 2, 3, 4 or more) hormones is tagged that is known to be implicated in a particular disease or disorder and the method is then used to identify/validate/screen a compound for suitability for treating or preventing that disease or disorder. Examples of such hormones and their respective diseases or disorders are provided herein and can be used in the methods of the invention accordingly. In some embodiments in which it is not suitable to tag the hormone itself, an enzyme involved in the synthesis of the hormone (i.e. a hormone synthesizing enzyme) or a precursor of the hormone (i.e. a hormone precursor) may be tagged instead. Tagging relates to tagging with a detectable marker, as described herein.

In some embodiments, if the compound increases or induces expression and/or secretion and/or production by EECs or by one or more subtypes thereof of one or more hormones, hormone precursors and/or hormone synthesizing enzymes known to be or suggested to be implicated in the disease or disorder, this suggests that the compound is a suitable candidate for treating or preventing the disease or disorder. In some embodiments, if the compound decreases or ceases expression and/or secretion and/or production by EECs or by one or more subtypes thereof of one or more hormones, hormone precursors and/or hormone synthesizing enzymes known to be or suggested to be implicated in the disease or disorder, this suggests that the compound is a suitable candidate for treating or preventing the disease or disorder. It will be clear to the skilled person whether an increase or a decrease is useful depending on the disease or disorder of interest, for example, based on the teaching provided herein.

In embodiments which describe a hormone precursor being implicated in a disease or disorder, this encompasses the hormone produced from the hormone precursor being implicated in the disease or disorder.

In some embodiments, an organoid is used which is not a reporter organoid. For example, the invention provides a method for identifying/screening/validating a compound for suitability for treating or preventing a disease or disorder, wherein the method comprises (i) contacting an organoid of the invention with the compound; and (ii) determining whether expression of an EEC-specific gene (e.g. a hormone, hormone precursor and/or hormone synthesizing enzyme) is affected. For example, expression levels of the one or more EEC-specific genes may be determined using qPCR or any other suitable method.

Also provided is a method for identifying/screening/validating a compound for suitability for treating or preventing a disease or disorder, wherein the method comprises (i) contacting an organoid of the invention with the compound; and (ii) determining whether the compound modulates expression of, agonises or antagonises in EECs or in one or more EEC subtypes one or more targets known to be or suggested to be implicated in the disease or disorder. In some embodiments, the compound is a candidate compound. In some embodiments, the target is a target identified herein as being expressed in EECs or in one or more subtypes thereof. In some embodiments, step ii) comprises determining whether the compound modulates expression of, agonises or antagonises in EECs or in one or more EEC subtypes a target as described herein known to be or suggested to be implicated in the disease or disorder. In some embodiments, step ii) comprises determining whether the compound modulates expression of a target known to be or suggested to be implicated in the disease or disorder. In some embodiments, step ii) comprises determining whether the compound agonises a target known to be or suggested to be implicated in the disease or disorder. In some embodiments, step ii) comprises determining whether the compound antagonises a target known to be or suggested to be implicated in the disease or disorder. For example, in some embodiments, the target is a cell surface receptor. Identifying agonists and/or antagonists of receptors expressed by EECs or one or more subtypes thereof, wherein the receptor is known to be or suggested to be implicated in the disease or disorder, is useful for identifying compounds suitable for treating or preventing the disease or disorder. In some embodiments, if the compound increases expression in EECs or in one or more subtypes thereof of the target, this suggests that the compound is a suitable candidate for treating or preventing the disease or disorder. In some embodiments, if the compound decreases expression in EECs or in one or more subtypes thereof of the target, this suggests that the compound is a suitable candidate for treating or preventing the disease or disorder. The skilled person will understand whether an increase or a decrease should be looked for depending on the disease or disorder of interest and the target being studied, for example, based on the teaching herein.

In some embodiments, the stem cells and/or cells with stem cell potential in the organoid for use in the invention have been differentiated to EECs by overexpression of the inducible transcription factor. This is the preferred method for differentiating the cells to EECs. In some embodiments, the stem cells and/or cells with stem cell potential in the organoid for use in the invention have been differentiated to EECs by other means. In some embodiments, the stem cells and/or cells with stem cell potential in the organoid for use in the invention have not been differentiated to EECs. The skilled person will understand which type of organoid to use depending on the method in question.

Also provided is a method for determining whether a patient will respond to a compound for treating or preventing a disease or disorder, wherein the method comprises (i) establishing an organoid of the invention from said patient; (ii) contacting the organoid with the compound; and (iii) determining whether secretion and/or expression and/or production of one or more EEC-specific proteins (e.g. hormones, hormone precursors and/or hormone synthesizing enzymes) is affected. The one or more EEC-specific proteins (e.g. hormones, hormone precursors and/or hormone synthesizing enzymes) are preferably one or more EEC-specific proteins that are implicated in the disease or disorder. In some embodiments, the compound is a candidate compound. In some embodiments, a reporter organoid is used which comprises one or more EEC-specific genes (e.g. hormones, hormone precursors and/or hormone synthesizing enzymes) tagged with a detectable marker, wherein the one or more EEC-specific proteins are implicated in the disease or disorder. In some embodiments, the reporter organoid additionally comprises other tagged EEC-specific genes, e.g. other tagged hormones, which are not implicated in the disease or disorder. In some embodiments, an intestinal organoid of the invention which is not a reporter organoid may be used in step i) of a method for determining whether a patient will respond to a compound for treating or preventing a disease or disorder. Step (iii) may then comprise determining whether the expression or production of one or more EEC-specific proteins (e.g. hormones, hormone precursors and/or hormone synthesizing enzymes) is affected, e.g. using qPCR or ELISA. In some embodiments, the method may further comprise treating said patient with the compound if the patient is identified as a patient who will respond to the compound for treating or preventing the disease or disorder.

In some embodiments, the organoid (e.g. the reporter organoid) is a disease organoid. For example, in some embodiments, the organoid has been obtained from a patient having a disease or disorder of interest. In some embodiments, the disease or disorder is a disease or disorder as described herein. In some embodiments, the organoid comprises a mutation in a gene and/or protein of interest.

In some embodiments, the disease or disorder is a disease or disorder of the intestine. In some embodiments, the disease or disorder is a disease or disorder of the small intestine. In some embodiments, the disease or disorder is a disease or disorder of the colon. In some embodiments, the disease or disorder is a disease or disorder affecting EECs. In some embodiments, the disease or disorder is a disease or disorder affecting one or more EEC subtypes, wherein the subtype is selected from the list of subtypes described herein. In some embodiments, the disease or disorder is a disease or disorder caused by an imbalance of one or more hormones and/or hormone precursors expressed and/or secreted and/or produced by EECs. For example, the one or more hormones and/or hormone precursors may be one or more hormones and/or hormone precursors as described herein. Hormones which are produced from the hormone precursors are also encompassed by this definition. In other words, in some embodiments, the disease or disorder is a disease or disorder caused by an imbalance of one or more hormones and/or hormone precursors produced by EECs.

In some embodiments, the disease or disorder is selected from a gastrointestinal disease or disorder (e.g. inflammatory bowel disease, or gut motility disorders, e.g. bowel movement disorders, IBS, Parkinson's disease, Gastroparesis), a metabolic disease or disorder (e.g. obesity, diabetes); a digestive disease or disorder (e.g. abnormal bile or pancreatic enzyme secretion, high acid secretion in the stomach, e.g. caused by abnormal parietal cell activity in the stomach, e.g. indigestion or acid reflux); a disease or disorder relating to food intake, e.g. an appetite-related disease or disorder (for example, obesity, anorexia, cancer cachexia or Prader-Willi syndrome), for example requiring appetite stimulation (e.g. anorexia or cancer cachexia) or appetite inhibition (e.g. obesity or Prader-Willi syndrome); a disease or disorder relating to anxiety and reward (e.g. obesity or anorexia); a neurodegenerative disease (for example, Parkinson's disease or Alzheimer's Disease); and a neuroendocrine tumour (for example, a small intestinal gastrinoma, e.g. Zollinger-Ellison syndrome). In some embodiments, diabetes is considered an appetite-related disease or disorder requiring appetite inhibition, and so can be included in the above list of appetite-related diseases or disorders. For example, in some embodiments, the disease or disorder is selected from a disease or disorder relating to food intake (e.g. obesity or anorexia), insulin release (e.g. diabetes), secretion of digestive enzymes (e.g. by the pancreas) or a gut motility disorder. In some embodiments, the disease or disorder is selected from obesity, anorexia, diabetes and a gut motility disorder. In some embodiments, the disease or disorder is a gut motility disorder. In some embodiments, the gut motility disorder is selected from the list consisting of irritable bowel syndrome (IBS), diabetes, Hirschsprung's Disease, bacterial overgrowth in the small intestine (for example, in the upper part of the small intestine), gastroparesis, chronic intestinal pseudo-obstruction and Parkinson's disease In some embodiments, the gut motility disorder is selected from the group consisting of irritable bowel syndrome, diabetes, Hirschsprung's Disease and bacterial overgrowth in the small intestine (for example, in the upper part of the small intestine). In some embodiments, the gut motility disorder is selected from a bowel movement disorder, Parkinson's disease and gastroparesis. In some embodiments, the bowel movement disorder is selected from gastroesophageal reflux disease, intestinal dysmotility, constipation and hirschsprung's disease. In some embodiments, the irritable bowel syndrome is irritable bowel syndrome characterised by alterations in gut motility. In some embodiments, the disease or disorder is diabetes. In some embodiments, the diabetes is type II diabetes. In some embodiments, the disease or disorder is a disease or disorder requiring appetite inhibition. In some embodiments, the disease or disorder is obesity, Prader-Willi syndrome or diabetes. In some embodiments, the disease or disorder is obesity. In some embodiments, the disease or disorder is Prader-Willi syndrome. In some embodiments, the disease or disorder is diabetes. In some embodiments, the diabetes is type II diabetes. In some embodiments, the disease or disorder is a disease or disorder requiring appetite stimulation. In some embodiments, the disease or disorder is anorexia or cancer cachexia. For example, in some embodiments, the disease or disorder is anorexia. In some embodiments, the disease or disorder is cancer cachexia. In some embodiments, the disease or disorder is a neurodegenerative disease or disorder. In some embodiments, the disease is Alzheimer's disease. In some embodiments, the disease is Parkinson's disease. In some embodiments, the disease or disorder is high acid secretion in the stomach. In some embodiments, the high acid secretion is caused by abnormal parietal cell activity in the stomach. In some embodiments, the disease or disorder is indigestion caused by high acid secretion in the stomach. In some embodiments, the disease or disorder is acid reflux caused by high acid secretion in the stomach. In some embodiments, the disease or disorder is IBS. In some embodiments, the disease or disorder is depression. In some embodiments, the disease or disorder is IBD. In some embodiments, the IBD is Crohn's disease. In some embodiments, the IBD is ulcerative colitis. In some embodiments, the list of diseases and disorders additionally comprises neuroendocrine cancer or a neuroendocrine tumour. In some embodiments, the treatment improves one or more symptoms selected from the group consisting of: diarrhoea, constipation, gas, nausea, acid reflux, regurgitation of food, bloating, abdominal discomfort, abdominal pain, malnutrition, loss of appetite, and weight loss. In some embodiments, the disease or disorder is selected from diarrhoea, constipation, gas, nausea, acid reflux, regurgitation of food, bloating, abdominal discomfort, abdominal pain, malnutrition, loss of appetite and weight loss.

For example, PYY is implicated in obesity. In some embodiments, a finding that contacting an organoid of the invention with a compound increases secretion and/or expression of PYY indicates that the compound is suitable for treating or preventing obesity. Thus, in some embodiments, the disease or disorder is obesity.

Ghrelin and Cerebellin 1 are implicated in appetite-related diseases and disorders (e.g. Howick, K. et al. 2017 and Gardiner, J. V. et al. 2010). In particular, Ghrelin and Cerebellin 1 are implicated in appetite stimulation. Ghrelin receptor agonists are currently being tested in clinical trials and show very positive results in terms of increasing appetite. In some embodiments, a finding that contacting an organoid of the invention with a compound increases secretion and/or expression of GHRL and/or Cerebellin 1 indicates that the compound is suitable for treating or preventing a disease or disorder which requires appetite stimulation, e.g. anorexia and/or cancer cachexia. Thus, in some embodiments, the disease or disorder is a disease or disorder treatable or preventable by inducing or increasing secretion and/or expression of Ghrelin and/or Cerebellin 1, e.g. anorexia or cancer cachexia. In some embodiments, the disease or disorder is anorexia. In some embodiments, the disease or disorder is cancer cachexia. Conversely, inhibition of Ghrelin or Cerebellin 1 would inhibit appetite (Altabas, V. and Zjac̆ić-Rotkvić, V. 2015). Thus, Ghrelin and Cerebellin are also implicated in appetite inhibition. Thus, in some embodiments, a finding that contacting an organoid of the invention with a compound decreases secretion and/or expression of GHRL and/or Cerebellin 1 indicates that the compound is suitable for treating or preventing a disease or disorder which requires appetite inhibition, e.g. obesity and/or diabetes and/or Prader-Willi syndrome. Thus, in some embodiments, the disease or disorder is a disease or disorder treatable or preventable by ceasing or decreasing secretion and/or expression of Ghrelin and/or Cerebellin 1, e.g. obesity, diabetes or Prader-Willi syndrome. In some embodiments, the disease or disorder is obesity. In some embodiments, the disease or disorder is diabetes. In some embodiments, the diabetes is type II diabetes. In some embodiments, the disease or disorder is Prader-Willi syndrome.

CCK, GLP-1, PPY and PYY are implicated in appetite-related diseases and disorders (e.g. Silva, A. D. and Bloom, S. R., 2012; Kim, G. W. et al., 2011; Shah, M. and Vella, A., 2014). In particular, CCK, GLP-1, PPY and PYY are implicated in appetite inhibition. In some embodiments, a finding that contacting an organoid of the invention with a compound increases secretion and/or expression and/or production of one or more of CCK, GLP-1, PYY and PPY and/or increases secretion and/or expression of GCG indicates that the compound is suitable for treating or preventing a disease or disorder which requires appetite inhibition, e.g. obesity and/or diabetes and/or Prader-Willi syndrome. Thus, in some embodiments, the disease or disorder is a disease or disorder treatable or preventable by inducing or increasing secretion and/or expression and/or production of one or more of CCK, GLP-1, PYY and PPY and/or GCG, e.g. obesity, diabetes or Prader-Willi syndrome. In some embodiments, the disease or disorder is obesity. In some embodiments, the disease or disorder is diabetes. In some embodiments, the diabetes is type II diabetes. In some embodiments, the disease or disorder is Prader-Willi syndrome. Conversely, CCK, GLP-1, PPY and PYY are also implicated in appetite stimulation. Thus, in some embodiments, a finding that contacting an organoid of the invention with a compound decreases secretion and/or expression and/or production of one or more of CCK, GLP-1, PYY and PPY and/or decreases secretion and/or expression of GCG indicates that the compound is suitable for treating or preventing a disease or disorder which requires appetite stimulation, e.g. anorexia and/or cancer cachexia. Thus, in some embodiments, the disease or disorder is a disease or disorder treatable or preventable by ceasing or decreasing secretion and/or expression and/or production of one or more of CCK, GLP-1, PYY and PPY and/or GCG, e.g. anorexia or cancer cachexia. In some embodiments, the disease or disorder is anorexia. In some embodiments, the disease or disorder is cancer cachexia.

Motilin, Angiotensin and Serotonin are implicated in gut motility diseases and disorders (e.g. Kitazawa, T. and Kaya, H. et al. 2019; Patten, G. S. and Abeywardena, M. Y. 2017). In some embodiments, a finding that contacting an organoid of the invention with a compound increases secretion and/or expression of one or more of Motilin, Angiotensin and/or Serotonin indicates that the compound is suitable for treating or preventing a gut motility disorder, for example, a bowel movement disorder, Parkinson's disease or gastroparesis. The Motilin agonist erythromycin is clinically used to stimulate peristalsis. Thus, in some embodiments, the disease or disorder is a disease or disorder treatable or preventable by inducing or increasing secretion and/or expression of one or more of Motilin, Angiotensin and/or Serotonin, e.g. a gut motility disorder (for example, a bowel movement disorder, Parkinson's disease (e.g. Jost, W. H. 1997) or gastroparesis). In some embodiments, the disease or disorder is a gut motility disorder. In some embodiments, the disease or disorder is a bowel movement disorder. In some embodiments, the disease or disorder is Parkinson's disease. In some embodiments, the disease or disorder is gastroparesis. Examples of bowel movement disorders are gastroesophageal reflux disease, intestinal dysmotility, constipation, hirschsprung's disease. Thus, in some embodiments, the disease or disorder is gastroesophageal reflux disease. In some embodiments, the disease or disorder is intestinal dysmotility. In some embodiments, the disease or disorder is constipation. In some embodiments, the disease or disorder is hirschsprung's disease.

Motilin is implicated in appetite-related diseases and disorders. In particular, Motilin increases hunger (e.g. Zhao, D. et al. 2018; Deloose, E. et al. 2016). In some embodiments, a finding that contacting an organoid of the invention with a compound decreases or ceases expression and/or secretion of Motilin indicates that the compound is suitable for treating or preventing a disease or disorder which requires appetite inhibition, e.g. obesity, diabetes or Prader-Willi syndrome. Thus, in some embodiments, the disease or disorder is a disease or disorder treatable or preventable by decreasing or ceasing secretion and/or expression of Motilin. In some embodiments, the disease or disorder is obesity. In some embodiments, the disease or disorder is diabetes. In some embodiments, the diabetes is type II diabetes. In some embodiments, the disease or disorder is Prader-Willi syndrome. Conversely, in some embodiments, a finding that contacting an organoid of the invention with a compound increases or induces expression and/or secretion of Motilin indicates that the compound is suitable for treating or preventing a disease or disorder which requires appetite stimulation, e.g. anorexia or cancer cachexia. Thus, in some embodiments, the disease or disorder is a disease or disorder treatable or preventable by increasing or inducing secretion and/or expression of Motilin. In some embodiments, the disease or disorder is anorexia. In some embodiments, the disease or disorder is cancer cachexia.

Motilin is also implicated in diabetes. In particular, Motilin increases insulin release (e.g. Suzuki, H. et al. 1998; Christofides, N. D. et al. 1979) and so increasing Motilin is helpful for treating diabetes due to insulin release. In some embodiments, a finding that contacting an organoid of the invention with a compound increases or induces expression and/or secretion of Motilin indicates that the compound is suitable for treating or preventing diabetes, in particular type II diabetes. Thus, in some embodiments, the disease or disorder is a disease or disorder treatable or preventable by increasing or inducing secretion and/or expression of Motilin. In some embodiments, the disease or disorder is diabetes. In some embodiments, the diabetes is type II diabetes. In some embodiments, the treating or preventing comprises release of insulin.

Motilin is implicated in biliary movement disorders (Luiking Y et al. 1998). In particular, Motilin increases gallbladder emptying (Luiking Y et al. 1998; Deloose, E. et al. 2019). In some embodiments, a finding that contacting an organoid of the invention with a compound increases or induces expression and/or secretion of Motilin indicates that the compound is suitable for treating or preventing a biliary movement disorder. Thus, in some embodiments, the disease or disorder is a disease or disorder treatable or preventable by increasing or inducing secretion and/or expression of Motilin. In some embodiments, the disease or disorder is a biliary movement disorder. In some embodiments, the disease or disorder is biliary dyskinesia.

Accordingly, Motilin is implicated in a wide variety of diseases and disorders (Deloose, E. et al. 2019) and so modulating Motilin expression and/or secretion using a compound of the invention which targets a target on a human EEC, as described herein, is useful for treating or preventing a wide variety of diseases and disorders, including gut motility disorders, diseases and disorders requiring appetite stimulation or appetite inhibition, diabetes (in particular type II diabetes) and biliary movement disorders.

GLP-1 is implicated in gut motility diseases and disorders (e.g. Hellstrom, P. M. et al. 2008; Marathe, C. S. et al. 2011). In some embodiments, a finding that contacting an organoid of the invention with a compound decreases secretion and/or expression of GCG and/or production of GLP-1 indicates that the compound is suitable for treating or preventing a gut motility disorder, for example, a bowel movement disorder, Parkinson's disease or gastroparesis. GCG is processed to GLP-1. GLP-1 reduces gastric emptying and motility. Thus, in some embodiments, the disease or disorder is a disease or disorder treatable or preventable by ceasing or reducing secretion and/or expression of GCG and/or production of GLP-1, e.g. a gut motility disorder (for example, a bowel movement disorder, Parkinson's disease or gastroparesis). In some embodiments, the disease or disorder is a gut motility disorder. In some embodiments, the disease or disorder is a bowel movement disorder. In some embodiments, the disease or disorder is Parkinson's disease. In some embodiments, the disease or disorder is gastroparesis. Examples of bowel movement disorders are gastroesophageal reflux disease, intestinal dysmotility, constipation, hirschsprung's disease. Thus, in some embodiments, the disease or disorder is gastroesophageal reflux disease. In some embodiments, the disease or disorder is intestinal dysmotility. In some embodiments, the disease or disorder is constipation. In some embodiments, the disease or disorder is hirschsprung's disease.

GLP-1 and GIP are implicated in diseases and disorders associated with insulin secretion (e.g. Jones, B. et al. 2018; Christensen, M. B. 2016; MacDonald, P. E. et al. 2002; Jones, B. et al. 2018; Eiel, J. N. et al. 2018; Kolik, J. et al. 2014). For example, GLP-1 and GIP are implicated in diabetes, e.g. type II diabetes. In some embodiments, a finding that contacting an organoid of the invention with a compound increases secretion and/or expression of GCG and/or GIP and/or increases production of GLP-1 indicates that the compound is suitable for treating or preventing diabetes, e.g. type II diabetes. GLP-1 agonists are widely used for T2D (type II diabetes) treatment. Thus, in some embodiments, the disease or disorder is diabetes, e.g. type II diabetes.

GLP-1 and GIP are implicated in neurodegenerative diseases and disorders, such as Parkinson's Disease and Alzheimer's Disease (e.g. Ji, C. et al. 2016; Grieco, M. et al., 2019; Athuada, D. and Foltynie, T. 2016; Zhang, Z. Q. and Holscher, C. 2020; Faivre, E. and Holscher, C. 2013). In some embodiments, a finding that contacting an organoid of the invention with a compound increases secretion and/or expression of GCG and/or GIP and/or increases production of GLP-1 indicates that the compound is suitable for treating or preventing a neurodegenerative disease or disorder, e.g. Parkinson's Disease or Alzheimer's Disease. Several clinical trials have shown encouraging effects in patients with Alzheimer's or Parkinson's disease using GLP-1 mimetics or GLP-1 receptor agonists or using a combination of GLP-1 and GIP mimetics. Thus, in some embodiments, the disease or disorder is a disease or disorder treatable or preventable by inducing or increasing secretion and/or expression of GCG and/or GIP and/or increasing or inducing production of GLP-1, e.g. diabetes (e.g. type II diabetes) or a neurodegenerative disease (e.g. Parkinson's Disease or Alzheimer's Disease). In some embodiments, the disease or disorder is diabetes (e.g. type II diabetes). In some embodiments, the disease or disorder is a neurodegenerative disease. In some embodiments, the disease or disorder is Parkinson's disease. In some embodiments, the disease or disorder is Alzheimer's disease.

Somatostatin is implicated a wide variety of diseases and disorders as described herein. Somatostatin inhibits secretion of most intestinal hormones, including GLP-1 (e.g. Chisholm, C and Greenberg, G. R. 2002). Thus, in some embodiments, the disease or disorder is a disease or disorder as described herein. Thus, a finding that contacting an organoid of the invention with a compound decreases or ceases secretion and/or expression of Somatostatin indicates that the compound is suitable for treating or preventing a disease or disorder treatable or preventable by increasing or inducing secretion of a hormone and/or hormone precursor by an EEC. Inhibition of Somatostatin may for example enhance GLP-1 release, useful for type 2 diabetes. For example, in some embodiments, a finding that contacting an organoid of the invention with a compound decreases secretion and/or expression of Somatostatin indicates that the compound is suitable for treating or preventing diabetes, e.g. type II diabetes. Similarly, in some embodiments, a finding that contacting an organoid of the invention with a compound ceases secretion and/or expression of Somatostatin indicates that the compound is suitable for treating or preventing diabetes, e.g. type II diabetes. Thus, in some embodiments, the disease or disorder is a disease or disorder treatable or preventable by ceasing or decreasing secretion and/or expression of Somatostatin. For example, in some embodiments, the disease or disorder is a disease or disorder treatable or preventable by inhibiting secretion and/or expression of Somatostatin. For example, in some embodiments, the disease or disorder is a disease or disorder treatable or preventable by increasing or inducing expression and/or secretion and/or production of one or more hormones and/or hormone precursors by one or more EECs and/or increasing or inducing production of one or more hormones from one or more hormone precursors by one or more EECs. For example, in some embodiments, the disease or disorder is a disease or disorder treatable or preventable by increasing or inducing secretion of one or more hormones and/or hormone precursors by one or more EECs and/or increasing or inducing production of one or more hormones from one or more hormone precursors by one or more EECs. In some embodiments, the one or more hormones and/or hormone precursors are selected from Ghrelin, Cerebellin 1, CCK, GCG, GLP-1, PYY, PPY, Motilin, Angiotensin, Serotonin, GIP, Gastrin and Secretin. Examples of disease and disorders treatable or preventable by increasing or inducing secretion and/or expression and/or production of one or more of these hormones or hormone precursors are described herein. For example, in some embodiments, the disease or disorder is selected from an appetite-related disease or disorder (e.g. obesity, anorexia, cancer cachexia, obesity, diabetes (in particular type II diabetes), Prader-Willi syndrome), a gut motility disease or disorder (e.g. a bowel movement disorder, Parkinson's disease or gastroparesis), diabetes (in particular type II diabetes), a neurodegenerative disease or disorder (e.g. Parkinson's disease or Alzheimer's disease) and depression. In some embodiments, the disease or disorder is an appetite-related disease or disorder. In some embodiments, the disease or disorder is obesity. In some embodiments, the disease or disorder is Prader-Willi syndrome. In some embodiments, the disease or disorder is anorexia. In some embodiments, the disease or disorder is cancer cachexia. In some embodiments, the disease or disorder is diabetes (in particular type II diabetes). In some embodiments, the disease or disorder is a gut motility disease or disorder. In some embodiments, the disease or disorder is a bowel movement disorder. In some embodiments, the disease or disorder is Parkinson's disease. In some embodiments, the disease or disorder is gastroparesis. In some embodiments, the disease or disorder is a neurodegenerative disease. In some embodiments, the disease or disorder is Parkinson's disease. In some embodiments, the disease or disorder is Alzheimer's disease. In some embodiments, the disease or disorder is depression. In some embodiments, the disease or disorder is a biliary movement disorder. In some embodiments, the disease or disorder is biliary dyskinesia. For example, decreasing or ceasing expression and/or secretion of Somatostatin may be useful for treating or preventing diseases or disorders that require an increase or induction in GLP-1 production for their treatment or prevention. Thus, in some embodiments, the disease or disorder is a disease or disorder treatable or preventable by increasing or inducing GLP-1 production. Examples of such diseases and disorders in which GLP-1 is implicated are provided herein. Thus, the treatment or prevention of diseases and disorders by decreasing or ceasing expression and/or secretion of Somatostatin using the various teachings of the invention (e.g. methods and uses) is encompassed by the invention. Conversely, a finding that contacting an organoid of the invention with a compound increases secretion and/or expression of Somatostatin indicates that the compound is suitable for treating or preventing a disease or disorder treatable or preventable by decreasing or ceasing secretion and/or expression and/or production of one or more hormones and/or hormone precursors by one or more EECs and/or decreasing or ceasing production of one or more hormones from one or more hormone precursors by one or more EECs. In some embodiments, the disease or disorder is a disease or disorder treatable or preventable by decreasing or ceasing secretion of one or more hormones and/or hormone precursors by one or more EECs and/or decreasing or ceasing production of one or more hormones from one or more hormone precursors by one or more EECs. In some embodiments, the one or more hormones and/or hormone precursors are selected from Ghrelin, Cerebellin 1, CCK, GCG, GLP-1, PYY, PPY, Motilin, Angiotensin, Serotonin, GIP, Gastrin and Secretin. Examples of disease and disorders treatable or preventable by decreasing or ceasing secretion and/or expression and/or production of one or more of these hormones or hormone precursors are described herein. For example, in some embodiments, the disease or disorder is selected from an appetite-related disease or disorder (e.g. obesity, anorexia, cancer cachexia, obesity, diabetes (in particular type II diabetes), Prader-Willi syndrome), a gut motility disease or disorder (e.g. a bowel movement disorder, Parkinson's disease or gastroparesis), high acid secretion in the stomach (e.g. indigestion an acid reflux) and IBS. In some embodiments, the disease or disorder is an appetite-related disease or disorder. In some embodiments, the disease or disorder is obesity. In some embodiments, the disease or disorder is Prader-Willi syndrome. In some embodiments, the disease or disorder is anorexia. In some embodiments, the disease or disorder is cancer cachexia. In some embodiments, the disease or disorder is diabetes (in particular type II diabetes). In some embodiments, the disease or disorder is a gut motility disease or disorder. In some embodiments, the disease or disorder is a bowel movement disorder. In some embodiments, the disease or disorder is Parkinson's disease. In some embodiments, the disease or disorder is gastroparesis. In some embodiments, the disease or disorder is high acid secretion in the stomach. In some embodiments, the disease or disorder is indigestion caused by high acid secretion in the stomach. In some embodiments, the disease or disorder is acid reflux caused by high acid secretion in the stomach. In some embodiments, the high acid secretion in the stomach is caused by abnormal parietal cell activity in the stomach. In some embodiments, the disease or disorder is IBS. In some embodiments, the disease or disorder is IBD. For example, increasing Somatostatin may be useful for treating or preventing diseases or disorders that require a decrease or cease in GLP-1 production for their treatment or prevention. Thus, in some embodiments, the disease or disorder is a disease or disorder treatable or preventable by decreasing or ceasing GLP-1 production. Examples of such diseases and disorders in which GLP-1 is implicated are provided herein. Thus, the treatment or prevention of diseases and disorders by increasing or inducing expression and/or secretion of Somatostatin using the various teachings of the invention (e.g. methods and uses) is encompassed by the invention.

Gastrin and Secretin and implicated in diseases and disorders involved with parietal cell activity in the stomach, e.g. high acid secretion in the stomach (e.g. Soll, A. H. and Walsh, J. H., 1979; Shimizu, K. et al. 1995; Lindstrom, E. et al. 2001). In some embodiments, a finding that contacting an organoid of the invention with a compound decreases secretion and/or expression of Gastrin and/or Secretin indicates that the compound is suitable for treating or preventing high acid secretion in the stomach. Thus, in some embodiments, the disease or disorder is a disease or disorder treatable or preventable by ceasing or decreasing secretion and/or expression of Gastrin and/or Secretin, e.g. high acid secretion in the stomach. Thus, in some embodiments, the disease or disorder is high acid secretion in the stomach. As mentioned above, Gastrin and Secretin are known to be involved in parietal cell activity in the stomach. Thus, in some embodiments, the high acid secretion in the stomach is caused by abnormal parietal cell activity in the stomach. In some embodiments, the disease or disorder is indigestion or acid reflux. In some embodiments, the disease or disorder is indigestion caused by high acid secretion in the stomach. In some embodiments, the disease or disorder is acid reflux caused by high acid secretion in the stomach.

Serotonin is implicated in IBS (e.g. Crowell, M. D., 2004; Tack, J. et al. 2011). Serotonin regulates bowel movement and activation of pain receptors. Serotonin is elevated in IBS. In some embodiments, a finding that contacting an organoid of the invention with a compound decreases secretion and/or expression of serotonin indicates that the compound is suitable for treating or preventing irritable bowel syndrome. The skilled person will understand that by “expression” of serotonin is meant production of serotonin. As explained elsewhere herein, serotonin is not encoded by a gene in an EEC but is instead synthesized by the enzyme TPH1. Thus, in some embodiments, a finding that contacting an organoid of the invention with a compound decreases expression of TPH1 indicates that the compound is suitable for treating or preventing irritable bowel syndrome. Thus, in some embodiments, the disease or disorder is a disease or disorder treatable or preventable by ceasing or decreasing secretion and/or production of Serotonin, e.g. IBS. Thus, in some embodiments, the disease or disorder is IBS. Serotonin is also implicated in IBD (Coates, M. D. et al. 2017; Levin, A. D and van den Brink G. R., 2014). In some embodiments, a finding that contacting an organoid of the invention with a compound decreases expression of TPH1 indicates that the compound is suitable for treating or preventing inflammatory bowel disease. In some embodiments, a finding that contacting an organoid of the invention with a compound decreases secretion and/or expression of serotonin indicates that the compound is suitable for treating or preventing inflammatory bowel disease. Thus, in some embodiments, the disease or disorder is IBD. In some embodiments, the IBD is Crohn's disease. In some embodiments, the IBD is ulcerative colitis. Serotonin is also implicated in depression (Blier, P. and Mansari, M. E. 2013; Israelyan N et al. 2019). Depression is often associated with a decrease in Serotonin. As mentioned elsewhere herein, the gut-brain axis means that hormonal changes in the gut can have effects in the brain. Serotonin is also implicated in diabetes (Paulmann, N. et al. 2009; Cataldo Bascunan, L. R. et al. 2019). Serotonin tends to be low in type II diabetes. In some embodiments, a finding that contacting an organoid of the invention with a compound increases or induces secretion and/or production of serotonin, for example, increases expression of TPH1, indicates that the compound is suitable for treating or preventing depression or diabetes (in particular type II diabetes). Thus, in some embodiments, the disease or disorder is a disease or disorder treatable or preventable by inducing or increasing secretion and/or production of Serotonin, e.g. depression and/or diabetes (in particular type II diabetes). In some embodiments, the disease or disorder is depression. In some embodiments, the disease or disorder is diabetes (in particular type II diabetes).

Examples of bowel movement disorders are gastroesophageal reflux disease, intestinal dysmotility, constipation, hirschsprung's disease. Thus, in some embodiments throughout the description which relate to a bowel movement disorder, the disease or disorder is gastroesophageal reflux disease. Thus, in some embodiments throughout the description which relate to a bowel movement disorder, the disease or disorder is intestinal dysmotility. Thus, in some embodiments throughout the description which relate to a bowel movement disorder, the disease or disorder is constipation. Thus, in some embodiments throughout the description which relate to a bowel movement disorder, the disease or disorder is hirschsprung's disease.

The description provided above of which compound is suitable for treating which disease or disorder can be applied throughout the description, in particular to the description of the identified targets in human EECs and the methods for treating or preventing diseases and disorders by targeting these targets.

In some embodiments, the disease or disorder is a disease or disorder treatable or preventable by increasing or inducing expression and/or secretion and/or production of a hormone implicated in the disease or disorder. In some embodiments, the disease or disorder is a disease or disorder treatable or preventable by decreasing or ceasing expression and/or secretion and/or production of a hormone implicated in the disease or disorder.

The diseases and disorders recited herein are not limiting. Other diseases and disorders in which hormones and/or hormone precursors expressed and/or secreted and/or produced by EECs are implicated may likewise be treated or prevented using the methods and uses provided by the invention.

In some embodiments, the treating or preventing comprises increasing and/or decreasing expression and/or secretion levels and/or production levels of one or more hormones, hormone precursors and/or hormone synthesizing enzymes implicated in the disease or disorder, e.g. as described herein. In some embodiments, the patient to be treated is a patient found to have an increased or decreased level of expression and/or secretion and/or production of one or more hormones, hormone precursors and/or hormone synthesizing enzymes implicated in the disease or disorder. Whether an increase or a decrease is relevant will be clear to the skilled person depending on which disease or disorder is of interest, based on the teaching provided herein.

In some embodiments, the disease or disorder is an EEC-related disease or disorder. For example, in some embodiments, the disease or disorder is a disease or disorder of EECs or of one or more EEC subtypes. In some embodiments, the disease or disorder affects one or more particular regions of the intestine, for example, as described herein.

In some embodiments, the method is conducted in two or more (e.g. 2, 3, 4, 5, 6, 7, 8, 9 or more) different organoids. For example, in some embodiments, a biobank of organoids is used in the methods described herein. For example, the methods described herein may be conducted using two or more (e.g. 2, 3, 4, 5, 6, 7, 8, 9 or more) organoids which are each established from a different region of the intestine. In some embodiments, the methods described herein are conducted in two or more (e.g. 2, 3, 4, 5, 6, 7, 8, 9, 10 or more) different types of EECs, for example, using organoids derived from different regions of the intestine. For example, if different results are found for organoids established from different regions of the intestine, these data can be used to help design therapeutic compounds that target the particular region of the intestine. Similarly, use of the biobank advantageously allows the methods described herein to be conducted for two or more (e.g. 2, 3, 4 or more) different hormones, hormone precursors and/or hormone synthesizing enzymes in the same type of EEC.

The description of embodiments relating to reporter organoids provided herein may be adapted for methods using intestinal organoids of the invention, for example, which are not reporter organoids, mutatis mutandis. In some embodiments, such intestinal organoids are obtained by or are obtainable by a method for enriching the population of EECs in an intestinal organoid, as described herein.

In some embodiments, the step of determining whether secretion of one or more hormones and/or hormone precursors is affected comprises detecting changes in hormone and/or hormone precursor secretion in the organoid. In some embodiments, the method comprises determining whether secretion of one or more hormones and/or hormone precursors is increased. In some embodiments, the method comprises determining whether secretion of one or more hormones and/or hormone precursors is decreased. In some embodiments, the step of determining whether secretion of one or more hormones and/or hormone precursors is affected comprises determining whether the type(s) of hormone and/or hormone precursor secreted changes.

In some embodiments, a method as provided herein further comprise comparing the results with a control. Any suitable control may be used, for example, a control as described herein.

In some embodiments, the step of determining whether secretion and/or expression and/or production of one or more EEC-specific proteins (e.g. hormones, hormone synthesizing enzymes and/or hormone precursors) is affected comprises comparing the secretion and/or expression and/or production to secretion and/or expression and/or production, respectively, from a control organoid of the invention which has not been contacted with the compound. In some embodiments, the step of determining whether secretion and/or expression and/or production is affected comprises comparing the result with a control organoid in which the gene has not been activated.

In some embodiments, the control organoid is established from the same region of the intestine as the organoid being contacted with the compound/in which the gene is being activated. The control organoid is preferably identical to the organoid being contacted with the compound/in which the gene is being activated, for example, it may be clonally identical. Preferably, the control organoid is cultured under identical conditions to the organoid being contacted with the compound/in which the gene is being activated. In embodiments in which the organoid being contacted with the compound is a reporter organoid, the control organoid is preferably also a reporter organoid.

In some embodiments, the control is a control (e.g. in a well of a multiwell plate) which comprises the culture medium but does not comprise any organoids. For example, such a control may serve as a subtraction background for media autofluorescence.

In some embodiments, the step of determining whether secretion and/or expression and/or production of one or more EEC-specific proteins (e.g. hormones, hormone precursors and/or hormone synthesizing enzymes) is affected comprises detecting the one or more EEC-specific proteins at two or more time points. In embodiments in which a reporter organoid is used in which the EEC-specific gene (e.g. hormone, hormone precursor or hormone synthesizing enzyme) is tagged with a fluorescent marker, the difference in fluorescent reporter gene expression can be detected by any suitable technique, for example, using confocal microscopy, a fluorescence microscope or a fluorescence plate reader. In some embodiments, a decrease in expression of the detectable marker in the cell, e.g. between two time points and/or compared to a control reporter organoid which has not been contacted with the compound, or in which the gene has not been activated, indicates that the compound or activation of the gene is causing an increase in secretion of the tagged hormone and/or tagged hormone precursor. In some embodiments, the decrease in expression of the detectable marker in the cell is a decrease in fluorescence in the cell cytoplasmic vesicles.

In some embodiments, an increase in secretion, e.g. compared to the control organoid and/or between the at least two time points, indicates that the protein or nucleic acid (e.g. DNA or RNA) targeted by the compound is involved in inducing or increasing hormone or hormone precursor secretion and identifies/validates the protein or nucleic acid as a target (e.g. a drug target) in the EECs or in one or more subtypes thereof. For example, in some diseases, it is desirable to increase hormone secretion, e.g. of one or more hormones and/or hormone precursors. In contrast, in other diseases, it is desirable to decrease hormone secretion, e.g. of one or more hormones. Accordingly, in some embodiments, a decrease in secretion, e.g. compared to the control organoid and/or between the at least two time points, indicates that the protein or nucleic acid targeted by the compound is involved in reducing hormone secretion and identifies/validates the protein or nucleic acid as a target in the EECs or in one or more subtypes thereof. Similarly, for a particular disease or disorder, it may be desirable to look for an increase or a decrease in secretion depending on the particular hormone of interest. For example, where the disease or disorder is an appetite-related disease or disorder, there are some hormones that increase hunger, whereas others decrease hunger. For example, the GHRL hormone induces appetite whereas the PYY hormone reduces appetite. Different outcomes will therefore be desirable depending on the disease or disorder of interest, for example, obesity compared to anorexia. Thus, in some embodiments, a finding that contacting a target with a compound increases GHRL secretion indicates that the target is a drug target for treating or preventing anorexia. In some embodiments, a finding that contacting a target with a compound increases PYY secretion indicates that the target is a drug target for treating or preventing obesity. A detailed description of which diseases and disorders can be treated or prevented by increasing or decreasing expression and/or secretion and/or production of one or more particular hormones, hormone precursors and/or hormone synthesizing enzymes is provided elsewhere herein. The skilled person will be able to use this information to determine if a target is a suitable drug target for treating or preventing a disease or disorder of interest.

In some embodiments, the target (e.g. the protein or nucleic acid) targeted by the compound is a receptor, for example, a cell surface receptor. In some embodiments, the target targeted by the compound is a transcription factor. The target is preferably a protein target.

In some embodiments, hormone and/or hormone precursor secretion by an organoid (e.g. by an organoid of the invention) increases when the organoid is exposed to Forskolin. Indeed, the inventors have found that exposure of organoids to Forskolin, a stimulator of adenylate cyclase increasing cAMP levels and thus of hormone secretion, greatly enhanced the detectable levels of hormones such as GLP-1 in the culture supernatant. Thus, the invention provides a method for increasing secretion of one or more hormones and/or hormone precursors by an intestinal organoid (e.g. an intestinal organoid of the invention), which comprises contacting the organoid with a substance or composition that increases cAMP levels (such as a stimulator of adenylate cyclase, e.g. Forskolin) and optionally detecting an increased level of hormone in the culture supernatant. The organoid is preferably an organoid in which the intestinal stem cells have been differentiated to EECs. In some embodiments, this method may be used as a positive control for a method as described herein. Thus, in some embodiments, a method as described herein further comprises the step of comparing the results to a positive control, for example, as described herein.

In the various methods of the invention described herein, the intestinal organoid may be a reporter organoid in some embodiments.

Also provided is a method for identifying/screening/validating a compound for suitability for controlling differentiation of one or more EEC subtypes, wherein the method comprises (i) contacting an organoid of the invention with the compound; and (ii) determining whether expression of one or more hormones, hormone precursors and/or hormone synthesizing enzymes is affected. If expression of one or more hormones, hormone precursors and/or hormone synthesizing enzymes is affected, the results may be used to determine which EEC population is made in response to the compound and/or whether the ratio of two or more EEC subtypes has been affected. This method may optionally be known as a differentiation screen. As mentioned above, the organoid contacted with the compound in step (i) preferably does not yet comprise EECs. In some embodiments, the organoid is contacted with the compound before or during the step of differentiating stem cells and/or cells with stem cell potential in the organoid to EECs. Thus, step (i) may comprise contacting an organoid of the invention with the compound and differentiating the stem cells and/or cells with stem cell potential in the organoid to EECs, e.g. by overexpressing the inducible transcription factor.

Also provided is a method for testing the effect of a compound on EECs or on one or more EEC subtypes, wherein the method comprises (i) contacting one or more organoids of the invention with the compound; and (ii) determining whether secretion and/or expression and/or production of one or more hormones, hormone precursors and/or hormone synthesizing enzymes is affected. In some embodiments, the method identifies the compound as a compound which controls differentiation of EECs or one or more subtypes thereof. Controlling differentiation of EECs or one or more subtypes thereof is described further herein and the description of this can be applied to these embodiments mutatis mutandis.

Also provided is a method for testing the effect of activation of a gene on EECs or on one or more subtypes thereof, wherein the method comprises (i) activating the gene in one or more organoids of the invention; and (ii) determining whether secretion and/or expression and/or production of one or more hormones, hormone precursors and/or hormone synthesizing enzymes is affected. In some embodiments, the method identifies the gene as a gene which controls differentiation of EECs or one or more subtypes thereof. Controlling differentiation of EECs or one or more subtypes thereof is described further herein and the description of this can be applied to these embodiments mutatis mutandis.

Also provided is a method for determining the function of a transcription factor on intestinal cells, wherein the method comprises generating an intestinal organoid of the invention in which the transcription factor is knocked out or overexpressed and determining whether the number of EECs and/or the numbers of EECs of one or more EEC subtypes produced by the intestinal organoid is affected and/or whether secretion and/or expression and/or production of one or more hormones, hormone precursors and/or hormone synthesizing enzymes is affected. The results may be compared to a control organoid in which the transcription factor has not been knocked out or overexpressed, respectively. In some embodiments, the intestinal cells are intestinal stem cells. In some embodiments, the intestinal cells are EECs. In embodiments which comprise overexpressing a transcription factor, this may be achieved by any suitable method, for example, by introducing a construct comprising a gene encoding the transcription factor into intestinal stem cells and/or intestinal cells with stem cell potential. The gene may be under control of an inducible promoter, for example, an inducible promoter different from or the same as the inducible promoter used for the inducible transcription factor for differentiating intestinal cells to EECs in embodiments in which such a transcription factor for differentiating intestinal cells to EECs is used. Alternatively, the endogenous gene encoding the transcription factor can be targeted with a construct comprising an inducible promoter. In alternative embodiments, the endogenous gene encoding the transcription factor may be knocked out by any suitable method. In some embodiments, the gene is knocked out using CRISPR/Cas9. The transcription factor is preferably knocked out or the overexpression construct is preferably introduced before clonal selection of the intestinal cells and before the stem cells are differentiated to EECs, for example, by overexpression of NEUROG3. In some embodiments in which the intestinal organoid is a reporter organoid, the transcription factor may be knocked out or the overexpression construct is introduced before or during the step of introducing the first detectable marker into the endogenous hormone locus. In some embodiments, the transcription factor is knocked out or the overexpression construct is introduced before or after or at the same time as the inducible transcription factor construct for differentiating intestinal stem cells to EECs is introduced into the intestinal cells. In some embodiments, the organoid is a reporter organoid. In some embodiments, the organoid is not a reporter organoid.

In some embodiments, a knockout construct for use in the invention is a CRISPR/Cas9 knockout construct.

In some embodiments, a method of the invention is carried out in vitro.

Also provided is a method for identifying a compound which preferentially induces (or increases) or preferentially represses (or ceases) expression and/or secretion and/or production of a first hormone, hormone precursor or hormone synthesizing enzyme compared to a second hormone, hormone precursor or hormone synthesizing enzyme, wherein the method comprises (i) contacting one or more reporter organoids of the invention with one or more compounds, wherein the one or more reporter organoids comprise the first hormone, hormone precursor or hormone synthesizing enzyme tagged with a first detectable marker and the second hormone, hormone precursor or hormone synthesizing enzyme tagged with a second detectable marker; and (ii) determining whether expression and/or secretion of the first and second hormones, hormone precursors and/or hormone synthesizing enzymes is affected. In some embodiments, step (ii) comprises determining whether the relative expression and/or secretion of the first and second hormones, hormone precursors and/or hormone synthesizing enzymes is affected. If the expression and/or secretion of the first hormone, hormone precursor or hormone synthesizing enzyme is preferentially induced or preferentially repressed compared to the second hormone, hormone precursor or hormone synthesizing enzyme, this identifies the compound as being capable of preferentially inducing or preferentially repressing, respectively, the production or secretion of the first hormone, hormone precursor or hormone synthesizing enzyme compared to the second hormone, hormone precursor or hormone synthesizing enzyme.

In some embodiments, the first and second hormones, hormone precursors and/or hormone synthesizing enzymes are tagged in the same reporter organoid. In some embodiments, the first and second hormones, hormone precursors and/or hormone synthesizing enzymes are tagged in different reporter organoids. Preferably, the different reporter organoids are identical apart from the identity of the tagged hormone, hormone precursor and/or hormone synthesizing enzyme, for example, they have been established from the same region of the intestine, for example, using the same cells and using the same conditions. For example, they are preferably established from clonally identical cells. In some embodiments in which different reporter organoids are used, the different reporter organoids may be contacted with the same one or more compounds in different experiments, either simultaneously or at different times, and the results are compared. The different reporter organoids are preferably cultured separately. In some embodiments, the detectable markers fused to the first and second hormones, hormone precursors and/or hormone synthesizing enzymes are different. In some embodiments, the detectable markers fused to the first and second hormones, hormone precursors and/or hormone synthesizing enzymes are the same. In embodiments in which the detectable markers fused to the first and second hormones, hormone precursors and/or hormone synthesizing enzymes are the same, the first and second tagged hormones, hormone precursors and/or hormone synthesizing enzymes are preferably in different reporter organoids and the different reporter organoids are preferably contacted with the one or more compounds in different experiments. In some embodiments, the method identifies the compound as a compound which controls differentiation of EECs or one or more subtypes thereof. Controlling differentiation of EECs or one or more subtypes thereof is described further herein and the description of this can be applied to these embodiments mutatis mutandis.

In some embodiments, determining whether a compound controls differentiation comprises determining whether the compound changes the expression of one or more hormones, hormone precursors and/or hormone synthesizing enzymes within an EEC subtype and/or preferably determines whether there is a change in the ratio of two or more EEC subtypes.

In some embodiments, an increase in expression, e.g. compared to the control organoid and/or between the at least two time points, indicates that the compound controls differentiation of EECs or one or more subtypes thereof. For example, initiation of expression or an increase in expression of one or more hormones, hormone precursors or hormone synthesizing enzymes may indicate that one EEC subtype has differentiated into another EEC subtype. In some embodiments, a decrease in expression, e.g. compared to the control organoid and/or between the at least two time points, indicates that the compound controls differentiation of EECs or one or more subtypes thereof. For example, ceasing of expression or a decrease in expression of one or more hormones, hormone precursors or hormone synthesizing enzymes may indicate that one EEC subtype has differentiated into another EEC subtype. For example, the method may comprise determining whether a compound induces or ceases production of a specific hormone, hormone precursor and/or hormone synthesizing enzyme.

In some embodiments, an increase in secretion and/or expression and/or production, e.g. compared to the control organoid and/or between the at least two time points, indicates that activation of the gene is involved in inducing or increasing secretion and/or expression and/or production of one or more hormones, hormone precursors and/or hormone synthesizing enzymes. A change in expression may identify the gene as a gene which controls differentiation of EECs or one or more subtypes thereof. For example, initiation of expression of, or an increase in expression of one or more hormones, hormone precursors and/or hormone synthesizing enzymes may indicate that one EEC subtype has differentiated into another EEC subtype. Advantageously, it may also identify/validate the gene as a drug target in EECs or in one or more subtypes thereof. In some embodiments, a decrease in secretion and/or expression and/or production, e.g. compared to the control organoid and/or between the at least two time points, indicates that activation of the gene is involved in reducing or ceasing secretion and/or expression and/or production of one or more hormones, hormone precursors and/or hormone synthesizing enzymes. A change in expression may identify the gene as a gene which controls differentiation of EECs or one or more subtypes thereof. For example, ceasing of or reducing expression of one or more hormones, hormone precursors and/or hormone synthesizing enzymes may indicate that one EEC subtype has differentiated into another EEC subtype. Advantageously, it may also identify/validate the gene as a drug target in EECs or in one or more subtypes thereof.

In some embodiments in which the method is being used to determine whether contacting with one or more compounds or activation of a gene controls differentiation of EECs or one or more subtypes thereof, the method comprises determining whether expression of one or more (e.g. 1, 2, 3, 4, 5 or more) hormones, hormone precursors and/or hormone synthesizing enzymes is affected. In some embodiments, a change in the types and/or levels of hormones, hormone precursors and/or hormone synthesizing enzymes expressed to the types and/or levels of hormones, hormone precursors and/or hormone synthesizing enzymes expressed by a different EEC subtype indicates that contacting with the compound or activation of the gene has resulted in differentiation of the starting EEC subtype to a different EEC subtype. The hormones, hormone precursors and hormone synthesizing enzymes expressed by different EEC subtypes are described in detail in the present application and so the skilled person would understand if the EEC subtype changes.

In some embodiments, the method comprises determining whether there is a change (e.g. an increase or decrease) in secretion and/or expression and/or production of one hormone, hormone precursor or hormone synthesizing enzyme compared to one or more (e.g. 2, 3, 4, 5 or more) other hormones, hormone precursors and/or hormone synthesizing enzymes.

In some embodiments, there may be no change in secretion and/or expression and/or production (e.g. no increase or decrease of a particular hormone, hormone precursor or hormone synthesizing enzyme or no change in the type of hormone, hormone precursor or hormone synthesizing enzyme), e.g. compared to the control organoid and/or between the at least two time points. Such a result may in some embodiments be a negative result. However, methods of the invention, for example, methods for identifying/validating/screening/testing, which obtain negative results are still encompassed within the scope of the invention.

The intestinal organoid used in the various methods and uses described herein is preferably an intestinal organoid of the invention. For example, the reporter organoid used in the various methods and uses described herein is preferably a reporter organoid of the invention. The embodiments described herein which relate to using a reporter organoid of the invention may be adapted to use an intestinal organoid of the invention which is not a reporter organoid and/or to use alternative techniques to determine expression and/or secretion and/or production. For example, they may use an intestinal organoid comprising an inducible transcription factor for differentiating intestinal stem cells and/or intestinal cells with stem cell potential to EECs which is not itself a reporter organoid of the invention. For example, such an organoid may be obtained by or obtainable by a method for enriching the population of EECs in an intestinal organoid, as described herein. For example, the step of determining whether expression of the EEC-specific gene, e.g. the hormone, hormone precursor and/or the hormone synthesizing enzyme has been affected may be carried out using qPCR rather than by detecting the detectable marker in the tagged reporter organoid. Similarly, the step of determining whether secretion of a hormone, hormone precursor or a hormone produced from a hormone precursor has been affected may be carried out using a calcium reporter as a proxy for induction of secretion, ELISA to measure secreted hormones and/or hormone precursors in the organoid supernatant or mass spectrometry of the supernatant to discover and quantify secreted products (secretomics), as described herein. Such techniques may also be used when a reporter organoid of the invention is used. The methods described herein may be adapted accordingly.

Any suitable compound may be used, for example, in the various methods of the invention. Thus the description of the compounds can be applied throughout this disclosure. In some embodiments, the compound is known to target a particular target on EECs or on one or more subtypes thereof. In some embodiments, the target is a target as described herein. In some embodiments, the compound is known to target the EECs but the target on the EECs is unknown. In some embodiments, the compound is not known to target the EECs. In some embodiments, the compound is a known ligand for the target of interest. In some embodiments, the compound is a candidate compound. In some embodiments, the compound is a natural ligand of the target. In some embodiments, the compound is a mimetic of a natural ligand of the target. In some embodiments, the compound is selected from a protein (e.g. an antibody), peptide, nucleic acid (e.g. a DNA or an RNA), a small molecule, drug, bacterial product or microorganism, e.g. one or more bacteria. In some embodiments, the compound is an antibody, for example, an antibody specific for the target of interest. Any suitable antibody can be used (e.g. a monoclonal antibody, a full length antibody, or an antibody fragment). In some embodiments, the antibody is a humanised antibody or a fully human antibody. In some embodiments, the antibody is a chimeric antibody. In some embodiments, the compound is a nucleic acid, such as a DNA or RNA. In some embodiments, the compound is an agonist of the target. In some embodiments, the compound is an antagonist of the target. In some embodiments, the compound is a small molecule, for example, a small molecule specific for the target of interest. In some preferred embodiments, the target is a cell surface receptor, for example, a GPCR. Thus, in some preferred embodiments, the target is a GPCR. In some embodiments, the target is a cell surface receptor that is not a GPCR. In some embodiments, the target is intracellular. In some such embodiments, contacting the reporter organoid with the compound comprises introducing the compound into the cells in the reporter organoid, e.g. using transfection, transduction or electroporation.

For example, in some embodiments, the compound is a natural ligand for the receptor, for example, a known ligand as described herein. In some embodiments, the compound is a variant or a fragment of a natural ligand for the receptor. For example, the polypeptide sequence of the variant may have at least 75% (e.g. at least 80%, 85%, 90%, 95%, 98%, 99%) sequence identity with the polypeptide sequence of the natural ligand. Methods for determining sequence identity are described herein. A fragment of a variant of the natural ligand may also be used. Preferably, the variant or fragment has the same effect on the receptor and/or the same effect on expression and/or secretion of one or more hormones and/or hormone precursors as the natural ligand. In some embodiments, if the natural ligand increases or induces expression and/or secretion of one or more hormones, hormone precursors and/or hormone synthesizing enzymes, the variant or fragment increases or induces expression and/or secretion of the one or more hormones, hormone precursors and/or hormone synthesizing enzymes. In some embodiments, if the natural ligand reduces or ceases expression and/or secretion of one or more hormones, hormone precursors and/or hormone synthesizing enzymes, the variant or fragment reduces or ceases expression and/or secretion of the one or more hormones, hormone precursors and/or hormone synthesizing enzymes.

In some embodiments, the compound is an agonist of the target, e.g. of the receptor. In some embodiments, the compound is an antagonist of the target, e.g. of the receptor. In some embodiments, the compound increases or induces expression and/or secretion and/or production of one or more hormones, hormone precursors and/or hormone synthesizing enzymes. In some embodiments, the compound reduces or ceases expression and/or secretion and/or production of one or more hormones, hormone precursors and/or hormone synthesizing enzymes. In some embodiments, the one or more hormones, hormone precursors and/or hormone synthesizing enzymes are one or more hormones, hormone precursors and/or hormone synthesizing enzymes implicated in the disease or disorder. In some embodiments, the compound increases or induces expression and/or secretion of one or more hormones, hormone precursors and/or hormone synthesizing enzymes. In some embodiments, the compound reduces or ceases expression and/or secretion of one or more hormones, hormone precursors and/or hormone synthesizing enzymes. In some embodiments, the one or more hormones, hormone precursors and/or hormone synthesizing enzymes are one or more hormones, hormone precursors implicated in the disease or disorder.

In some embodiments, the compound is an antibody that targets the receptor, e.g. an agonistic antibody or an antagonistic antibody.

In some embodiments, the compound is a bacterial product. For example, in some embodiments, in order to obtain the compound, bacteria are cultured in a suitable culture medium. In some embodiments, after a suitable period of time, the culture medium comprising the bacteria is centrifuged and the supernatant obtained. In some embodiments, the culture medium may be filtered to obtain the supernatant. In some embodiments, the supernatant comprises bacterial products, for example, selected from bacterial metabolites, short chain fatty acids, peptides derived from the bacteria and/or lipopolysaccharides (LPS). Thus, in some embodiments, the bacterial product is selected from one or more bacterial metabolites, short chain fatty acids, peptides and/or lipopolysaccharides. In some embodiments, a method of the invention as described herein comprises contacting an organoid of the invention, e.g. a reporter organoid, with the supernatant obtained from a bacterial culture. The supernatant therefore comprises the one or more compounds used in the method.

In some embodiments, the supernatant is obtained from a culture comprising no more than one strain of bacteria. In some embodiments, the supernatant is obtained from a culture comprising no more than one species of bacteria. In some embodiments, the supernatant is obtained from a culture comprising two or more (e.g. 2, 3, 4, 5 or more) strains of bacteria. In some embodiments, the two or more strains belong to the same species of bacteria. In some embodiments, two or more strains belong to different species of bacteria. In some embodiments, the two more strains belong to the same Genus of bacteria. In some embodiments, the two or more strains belong to different genera of bacteria. In some embodiments, bacteria of other strains or other species or other genera are not present in the culture. In some embodiments, the supernatant is obtained from a culture comprising two or more (e.g. 2, 3, 4, 5, 10, 15, 20, 30, 50 or more) species of bacteria. In some embodiments, the two or more species of bacteria are from the same Genus of bacteria. In some embodiments, the two or more species of bacteria are from different genera of bacteria. In some embodiments, the supernatant is obtained from a culture comprising two or more (e.g. 2, 3, 4, 5, 8, 10, 12, 15 or more) genera of bacteria.

In some embodiments, the supernatant is obtained from a culture comprising a microbiome sample. For example, in some embodiments, the microbiome sample is taken from a donor. The donor may be selected from a human or a non-human animal, for example a non-human mammal, for example, a mouse, rat, rabbit, guinea pig or hamster. Preferably, the donor is a human donor. In some embodiments, the donor is a healthy donor. In some embodiments, the donor has a disease or disorder, for example, a disease or disorder associated with the intestine, a disease or disorder associated with EECs or a disease or disorder as described herein. In some embodiments, the donor is an adult. In some embodiments, the donor is an infant or a fetus. In some embodiments, the microbiome sample is taken from a single donor. In some embodiments, the microbiome sample is taken from more than one (e.g. 2, 3, 4, 5, 10 or more) donor(s). Advantageously, the use of a microbiome sample taken from a donor allows products produced by the microbiome community to be used as the one or more compounds, for example, as a library of compounds. Thus, in some embodiments, the compound is a product produced by the microbiome, for example, a bacterial product.

Thus, in some embodiments, a library of bacterial products are used in the methods of the invention. For example, the library may be a library of bacterial products produced by the microbiome. Similarly, in some embodiments, a library of products produced by the microbiome may be used. Thus, in some embodiments, the one or more compounds are one or more compounds produced by the microbiome.

In some embodiments, the supernatant is divided into fractions. Preferably, the fractions do not all comprise the same compounds, e.g. the same bacterial products. For example, the fractions may comprise different compounds. For example, different combinations of compounds may be present in each fraction. In some embodiments, the supernatant is fractionated according to the size of the compound, e.g. according to the size of the bacterial products. In some embodiments, the supernatant is fractionated using LC-MS. Thus, in some embodiments, a reporter organoid is contacted with a fraction of the supernatant obtained from a bacterial culture. If a positive result is obtained, e.g. if secretion and/or expression of one or more tagged EEC-specific genes (e.g. one or more tagged hormones, tagged hormone precursors and/or tagged hormone synthesizing enzymes) is affected, the fraction may be investigated further to determine which one or more compounds in the fraction is/are responsible for achieving the effect (e.g. alone or in combination). For example, individual compounds from the fraction may be obtained, e.g. using HPLC, and used in a method of the invention, for example, either alone or in combination. The supernatant is preferably a supernatant as described herein. For example, in some embodiments, the supernatant is obtained from a culture comprising no more than one strain of bacteria. In some embodiments, the supernatant is obtained from a culture comprising two or more strains, species or genera of bacteria, for example, as described herein.

Thus, in some embodiments, the one or more compounds are one or more compounds obtained from or obtainable from supernatant obtained from a bacterial culture, or obtained from or obtainable from a fraction of said supernatant, e.g. as described herein.

The supernatant, fraction of supernatant or one or more compounds obtained from or obtainable from supernatant may be used in any of the methods described herein. In some embodiments, the supernatant, fraction of supernatant or one or more compounds obtained from or obtainable from supernatant are used in a differentiation screen or a secretion screen as described herein.

In some embodiments, the compound is one or more bacteria. For example, a population of bacteria may be used. In some embodiments, the population comprises at least 2, 5, 10, 50, 100, 300, 500, 1000 bacteria. The description of the supernatant embodiments may be adapted for the bacteria embodiments, mutatis mutandis. For example, in some embodiments, an organoid of the invention is cultured in the presence of one or more bacteria. In some embodiments, the bacteria are added to the culture medium in an assay of the invention after (e.g. 24 hours) differentiation of the stem cells and/or cells with stem cell potential has been induced. In some embodiments, the bacteria are administered to a patient. The bacteria are preferably live bacteria.

Thus, the invention provides a method for testing the effect of one or more bacteria, a supernatant from a bacterial culture or compounds derived from a bacterial culture on EECs or on one or more EEC subtypes, wherein the method comprises (i) contacting one or more organoids of the invention with the bacteria, the supernatant or isolated bacterial compounds; and (ii) determining whether secretion of one or more hormones and/or hormone precursors is affected and/or whether expression of one or more hormones, hormone precursors and/or hormone synthesizing enzymes is affected and/or whether production of one or more hormones from one or more hormone precursors is affected. In some embodiments, the organoid is a reporter organoid.

In some embodiments, the bacteria is of the Akkermansia genera. In some embodiments, the bacteria is Akkermansia muciniphila. In some embodiments, the Akkermansia muciniphila is selected from the Akkermansia muciniphila deposited as DSM 22959 in the DSMZ-German Collection of Microorganisms and Cell Cultures GmbH, the Akkermansia muciniphila deposited as ATCC BAA-835 or the Akkermansia muciniphila deposited as ATCC BAA-2869. In some embodiments, the Akkermansia muciniphila is the Akkermansia muciniphila deposited as DSM 22959 in the DSMZ-German Collection of Microorganisms and Cell Cultures GmbH. The inventors have shown upregulation of individual hormones (most strikingly GHRL) using Akkermansia in an assay method of the invention. CHGA, CCK, GCG, GHRL, MLN and TPH1 levels were all increased, most strikingly GHRL. Thus, in some embodiments, the invention provides the use of Akkermansia, e.g. Akkermansia muciniphila (or a supernatant from a culture thereof) for increasing the expression of one or more of (e.g. 1, 2, 3, 4, 5 or more or all 6 of) GHRL, MLN, CCK, GCG, TPH1 and ChgA in one or more EECs. For example, the invention provides a method for increasing the expression of one or more of (e.g. 1, 2, 3, 4, 5 or more or all 6 of) GHRL, MLN, CCK, GCG, TPH1 and ChgA comprising contacting one or more EECs with Akkermansia. Similarly, the invention provides a method for increasing the expression of one or more of (e.g. 1, 2, 3, 4, 5 or more or all 6 of) GHRL, MLN, CCK, GCG, TPH1 and ChgA in one of more EECs comprising culturing the one or more EECs in the presence of Akkermansia or in the presence of a supernatant from a culture of Akkermansia. For example, in some embodiments, the one or more EECs are in an intestinal organoid, e.g. an organoid of the invention, and Akkermansia is added to the culture medium. The EECs are preferably human EECs. In some embodiments, the hormones and/or hormone precursors are selected from one or more of (e.g. 1, 2, 3 or more or all 4 of) GHRL, MLN, CCK and GCG. Likewise, the invention provides a method of increasing the expression of one or more of (e.g. 1, 2, 3, 4, 5 or more or all 6 of) GHRL, MLN, CCK, GCG, TPH1 and ChgA by EECs comprising administering Akkermansia to a patient. For example, the invention provides a method of treating or preventing a disease or disorder in which one or more of GHRL, MLN, CCK, GCG, TPH1 and ChgA is implicated comprising administering Akkermansia to a patient. Preferably, the Akkermansia is Akkermansia muciniphila. In some embodiments, the invention provides a method of treating or preventing a gut motility disorder (e.g. as described herein), anorexia, cancer cachexia, obesity, diabetes or Prader-Willi syndrome comprising administering Akkermansia to a patient. For example, in some embodiments, the disease or disorder is a disease or disorder related to food intake, e.g. an appetite related disease or disorder. Similarly, the invention provides a method of treating or preventing a gut motility disorder (e.g. as described herein) or diabetes comprising administering Akkermansia to a patient. Likewise, the invention provides Akkermansia for use in treating or preventing a gut motility disorder, obesity, diabetes (in particular, type II diabetes), anorexia or cancer cachexia. In some embodiments, the invention provides Akkermansia for use in treating or preventing Prader-Willi syndrome. For example, in some embodiments, the disease or disorder is selected from a gut motility disorder, anorexia and cancer cachexia. In some embodiments, the disease or disorder is a gut motility disorder. In some embodiments, the disease or disorder is a bowel movement disorder. In some embodiments, the disease or disorder is Parkinson's disease. In some embodiments, the disease or disorder is gastroparesis. In some embodiments, the disease or disorder is diabetes (in particular type II diabetes). In some embodiments, the disease or disorder is obesity. In some embodiments, the disease or disorder is Prader-Willi syndrome. In some embodiments, the disease or disorder is anorexia. In some embodiments, the disease or disorder is cancer cachexia. In some embodiments, the disease or disorder is a neurodegenerative disease or disorder. In some embodiments, the disease or disorder is Parkinson's disease. In some embodiments, the disease or disorder is Alzheimer's disease. In some embodiments, the disease or disorder is a biliary movement disorder. In some embodiments, the disease or disorder is biliary dyskinesia. In some embodiments, the disease or disorder is depression. These diseases and disorders may be treated or prevented using the hormones found to be increased by Akkermansia. In some embodiments, the treating or preventing comprises increasing the expression of one or more of (e.g. 1, 2, 3, 4, 5 or more or all 6 of) GHRL, MLN, CCK, GCG, TPH1 and ChgA by EECs. In some embodiments, the compound is the supernatant from a culture of Akkermansia.

The bacteria is preferably formulated so that it is suitable for administration to a patient and so that it is suitable for administration to or delivery to the gut. For example, it may be formulated as a pharmaceutical composition as described herein, for example, as a capsule, a tablet or a liquid suitable for oral administration. In some embodiments, it is formulated so that it is suitable for oral administration. In some embodiments, it is formulated as a capsule. In some embodiments, it is formulated as a tablet. In some embodiments, it is formulated as a liquid. Thus, in some embodiments, the treating or preventing comprises oral administration of the bacteria. The description provided herein regarding targeting to the gut or targeting specifically to the gut may be applied to the bacterial embodiments, mutatis mutandis.

A patient as described herein is preferably a human patient.

The invention provides a compound as described herein for use in medicine. Similarly, a compound as described herein for use as a medicament is provided. Also provided is a compound as described herein for use in treating a disease or disorder as described herein. The compound is a compound which has preferably been identified/validated by a method of the invention as being suitable for such use. In some embodiments, the compound is a bacterial metabolite, preferably identified using a method as described herein, or a precursor of a bacterial metabolite.

In some embodiments, the step of contacting one or more organoids of the invention with one or more compounds comprises contacting the one or more organoids with a library of compounds, for example, a library of known compounds (e.g. known drugs), a library of unknown compounds, or a library comprising known and unknown compounds. Thus, in some embodiments, a library of compounds is used. Thus, the one or more compounds may be a library of compounds.

Any suitable library or libraries may be used. For example, antibody libraries, antibody fragment libraries, small molecule libraries, peptide phage display libraries, peptide libraries (e.g. LOPAP™, Sigma Aldrich), lipid libraries (e.g. BioMol), synthetic compound libraries (e.g. LOP AC™, Sigma Aldrich), natural compound libraries (e.g. Specs, TimTec), plant extract libraries, libraries of gut metabolites, or chemical libraries may all be used in the present invention. Furthermore, genetic libraries can be used that induce or repress the expression of one of more genes in the cells. These genetic libraries comprise, for example, cDNA libraries, antisense libraries, and siRNA or other non-coding RNA libraries.

In some embodiments, the organoids are exposed to multiple concentrations of a test compound for a certain period of time. At the end of the exposure period, the cultures are evaluated.

The use of a library of compounds advantageously allows high throughput screening for compounds that achieve the desired results, e.g. which affect hormone expression and/or secretion. In some embodiments, multiple organoids are each contacted with a different member of the library. For example, in some embodiments, at least 2, 5, 10, 20, 30, 50, 100 or 500 organoids are each contacted with a different member of the library. In some embodiments, individual organoids are contacted with two or more (e.g. 2, 5, 10 or more) members of the library. If a positive result is obtained, e.g. if secretion and/or expression and/or production of one or more hormones, hormone precursors and/or hormone synthesizing enzymes is affected, the sample containing the two or more members of the library may be investigated further to determine which member of the library is responsible for achieving the effect.

For high-throughput purposes, said organoids of the invention are preferably cultured in multiwell plates such as, for example, 96 well plates or 384 well plates. Libraries of compounds can be used to identify a compound that affects said organoids. The library of compounds may be a library as described herein. Culturing in multiwell plates is also advantageous for contacting a biobank of organoids with one or more compounds.

The organoids are useful for a wide variety of drug discovery purposes. It will be understood by the skilled person that the organoids of the invention would be widely applicable as drug screening tools for the diseases and disorders described herein. Thus, the compound of the invention may be a drug, or may be formulated as a drug.

In some embodiments, a cell biopsy from a patient of interest, can be cultured using culture media and methods of the invention to generate one or more organoids and then treated with a compound or a library. It is then possible to determine which compounds effectively modify, kill and/or treat the patient's cells, e.g. by modulating expression and/or secretion of an EEC-gene of interest, e.g. a hormone, hormone precursor and/or hormone synthesizing enzyme. This allows specific patient responsiveness to a particular drug to be tested thus allowing treatment to be tailored to a specific patient. Thus, this allows a personalized medicine approach.

The ability to obtain a useful organoid of the invention in short time periods (days) shows that the organoids would be highly useful for testing individual patient responses to specific drugs and tailoring treatment according to the responsiveness. In some embodiments, wherein the organoid is obtained from a biopsy from a patient, the organoid is cultured for less than 21 days, for example less than 14 days, less than 13 days, less than 12 days, less than 11 days, less than 10 days, less than 9 days, less than 8 days, less than 7 days, less than 6 days, (etc).

The added advantage of using the organoids for identifying drugs in this way is that it is also possible to screen normal organoids (organoids derived from healthy tissue) to check which drugs and compounds have minimal effect on healthy tissue. This allows screening for drugs with minimal off-target activity or unwanted side-effects.

Drugs for any number of diseases can be screened in this way. For example the organoids of the invention can be used for screening for drugs for any of the diseases and disorders described herein.

Therefore, the invention provides a method for screening for or preparing a therapeutic or prophylactic pharmaceutical drug or cosmetic, wherein the method comprises:
i) contacting an organoid of the invention with a compound (or a library of compounds),
ii) evaluating said organoids for any effects (e.g. a change in secretion and/or expression and/or production of one or more hormones, hormone precursors and/or hormone synthesizing enzymes);
iii) identifying the compound that causes said effects as a potential drug or cosmetic; and optionally
iv) preparing said compound as a pharmaceutical or cosmetic.

In some embodiments, the therapeutic or prophylactic pharmaceutical drug is a pharmaceutical composition as described herein.

In some embodiments, computer- or robot-assisted culturing and data collection methods are employed to increase the throughput of the screen. In some embodiments, the organoid is derived from a patient biopsy. In some embodiments, the compound that causes a desired effect on the reporter organoid is administered to said patient.

Accordingly, in one aspect, there is provided a method of treating a patient comprising:
(a) obtaining a biopsy from the diseased tissue of interest in the patient;
(b) obtaining an organoid of the invention from the biopsy using a method as described herein;
(c) screening for a suitable drug using a screening method of the invention; and
(d) treating said patient with the drug obtained in step (c).

In some embodiments, the drug or cosmetic is used for treating, preventing or ameliorating symptoms of a disease or disorder described herein.

Patient-specific organoids obtained from diseased and/or normal tissue can be used for target validation of compounds identified in high throughput screens. The same applies for the validation of compounds that were identified as possible therapeutic drugs in high throughput screens. The use of primary patient material differentiated in the organoid culture system can be useful to test for false positives, etc. from high throughput drug discovery cell line studies.

In some embodiments, the organoid of the invention can be used for validation of compounds that have been identified as possible drugs or cosmetics in a high-throughput screen.

The organoids provided herein may be of use in a method as described herein, for example, for generating a cell atlas, for performing differentiation screens (e.g. using a tagged hormone to detect which cell population is made in response to a particular stimulus), for secretion screens (e.g. determining how release of a specific hormone is triggered), or for drug discovery screens or in any other method described herein. The organoids described herein are all provided by the present invention.

The invention provides the use of an organoid of the invention in a drug discovery screen; a (drug) target validation; a (drug) target discovery; a toxicity assay; for personalised medicine; as an ex vivo cell/organ model, e.g. such as a disease model; for research of tissue embryology, cell lineages, and differentiation pathways; for research to identify the chemical and/or neuronal signals that lead to the release of the respective hormones; for gene expression studies including recombinant gene expression; for research of the diseases and disorders described herein. These uses are non-limiting.

Cells and organoids cultured according to the media and methods of the invention are thought to faithfully represent the in vivo situation. This is true both for organoids grown from normal tissue and for organoids grown from diseased tissue. Therefore, as well as providing normal ex vivo cell/organ models, the organoids of the invention can be used as ex vivo disease models.

In some embodiments in which the compound is said to target EECs, the compound may be formulated to target EECs, for example, it may be present in a pharmaceutical composition that targets EECs.

In embodiments which describe methods of treating or preventing a disease or disorder, the method preferably comprises administering a therapeutically effective amount of the compound. A “therapeutically effective amount” refers to an amount of a therapeutic compound that is sufficient, when administered to a subject/patient suffering from or susceptible to a disease, disorder, and/or condition, to treat, diagnose, prevent, and/or delay the onset of the symptom(s) of the disease, disorder, and/or condition. It will be appreciated by the skilled person that a therapeutically effective amount is typically administered via a dosing regimen comprising at least one unit dose.

“Treating or preventing” as used throughout this disclosure refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The skilled person will understand the conditions under which to perform the various methods of the invention. For example, the skilled person will understand how to choose appropriate culture medium for performing the various steps of the assays. Thus, in the various steps of the various methods described herein, the organoid is preferably cultured in a suitable culture medium.

Sensors

In some embodiments, a sensor is used in combination with organoids of the invention, for example, in combination with tagged hormones from one or more reporter organoids, or a biobank of the invention to study functional signalling responses (e.g. hormone secretion) in EECs. The cells are preferably human cells, for example, human EECs or human EEC subtypes. Thus, the invention provides a method for studying intracellular signalling in one or more EECs or in one or more subtypes thereof (e.g. in one or more human EEC subtypes), wherein the method comprises (i) contacting one or more intestinal organoids of the invention comprising a sensor of intracellular signalling with a compound; and (ii) determining whether the sensor is affected. For example, the method may be used for identifying/screening/validating the compound to determine if it affects intracellular signalling in the one or more EECs (e.g. in one or more human EEC subtypes), and optionally determining the subtype of EECs affected by the compound.

Advantageously, the method may be used to identify subtypes of EECs affected by a particular compound. For example, the method may comprise determining whether the cell in which the sensor is affected is positive for or negative for a cell subtype-specific marker, and determining the identity of the affected cell accordingly. For example, the method may comprise determining whether the cell in which the sensor is affected expresses or secretes a tagged hormone, tagged hormone synthesizing enzyme and/or tagged hormone precursor from the reporter organoid. The identity of the cell subtype expressing the tagged hormone, tagged hormone synthesizing enzyme or tagged hormone precursor can be determined by the identity of the tagged hormone, tagged hormone synthesizing enzyme and/or tagged hormone precursor that is expressed and so if the sensor is affected in the same cell as the tagged hormone, tagged hormone synthesizing enzyme and/or tagged hormone precursor is expressed, it can be determined that the compound affects signalling in that cell subtype. Similarly, if the sensor is affected in a different cell from the cell in which the tagged hormone, tagged hormone synthesizing enzyme or tagged hormone precursor is expressed, it can be determined that the compound does not affect signalling in that cell subtype. In some embodiments, a further detectable marker is expressed in all EECs in the organoids. In some embodiments, this shows that the sensor is affected in EECs but not in the EEC subtype in which the tagged hormone, tagged hormone synthesizing enzyme or tagged hormone precursor is expressed. It may be possible to derive the EEC subtype in which the compound affects signalling by a process of elimination. For example, if there are only two EEC subtypes in the organoid being studied and the sensor is affected in cells marked as EECs by the further detectable marker but is not affected in the cell subtype in which the tagged hormone, tagged hormone synthesizing enzyme or tagged hormone precursor is expressed, it suggests that the compound affects signalling in the other EEC subtype. For example, in embodiments in which the organoid is a colon organoid, the EECs comprise L-cells and serotonin-producing ECs. If the compound is found to affect signalling in EECs but is found not to affect signalling in serotonin-producing ECs comprising the tagged hormone synthesizing enzyme TPH1, it can be derived that the compound affects signalling in L-cells.

For example, in some embodiments, the compound is beta-ionone and the sensor is a calcium sensor. Beta-ionone is an agonist of the olfactory receptor OR51E2. The mouse homologue of OR51E2 had been reported to be expressed in mouse EECs (Fleischer et al., 2015; Jovancevic et al., 2017). However, prior to the invention, it was not known which human EECs OR51E2 is expressed in. Olfactory genes in humans and mice are very different as these genes quickly evolve. Therefore, the homology of these sequences tend to be low, and it is difficult to project expression profiles of mouse olfactory genes on humans. The inventors have found two olfactory receptors that are abundantly expressed in human EECs, OR51E1 and OR51E2. When reporter organoids derived from the colon in which OR51E2 was overexpressed were stimulated with beta-ionone, a ligand of OR51E2, the inventors observed calcium sparking in EECs that were TPH1-negative. This provided proof-of-concept that sensors combined with hormone reporters from EEC-TAG can aid the study of functional signalling responses in human EEC subtypes. Thus, calcium reporters can be combined as a measure of hormone activation with the knowledge of the expression levels of targets such as olfactory receptors in subpopulations of EECs. Thus, the use of a reporter organoid in conjunction with a calcium reporter allows for screening for activators of targets such as cell surface receptors, e.g. olfactory receptors, and such methods are provided herein. In some embodiments, the calcium assay is conducted in an intestinal organoid which is not itself a reporter organoid and a reporter organoid is used to validate which EEC subtype the calcium is being activated in and so which hormones are being activated. In some embodiments, the method is conducted in a reporter organoid and if the calcium signal is detected in one or more individual cells together with the detectable marker linked to a hormone, hormone precursor and/or hormone synthesizing enzyme from the reporter organoid (e.g. the fluorescent tag), detection of the detectable marker allows a determination to be made as to which EEC subtype the calcium sparking is occurring in. This can be extrapolated to which EEC subtype hormone secretion is being activated in.

In addition, the invention provides beta-ionone as a regulator of hormone secretion. For example, in some embodiments, the invention provides a method of regulating hormone secretion comprising contacting an EEC with beta-ionone. In some embodiments, the regulating hormone secretion is increasing hormone secretion.

In some embodiments, the sensor is a calcium sensor or a cAMP sensor. In some embodiments, a calcium sensor or cAMP sensor is introduced into an organoid of the invention. In some embodiments, the calcium sensor or cAMP sensor is stably introduced. For example, stable introduction may be effected using lentiviral transduction. Advantageously, the gene encoding the sensor (for example, the calcium sensor or cAMP sensor) may be linked to a detectable marker gene, e.g. a fluorescent gene. In some embodiments, this allows integration into the organoid genome to be detected. In some embodiments, the detectable marker allows detection of calcium binding. For example, a change in the folding of the calcium reporter upon calcium binding may activate the bound fluorescent protein. In some embodiments, the sensor is a calcium sensor. In some embodiments, the calcium sensor is a Ca2+ sensor, e.g. GCaMP3 (Borges-Pereira, L. et al., 2014), or a sensor based thereon, e.g. Ca-FLITS. For example, in some embodiments, a fluorescent marker-Ca-FLITS construct is stably introduced into the organoid using lentiviral transduction. In some embodiments, the organoid is a reporter organoid.

The invention therefore provides a method for studying intracellular calcium signalling or intracellular cAMP signalling in EECs or in one or more subtypes thereof comprising contacting one or more organoids of the invention with a compound and determining whether an increase or decrease in intracellular calcium, e.g. a calcium spike or calcium trough, is detected. Calcium sparking is the hallmark of EEC activation and hormone secretion. Most hormone secretion is calcium mediated, for example, by calcium sparks. Calcium sparking can therefore be another readout to measure hormone secretion. Advantageously, combining calcium sensors with hormone reporters therefore makes it possible to measure hormone secretion in individual EEC subtypes. In some embodiments, the sensor is a cAMP sensor, for example, EPAC (Robichaux, W. and Cheng, X. “Intracellular cAMP Sensor EPAC: Physiology, Pathophysiology and Therapeutics Development; Physiological Reviews, 98(2): 919-1053, 2018).

Calcium signalling and cAMP signalling are mediators of hormone secretion (Goldspink et al., 2018). Thus, advantageously, a method that detects intracellular calcium signalling (e.g. calcium sparks) and/or intracellular cAMP signalling in response to contacting an organoid with a compound may be indicative of the ability of the compound to affect hormone secretion.

Accordingly, the invention similarly provides a method for determining whether a compound affects secretion of one or more hormones and/or hormone precursors in EECs or in one or more subtypes thereof, wherein the method comprises (i) contacting one or more organoids of the invention which comprise a calcium sensor and/or a cAMP sensor with the compound, and (ii) determining whether an increase in intracellular calcium and/or cAMP or a decrease in intracellular calcium and/or cAMP is detected. In some embodiments, the increase in intracellular calcium is a calcium spike. In some embodiments, an increase in intracellular cAMP is a cAMP spike. In some embodiments, the organoid is a reporter organoid and then the method optionally further comprises determining whether the cell in which the sensor is affected expresses a tagged hormone, tagged hormone synthesizing enzyme or tagged hormone precursor from the reporter organoid, determining the subtype of the EEC in which the sensor is affected and accordingly determining that the compound affects signalling (and hence secretion) in that EEC subtype. In some embodiments, if an increase in intracellular calcium or cAMP is detected, for example, a calcium or cAMP spike, it indicates that the compound causes secretion of hormones and/or hormone precursors. In some embodiments, if a reduction in intracellular calcium or cAMP is detected, it indicates that the compound prevents secretion of hormones and/or hormone precursors. This can then be validated by using the reporter organoid of the invention to determine if the compound affects secretion, for example, if it causes or prevents secretion of one or more tagged hormones and/or hormone precursors. For example, a decrease in fluorescence from the hormone marker in the EEC or EEC subtype between two time points or compared to a control organoid that has not been contacted with the compound would indicate secretion of the hormone. Accordingly, the method may further comprise the step of determining that the compound affects secretion of one or more hormones and/or hormones precursors in a particular EEC subtype. For example, the method may determine that the compound increases secretion in a particular EEC subtype. Alternatively, the method may determine that the compound decreases secretion in a particular EEC subtype.

The ability to correlate hormone secretion dynamics to compounds in individual cell subtypes, for example, in individual EEC subtypes, advantageously assists the discovery of compounds that are therapeutic agents.

Preferably, the EECs and EEC subtypes described herein are human EECs and human EEC subtypes.

The methods described herein which use one or more organoids may optionally further comprise the steps of making the one or more organoids.

In some embodiments, a method or use of the invention is carried out in vitro. This may apply to any method or use of the invention, as appropriate. For example, it may apply to the use of one or more compounds for modulating hormone expression or for controlling differentiation. Generally, the methods of the invention which relate to organoids are carried out in vitro.

EEC Atlas

The invention provides, for the first time, a human EEC atlas. Single cell transcriptomics presents a powerful technique to assess heterogeneity among cell populations and identifies genes specific to individual cell types (Haber et al., 2017; Parikh et al., 2019). Due to the paucity of EECs, studies in mice have utilized reporter mice to enrich for hormone-producing cells when performing single cell RNA sequencing. This approach cannot be used for primary human EECs, which, prior to the present invention, made the generation of a detailed atlas from small intestinal tissue challenging. Advantageously, the differentiated organoids (e.g. the differentiated reporter organoids) of the invention comprise a high percentage of EECs and the tagged EEC-specific genes (e.g. tagged hormones) allow single EECs to be obtained and the subtypes of these single EECs to be identified, thus enabling single cell transcriptomics to be used to generate a human EEC atlas for the first time.

The inventors have generated a high-resolution transcriptomic and proteomic profile of human EECs from three locations along the gastrointestinal tract, including a first assessment of their secreted products. This dataset yields new hormones, transcription factors and receptors (amongst other new genes/proteins), and can be mined for novel therapeutic targets. The expression atlas highlights key differences with murine counterparts, stressing the importance of performing studies on EEC function in a human model system, for example, in intestinal organoids or intestinal reporter organoids of the invention.

The invention provides a method for generating an EEC RNA expression atlas, comprising (i) obtaining EECs from one or more organoids of the invention which expresses a detectable marker (e.g. a fluorescent marker) linked to an EEC-specific gene, (ii) performing single cell mRNA sequencing, (iii) analysing the transcriptome data (e.g. using a clustering method, e.g. a clustering method based on k-medoids, e.g. RaceID3 (Herman et al., 2018)) to determine the expression levels of genes expressed in single cells, and (iv) generating an EEC atlas using the information obtained in step (iii). In some embodiments, step (i) comprises obtaining EECs from reporter organoids established from the duodenum, from the ileum and/or from the colon.

In some embodiments, the intestinal organoid used in step (i) is a reporter organoid of the invention. In some embodiments, the intestinal organoid used in step (i) is not a reporter organoid. In some embodiments, the intestinal organoid used in step (i) is an organoid obtained by or obtainable by a method for enriching the population of EECs in an intestinal organoid. Thus, in some embodiments, the detectable marker is linked to the EEC-specific gene at its endogenous locus. In some embodiments, the detectable marker is linked to the transcription factor in the overexpression construct. The transcription factor in the overexpression construct is the transcription factor for differentiating intestinal stem cells and/or intestinal cells with stem cell potential to EECs.

In some embodiments, the gene that identifies EECs in step (i) is a gene that is expressed broadly in EECs, e.g. that is expressed in EECs of all EEC subtypes. In some embodiments, the gene that identifies EECs in step (i) is selected from the group consisting of a hormone, hormone synthesizing enzyme or hormone precursor expressed or secreted by EECs or by EECs of one or more subtypes, the CHGA gene, the NEUROG3 gene, the ATOH1/MATH1 gene or the NEUROD1 gene. In some embodiments, the gene that identifies EECs in step (i) is a hormone, hormone synthesizing enzyme or hormone precursor expressed or secreted by EECs or by EECs of one or more subtypes. For example, in some embodiments, the gene that identifies EECs in step (i) is a hormone or hormone precursor secreted by EECs or by EECs of one or more subtypes. In some embodiments, the gene that identifies EECs in step (i) is selected from the group consisting of: the NEUROG3 gene, the ATOH1/MATH1 gene or the NEUROD1 gene. In some embodiments, the gene that identifies EECs in step (i) is the CHGA gene. In some embodiments, the gene that identifies EECs in step (i) is the NEUROG3 gene. In some embodiments, contaminant non-EEC cell types are present in the intestinal cell atlas, for example, one or more of FABP1-positive enterocytes, OLFM4-positive stem cells, MUC2-positive goblet cells, LYZ/MMP7-positive Paneth cells and/or progenitor populations.

In some embodiments, the method comprises selecting for EECs and optionally their progenitors by positively selecting for expression of an EEC marker, for example, the generic EEC marker CHGA, and negatively selecting against markers for the contaminant non-EEC cells, for example, one or more of MUC2, FABP1, LYZ and OLFM4. In some embodiments, the selection for EEC cells is performed using transcriptome data and is performed after step (iii) and before step (iv) or after step (iv). In some embodiments, the selection for EEC cells is performed in step (i).

In some embodiments, the cells are further subclustered by sorting from organoids carrying one or more tagged hormones, tagged hormone precursors and/or tagged hormone synthesizing enzymes using a reporter organoid of the invention. This enables heterogeneity among the different EEC subtypes to be identified.

The in vitro identity of the EECs identified in the cell atlas may be validated by comparing the information in the atlas with mRNA signatures for EECs contained in the large single cell-dataset obtained from healthy and diseased human small intestines of various ages at the Sanger Center.

In some embodiments, the gene that identifies EECs in step (i) is a gene that is expressed in one or more EEC subtypes and identifies the one or more EEC subtypes. For example, in some embodiments, the gene that identifies EECs in step (i) is a gene that is expressed in one EEC subtype but is not expressed in any other EEC subtypes.

Accordingly, the invention provides a method for generating an EEC subtype RNA expression atlas, comprising (i) obtaining cells of a subtype of EEC by obtaining cells from a reporter organoid of the invention which express the detectable marker (e.g. fluorescent marker) linked to an EEC-specific gene (e.g. a hormone, hormone synthesizing enzyme or hormone precursor) that identifies that EEC subtype, (ii) performing single cell RNA sequencing, (iii) analysing the transcriptome data (for example, using a method as described herein) to determine the expression levels of genes expressed in single cells, and (iv) generating an EEC atlas using the information obtained in (iii).

In some embodiments, the step of obtaining the EECs or the cells of the one or more EEC subtypes comprises isolating the EECs from all other cell types.

Preferably, the expression level of all or substantially all the genes in the single cells is determined. In some embodiments, the expression level of at least 50% (e.g. at least 60%, 70%, 80%, 90%, 95%, 98%, 99% or 100%) of the genes in the single cells is determined.

The method for obtaining cells of the EEC subtype may be a method as described herein. In some embodiments, the atlas is generated for a single EEC subtype. In some embodiments, the method is repeated for one or more additional EEC subtypes to generate the EEC atlas. Thus, in some embodiments, the atlas is generated for multiple EEC subtypes. In some embodiments, the method is repeated for each different EEC subtype to generate the EEC atlas. In some embodiments, reporter organoids are used to enrich for rare EEC subtypes. For example, in some embodiments, the hormones are sorted for in order to enrich for cells expressing the hormone. Examples of cells that may be enriched for in some embodiments are Somatostatin+ (D) cells, M/X cells (which are rare in the ileum, but not in the duodenum), G/K cells (which are rare in the ileum, but not in the duodenum) and L-cells (which are rare in the duodenum, but not in the ileum).

In some embodiments, an EEC atlas is generated for EECs or subtypes of EECs obtained from organoids established from one or more of (e.g. 1, 2, 3, 4, 5, 6, 7 or all of) the proximal small intestine (duodenum), the distal small intestine (ileum), the ascending colon, the descending colon, the transverse colon, the sigmoid colon, the rectum and the jejunum. Advantageously, the method may be carried out for each of duodenal, ileal and colon organoids. This allows EECs, for example, all EECs or one or more EEC subtypes, from each of these regions to be studied.

In some embodiments, following RNA sequencing of single cells, the transcriptome data is analysed using a clustering method, for example, a clustering method based on k-medoids, e.g. RaceID3 (Herman et al., 2018).

In some embodiments, following analysis of the transcriptome data, the data may be filtered, for example using a cut off of a suitable number of uniquely expressed transcripts per cell, e.g. at least 2000, before generating the cell type atlas.

In some embodiments, the EEC atlas comprises or consists of mRNA expression level information. In some embodiments, the EEC atlas is generated as a t-SNE map.

Transcriptomics of pooled cells has an even superior sensitivity compared to single cell RNA sequencing. Again, this approach was not possible using primary human EECs. Some EEC subtypes are very rare in vivo, for example, the EECs that make a lot of PPY (pancreatic polypeptide Y). Huge amounts of single cell RNA sequencing from tissue would be required, probably from at least one million cells, which is currently not feasible. The provision of organoids of the invention, which comprise more than 10% EECs (for example, which in some embodiments comprise more than 50% EECs), makes transcriptomics approaches possible for the first time. The use of the reporter organoids of the invention allows transcriptomic signatures of individual cell types to be generated and markers expressed at the RNA level specific to individual EEC types to be identified for the first time. For example, the use of a reporter organoid of the invention is the only way to perform bulk analyses on EEC subtypes. It allows pooling the same EEC subtype for high-definition RNA profiling. In mouse, this could be done with transgenic mice models that exist for a few hormones, but such models do not exist for humans. With bulk analyses, it is possible to identify many more genes than with a single cell.

Accordingly, also provided is a method for generating an EEC RNA expression atlas, comprising (i) obtaining a population of cells of a subtype of EEC by obtaining cells from a reporter organoid of the invention which express a detectable marker linked to an EEC-specific gene (e.g. a hormone, hormone synthesizing enzyme or hormone precursor) that identifies that EEC subtype, (ii) performing RNA sequencing on the population of cells obtained in step (i); (iii) repeating steps (i) and (ii) for one or more other EEC subtypes (for example, for all other EEC subtypes); and (iv) identifying uniquely expressed markers from the RNA sequencing dataset for the different EEC subtypes.

In some embodiments, the cells used in a method for generating an EEC RNA expression atlas are EC-cells, L-cells and/or M-X cells. EC-cells, L-cells and M-X cells may be identified and obtained using reporter organoids of the invention in which hormones, hormone synthesizing enzymes and/or hormone precursors expressed in these cell types are tagged, as described herein.

In some embodiments, in addition to using cells from one or more EEC subtypes, a population comprising EECs (e.g. a population comprising EECs of all subtypes) are additionally used (for example, by obtaining these from reporter organoids in which a broadly expressed EEC-specific gene is tagged, e.g. CHGA).

As a control, non-EECs from the intestine may be used (for example, by obtaining these from reporter organoids in which a broadly expressed EEC-specific gene is tagged (e.g. CHGA) and selecting cells which do not express the tagged gene (e.g. selecting CHGA negative cells)). Thus, in some embodiments, the methods comprise comparing the results to a control, and for example, including only genes that are expressed in EECs and not expressed in non-EECs from the intestine in the cell atlas. For example, the method may comprise excluding any genes expressed in non-EECs from the intestine from the cell atlas.

The invention further provides a method for generating an EEC protein expression atlas, comprising (i) obtaining a population of EECs of a subtype of EEC by obtaining cells from a reporter organoid of the invention which express a detectable marker linked to an EEC-specific gene (e.g. a hormone, hormone synthesizing enzyme or hormone precursor) that identifies EECs of that subtype; (ii) conducting intracellular proteomic studies on the population of EECs; and (iii) generating an EEC atlas using the information obtained in (ii). The method may further comprise repeating steps (i) and (ii) for one or more additional EEC subtypes, advantageously, for all additional EEC subtypes.

In some embodiments, the atlas is a human atlas or a non-human mammalian atlas, e.g. of a small mammal, for example, a mouse, rat, hamster, guinea pig or rabbit. The atlas is preferably a human atlas. In some embodiments, the atlas is not a mouse atlas.

An EEC atlas obtained by or obtainable by the method of the invention is also provided. In some embodiments, the EEC atlas is an EEC subtype atlas. For example, the invention provides a human EEC atlas or a human EEC subtype atlas. In some embodiments, the invention provides a human EEC atlas or a human EEC subtype atlas obtained by or obtainable by the method of the invention. The EEC subtype may be an EEC subtype as described herein. In some embodiments, the atlas is an RNA expression atlas. In some embodiments, the atlas is a protein expression atlas.

Uses of the Cell Atlas

The transcriptional networks generating the different EEC subtypes have been well worked out in mice (Beucher et al., 2012; Gehart et al., 2019; Gross et al., 2016; Piccand et al., 2019). These networks could result from a stochastically acting system that generates fixed ratios of different EECs. This would explain why organoids generate rather conserved ratios of individual subtypes when compared to their tissue of origin, even in the absence of mesenchymal and luminal factors (Beumer et al., 2018).

Taken together, the organoids (e.g. the reporter organoids) and EEC atlas of the invention provide rich resources for identifying regulators of human EEC development. In particular, they allow factors to be identified which control the differentiation of EEC progenitors to their various EEC subtypes. Identifying such factors is useful because the ability to enrich for specific EEC subsets in organoids would enhance the applicability of the system.

Also provided is a method for identifying one or more genes expressed by a human EEC, comprising analysing a human EEC atlas to determine which genes are expressed. The method optionally comprises determining which genes are expressed by a particular subtype of human EECs. In some embodiments, the method comprises determining which genes are expressed by a particular subtype of human EECs but are not expressed by one or more (e.g. 2, 3, 4, 5, or more or all) other subtypes of EECs. For example, the method may optionally comprise selecting for a particular hormone identified herein as being produced by a particular EEC subtype and identifying one or more genes expressed in that EEC subtype using the human EEC atlas. In some embodiments, the method comprises determining which genes are expressed by EECs but are not expressed by non-EECs in the intestine. In some embodiments, the method comprises determining which genes are expressed by one or more subtypes of EECs but are not expressed by other EEC subtypes and are not expressed by non-EECs in the intestine.

Also provided is a method for identifying one or more EEC-specific genes. Such genes may be of use in a reporter organoid of the invention. For example, the method may comprise comparing the EEC RNA expression atlas or EEC protein expression atlas, for example, as described herein, with an intestinal cell atlas comprising cells from the intestine other than EECs and identifying genes and/or proteins expressed in the EECs but not in the other cells. Preferably the intestinal cell atlas comprises all cells from the intestine other than EECs.

In some embodiments, gene expression is detected at the RNA level. In some embodiments, gene expression is detected at the protein level.

In some embodiments, the EEC subtype is a Motilin expressing cell. The hormone Motilin is expressed in human EECs but is not expressed in mice. Instead, it is only a pseudogene in mice. In some embodiments, the fluorescent marker in the reporter organoid of step (i) is linked to Motilin. In some embodiments, the EEC cell expressing Motilin is an M-X cell. Thus, in some embodiments, the invention provides a method for generating a cell atlas of a Motilin-expressing M-X cell, for example, of a human Motilin-expressing M-X cell.

The human EEC atlas may be compared with an EEC atlas from another organism, e.g. with the previously published mouse EEC atlas (Gehart et al., 2019) to identify differences in EECs between the two species. For example, this allows identification of genes and proteins expressed in human EECs but not in the other species, e.g. mouse. Accordingly, in some embodiments, the method may be used to identify one or more genes expressed by a human EEC but not expressed by an EEC from a different organism, comprising comparing a human EEC atlas to an EEC atlas from the different organism and identifying one or more genes expressed by the human EEC that are not expressed in an EEC from the different organism. The method may further comprise the steps of constructing the human EEC atlas and optionally the EEC atlas from the different organism. In some embodiments, the different organism is a mouse.

In some embodiments, the gene identified as an EEC-specific gene or as an EEC gene expressed in human EECs but not in EECs from one or more other organisms is a hormone, a hormone synthesizing enzyme or a hormone precursor. In some embodiments, such gene is a hormone. In some embodiments, such gene is a cell surface receptor. In some embodiments, such gene is a transcription factor.

Applications Involving Targets Expressed by Human EECs

The inventors have identified a number of cell types using the human EEC atlas analysis that are not previously known in humans. These cell types have been found to express particular targets, such as hormones, receptors or transcription factors. In addition, a number of hormones, receptors and transcription factors have been identified as being expressed in human EECs for the first time (either at the RNA level, at the protein level, or both). These newly identified targets are useful for identifying and obtaining the cell types and can be used as markers for the cell types. They may also be useful as drug targets. Due to the scarcity of EECs in human tissue in vivo, it has been complicated to validate EEC markers prior to the present invention. Thus, the organoid-based EEC atlas of the invention, together with the organoids of the invention, provide a very useful tool for identifying and validating expression of novel genes and identifying/validating putative drug targets. For example, in some embodiments, one or more of the targets newly identified by the present invention is used in the methods and uses of the invention described herein, for example, as described for the reporter organoids. For example, they may be used as a target or as a compound in the methods and uses of the invention. The newly identified targets may also be studied in methods which use conventional techniques which do not utilise a reporter organoid of the invention. For example, such targets may also be studied in cell cultures that do not comprise organoids or the targets may be isolated from the cells for further analysis. Such methods are likewise included within the scope of the invention.

The description provided in the present application is intended to be read as a whole because the embodiments and description provided throughout the description are interrelated. For example, the embodiments mentioned elsewhere in the description can be applied to the methods and other aspects in this section mutatis mutandis. Likewise, the embodiments mentioned in the methods and other aspects in this section can be applied to the methods and other aspects described elsewhere in the description, mutatis mutandis. For example, the description of which compounds are suitable for treating which diseases and disorders can be applied to the various aspects throughout the description.

Throughout the description we describe compounds that target the targets described herein. Throughout the description we also describe how to identify compounds that target the targets described herein and how to identify compounds which are suitable for treating the diseases and disorders described herein. In particular, the description provides a detailed discussion as to which diseases and disorders are treatable or preventable by increasing or inducing expression and/or secretion and/or production of a particular hormone, hormone precursor and/or hormone synthesizing enzyme. Similarly, the description provides a detailed discussion as to which diseases and disorders are treatable or preventable by decreasing or ceasing expression and/or secretion and/or production of a particular hormone, hormone precursor and/or hormone synthesizing enzyme. These disclosures throughout the description should be read together as necessary as they would by a person of skill in the art.

In some embodiments, the target for use in the invention is any target identified herein in the examples, figures or description. The target is preferably a target that has been identified using the cell atlas generated by the inventors. In some embodiments, the target is a target identified in the description. The target may be a protein, an RNA or a gene. Preferably, the target is a protein. The target is preferably expressed by a human EEC and is targeted in a human EEC.

Where expression has been identified at the RNA level using an EEC atlas provided by the invention, it would be straightforward for the skilled person to validate whether expression is also present at the protein level using an organoid of the invention (for example, using a reporter organoid of the invention). Techniques for validating expression at the protein level are well known in the art, for example, using immunofluorescence or mass spectrometry. Examples of such techniques are described herein. Accordingly, in some embodiments, expression is detected at the RNA level. In some embodiments, expression is detected at the protein level.

In some embodiments, the invention provides mining the human EEC atlas to identify one or more novel therapeutic targets. For example, use of the EEC atlas to identify a potential therapeutic target on an EEC is provided herein. For example, there is provided a method for identifying a potential therapeutic target on an EEC comprising identifying a suitable target expressed in an EEC using an EEC atlas of the invention. In some embodiments, the potential therapeutic target is a protein, RNA or gene that is expressed in one or more (e.g. only 1 or 2 or fewer, 3 or fewer, 4 or fewer, 5 or fewer) EEC subtypes or in one or more EECs from a particular region of the intestine. In some embodiments, the potential therapeutic target is a protein, RNA or gene that is expressed specifically in EECs or in one or more EEC subtypes. For example, in some embodiments, the potential therapeutic target is a protein, RNA or gene expressed in EECs but not expressed in non-EECs. In some embodiments, the non-EECs are cells from the intestine that are not EECs. In some embodiments, the potential therapeutic target is a protein, RNA or gene that is expressed in one or more (e.g. only 1 or 2 or fewer, 3 or fewer, 4 or fewer, 5 or fewer) EEC subtypes but not in any other EEC subtypes. In some embodiments, the potential therapeutic target is a protein, RNA or gene that is expressed in one or more EEC subtypes found in one region of the intestine but is not expressed in EEC subtypes found in other regions of the intestine. In some embodiments, the potential therapeutic target is a protein, RNA or gene which is also expressed on one or more non-EEC cells. In more preferred embodiments, the potential therapeutic target is a protein, RNA or gene which is not also expressed on one or more non-EEC cells. In some embodiments, the target is a target as described herein. Preferably, the EECs are human EECs. The target is preferably a protein.

In some embodiments, the potential therapeutic target is a cell surface receptor, for example, a GPCR. Thus, in some embodiments, the target is a cell surface receptor that is a GPCR. In some embodiments, the target is a cell surface receptor that is not a GPCR. In some embodiments, the potential therapeutic target is selected from a cell surface receptor, an intracellular receptor, a transcription factor, a hormone, a hormone precursor, a hormone synthesizing enzyme, a signalling protein, a kinase, and a Snare protein. In some embodiments, the potential therapeutic target is an intracellular protein, for example, an intracellular receptor. In some embodiments, the potential therapeutic target is a transcription factor. In some embodiments, the potential therapeutic target is a hormone. In some embodiments, the potential therapeutic target is a signalling protein. In some embodiments, the potential therapeutic target is a kinase. In some embodiments, the potential therapeutic target is a Snare protein. In some embodiments, the potential therapeutic target is any protein that could regulate secretion and/or expression of one or more hormones, hormone precursors and/or hormone synthesizing enzymes.

Embodiments which describe therapeutic targets or potential therapeutic targets may also be applied to embodiments which describe targets, mutatis mutandis.

The method may subsequently comprise targeting the identified potential therapeutic target (for example, targeting an identified cell surface receptor, e.g. a GPCR). In some embodiments, the potential therapeutic target is contacted with one or more compounds. Examples of suitable compounds are described elsewhere herein. For example, in some embodiments, the one or more compounds are known ligands of the potential therapeutic target, e.g. they are known ligands of the identified cell surface receptor. In some embodiments, the one or more compounds are not known to be ligands of the potential therapeutic target. In some embodiments, a mixture of known ligands of the potential therapeutic target and compounds not known to be ligands of the potential therapeutic target are used.

The potential therapeutic target is preferably targeted in situ in the EEC, for example, when it is targeted with a known ligand of the potential therapeutic target. For example, the EEC may be in an organoid of the invention. In some embodiments, the organoid is a reporter organoid. Thus, in some embodiments, the method comprises contacting the EEC, e.g. contacting the organoid, with one or more compounds. The method may further comprise assessing the effect of contacting the EEC with the one or more compounds (e.g. with the one or more known ligands). For example, the method may comprise determining if hormone secretion is affected. For example, the method may comprise determining if the compound affects one or more of protein expression, gene expression, protein secretion (e.g. secretion of one or more hormones and/or hormone precursors), cell function, cell identity (e.g. cell differentiation), cell morphology and cell viability. In some embodiments, the method may comprise determining if expression and/or secretion and/or production of one or more hormones, hormone precursors and/or hormone synthesizing enzymes is affected, for example, using a reporter organoid of the invention. For example, the identified potential therapeutic target may be used in a method for identifying/validating a drug target of interest in EECs, as described herein. Likewise, the identified potential therapeutic target may be used in a method for testing the effect of a compound on EECs, as described herein. Similarly, the identified potential therapeutic target may be used in a method for identifying/screening/validating a compound for suitability for treating or preventing a disease or disorder, as described herein. Thus, the identified target may be used in any of the methods described herein, as appropriate.

The invention provides an isolated population of human EECs expressing one or more (e.g. 1, 2, 3, 4 or more) targets. The EECs in the isolated population may be of one or more EEC subtypes, for example, 1, 2, 3, 4, 5, 6, 7 or more EEC subtypes. In some embodiments, the isolated population comprises EECs of all EEC subtypes. Also provided is an isolated human EEC expressing one or more (e.g. 1, 2, 3, 4 or more) targets. In some embodiments, the isolated population of cells or the isolated cell is obtained from or is obtainable from an organoid of the invention, for example, from a reporter organoid of the invention. In some embodiments, a single cell is obtained and is clonally expanded to form a population of cells. In some embodiments, a population of cells is obtained. Isolated single cells and populations of cells according to the invention may be useful for studying EEC biology. The ability to isolate single cells makes it possible to measure the transcriptome of individual EECs, unravelling their heterogeneity. This allows assigning of e.g. receptors to EEC subtypes to derive insights on drugs for targeted hormone release. It also allows assigning the impact of a treatment to a subgroup of EECs (e.g., treating with BMP and observing a subset of cells changing their expression profile).

The invention further provides the use of expression of one or more (e.g. 1, 2, 3, 4 or more) targets as one or more markers for human EECs. For example, the invention provides a method for isolating one or more EECs or for isolating a population of EECs comprising selecting cells which express one or more (e.g. 1, 2, 3, 4 or more) targets and isolating the selected cells. Similarly, the invention provides a method for identifying one or more EECs or for identifying a population of EECs comprising selecting cells which express one or more (e.g. 1, 2, 3, 4 or more) targets. The cells are preferably isolated or selected from a population of intestinal cells, e.g. from an intestinal tissue sample, an intestinal cell culture or from an intestinal organoid, e.g. as described herein. Methods for identifying or isolating cells using markers are well known in the art. The skilled person will understand which techniques to use according to the nature of the type of marker, for example, cell surface protein (e.g. cell surface receptor), an intracellular protein or hormone using the teaching available in the art combined with the teaching described herein. For example, it is straightforward to isolate cells using cell surface proteins, e.g. cell surface receptors. In embodiments in which the marker target is an intracellular protein, for example, an intracellular transcription factor, enzyme, or hormone, these may be used as markers by generating reporter constructs in which the intracellular marker is tagged at its endogenous locus with a detectable marker, for example, as described herein in a method of making a reporter organoid. The skilled person would be able to do this based on the teaching provided in the present application. Thus, in some embodiments, the marker is an EEC-specific gene as described herein.

The invention further provides the use of one or more targets as described herein as one or more drug targets. For example, the invention provides the use of a target as described herein as a drug target. For example, as described above, the invention provides a method for identifying/validating a drug target of interest in EECs, wherein the method comprises (i) contacting one or more intestinal organoids of the invention with a compound specific for the target; and (ii) determining whether secretion and/or expression and/or production of one or more hormones, hormone precursors and/or hormone synthesizing enzymes is affected.

The invention further provides a method for identifying/validating a drug target of interest in EECs, wherein the method comprises (i) contacting one or more intestinal organoids of the invention with a compound specific for the target; and (ii) determining whether the compound has an effect on the organoid. In some embodiments, if the effect on the organoid is an effect that would be beneficial in treating or preventing a disease or disorder, the drug target is identified or validated as a drug target for that disease or disorder.

Also provided is a method for identifying/screening/validating a compound for targeting a target, e.g. a target as described herein on EECs, wherein the method comprises (i) contacting an organoid of the invention with the compound; ii) determining whether secretion and/or expression and/or production of one or more hormones, hormone precursors and/or hormone synthesizing enzymes is affected. In some embodiments, a reporter organoid is used which comprises a tagged hormone, tagged hormone synthesizing enzyme and/or tagged hormone precursor whose expression and/or secretion is known to be affected (e.g. increased or decreased) by the target. In some embodiments, the target is a cell surface receptor, for example, a GPCR.

The EECs described in the various methods of the invention are preferably human EECs.

For example, also provided is a method for identifying/screening/validating a compound for suitability for treating or preventing a disease or disorder, wherein the method comprises (i) contacting an intestinal organoid of the invention with the compound; and (ii) determining whether the compound modulates expression or secretion of, agonises or antagonises in EECs a target, e.g. a target as described herein, implicated in the disease or disorder. In some embodiments, the organoid is a reporter organoid.

Also provided is a method for identifying/screening/validating a compound for suitability for treating or preventing a disease or disorder, wherein the method comprises (i) contacting an intestinal organoid of the invention with the compound; and (ii) determining whether secretion and/or expression and/or production of one or more hormones, hormone precursors and/or hormone synthesizing enzymes is affected. In some embodiments, hormone secretion and/or expression and/or production is affected through action of the compound on a target, e.g. a target as described herein. In some embodiments, the target is the tagged hormone, tagged hormone synthesizing enzyme or tagged hormone precursor in the reporter organoid. In some embodiments in which the target is a cell surface receptor (e.g. a GPCR), the compound is a known ligand of the receptor, for example, it is a compound known to target the receptor.

Also provided is a compound identified and/or validated by a method as described herein for use in treating or preventing the disease or disorder, for example, wherein the treating or preventing comprises targeting the compound to the gut.

The invention further provides a method for treating or preventing a disease or disorder comprising targeting a target which is found to affect secretion and/or expression and/or production of one or more hormones, hormone precursors and/or hormone synthesizing enzymes, wherein the one or more hormones, hormone precursors and/or hormone synthesizing enzymes is implicated in the disease or disorder. For example, the invention provides a method for treating or preventing a disease or disorder comprising administering a compound that targets a target in a human EEC, wherein the target is found to affect secretion and/or expression and/or production of one or more hormones, hormone precursors and/or hormone synthesizing enzymes, wherein the one or more hormones, hormone precursors and/or hormone synthesizing enzymes is implicated in the disease or disorder.

The invention further provides a method for treating or preventing a disease or disorder comprising targeting a target in a human EEC, wherein the disease or disorder is a disease or disorder in which one or more hormones expressed and/or secreted and/or produced by or produced from a hormone precursor secreted by the human EEC in which the target is expressed is implicated. The treating or preventing preferably comprises increasing, inducing, decreasing or ceasing expression and/or secretion and/or production of one or more hormones, hormone precursors and/or hormone synthesizing enzymes expressed and/or secreted and/or produced by, or produced from a hormone precursor secreted by, the human EEC in which the target is expressed.

The invention further provides a method for treating or preventing a disease or disorder comprising administering a compound found to affect secretion and/or expression and/or production of one or more hormones, hormone precursors and/or hormone synthesizing enzymes when used to target an EEC (e.g. when used to target an organoid of the invention), wherein the one or more hormones, hormone precursors and/or hormone synthesizing enzymes is implicated in the disease or disorder. For example, the invention provides a method for treating or preventing a disease or disorder comprising administering a compound which affects secretion and/or expression and/or production of one or more hormones, hormone precursors and/or hormone synthesizing enzymes by an EEC, wherein the one or more hormones, hormone precursors and/or hormone synthesizing enzymes is implicated in the disease or disorder. The EEC is preferably a human EEC. In some embodiments, the compound is found to affect secretion and/or expression and/or production of one or more hormones, hormone precursors and/or hormone synthesizing enzymes when used to target an EEC using a method of the invention.

Thus, the invention provides a method for treating or preventing a disease or disorder comprising administering a compound which affects secretion and/or production of one or more hormones and/or hormone precursors and/or expression of one or more hormones, hormone precursors and/or hormone synthesizing enzymes when used to target a target in a human EEC, wherein the one or more hormones, hormone precursors and/or hormone synthesizing enzymes is implicated in the disease or disorder.

Likewise, the invention provides a method for treating or preventing a disease or disorder comprising administering a compound that targets a target in a human EEC and which modulates expression and/or secretion and/or production of one or more hormones, hormone precursors and/or hormone synthesizing enzymes when used to target the target in a human EEC, wherein the disease or disorder is treatable or preventable by modulating the expression and/or secretion and/or production of the one or more hormones, hormone precursors and/or hormone synthesizing enzymes. Likewise, the invention provides a compound that targets a target in a human EEC and which modulates expression and/or secretion and/or production of one or more hormones, hormone precursors and/or hormone synthesizing enzymes when used to target the target in a human EEC for use in treating a disease or disorder, wherein the disease or disorder is treatable or preventable by modulating the expression and/or secretion and/or production of the one or more hormones, hormone precursors and/or hormone synthesizing enzymes.

For example, the invention provides a method for treating or preventing a disease or disorder comprising administering a compound that targets a target in a human EEC and which increases or induces expression and/or secretion and/or production of one or more hormones, hormone precursors and/or hormone synthesizing enzymes when used to target the target in a human EEC, wherein the disease or disorder is treatable or preventable by increasing or inducing expression and/or secretion and/or production of the one or more hormones, hormone precursors and/or hormone synthesizing enzymes. Thus, the invention provides a compound that targets a target in a human EEC and which increases or induces expression and/or secretion and/or production of one or more hormones, hormone precursors and/or hormone synthesizing enzymes when used to target the target in a human EEC, wherein the disease or disorder is treatable or preventable by increasing or inducing expression and/or secretion and/or production of the one or more hormones, hormone precursors and/or hormone synthesizing enzymes.

For example, the invention provides a method for treating or preventing a disease or disorder comprising administering a compound that targets a target in a human EEC and which decreases or ceases expression and/or secretion and/or production of one or more hormones, hormone precursors and/or hormone synthesizing enzymes when used to target the target in a human EEC, wherein the disease or disorder is treatable or preventable by decreasing or ceasing expression and/or secretion and/or production of the one or more hormones, hormone precursors and/or hormone synthesizing enzymes. Thus, the invention provides a compound that targets a target in a human EEC and which decreases or ceases expression and/or secretion and/or production of one or more hormones, hormone precursors and/or hormone synthesizing enzymes when used to target the target in a human EEC, wherein the disease or disorder is treatable or preventable by decreasing or ceasing expression and/or secretion and/or production of the one or more hormones, hormone precursors and/or hormone synthesizing enzymes.

The skilled person will understand whether an induction or increase in secretion and/or expression and/or production or a ceasing or decrease in secretion and/or expression and/or production of one or more particular hormones, hormone precursors and/or hormone synthesizing enzymes is relevant depending on the disease or disorder of interest.

In some embodiments, an induction or increase in secretion and/or expression and/or production of one or more particular hormones is relevant. In some embodiments, a cease or decrease in secretion and/or expression and/or production of one or more particular hormones is relevant. In some embodiments, an induction or increase in secretion and/or expression of one or more particular hormone precursors is relevant. In some embodiments, a cease or decrease in secretion and/or expression of one or more particular hormone precursors is relevant. In some embodiments, an induction or increase in expression of one or more particular hormone synthesizing enzymes is relevant. In some embodiments, a cease or decrease in expression of one or more particular hormone synthesizing enzymes is relevant.

The invention further provides a method for treating or preventing a disease or disorder comprising administering a compound found to affect secretion and/or expression and/or production of one or more hormones, hormone precursors and/or hormone synthesizing enzymes when used to target a target, e.g. a target as described herein, wherein the one or more hormones, hormone precursors and/or hormone synthesizing enzymes is implicated in the disease or disorder. In some embodiments, the compound is found to affect secretion and/or expression and/or production in a method for identifying/validating a target (e.g. a drug target) in EECs, as described herein. In some embodiments, the compound is found to affect secretion and/or expression and/or production in a method for identifying/screening/validating a compound for suitability for treating or preventing a disease or disorder, as described herein. In some embodiments, the compound is found to affect secretion. In some embodiments, a hormone produced from a hormone precursor is implicated in the disease or disorder. In some embodiments, production of the hormone produced from the hormone precursor is assessed.

The invention further provides a method for treating or preventing a disease or disorder comprising targeting a target, e.g. a target as described herein, in one or more human EECs.

Accordingly, the invention provides a method for treating or preventing a disease or disorder comprising administering a compound that targets a target, e.g. a target described herein, in one or more human EECs. Similarly, the invention provides a compound that targets a target, e.g. a target as described herein, in one or more human EECs for use in a method for treating or preventing a disease or disorder.

Similarly, the invention provides a method for treating or preventing a disease or disorder comprising administering a compound that targets a target in a human EEC and modulates expression and/or secretion and/or production of one or more hormones, hormone precursors and/or hormone synthesizing enzymes when used to target the target in a human EEC. Likewise, the invention provides a compound that targets a target in a human EEC and which modulates expression and/or secretion and/or production of one or more hormones, hormone precursors and/or hormone synthesizing enzymes when used to target the target in a human EEC, for use in treating a disease or disorder.

The invention provides a method for treating or preventing a disease or disorder comprising administering a compound that targets a target as described herein, wherein the method comprises targeting the compound to the gut. Similarly, the invention provides a compound that targets a target as described herein for use in a method for treating or preventing a disease or disorder, wherein the treating or preventing comprises targeting the compound to the gut.

In some embodiments, the treating or preventing comprises modulating expression and/or secretion and/or production of one or more hormones. In some embodiments, the treating or preventing comprises increasing or inducing expression and/or secretion and/or production of one or more hormones. In some embodiments, the treating or preventing comprises decreasing or ceasing expression and/or secretion and/or production of one or more hormones. In some embodiments, the treating or preventing comprises increasing or inducing expression and/or secretion and/or production of one or more hormones and decreasing or ceasing expression and/or secretion and/or production of one or more other hormones. In some embodiments, the treating or preventing comprises modulating expression and/or secretion of one or more hormone precursors. In some embodiments, the treating or preventing comprises increasing or inducing expression and/or secretion of one or more hormone precursors. In some embodiments, the treating or preventing comprises decreasing or ceasing expression and/or secretion of one or more hormone precursors. In some embodiments, the treating or preventing comprising modulating expression of one or more hormone synthesizing enzymes. In some embodiments, the treating or preventing comprises increasing or inducing expression of one or more hormone synthesizing enzymes. In some embodiments, the treating or preventing comprises decreasing or ceasing expression of one or more hormone synthesizing enzymes.

In embodiments which describe the suitability of a compound for treating a disease or disorder due to its ability to modulate expression of one or more hormones, hormone precursors and/or hormone synthesizing enzymes, it will be understood that expression at the protein level is relevant.

The invention further provides a method for treating or preventing a disease or disorder comprising administering a compound that targets a target, e.g. a target as described herein, in one or more human EECs, wherein the target is implicated in the disease or disorder. For example, in some embodiments, the target may be a marker for cells having the disease or disorder or at risk of having the disease or disorder. In some embodiments, the cell marker may be used to target delivery of therapies to said cells. In some embodiments, the therapy involves delivering a drug which treats or prevents the disease or disorder in the cell. In some embodiments, the therapy involves delivering a drug which kills the cell. In some embodiments, the target may be implicated in the disease or disorder. For example, in some embodiments, the target is implicated in the pathogenesis of the disease or disorder. In some embodiments, the target affects secretion and/or expression and/or production of a hormone, hormone precursor or hormone synthesizing enzyme implicated in the disease or disorder. In some embodiments, the treatment or prevention comprises modulating expression or secretion of, agonising or antagonising the target, e.g. in human EECs.

The invention further provides a method for treating or preventing a disease or disorder comprising targeting EECs or one or more EEC subtypes found to express a first target, e.g. a target as described herein, that is implicated in the disease or disorder. In some embodiment, the targeting comprises administering a compound that targets the EECs or the one or more EEC subtypes. For example, in some embodiments, the compound kills the EECs or the one or more EEC subtypes that express that first target. In some embodiments, the compound targets the first target. In some embodiments, the compound does not target the first target but instead targets a different target on the EEC or the one or more EEC subtypes. In some embodiments, the targeting comprises administering radiotherapy directed to the EECs or the one or more EEC subtypes.

In some embodiments, the treating or preventing comprises increasing expression and/or secretion of or agonising a target, e.g. a target as described herein. In some embodiments, the increasing expression comprises administering the target to the patient. In some embodiments, the treating or preventing comprises contacting human EECs or one or more human EEC subtypes with the target, for example, in the form of a DNA expression construct comprising a gene encoding the target. In some embodiments, the treating or preventing comprises increasing the expression of the target in EECs or in one or more EEC subtypes in the patient, for example, increasing expression of the endogenous gene encoding the target. The target or a compound which increases expression of the target may be administered to the patient by any suitable method. Such methods are well known in the art. Thus, in some embodiments, targeting a target is increasing expression or secretion of a target.

In some embodiments, the treating or preventing comprises decreasing expression and/or secretion of or antagonising a target, e.g. a target as described herein. For example, the decreasing expression may comprise administering an antagonist of the target to the patient. In some embodiments, the treating or preventing comprises contacting human EECs or one or more human EEC subtypes with the antagonist of the target. In some embodiments, the treating or preventing comprises decreasing the expression of the target in EECs or in one or more EEC subtypes in the patient. Examples of antagonists of targets are well known in the art. In some embodiments, the treating or preventing prevents the target from being expressed. For example, in some embodiments, the treating or preventing comprises administering a construct that knocks out the target or decreases expression of the endogenous gene encoding the target. In some embodiments, the treating or preventing renders existing target DNA, RNA and/or protein non-functional. In some embodiments, the treating or preventing destroys the existing target DNA, RNA and/or protein. Thus, in some embodiments, targeting a target is decreasing expression or secretion of a target.

In some embodiments, the target is a cell surface receptor. For example, the invention similarly provides a method for treating or preventing a disease or disorder comprising administering a compound which targets a cell surface receptor on an EEC, wherein the EEC expresses and/or secretes and/or produces one or more (e.g. two or more) hormones, hormone precursors and/or hormone synthesizing enzymes implicated in the disease or disorder. Similarly, there is provided a compound for use in treating or preventing a disease or disorder, for example, wherein the treating or preventing comprises targeting a cell surface receptor on an EEC in order to modulate expression and/or secretion and/or production of one or more (e.g. two or more) hormones, hormone precursors and/or hormone synthesizing enzymes implicated in the treatment or prevention of the disease or disorder. In some embodiments, the expression and/or secretion and/or production of two or more hormones, hormone precursors and/or hormone synthesizing enzymes is modulated. This may advantageously provide a synergistic response.

In some embodiments, the target is a cell surface receptor. For example, the invention similarly provides a method for treating or preventing a disease or disorder comprising administering a compound which targets a cell surface receptor on an EEC, wherein the EEC expresses and/or secretes and/or produces one or more (e.g. two or more) hormones and/or hormone precursors implicated in the disease or disorder. Similarly, there is provided a compound for use in treating or preventing a disease or disorder, for example, wherein the treating or preventing comprises targeting a cell surface receptor on an EEC in order to modulate expression and/or secretion and/or production of one or more (e.g. two or more) hormones and/or hormone precursors implicated in the treatment or prevention of the disease or disorder. In some embodiments, the expression and/or secretion and/or production of two or more hormones and/or hormone precursors is modulated. This may advantageously provide a synergistic response.

The disease or disorder is preferably a disease or disorder as described herein. Where the invention provides a method of treating or preventing, the invention similarly provides a compound for use in the method of treating or preventing. The invention further provides a compound as described herein for use in therapy.

In some embodiments, the compound induces or increases expression and/or secretion and/or production of a hormone. For example, in some embodiments, the compound induces or increases expression of a hormone. For example, in some embodiments, the compound induces or increases secretion of a hormone. For example, in some embodiments, the compound induces or increases production of a hormone. In some embodiments, the compound decreases or ceases expression and/or secretion and/or production of a hormone. For example, in some embodiments, the compound decreases or ceases expression of a hormone. For example, in some embodiments, the compound decreases or ceases secretion of a hormone. For example, in some embodiments, the compound decreases or ceases production of a hormone. In some embodiments, the hormone is a hormone as described herein. In some embodiments, the hormone is Ghrelin. In some embodiments, the hormone is Cerebellin 1. In some embodiments, the hormone is CCK. In some embodiments, the hormone is GLP-1. In some embodiments, the hormone is PPY. In some embodiments, the hormone is PYY. In some embodiments, the hormone is Motilin. In some embodiments, the hormone is Angiotensin. In some embodiments, the hormone is Serotonin. In some embodiments, the hormone is GIP. In some embodiments, the hormone is Somatostatin. In some embodiments, the hormone is Gastrin. In some embodiments, the hormone is Secretin. In some embodiments, the hormone is Serotonin.

In some embodiments, the compound induces or increases expression and/or secretion of a hormone precursor. For example, in some embodiments, the compound induces or increases expression of a hormone precursor. For example, in some embodiments, the compound induces or increases secretion of a hormone precursor. In some embodiments, the compound decreases or ceases expression and/or secretion of a hormone precursor. For example, in some embodiments, the compound decreases or ceases expression of a hormone precursor. For example, in some embodiments, the compound decreases or ceases secretion of a hormone precursor. In some embodiments, the hormone precursor is GCG.

In some embodiments, the compound induces or increases expression of a hormone synthesizing enzyme. In some embodiments, the compound decreases or ceases expression of a hormone synthesizing enzyme. In some embodiments, the hormone synthesizing enzyme is TPH1.

It will be understood that in embodiments which describe the compound modulating expression and/or secretion and/or production of a hormone, hormone precursor and/or hormone synthesizing enzyme, the compound modulates expression and/or secretion and/or production of the hormone, hormone precursor and/or hormone synthesizing enzyme by EECs, e.g. human EECs, and/or modulates production of a hormone from a hormone precursor secreted by EECs, e.g. by human EECs. Thus, in preferred embodiments, this is achieved by targeting a target as described herein in human EECs. Thus, in preferred embodiments, the compound modulates expression and/or secretion and/or production of a hormone, hormone precursor and/or hormone synthesizing enzyme when used to target a target in a human EEC. Similarly, in preferred embodiments, the compound modulates expression and/or secretion and/or production of a hormone, hormone precursor and/or hormone synthesizing enzyme when used to target a target in human EECs. The target is preferably a target as described herein.

In some embodiments, a compound which targets a target, e.g. a cell surface receptor, is a compound which binds the target. For example, in some embodiments, the compound binds the target and agonises the target, e.g. is a cell surface receptor agonist. For example, in some embodiments, the compound binds the target and antagonises the target, e.g. it is a cell surface receptor antagonist. The compound targets the target in an EEC, preferably a human EEC. In some embodiments, the compound also targets the target in a non-EEC, however, the target in the EEC is also targeted. In some embodiments, the compound targets the target only in EECs and not in non-EECs.

Preferably, the compound does not substantially target any other targets described herein in the same EEC. In some embodiments, the compound does not substantially target any other targets described herein. In some embodiments, the compound does not substantially target any other targets. Preferably, the compound does not substantially bind to any other targets described herein in the same EEC. In some embodiments, the compound does not substantially bind to any other targets described herein. In some embodiments, the compound does not substantially bind to any other targets. In embodiments in which the target is a cell surface receptor, preferably the compound does not substantially bind to any other cell surface receptor targets described herein on the same EEC. In some embodiments, the compound does not substantially bind to any other cell surface receptor targets described herein. In some embodiments, the compound does not substantially bind to any other cell surface receptors.

In some embodiments, targeting a transcription factor comprises overexpressing the transcription factor. This may be achieved by any suitable method, for example, using a construct comprising a gene encoding the transcription factor. The gene may be under control of a constitutive or an inducible promoter. Alternatively, the endogenous gene encoding the transcription factor can be targeted with a construct comprising a constitutive or inducible promoter. In some embodiments, targeting a transcription factor comprises knocking out or decreasing expression of the transcription factor. For example, the endogenous gene encoding the transcription factor may be knocked out or inhibited by any suitable method. In some embodiments, the gene is knocked out using CRISPR/Cas9 technology. The skilled person will be able to design suitable gRNAs and optionally primers to target transcription factors described herein as their sequences are known. In some embodiments, the transcription factor is targeted with an inhibitor. For example, in some embodiments, the compound decreases or ceases expression of the transcription factor. For example, in some embodiments, the compound inhibits expression of the transcription factor. In some embodiments, the transcription factor is targeted with an activator. For example, in some embodiments, the compound increases or induces expression of the transcription factor. Such constructs, inhibitors and activators may be used as a compound in the various aspects of the invention, for example, the in vitro embodiments described herein. For example, as described elsewhere herein, the compound used to target the transcription factor may be identified using a method of the invention, e.g. in which the transcription factor is tagged with a detectable marker in a reporter organoid of the invention. In some embodiments, the method may further comprise detecting whether the expression and/or secretion and/or production of one or more hormones, hormone precursors and/or hormone synthesizing enzymes is affected by modulating expression of the transcription factor. In some embodiments, the reporter organoid comprises the tagged transcription factor and one or more tagged hormones, hormone synthesizing enzymes and/or hormone precursors. This method may be adapted to identify compounds for targeting any target of the invention, e.g. by using a reporter organoid of the invention in which the target is tagged with a detectable marker.

The patient affected by the disease or disorder is preferably a human patient. Accordingly, the methods of treating or preventing described herein are preferably carried out in a human patient. In some embodiments, the patient to be treated is a patient identified as having a disease or disorder of one or more of their EECs, for example of one or more EEC subtypes. In some embodiments, the patient to be treated is a patient identified as having a target, e.g. a target as described herein, in their EECs, for example in one or more EEC subtypes. For example, in some embodiments, the target may be a mutated RNA or protein, for example, as identified by the invention. In some embodiments, the patient to be treated is a patient identified as having abnormal hormone secretion and/or expression and/or production by one or more of their EECs, for example by one or more EEC subtypes. In some embodiments, the patient to be treated is a patient identified as having abnormal hormone production from a hormone precursor secreted by one or more EEC subtypes. For example, in some embodiments, the patient may exhibit an increase in secretion and/or expression and/or production of one or more (e.g. 1, 2, 3, 4, 5 or more) hormones and/or hormone precursors from their EECs, for example, from one or more (e.g. 1, 2, 3, 4, 5 or more) EEC subtypes. The increase may be compared to a healthy individual who does not have the disease or disorder or for example, compared to a population of comparable healthy individuals who do not have the disease or disorder. For example, in some embodiments, the patient may exhibit a decrease in secretion and/or expression and/or production of one or more (e.g. 1, 2, 3, 4, 5 or more) hormones and/or hormone precursors from their EECs, for example, from one or more (e.g. 1, 2, 3, 4, 5 or more) EEC subtypes. The decrease may be compared to a healthy individual who does not have the disease or disorder or for example, compared to a population of comparable healthy individuals who do not have the disease or disorder. In some embodiments, the patient is identified as a patient who would benefit from modulation of secretion and/or expression and/or production of one or more (e.g. 1, 2, 3, 4, 5 or more) hormones and/or hormone precursors from their EECs, for example, from one or more (e.g. 1, 2, 3, 4, 5 or more) EEC subtypes, in order to treat the disease or disorder. For example, in some embodiments, the beneficial modulation is an increase of secretion and/or expression and/or production of one or more hormones and/or hormone precursors. In some embodiments, the beneficial modulation is a decrease of secretion and/or expression and/or production of one or more hormones and/or hormone precursors. In some embodiments, the beneficial modulation is an increase of secretion and/or expression and/or production of one or more hormones and/or hormone precursors and a decrease in secretion and/or expression and/or production of one or more other hormones and/or hormone precursors. In some embodiments, expression and/or secretion is relevant. In some embodiments, secretion is relevant and expression is not relevant. The methods may be adapted accordingly.

In some embodiments, the patient is a patient who has been identified as a patient who will respond to the compound for treating or preventing the disease or disorder using a method of the invention. For example, in some embodiments, the patient is a patient identified using a method for determining whether a patient will respond to a compound for treating or preventing a disease or disorder, wherein the method comprises (i) establishing an organoid of the invention from said patient; (ii) contacting the organoid with the compound; and (iii) determining whether secretion and/or expression and/or production of one or more hormones, hormone precursors and/or hormone synthesizing enzymes is affected.

In some embodiments, the invention provides the compound for use in treating or preventing the disease or disorder. In some embodiments, the treating or preventing comprises targeting one or more targets as described herein in human EECs, e.g. in one or more EEC subtypes, with the compound.

In some embodiments, the treating or preventing a disease or disorder comprises administering a therapeutic compound to a patient, wherein the therapeutic compound is targeted to the gut. In some embodiments, the treating or preventing a disease or disorder comprises administering a therapeutic compound to a patient, wherein the therapeutic compound is targeted to human EECs in the gut or to one or more human EEC subtypes in the gut. In some embodiments, the therapeutic compound is targeted specifically to the gut or is targeted specifically to human EECs or specifically to one or more human EEC subtypes in the gut. For example, in some embodiments, the treating or preventing comprises targeting a target in human EECs or in one or more human EEC subtypes.

Accordingly, in some embodiments, the treating or preventing comprises targeting the compound to the gut. For example, in some embodiments, the treating or preventing comprises oral administration of the compound.

The inventors' findings that a particular target is expressed in human EECs or in a human EEC subtype opens up the possibility of targeting therapeutic compounds which affect the target to the gut. For example, the therapeutic compound may target the identified target (e.g. as an agonist or antagonist) or it may affect the expression of the target (e.g. it may increase or decrease expression). Similarly, it opens up the possibility of targeting therapeutic compounds to the gut which are already known to treat a disease or disorder in which the target is implicated in the pathogenesis of the disease or disorder. For example, in some embodiments, the disease or disorder is a disease or disorder in which the target is implicated. Examples of such diseases and disorders are described herein. Similarly, in some embodiments, the disease or disorder is a disease or disorder in which a hormone that is affected by targeting the target is implicated. Examples of such diseases and disorders are likewise described herein.

Accordingly, in some embodiments, the therapeutic compound is targeted to the gut. Targeting to the gut may be achieved by any suitable method. Examples of such methods are well known in the art, for example, oral administration. In some embodiments, the therapeutic compound is targeted to human EECs in the gut or to one or more particular human EEC subtypes in the gut. Targeting to human EECs or to one or more particular human EEC subtypes may likewise be achieved by any suitable method. For example, antibody-mediated targeting may be used, wherein the therapeutic compound is linked to an antibody that is specific for a marker specific for human EECs or for the one or more human EEC subtypes of interest. Thus, in some embodiments, the therapeutic compound is targeted specifically to the gut or is targeted specifically to human EECs in the gut or specifically to one or more particular human EEC subtypes in the gut.

As discussed above, the inventors' findings that a particular target is expressed in human EECs or in a human EEC subtype opens up the possibility of targeting therapeutic compounds which are associated with the particular target to the gut (e.g. compounds with target the target, for example, as described herein). Such methods are provided herein. The intestinal organoids of the invention are an in vitro model of the gut, and so provide an in vitro system to study the newly identified targets in vitro and, if desired, to identify new compounds which target these.

In embodiments which describe the therapeutic use of a compound, the compound is preferably formulated to be acceptable for administration to a patient, e.g. to a human patient. For example, the compound is preferably formulated as a pharmaceutical composition. Accordingly, also provided is a pharmaceutical composition comprising a therapeutic compound as described herein. The invention also provides a pharmaceutical composition comprising a compound as described herein. In some embodiments, the pharmaceutical composition is formulated to target the gut. For example, in some embodiments, the compound or pharmaceutical composition is formulated so it is suitable for administration to or delivery to the gut. In some embodiments, the compound or pharmaceutical composition is formulated so it is suitable for administration to the gut. In some embodiments, the compound or pharmaceutical composition is formulated so it is suitable for delivery to the gut. In some embodiments, the compound or pharmaceutical composition is formulated so it is suitable for administration or delivery to the small intestine. In some embodiments, the compound or pharmaceutical composition is formulated so it is suitable for administration or delivery to the colon. In some embodiments, the compound or pharmaceutical composition is formulated to target human EECs or one or more human EEC subtypes in the gut. For example, in some embodiments, the compound or pharmaceutical composition is formulated so it is suitable for enteral administration. For example, in some embodiments, the compound or pharmaceutical composition is formulated so it is suitable for oral, sublingual or rectal administration. In some embodiments, the compound or pharmaceutical composition is formulated so it is suitable for oral administration. In some embodiments, the compound or pharmaceutical composition is formulated so it is suitable for sublingual administration. In some embodiments, the compound or pharmaceutical composition is formulated so it is suitable for rectal administration. In some embodiments, the pharmaceutical composition is formulated so that it is absorbed by or preferentially retained in the gut. For example, in some embodiments, the pharmaceutical composition is formulated so that it is absorbed by the gut. For example, in some embodiments, the pharmaceutical composition is formulated so that it is preferentially retained by the gut. In some embodiments, the pharmaceutical composition is formulated so it is suitable for oral administration and is absorbed by or preferentially retained in the gut. The pharmaceutical composition may take any suitable form. For example, in some embodiments, the pharmaceutical composition is a solid form, for example a tablet. In some embodiments, the pharmaceutical composition is a liquid. In some embodiments, the pharmaceutical composition is formulated as a capsule. In some embodiments, the pharmaceutical composition is a suppository, in particular a rectal suppository.

The various targets described herein on EECs are, generally, proteins that are already known because they exist elsewhere in the body. Therefore, in some embodiments, it is advantageous to target the compound so that it acts only on the target in EECs rather than acting on the target elsewhere in the body. However, some EEC subtype specific receptors are in the intestine only. In some embodiments, the pharmaceutical composition is formulated to target specifically the gut or to target specifically human EECs in the gut or specifically one or more particular human EEC subtypes in the gut. For example, in some embodiments, the pharmaceutical composition is formulated to target specifically the gut. In some embodiments, the pharmaceutical composition is formulated so that the compound can act in the gut but cannot act elsewhere in the body. For example, in some embodiments, the pharmaceutical composition is formulated so that the compound is released in the gut but is not released elsewhere in the body. Various methods for effecting gut specific targeting are known in the art. Oral administration is an efficient method of gut specific targeting. In some embodiments, a compound for use in the invention is modified using known techniques (e.g. see Gavhane, Y. N. and Yadav, A. V. (2012); Charmot, D. (2012); Amidon, S. et al., (2015); Cao et al. (2017)) to achieve intestinal targeting of the compound. In some embodiments, a compound for use in the invention is modified using known techniques (e.g. see Gavhane, Y. N. and Yadav, A. V. (2012); Charmot, D. (2012); Amidon, S. et al., (2015); Cao et al. (2017)) so that it is efficiently degradable by the liver so that it has no bloodstream circulation or very little bloodstream circulation. In some embodiments, such modified compounds are administered orally, for example, as a tablet, capsule or a liquid, so that they are absorbed by or preferentially retained in the gut. For example, in some embodiments, the compound is administered as a tablet. For example, in some embodiments, the compound is administered as a capsule. For example, in some embodiments, the compound is administered as a liquid. Thus, in some embodiments, the pharmaceutical composition is formulated so that the compound can act in the gut but has little effect (e.g. de minimis effect) elsewhere in the body. In some embodiments, the therapeutic effect in a method of treating or preventing provided herein arises from targeting the target in one or more EECs. For example, in some embodiments, the therapeutic effect in a method of treating or preventing provided herein does not arise from targeting the target in a location other than the gut, for example, in a location other than an EEC. In some embodiments, the therapeutic effect in a method of treating or preventing provided herein arises from targeting the target in one or more EECs in combination with targeting the target in a location other than the gut, for example, in a location other than an EEC. As a further example, many of the cell surface receptors on EECs are located on the lumen side of the intestine because their purpose is to sense the content of the intestine. These cell surface receptors do not, therefore, face the bloodstream. Therefore, if a compound is retained in the intestine and is not exposed to the bloodstream, it can still have its effects.

Thus, in some embodiments, the treating or preventing comprises targeting the compound specifically to the gut, or targeting specifically human EECs in the gut or specifically one or more particular human EEC subtypes in the gut, e.g. as described herein. For example, in some embodiments, the treating or preventing comprises targeting the compound specifically to the gut.

A gut-brain axis exists in mammals, e.g. in humans. A number of studies have found evidence of signalling from the gut to the brain (through vagal neurons) (Bellono et al. 2017; Kaelberer et al 2018; Raybould et al, 2007). Gut peptides have been found to be produced in the brain (Gardiner et al. 2010), which suggests that hormone-producing cells in the brain are similar to EECs in terms of the hormone biology, for example, that they have the same or similar receptors to EECs. In general, for hormones which have a known function in the brain, modulating hormone production by the gut is a better option than administering hormones to the brain, because it is easier, better for the patient and is potentially less toxic. Thus, the present invention opens up the possibility of treating or preventing diseases or disorders in which the brain is implicated by targeting a target on a human EEC, e.g. to modulate the expression and/or secretion (or production) of one or more hormones, hormone precursors and/or hormone synthesizing enzymes.

As the various targets described herein are generally proteins, e.g. cell surface receptors, that are known to exist elsewhere in the body, the function of many of these targets is already known. For example, for olfactory receptors, it is already known in part what these do. In particular, it is known that they are responsible for oderant detection and that their downstream signalling involves a calcium influx. Thus, it is likely that targeting an olfactory receptor in the intestine will result in hormone release. The invention therefore opens up the possibility of targeting these targets in human EECs to obtain various effects, many of which are already known in the art or are plausible from what is already known. The present invention makes it possible to achieve these effects in a new and inventive way.

In some embodiments, the pharmaceutical composition is suitable for treating or preventing a disease or disorder selected from a disease or disorder as described herein. In some embodiments, the therapeutic compound is a drug already known to be effective in targeting the disease or disorder of interest. In some embodiments, the therapeutic compound is a compound that has been identified, screened or validated by a method as described herein.

In some embodiments, a pharmaceutical composition as described herein comprises one or more components in addition to the one or more compounds, e.g. they typically include one or more pharmaceutically acceptable carrier(s) and/or excipient(s).

The invention provides a method for modulating expression and/or secretion and/or production of one or more hormones, hormone precursors and/or hormone synthesizing enzymes comprising targeting a target in the EEC with a compound. In some embodiments, the target is a cell surface receptor. In some embodiments, the target is a transcription factor. For example, the invention provides a method for modulating expression and/or secretion and/or production of one or more hormones, hormone precursors and/or hormone synthesizing enzymes comprising targeting a cell surface receptor on human EECs. For example, the invention provides a method for modulating expression and/or secretion and/or production of one or more hormones, hormone precursors and/or hormone synthesizing enzymes expressed by an EEC comprising targeting a cell surface receptor on the EEC with a compound. For example, the invention provides a method for modulating expression and/or secretion and/or production of one or more (e.g. 2, 3, 4 or more) hormones, hormone precursors and/or hormone synthesizing enzymes comprising contacting a cell surface receptor on EECs or one or more EEC subtypes with a compound. Similarly, the invention provides a method for modulating expression and/or secretion and/or production of one or more hormones, hormone precursors and/or hormone synthesizing enzymes expressed by an EEC comprising contacting the EEC with a compound. Also provided is a compound for use in modulating expression and/or secretion and/or production of one or more hormones, hormone precursors and/or hormone synthesizing enzymes by targeting a target in the EEC. In some embodiments, the target is a cell surface receptor. Also provided is a compound for use in modulating expression and/or secretion and/or production of one or more hormones, hormone precursors and/or hormone synthesizing enzymes expressed by an EEC, for example, by targeting a cell surface receptor on the EEC.

In some embodiments, the modulating is increasing or inducing the expression and/or secretion and/or production of the one or more hormones, hormone precursors and/or hormone synthesizing enzymes. In some embodiments, the modulating is reducing or ceasing the expression and/or secretion and/or production of the one or more hormones, hormone precursors and/or hormone synthesizing enzymes. For example, in some embodiments, the modulating is inhibiting expression and/or secretion and/or production of the one or more hormones, hormone precursors and/or hormone synthesizing enzymes. In some embodiments, the modulating is increasing or inducing the expression and/or secretion and/or production of one or more hormones, hormone precursors and/or hormone synthesizing enzymes and decreasing or ceasing the expression and/or secretion and/or production of the one or more hormones, hormone precursors and/or hormone synthesizing enzymes. For example, in some embodiments, the modulating is increasing or inducing the expression and/or secretion and/or production of one or more hormones, hormone precursors and/or hormone synthesizing enzymes and decreasing or ceasing the expression and/or secretion and/or production of one or more other hormones, hormone precursors and/or hormone synthesizing enzymes.

Any suitable compound may be used in the invention. Examples of compounds are provided elsewhere herein.

The invention provides a method for identifying a compound that targets a target of interest. In some embodiments, the target of interest is a cell surface receptor. For example, a compound or a library of compounds can be screened in a method of the invention. For example, expression and/or secretion and/or production of one or more hormones, hormone precursors and/or hormone synthesizing enzymes may then be assessed, e.g. using an organoid of the invention. If a change in expression and/or secretion and/or production is detected upon contacting with a compound compared to the expression and/or secretion and/or production in an organoid that has not been contacted with the compound, the target of interest (e.g. the receptor) can be knocked out and the method can be conducted again to see whether the change no longer occurs. If the change no longer occurs, it indicates that the knocked out target (e.g. the receptor) is targeted by the compound. For example, the invention provides a method for identifying a compound which targets a cell surface receptor of interest, wherein the method comprises (i) contacting one or more organoids of the invention with one or more compounds; (ii) determining whether secretion and/or expression and/or production of one or more hormones, hormone precursors and/or hormone synthesizing enzymes is affected; (iii) if an effect is determined, knocking out the cell surface receptor of interest in the organoid and repeating steps (i) and (ii) with the knock-out organoid, wherein a finding that the effect no longer occurs indicates that the compound targets the cell surface receptor. Accordingly, the various methods described herein may incorporate a further step of validating that the compound targets the target by knocking out the target and repeating the experiment, e.g. as described herein. If the effect is no longer obtained, this can be used to validate that the compound targets the target. As mentioned elsewhere herein, the organoids provided by the invention advantageously allow high throughput screening of compound libraries. For example, in some embodiments, an antibody library specific for the target of interest (e.g. the cell surface receptor) is generated and screened using a method of the invention in order to identify a compound that targets the target of interest. Advantageously, the organoids of the invention enable the desired effect of the compound, e.g. an increase or induction of expression and/or secretion and/or production of one or more particular hormones, hormone precursors and/or hormone synthesizing enzymes and/or a decrease or cease of expression and/or secretion and/or production of one or more particular hormones, hormone precursors and/or hormone synthesizing enzymes to be screened for, for example, according to the disease or disorder of interest. Other strategies for identifying ligands of a cell surface receptor include partly computational approaches to predict ligands, such as described in Foster et al. 2019. Such ligands may be used as a compound in the present invention. Ligands identified by such methods are encompassed within the scope of the invention and may be used as one or more compounds of and in the invention.

In some embodiments, the compound is a known ligand for the cell surface receptor target. For example, in some embodiments, a compound which targets SCTR is Secretin (SCT). In some embodiments, a compound which targets NPY1R is selected from Peptide YY (PYY) and Neuropeptide Y (NPY). For example, in some embodiments, it is PYY. In some embodiments, it is NPY. In some embodiments, a compound which targets MC1R is selected from a melanocortin, ACTH and MSH. In some embodiments, it is a melanocortin. In some embodiments it is ACTH. In some embodiments it is MSH. In some embodiments, a compound which targets GABBR2 is GABA. In some embodiments, a compound which targets IL-20RA is selected from IL-10, IL-19, IL-20, IL-24 and IL-26. In some embodiments, it is IL-10. In some embodiments, it is IL-19. In some embodiments, it is IL-20. In some embodiments, it is IL-24. In some embodiments, it is IL-26. In some embodiments, a compound which targets GRPR is Gastrin-releasing-peptide (GRP). In some embodiments, a compound which targets TSHR is TSH. In some embodiments, a compound which targets GPR68 is Cocaine-and-amphetamine-regulated transcript protein (CARPT). In some embodiments, a compound which targets GPR37 is head activator (HA). In some embodiments, a compound which targets DRD2 is Dopamine. In some embodiments, a compound which targets OR51E1 is nonionic acid. In some embodiments, a compound which targets OR51E2 is beta-ionone. In some embodiments, a compound which targets GRP68 is CART. Fragments of such compounds may alternatively be used.

In some embodiments, a compound which targets SCTR comprises a variant of the Secretin sequence shown in SEQ ID NO:70. In some embodiments, a compound which targets NPY1R comprises a variant of the Peptide YY (PYY) sequence shown in SEQ ID NO:71. In some embodiments, a compound which targets NPY1R comprises a variant of the Neuropeptide Y (NPY) sequence shown in SEQ ID NO:72. In some embodiments, a compound which targets MC1R comprises a variant of the ACTH sequence shown in SEQ ID NO:73. In some embodiments, a compound which targets MC1R comprises a variant of the alpha-MSH sequence shown in SEQ ID NO:74. In some embodiments, a compound which targets MC1R comprises a variant of the beta-MSH sequence shown in SEQ ID NO:75. In some embodiments, a compound which targets MC1R comprises a variant of the gamma-MSH sequence shown in SEQ ID NO:76. In some embodiments, a compound which targets IL-20RA comprises a variant of the IL-10 sequence shown in SEQ ID NO:77. In some embodiments, a compound which targets IL-20RA comprises a variant of the IL-19 sequence shown in SEQ ID NO:78. In some embodiments, a compound which targets IL-20RA comprises a variant of the IL-20 sequence shown in SEQ ID NO:79. In some embodiments, a compound which targets IL-20RA comprises a variant of the IL-24 sequence shown in SEQ ID NO:80. In some embodiments, a compound which targets IL-20RA comprises a variant of the IL-26 sequence shown in SEQ ID NO:81. In some embodiments, a compound which targets GRPR comprises a variant of the Gastrin-releasing-peptide (GRP) sequence shown in SEQ ID NO:82. In some embodiments, a compound which targets TSHR comprises a variant of the TSH alpha subunit shown in SEQ ID NO:83. In some embodiments, a compound which targets TSHR comprises a variant of the TSH beta subunit shown in SEQ ID NO:84. In some embodiments, a compound which targets GPR68 comprises a variant of the Cocaine-and-amphetamine-regulated transcript protein (CARPT) shown in SEQ ID NO:85. In some embodiments, a compound which targets GPR68 comprises a variant of the Cocaine-and-amphetamine-regulated transcript protein (CARPT) shown in SEQ ID NO:86. In some embodiments, a compound which targets GPR37 comprises a variant of the head activator (HA) sequence shown in SEQ ID NO:87. In some embodiments, the polypeptide sequence of the variant has at least 75% (e.g. at least 80%, 85%, 90%, 95%, 98%, 99%) sequence identity with the polypeptide sequence provided in the respective SEQ ID NO. Methods for determining sequence identity are described herein. A fragment of a variant may alternatively be used.

Targeting one or more cell surface receptors in order to affect the secretion and/or expression and/or production of one or more hormones, hormone precursors and/or hormone synthesizing enzymes may be advantageous over methods which target hormones directly, for example, which target the stability of an individual hormone, or which target the receptor of a specific hormone. For example, by targeting a cell surface receptor, a hormone profile may be modulated. For example, multiple hormones, hormone precursors and/or hormone synthesizing enzymes (e.g. 2, 3, 4, 5 or more) may be modulated. The modulation may comprise, for example, inducing, increasing, reducing and/or ceasing expression and/or secretion and/or production of the multiple hormones, hormone precursors and/or hormone synthesizing enzymes. Targeting the cell surface receptor may have different effects on different hormones, hormone precursors and/or hormone synthesizing enzymes within the cell. For example, the secretion of a first hormone and/or hormone precursor may be increased or induced, whereas the secretion of a second hormone and/or hormone precursor may be decreased or ceased. Similarly, the expression of a first hormone, hormone precursor and/or hormone synthesizing enzyme may be increased or induced, whereas the expression of a second hormone, hormone precursor and/or hormone synthesizing enzyme may be decreased or ceased. For example, the ability to affect hormone profiles allows the synergistic effects of multiple hormones to be obtained by targeting a cell surface receptor.

The invention provides a method for treating or preventing a gut motility disorder comprising administering a compound that targets a target in an M/X cell. In some embodiments, the target is a cell surface receptor. In some embodiments, targeting the cell surface receptor induces release of Motilin and Angiotensin. These both have a function in motility control in the gut, thus opening up the possibility of a synergistic response. Also provided is a compound that targets a target on an M/X cell, e.g. a cell surface receptor, for use in treating or preventing a gut motility disorder.

Similarly, the invention provides a method for treating or preventing an appetite-related disorder, e.g. anorexia or obesity (preferably anorexia) comprising administering a compound that targets a target on an M/X cell, e.g. a cell surface receptor. In some embodiments, the appetite-related disorder is selected from anorexia and cancer cachexia. In some embodiments, targeting the cell surface receptor induces release of Ghrelin and Cerebellin 1. Both Ghrelin and Cerebellin 1 are orexigenic peptides that stimulate appetite. The ability to affect two hormones opens up the prospect of a synergistic response. Also provided is a compound that targets a target on an M/X cell, e.g. a cell surface receptor, for use in treating or preventing an appetite-related disorder, e.g. anorexia.

In some embodiments, the receptor on the M/X cell is selected from ASGR1, EPHA4, FFAR4, GFRA3, NTRK2, SSTR2, TNFRSF21, ABCC8, CD44, CALY, PVR and IL20RA. In some embodiments, the compound is selected from IL-10, IL-19, IL-20, IL-24 and IL-26. These target the IL20RA.

In some embodiments, the compound targets a cell surface receptor which is present on only one EEC subtype. In some embodiments, the compound targets a cell surface receptor which is present on more than one (e.g. 2, 3, 4 or more) EEC subtypes. For example, the invention similarly provides a method for treating or preventing a disease or disorder comprising administering a compound which targets a cell surface receptor on more than one EEC subtype, wherein the EEC subtypes express and/or secrete one or more (e.g. 2, 3, 4 or more) hormones and/or hormone precursors implicated in the treatment or prevention of the disease or disorder. Similarly, there is provided a compound for use in treating or preventing a disease or disorder, for example, wherein the treating or preventing comprises targeting a cell surface receptor which is present on more than one (e.g. 2, 3, 4, or more) EEC subtypes in order to modulate expression and/or secretion of one or more hormones and/or hormone precursors, implicated in the treatment or prevention of the disease or disorder.

For example, in some embodiments, the cell surface receptor is the SCTR. The SCTR is expressed by both K/G-cells and L-cells. In some embodiments, targeting this receptor induces the secretion of GLP-1 and GIP. Both GLP-1 and GIP are incretins that collaboratively enhance insulin secretion. Accordingly, in some embodiments, the disease or disorder is diabetes, e.g. type II diabetes. In some embodiments, the compound is a compound that targets the SCTR. In some embodiments, the compound is Secretin. Thus, in some embodiments, there is provided a method for treating or preventing diabetes comprising administering a compound that targets the SCTR (e.g. Secretin) to a patient. Similarly, there is provided a compound that targets the SCTR (e.g. Secretin) for use in treating or preventing diabetes. In some embodiments, the treating or preventing diabetes comprises increasing or inducing expression and/or secretion and/or production of GLP-1 and GIP. In some embodiments, the treating or preventing diabetes comprises increasing or inducing secretion of GLP-1 and GIP. GLP-1 and GIP may also collaboratively treat or prevent neurodegenerative diseases or disorders, for example, Parkinson's disease and Alzheimer's disease. Accordingly, in some embodiments, the disease or disorder is a neurodegenerative disease or disorder, for example, Parkinson's disease or Alzheimer's disease. Thus, in some embodiments, there is provided a method for treating or preventing a neurodegenerative disease or disorder (e.g. Parkinson's disease or Alzheimer's disease) comprising administering a compound that targets the SCTR (e.g. Secretin) to a patient. Similarly, there is provided a compound that targets the SCTR (e.g. Secretin) for use in treating or preventing a neurodegenerative disease or disorder (e.g. Parkinson's disease or Alzheimer's disease). In some embodiments, the treating or preventing the neurodegenerative disease or disorder comprises increasing or inducing expression and/or secretion and/or production of GLP-1 and GIP.

The methods of the invention may, in some embodiments, be conducted in an organoid of the invention. In some embodiments, the methods of the invention are conducted in a culture of cells comprising EECs or comprising one or more EEC subtypes. In some embodiments, the methods of the invention are conducted in vitro. In some embodiments, the methods are conducted in vivo in a patient.

The invention further provides a method for modulating a physiological effect comprising modulating expression or secretion of, or agonising or antagonising one or more targets in human EECs. In some embodiments, the one or more targets are one or more targets as described herein. In some embodiments, the modulating a physiological effect is increasing a physiological effect, for example, stimulating a physiological effect. In some embodiments, the modulating a physiological effect is decreasing a physiological effect, for example, repressing a physiological effect. In some embodiments, the modulating expression is increasing expression. In some embodiments, the modulating expression is decreasing expression.

The invention provides a method for investigating the function of one or more targets as described herein comprising knocking out the target in an intestinal organoid of the invention and determining whether there is a change in the organoid compared to an organoid in which the target has not been knocked out. In some embodiments, the organoid is a reporter organoid and the method comprises assessing whether there is a change in expression and/or secretion (as appropriate) of the one or more tagged EEC-specific genes, e.g. of the one or more tagged hormones, hormone precursors and/or hormone synthesizing enzymes compared to an organoid in which the target has not been knocked out.

In some embodiments, a method of the invention comprises targeting two or more targets on human EECs. The two or more targets may be on the same cell subtype or may be on different cell subtypes. For example, a method of the invention may comprise targeting two or more cell surface receptors on human EECs.

In embodiments in which intestinal organoids are used in methods described herein, the intestinal organoids may be any suitable intestinal organoid, for example, an intestinal organoid of the invention. In some embodiments, a reporter intestinal organoid of the invention is used.

Newly Identified Targets

As mentioned above, the inventors have identified a number of targets of interest in human EECs or one or more human EEC subtypes using the human EEC atlas described herein. EECs are hormone producing cells. The inventors have shown, using the organoid technology provided by the present invention, that production of hormones can be modulated by targeting targets on human EECs. In particular, they have shown that hormone production can be modulated by targeting GPCRs, by targeting other types of cell surface receptors and by targeting transcription factors. The various practical applications of targets that are described herein can be applied to each of the identified targets that are described herein.

EECs sense chemicals via their cell surface receptors. Release of hormones by the EECs is modulated in response. Thus, targeting cell surface receptors on EECs can be expected to modulate expression and/or secretion and/or production of one or more hormones, hormone precursors and/or hormone synthesizing enzymes by the EECs.

In some embodiments, the human EECs are human L-cells and/or the target is the Secretin receptor (SCTR). The inventors have surprisingly found that human EECs can sense extracellular Secretin. Using the reporter organoids described herein, the inventors have surprisingly shown that Secretin can stimulate L-cells to secrete GCG (which is then processed to GLP-1). As mentioned above in the discussion of practical applications, the invention provides a method for modulating expression and/or secretion of one or more hormones, hormone precursors and/or hormone synthesizing enzymes comprising targeting a cell surface receptor on human EECs. Thus, the invention provides a method for modulating expression and/or secretion of GCG comprising targeting the SCTR in human EECs. The invention also provides a method for modulating GLP-1 production comprising targeting the Secretin receptor (SCTR) in human EECs. In some embodiments, the human EECs are human L-cells. In some embodiments, the method comprises targeting SCTR with an SCTR agonist. In some embodiments, the method comprises targeting SCTR with an SCTR antagonist. For example, there is provided a method for increasing GLP-1 production comprising contacting an L-cell with a compound that targets the SCTR. In some embodiments, the L-cell is in an intestinal organoid of the invention. In some embodiments, the intestinal organoid is a reporter organoid. In some embodiments, the L-cell is in a patient. Accordingly, in some embodiments, the method comprises targeting delivery of a compound that targets the SCTR to the patient's gut. In some embodiments, there is provided a method for increasing GLP-1 production comprising contacting an intestinal organoid with a compound that targets the SCTR.

As mentioned above in the discussion of practical applications of targets, the invention provides a method for modulating a physiological effect comprising modulating expression or secretion of, or agonising or antagonising one or more targets in a human EEC. Thus, in some embodiments, the physiological effect is production of GLP-1 by human EECs and the target is the SCTR. Accordingly, the invention provides a method for modulating production of GLP-1 comprising modulating expression of or agonising or antagonising the SCTR in a human EEC.

The inventors' surprising finding that modulation of SCTR in L-cells modulates GLP-1 production opens the way for therapies which target the SCTR in order to treat or prevent a disease or disorder in which GLP-1 is implicated. As mentioned above in the discussion of the practical applications of targets identified by the invention, the invention provides a method for treating or preventing a disease or disorder comprising administering a compound found to affect secretion of a hormone precursor when used to target a target in a human EEC, wherein the hormone precursor is implicated in the disease or disorder. Thus, in some embodiments, the target is the SCTR and the disease or disorder is a disease or disorder in which GLP-1 is implicated. The hormone precursor in this case is GCG. It is also implicated in the disease or disorder because it is processed to GLP-1, which is implicated in the disease or disorder. Thus, in some embodiments, the hormone and/or hormone precursor are GLP-1 and/or GCG, respectively. Thus, the invention provides a method of treating or preventing a disease or disorder in which GLP-1 is implicated comprising administering a compound that targets the SCTR in human EECs (e.g. an agonist or an antagonist of the SCTR). Similarly, the invention provides a compound that targets the SCTR in human EECs for use in a method of treating or preventing a disease or disorder in which GLP-1 is implicated. In some embodiments, the disease or disorder in which GLP-1 is implicated is a disease or disorder in which a decrease in production of GLP-1 is implicated. In some embodiments, the disease or disorder is a disease or disorder treatable or preventable by inducing or increasing GLP-1 production. Examples of such diseases and disorders are described herein. In some embodiments, the disease or disorder is diabetes, such as type 2 diabetes (Sharma et al., 2018). In some embodiments, the disease or disorder is an appetite-related disease or disorder requiring appetite inhibition. In some embodiments, the disease or disorder is obesity. In some embodiments, the disease or disorder is Prader-Willi syndrome. In some embodiments, the disease or disorder is a neurodegenerative disease, for example, Parkinson's disease or Alzheimer's Disease. People with Parkinson's disease commonly display impaired glucose tolerance which can induce brain insulin resistance. A number of preclinical studies have demonstrated that GLP-1s are beneficial when tested in animal models of Parkinson's Disease, and it is thought that GLP-1 affects neurological and cognitive functions, as well as its regulatory effect on glucose metabolism. In some embodiments, a compound that targets the SCTR is used to treat insulin resistance, for example, in type 2 diabetes or in Parkinson's Disease. Accordingly, the invention provides a method for treating or preventing diabetes, obesity or a neurodegenerative disease or disorder such as Parkinson's Disease or Alzheimer's Disease, wherein the method comprises administering a compound that targets the SCTR on human EECs to a patient, wherein the compound induces or increases GLP-1 production by the human EECs. For example, the compound may be an SCTR agonist. In some embodiments, the disease or disorder is selected from an appetite-related disorder, e.g. an appetite-related disorder requiring appetite inhibition (e.g. obesity, diabetes or Prader-Willi syndrome), diabetes (e.g. type II diabetes) and/or a neurodegenerative disease or disorder (e.g. Parkinson's Disease or Alzheimer's Disease). In some embodiments, the hormone precursor is GCG and/or the hormone is GLP-1. In some embodiments, the disease or disorder is a disease or disorder treatable or preventable by ceasing or decreasing GLP-1 production. Examples of such diseases and disorders are described herein.

As mentioned above in the discussion of practical applications of the targets, the invention provides a method for modulating a physiological effect comprising modulating expression or secretion of, or agonising or antagonising one or more targets in a human EEC. Thus, in some embodiments, the target is the SCTR and the physiological effect is insulin resistance.

Also provided is a pharmaceutical composition comprising a therapeutic compound which affects the SCTR in a human EEC, wherein the pharmaceutical composition is formulated to be administered or delivered to the gut.

Any suitable compound may be used to target the SCTR, for example, a compound as described herein. In some embodiments, the compound that targets the SCTR is Secretin. The use of variants and fragments of Secretin, or fragments of a variant of Secretin are also encompassed by the invention. In some embodiments, the variant is a polypeptide sequence having at least 75% (e.g. at least 80%, 85%, 90%, 95%, 98%, 99%) sequence identity with the polypeptide sequence of the Secretin sequence shown in SEQ ID NO:70. Variants and fragments of compounds are described elsewhere herein. The variant or fragment preferably induces or increases GLP-1 production by human EECs (e.g. by human L-cells) when used to contact the SCTR. In some embodiments, the compound that targets the SCTR is a mimetic of Secretin. A pharmaceutical composition of the invention may comprise a compound as described herein, e.g. Secretin or a variant or fragment thereof.

A Secretin stimulation test is commonly used in diagnostics of Zollinger-Ellison syndrome patients that suffer from gastrin-producing tumors (Berna et al., 2006). In some embodiments, the physiological effect is blood gastrin levels, the human EECs are human G-cells and the target is the SCTR. Secretin normally represses blood gastrin by inhibiting the secretion of gastrin from the stomach G-cells (the major site of Gastrin production), likely through modulating the luminal pH. In contrast, patients suffering from small intestinal gastrinoma show sharp increases in serum gastrin upon secretin administration. The data presented herein suggest this to occur through the broad SCTR-expression among small intestinal gastrin-producing G-cells. Accordingly, in some embodiments, the disease or disorder is a neuroendocrine tumour (for example, a small intestinal gastrinoma, e.g. Zollinger-Ellison syndrome). Accordingly, the invention provides a method for treating or preventing a neuroendocrine tumour (e.g. Zollinger-Ellison syndrome), comprising targeting the SCTR on small intestinal gastrin-producing G-cells. In some embodiments, by targeting the SCTR on small intestinal gastrin-producing G-cells, gastrin expression and/or secretion is reduced relative to a small intestinal gastrin-producing G cell from the same individual in which the SCTR has not been targeted. In some embodiments, targeting the SCTR on small intestinal gastrin-producing G-cells results in prevention of gastrin production. In some embodiments, targeting comprises targeting a downstream pathway of the SCTR to reduce or prevent gastrin secretion. In some embodiments, the targeting comprises contacting the SCTR with an SCTR agonist. For example, in some embodiments, the compound is an SCTR agonist. In some embodiments, the human EECs are G-cells.

Methods for making receptor agonists are well known in the art, for example, agonistic antibodies. A suitable agonist may be identified using a receptor organoid of the present invention. For example, in some embodiments, there is provided a method for identifying an agonist of the SCTR on small intestinal gastrin-producing G cells, wherein the method comprises (i) contacting a reporter organoid established from a patient with a neuroendocrine tumour (e.g. with Zollinger-Ellison syndrome) and comprising small intestinal gastrin-producing G cells in which the hormone gastrin is linked to a fluorescent marker, with a compound; ii) detecting changes in gastrin secretion. The method may optionally further comprise comparing the result with a control organoid which has not been contacted with the compound. For example, the control organoid may be a clonally identical reporter organoid from the same patient. In some embodiments, the method comprises detecting the tagged gastrin hormone at two or more time points, wherein a reduction in loss of intracellular fluorescence from the fluorescent-tagged gastrin hormone, for example, compared with a control organoid, indicates a reduction in gastrin secretion and identifies the compound as an agonist.

The invention further provides a method for identifying/screening/validating a compound for targeting the SCTR on human EECs (e.g. on human L-cells), wherein the method comprises (i) contacting a reporter organoid comprising L-cells in which the hormone precursor GCG is linked to a fluorescent marker, with the compound; ii) detecting whether expression and/or secretion of GCG is affected, for example, by detecting changes in secretion of the tagged GCG hormone precursor at two or more time points; wherein the loss of intracellular fluorescence from the fluorescent-tagged GCG hormone precursor indicates GCG secretion. In some embodiments, the two time points are about 12 hours apart. The method may optionally further comprise comparing the result with a control organoid which has not been contacted with the compound or with an organoid which has been treated with a substance or composition that increases cAMP levels (such as stimulator of adenylate cyclase, e.g. Forskolin). In some embodiments, the reporter organoid is contacted with the compound for a period of between 12-32 hours, for example, 20-28 hours, for example about 24 hours. In some embodiments, the compound is Secretin.

The invention further provides a method for identifying/screening/validating a compound for targeting the SCTR on human EECs (e.g. on human L-cells), wherein the method comprises (i) contacting a reporter organoid comprising L-cells in which the hormone GLP-1 is linked to a fluorescent marker, with the compound; ii) detecting whether expression and/or secretion of GLP-1 is affected, for example, by detecting changes in secretion of the tagged GLP-1 hormone at two or more time points; wherein the loss of intracellular fluorescence from the fluorescent-tagged GLP-1 hormone indicates GLP-1 secretion. In some embodiments, the two time points are about 12 hours apart. The method may optionally further comprise comparing the result with a control organoid which has not been contacted with the compound or with an organoid which has been treated with a substance or composition that increases cAMP levels (such as stimulator of adenylate cyclase, e.g. Forskolin). In some embodiments, the reporter organoid is contacted with the compound for a period of between 12-32 hours, for example, 20-28 hours, for example about 24 hours. In some embodiments, the compound is Secretin.

The finding that EEC-derived Secretin regulates GLP-1 production by EECs demonstrates the ability of EECs to cross-communicate with one another. The inventors' finding herein that EECs express multiple receptors for EEC hormones likewise supports the ability of EECs to cross-communicate with one another.

The invention similarly provides a method for identifying or isolating one or more human EECs or for identifying or isolating a population of human EECs comprising selecting cells which express one or more targets expressed by a human EEC. In some embodiments, the human EECs are human L-cells and the target is the Secretin receptor SCTR. In some embodiments, high expression of SCTR is used as a marker for human L-cells. In some embodiments, a high level of SCTR is expressed or a high level of SCTR expression is selected for. For example, in some embodiments, human EECs with the highest level of SCTR expression are selected in order to select human L-cells. In some embodiments, the high level of expression of SCTR is 1 or more SCTR transcripts for each 3000 total transcripts in the cell, wherein the total transcripts relate to the transcripts of any genes in the cell. In some embodiments, SCTR is expressed or expression of SCTR is selected for.

In some embodiments, the human EECs are human EECs producing GCG and/or GAST/GIP and/or the target is the SCTR. For example, in some embodiments, the human EECs are human K/G cells. The inventors have found that SCTR expression is low in ECs and enriched in EECs producing GCG and/or GAST/GIP. Accordingly, the invention provides a method of identifying EECs producing GCG and/or GAST/GIP comprising identifying cells expressing the SCTR marker. Similarly, the invention provides a method of obtaining EECs producing GCG and/or GAST/GIP comprising selecting cells expressing the SCTR marker and isolating said cells. Use of SCTR as a marker for EECs producing GCG and/or GAST/GIP is similarly provided. An isolated EEC producing GCG and/or GAST/GIP is also provided.

An isolated human L-cell, GCG+ or GAST+/GIP+ cell that expresses the SCTR is also provided, as is an isolated population of such cells. In some embodiments, the isolated cell additionally expresses CHGA. An isolated population of such cells is similarly provided.

SCTR is a GPCR signaling through cAMP. The inventors have found that Secretin induces swelling of organoids similar to Forskolin. Accordingly, in some embodiments, there is provided a method for testing the effect of Secretin on EECs, wherein the method comprises (i) contacting an intestinal organoid with Secretin; ii) determining if the organoid swells. The method may further comprise determining the extent of organoid swelling compared to swelling in an organoid contacted with Forskolin. In addition, the invention provides a method for identifying/screening/validating a compound for targeting the SCTR in human EECs, comprising (i) contacting an intestinal organoid with the compound and (ii) determining if the organoid swells. The method may further comprise the step of validating that the compound targets the SCTR by repeating the method in an intestinal organoid which comprises an inactivated SCTR and determining if organoid swelling no longer occurs. If it no longer occurs, it suggests that the compound acts via the SCTR. In some embodiments, the SCTR is inactivated by way of mutation. In some embodiments, the SCTR is inactivated by way of functional blocking, e.g. by prior binding of an antagonist. The intestinal organoid used for validating that the compound targets the SCTR is preferably established from the same cells and cultured under the same conditions in the same media as the intestinal organoid used in the method for identifying/screening/validating.

In some embodiments, the human EECs are human M-X cells and/or the target is IL20-RA. IL20-RA is the receptor for cytokines of the IL10-family (IL20-RA). IL20-RA is proposed to link a sensory mechanism for pathogens to a motility response expelling such infection. Of note, irritable bowel syndrome (IBS), characterized by alterations in gut motility, is associated with a reduction in the IL20-RA-ligand IL-10 (Gonsalkorale et al., 2003), and the findings described herein show it is potentially mediated through M-X cells. The inventors now show through the use of the organoid technology provided by the present invention that stimulation of the IL20-RA leads to secretion of Motilin. Accordingly, in some embodiments, the one or more hormones is Motilin. Accordingly, in some embodiments, the physiological effect is gut motility. In some embodiments, the disease or disorder is a pathogen infection, e.g. of the intestine. The invention provides a method for linking presence of pathogens to a motility response expelling such infection by using the cytokine receptor IL-20RA as a sensory mechanism. In some embodiments, the invention provides a method for treating or preventing a pathogen infection comprising targeting the IL20-RA in human M-X cells. Similarly, the invention provides a method for expelling a pathogenic infection in a patient's intestine, comprising administering a compound that targets the cytokine receptor IL-20RA to the patient. Preferably, administration of the compound stimulates the motility response in the intestine. In some embodiments, the compound is an agonist of IL-20RA. In some embodiments, the compound is an antagonist of IL-20RA. In some embodiments, the compound is IL-10. In some embodiments, the disease or disorder is a disease or disorder in which Motilin is implicated. In some embodiments, the disease or disorder is a gut motility disease or disorder, for example, irritable bowel syndrome (IBS). In some embodiments, the gut motility disorder is selected from a bowel movement disorder, Parkinson's disease or gastroparesis. In some embodiments, the invention provides a method for treating or preventing a gut motility disease or disorder comprising targeting the IL20-RA in human M-X cells with an agonist. For example, the invention provides a method for treating or preventing a gut motility disease or disorder comprising administering a compound that agonises the IL20-RA in human M-X cells to a patient. In some embodiments, the compound increases or induces expression and/or secretion of Motilin. Motilin is implicated in hunger. Therefore, the invention provides a method for treating or preventing a disease or disorder requiring appetite stimulation comprising administering a compound that targets the IL20-RA in human M-X cells to a patient. In some embodiments, the disease or disorder is anorexia. In some embodiments, the disease or disorder is cancer cachexia. Motilin is also implicated in insulin release. Therefore, the invention provides a method for treating or preventing diabetes comprising administering a compound that targets the IL20-RA in human M-X cells to a patient. In some embodiments, the diabetes is type II diabetes. Motilin is also implicated in increased gallbladder emptying. Therefore, the invention provides a method for treating or preventing a biliary movement disorder comprising administering a compound that targets the IL20-RA in human M-X cells to a patient. In some embodiments, the disease or disorder is biliary dyskinesia. In some embodiments, the compound decreases or ceases expression and/or secretion of Motilin. The invention provides a method for treating or preventing a disease or disorder requiring appetite inhibition comprising administering a compound that targets the IL20-RA in human M-X cells to a patient. In some embodiments, the disease or disorder is obesity. In some embodiments, the disease or disorder is diabetes. In some embodiments, the disease or disorder is type II diabetes. In some embodiments, the disease or disorder is Prader-Willi syndrome. In some embodiments, the agonistic compound is the natural ligand IL-10. In some embodiments, one or more cytokines are used as the one or more agonistic compounds. For example, in some embodiments, the one or more compounds comprise one or more of (e.g. 1, 2, 3, 4 or more or all 5 of) IL-10, IL-19, IL-20, IL-24 and IL-26. Thus, in some embodiments, the invention comprises using a cocktail of cytokines as described herein.

In some embodiments, the human EECs are human M-X cells and/or the target is the BMP receptor. The inventors have shown, using the organoid technology provided herein, that activating BMP signalling in human M-X cells increases Motilin expression and decreases Ghrelin expression. In some embodiments, the disease or disorder is a disease or disorder in which Motilin is implicated. For example, Motilin is implicated in gut motility disorders. Therefore, in some embodiments, the disease or disorder is a gut motility disorder, e.g. as described herein. For example, in some embodiments, the disease or disorder is a bowel movement disorder, Parkinson's disease or gastroparesis. Motilin is also implicated in hunger. Thus, in some embodiments, the disease or disorder is a disease or disorder requiring appetite stimulation. In some embodiments, the disease or disorder is anorexia. In some embodiments, the disease or disorder is cancer cachexia. In some embodiments, the disease or disorder is a disease or disorder requiring appetite inhibition. In some embodiments, the disease or disorder is obesity. In some embodiments, the disease or disorder is diabetes. In some embodiments, the disease or disorder is type II diabetes. In some embodiments, the disease or disorder is Prader-Willi syndrome. In addition, Motilin is associated with diabetes, in particular type II diabetes. Thus, in some embodiments, the disease or disorder is diabetes. In some embodiments, the diabetes is type II diabetes. Motilin is also associated with increased gallbladder emptying. Thus, in some embodiments, the disease or disorder is a biliary movement disorder. In some embodiments, the disease or disorder is biliary diskinesia. In some embodiments, the disease or disorder is a disease or disorder in which Ghrelin is implicated. Increasing ghrelin is implicated in diseases and disorders requiring appetite stimulation, e.g. anorexia and cancer cachexia. Conversely, a decrease in ghrelin would be useful for treating or preventing a disease or disorder requiring appetite inhibition, for example, obesity or diabetes or Prader-Willi syndrome. Thus, in some embodiments, the disease or disorder is a disease or disorder requiring appetite inhibition. For example, in some embodiments, the disease or disorder is obesity or diabetes. In some embodiments, the diabetes is type II diabetes. In some embodiments, the disease or disorder is Prader-Willi syndrome. In some embodiments, the compound used to target the BMP receptor and activate BMP signalling is a BMP pathway activator, for example, as described herein. In some embodiments, the compound used to target the BMP receptor is a compound that inhibits BMP signalling, and so is a BMP inhibitor. Examples of suitable BMP inhibitors for use in the invention are described herein. A BMP inhibitor would be useful for increasing Ghrelin expression and would therefore be useful for treating diseases and disorders requiring appetite stimulation, e.g. anorexia and cancer cachexia. Thus, in some embodiments, the disease or disorder is a disease or disorder requiring appetite stimulation, e.g. anorexia or cancer cachexia. Thus, in some embodiments, the disease or disorder is an appetite-related disease or disorder. In some embodiments, the physiological effect is gut motility. In some embodiments, the physiological effect is appetite. In some embodiments, the one or more hormones are Motilin and/or Ghrelin. In some embodiments, the compound is a BMP pathway activator, e.g. as described herein. In some embodiments, the compound increases or induces expression and/or secretion of Motilin. In some embodiments, the compound decreases or ceases expression and/or secretion of Motilin. In some embodiments, the compound increases or induces expression and/or secretion of Ghrelin. In some embodiments, the compound decrease or ceases expression and/or secretion of Ghrelin.

In some embodiments, the target is a cell surface receptor selected from Plexin B1 (PLXNB1), Solute Carrier Family 22 Member 17 (SLC22A17), Frizzled Class Receptor 3 (FZD3), Receptor Tyrosine Kinase Like Orphan Receptor 1 (ROR1), Leptin receptor (LEPR), Nischarin (NISCH), EPH Receptor B3 (EPHB3), Dual Serine/Threonine And Tyrosine Protein Kinase (DSTYK), Erythropoietin Receptor (EPOR), Cholinergic Receptor Muscarinic 3 (CHRM3), Intelectin 1 (ITLN1), Inositol 1,4,5-Trisphosphate Receptor Type 2 (ITPR2), ABL Proto-Oncogene 2, Non-Receptor Tyrosine Kinase (ABL2), Adhesion G Protein-Coupled Receptor L1 (ADGRL1), Polycystin 2, Transient Receptor Potential Cation Channel (PKD2), GDNF Family Receptor Alpha 1 (GFRA1), Repulsive guidance molecule B (RGMB), Kringle Containing Transmembrane Protein 1 (KREMEN1), ABL Proto-Oncogene 1, Non-Receptor Tyrosine Kinase (ABL1), Eukaryotic Translation Elongation Factor 1 Alpha 1 (EEF1A1), Gamma-Aminobutyric Acid Type B Receptor Subunit 2 (GABBR2), Melanocortin 1 Receptor (MC1R), GDNF Family Receptor Alpha 3 (GFRA3), Calcyon Neuron Specific Vesicular Protein (CALY), Receptor activity modifying protein 1 (RAMP1), Adhesion G Protein-Coupled Receptor G1 (ADGRG1), Transforming Growth Factor Beta Receptor 3 (TGFBR3), Protein Tyrosine Kinase 7 (PTK7), G Protein-Coupled Receptor 160 (GPR160), PVR Cell Adhesion Molecule (PVR), Seizure Related 6 Homolog Like 2 (SEZ6L2), Fc Fragment Of IgG Receptor And Transporter (FCGRT), Olfactory Receptor Family 51 Subfamily E Member 1 (OR51E1), Mucolipin 3 (MCOLN3), Transient Receptor Potential Cation Channel Subfamily A Member 1 (TRPA1), Neuropeptide Y Receptor Y1 (NPY1R), GPR160, Free fatty acid receptor 2 (FFAR2), Adhesion G Protein-Coupled Receptor G1 (ADGRG1), Gamma-Aminobutyric Acid Type A Receptor Subunit Beta3 (GABRB3), Somatostatin Receptor 1 (SSTR1), Solute Carrier Family 7 Member 1 (SLC7A1), FLVCR Heme Transporter 1 (FLVCR1), ERBB Receptor Feedback Inhibitor 1 (ERRFI1), GABA Type A Receptor-Associated Protein (GABARAP), Guanylate Cyclase 2C (GUCY2C), Cornichon Family AMPA Receptor Auxiliary Protein 2 (CNIH2), cluster of differentiation 36 (CD36), Adhesion G Protein-Coupled Receptor G4 (ADGRG4), G protein-coupled receptor 68 (GPR68), Thyroid Stimulating Hormone Receptor (TSHR), Free fatty acid receptor 2 (FFAR2), G Protein-Coupled Bile Acid Receptor 1 (GPBAR1), NPY1R, Calcium Sensing Receptor calcium-sensing receptor (CASR), Gamma-Aminobutyric Acid Type B Receptor Subunit 2 (GABBR2), GPR68, TSHR, Dopamine receptor D2 (DRD2), ADGRG4, OR51E1, Secretin receptor (SCTR), Somatostatin Receptor 2 (SSTR2), GUCY2C, Cluster of differentiation 44 (CD44), Cornichon Family AMPA Receptor Auxiliary Protein 2 (CNIH2), Neuronal acetylcholine receptor subunit alpha-5 (CHRNA5), CALY, ATP Binding Cassette Subfamily C Member 8 (ABCC8), Adenosylhomocysteinase Like 1 (AHCYL1), Gastrin Releasing Peptide Receptor (GRPR), GPBAR1, ABCC8, GUCY2C, CALY, SCTR, Fibroblast Growth Factor Receptor 1 (FGFR1), SSTR2, CD44, Interleukin 17 Receptor B (IL17RB), CASR, Protein Tyrosine Phosphatase Receptor Type N2 (PTPRN2), ADGRG1, KIT Proto-Oncogene, Receptor Tyrosine Kinase (KIT), Melanoma-associated antigen D1 (MAGED1), G Protein-Coupled Receptor 35 (GPR35), Transforming Growth Factor Beta Receptor 3 (TGFBR3), PVR, CNIH2, Polycystin 1 (PKD1), Insulin Receptor (INSR), Transmembrane Protein 97 (TMEM97), Claudin 3 (CLDN3), GABARAP, SEZ6L2, Integrin Subunit Beta 1 (ITGB1), SSTR5, G Protein-Coupled Receptor 162 (GPR162), FFAR2, GPBAR1, GPR162, GPR37, ADGRD1, ADGRA2, Melanocortin 1 Receptor (MC1R), olfactory receptor family 7 subfamily E member 47 pseudogene (OR7E47P), Angiotensin II Receptor Type 1 (AGTR1), CASR, SCTR, SSTR5, SSTR2, Asialoglycoprotein Receptor 1 (ASGR1), EPH Receptor A4 (EPHA4), Free fatty acid receptor 4 (FFAR4), GFRA3, neurotrophic receptor tyrosine kinase 2 (NTRK2), SSTR2, TNF Receptor Superfamily Member 21 (TNFRSF21), ABCC8, CD44, CALY, PVR and Interleukin 20 Receptor Subunit Alpha (IL20RA).

In some embodiments, the target is a cell surface receptor selected from PLXNB1, SLC22A17, FZD3, ROR1, LEPR, NISCH, EPHB3, DSTYK, EPOR, CHRM3, ITLN1, ITPR2, ABL2, ADGRL1, PKD2, GFRA1, RGMB, KREMEN1, ABL1, EEF1A1, GABBR2 and MC1R. Such cell surface receptors are expressed broadly on EECs.

In some embodiments, the target is a cell surface receptor selected from GFRA3, CALY, RAMP1, ADGRG1, TGFBR3, PTK7, GPR160, PVR, SEZ6L2 and FCGRT. Such cell surface receptors are expressed on D-cells. In some embodiments, the hormone (e.g. the tagged hormone) is SST.

In some embodiments, the target is a cell surface receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the cell surface receptor is a GPCR selected from FFAR2, GPBAR1, NPY1R, CASR, GABBR2, GPR68, TSHR, DRD2, ADGRG4 and OR51E1. Such cell surface receptors are expressed on EC-cells. In some embodiments, the hormone synthesizing enzyme (e.g. the tagged hormone synthesizing enzyme) is TPH1.

In some embodiments, the target is a cell surface receptor selected from SCTR, SSTR2, GUCY2C, CD44, CNIH2, CHRNA5, CALY, ABCC8, AHCYL1 and GRPR. Such cell surface receptors are expressed on K/G-cells. In some embodiments, the one or more hormones (e.g. the one or more tagged hormones) are GIP and/or GAST.

In some embodiments, the target is a cell surface receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the cell surface receptor is a GPCR selected from FFAR2, GPBAR1, GPR162, GPR37, ADGRD1, ADGRA2, MC1R, OR7E47P, AGTR1, CASR, SCTR, SSTR5 and SSTR2. Such cell surface receptors are expressed on L-cells. In some embodiments, the one or more hormones (e.g. the one or more tagged hormones) are GCG and/or NTS.

In some embodiments, the target is a cell surface receptor selected from ASGR1, EPHA4, FFAR4, GFRA3, NTRK2, SSTR2, TNFRSF21, ABCC8, CD44, CALY, PVR and IL20RA. Such cell surface receptors are expressed on M/X-cells. In some embodiments, the one or more hormones (e.g. the one or more tagged hormones) are MLN and/or GHRL.

In some embodiments of the various methods and uses described herein, the human EECs are EECs of all subtypes or of more than one EEC subtype. In some embodiments, the target is a target broadly expressed in human EECs, as described herein. In some embodiments, the target is a target expressed in all human EECs. In some embodiments, the target is a target expressed in more than one EEC subtype. In some embodiments, the target is a target in human EECs as described herein. In some embodiments, the target is a receptor selected from PLXNB1, SLC22A17, FZD3, ROR1, LEPR, NISCH, EPHB3, DSTYK, EPOR, CHRM3, ITLN1, ITPR2, ABL2, ADGRL1, PKD2, GFRA1, RGMB, KREMEN1, ABL1, EEF1A1, GABBR2 and MC1R. The inventors have found that such cell surface receptors are expressed broadly on EECs. In some embodiments, the compound is a compound that targets a target in EECs. In some embodiments, the compound is a compound which targets a cell surface receptor on EECs. In some embodiments, the compound is a melanocortin, ACTH or MSH. These bind the MC1R receptor. In some embodiments, the compound is GABA. GABA binds the GABBR2 receptor. In some embodiments, the compound targets a target in EECs. For example, in some embodiment, the target is expressed in all EEC subtypes. The invention encompasses targeting one or more such targets in human EECs in order to treat or prevent a disease or disorder treatable or preventable by modulating expression and/or secretion and/or production of one or more hormones by the EECs. Accordingly, in some embodiments, the disease or disorder is a disease or disorder as described herein. In some embodiments, the one or more hormones are one or more hormones as described herein. Methods of treating or preventing such diseases and disorders and compounds for use in treating or preventing such diseases and disorders are provided by the invention, as described above for the various practical applications of the targets. In some embodiments, the disease or disorder is an appetite-related disease or disorder. In some embodiments, the disease or disorder is obesity. In some embodiments, the disease or disorder is Prader-Willi syndrome. In some embodiments, the disease or disorder is anorexia. In some embodiments, the disease or disorder is cancer cachexia. In some embodiments, the disease or disorder is diabetes (in particular type II diabetes). In some embodiments, the disease or disorder is a gut motility disease or disorder. In some embodiments, the disease or disorder is a bowel movement disorder. In some embodiments, the disease or disorder is Parkinson's disease. In some embodiments, the disease or disorder is gastroparesis. In some embodiments, the disease or disorder is a neurodegenerative disease. In some embodiments, the disease or disorder is Parkinson's disease. In some embodiments, the disease or disorder is Alzheimer's disease. In some embodiments, the disease or disorder is depression. In some embodiments, the disease or disorder is a biliary movement disorder. In some embodiments, the disease or disorder is biliary dyskinesia. In some embodiments, the disease or disorder is high acid secretion in the stomach. In some embodiments, the disease or disorder is indigestion caused by high acid secretion in the stomach. In some embodiments, the disease or disorder is acid reflux caused by high acid secretion in the stomach. In some embodiments, the high acid secretion in the stomach is caused by abnormal parietal cell activity in the stomach. In some embodiments, the disease or disorder is IBS. In some embodiments, the disease or disorder is IBD. In some embodiments, the disease or disorder is ulcerative colitis. In some embodiments, the disease or disorder is Crohn's disease.

In some embodiments, the compound induces or increases expression and/or secretion and/or production of a hormone. For example, in some embodiments, the compound induces or increases expression of a hormone. For example, in some embodiments, the compound induces or increases secretion of a hormone. For example, in some embodiments, the compound induces or increases production of a hormone. In some embodiments, the compound decreases or ceases expression and/or secretion and/or production of a hormone. For example, in some embodiments, the compound decreases or ceases expression of a hormone. For example, in some embodiments, the compound decreases or ceases secretion of a hormone. For example, in some embodiments, the compound decreases or ceases production of a hormone. In some embodiments, the hormone is Ghrelin. In some embodiments, the hormone is Cerebellin 1. In some embodiments, the hormone is CCK. In some embodiments, the hormone is GLP-1. In some embodiments, the hormone is PPY. In some embodiments, the hormone is PYY. In some embodiments, the hormone is Motilin. In some embodiments, the hormone is Angiotensin. In some embodiments, the hormone is Serotonin. In some embodiments, the hormone is GIP. In some embodiments, the hormone is Somatostatin. In some embodiments, the hormone is Gastrin. In some embodiments, the hormone is Secretin. In some embodiments, the hormone is Serotonin. In some embodiments, the hormone is NPW. In some embodiments, the hormone is VGF.

In some embodiments, the compound induces or increases expression and/or secretion of a hormone precursor. For example, in some embodiments, the compound induces or increases expression of a hormone precursor. For example, in some embodiments, the compound induces or increases secretion of a hormone precursor. In some embodiments, the compound decreases or ceases expression and/or secretion of a hormone precursor. For example, in some embodiments, the compound decreases or ceases expression of a hormone precursor. For example, in some embodiments, the compound decreases or ceases secretion of a hormone precursor. In some embodiments, the hormone precursor is GCG.

In some embodiments, the compound induces or increases expression of a hormone synthesizing enzyme. In some embodiments, the compound decreases or ceases expression of a hormone synthesizing enzyme. In some embodiments, the hormone synthesizing enzyme is TPH1.

In some embodiments, the target is PLXNB1. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is SLC22A17. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is FZD3. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is ROR1. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is LEPR. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is NISCH. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is EPHB3. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is DSTYK. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is EPOR. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is CHRM3. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is ITLN1. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is ITPR2. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is ABL2. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is ADGRL1. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is PKD2. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is GFRA1. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is RGMB. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is KREMEN1. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is ABL1. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is EEF1A1. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is GABBR2. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is MC1R. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments of the various methods and uses described herein, the human EEC is a D-cell. In some embodiments, the compound is a compound which targets a target in a D cell. In some embodiments, the compound is a compound which targets a cell surface receptor on a D cell. In some embodiments, the target is a target in a D-cell as described herein. In some embodiments, the target is a receptor selected from GFRA3, CALY, RAMP1, ADGRG1, TGFBR3, PTK7, GPR160, PVR, SEZ6L2 and FCGRT. The inventors have found that such cell surface receptors are expressed on D-cells. D-cells secrete SST (somatostatin). In some embodiments, the hormone is somatostatin. In some embodiments, the hormones are somatostatin and/or GCG (or GLP-1). Somatostatin inhibits secretion of most intestinal hormones, including GLP-1. Inhibition of Somatostatin can therefore, for example, enhance GLP-1 release. GLP-1 agonists are widely used for treatment of type 2 diabetes. Accordingly, in some embodiments, the disease or disorder is diabetes, e.g. type II diabetes. Thus, in some embodiments, a method for treating or preventing type II diabetes comprises administering a compound that targets a cell surface receptor on a D cell. Similarly, in some embodiments, there is provided a compound that targets a cell surface receptor on a D-cell for use in treating or preventing type II diabetes. In some embodiments, the compound reduces expression of somatostatin by the D cell. In some embodiments, a method for treating or preventing a disease or disorder in which Somatostatin is implicated comprises administering a compound that targets a target in a D-cell. In some embodiments, the invention provides a compound that targets a target in a D-cell for use in treating or preventing a disease or disorder in which Somatostatin is implicated. In some embodiments, the compound targets the transcription factor HHEX. In some embodiments, the compound is a knockout construct for the transcription factor HHEX or is an HHEX inhibitor. As described elsewhere herein, the inventors have found that knocking out HHEX inhibits production of Somatostatin. In some embodiments, the physiological effect is secretion of intestinal hormones, in particular inhibition of secretion of intestinal hormones. For example, in some embodiments, the physiological effect is inhibiting or decreasing secretion of one or more intestinal hormones. In some embodiments, the physiological effect is increasing or inducing secretion of one or more intestinal hormones. In some embodiments, the disease or disorder is a disease or disorder in which one or more of somatostatin, GCG and GLP-1 is implicated. In particular, in some embodiments, the disease or disorder is a disease or disorder in which somatostatin is implicated. In some embodiments, the disease or disorder is a disease or disorder in which one or more of Ghrelin, Cerebellin 1, CCK, GCG, GLP-1, PYY, PPY, Motilin, Angiotensin, Serotonin, GIP, Gastrin and Secretin is implicated. Examples of such diseases are described herein. Thus, modulating the expression and/or secretion of Somatostatin can be used to treat or prevent a wide variety of diseases.

In some embodiments, the compound induces or increases expression and/or secretion of somatostatin. For example, in some embodiments, the compound induces or increases expression of somatostatin. For example, in some embodiments, the compound induces or increases secretion of somatostatin. In some embodiments, the compound decreases or ceases expression and/or secretion of somatostatin. For example, in some embodiments, the compound decreases or ceases expression of somatostatin. For example, in some embodiments, the compound decreases or ceases secretion of somatostatin.

In some embodiments, the compound induces or increases expression and/or secretion of GCG. For example, in some embodiments, the compound induces or increases expression of GCG. For example, in some embodiments, the compound induces or increases secretion of GCG. In some embodiments, the compound decreases or ceases expression and/or secretion of GCG. For example, in some embodiments, the compound decreases or ceases expression of GCG. For example, in some embodiments, the compound decreases or ceases secretion of GCG.

In some embodiments, the compound induces or increases production of GLP-1. In some embodiments, the compound decreases or ceases production of GLP-1.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a disease or disorder in which somatostatin is implicated.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a disease or disorder treatable or preventable by ceasing or decreasing secretion and/or expression of Somatostatin.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a disease or disorder treatable or preventable by increasing or inducing secretion of one or more hormones and/or hormone precursors by one or more EECs and/or increasing or inducing production of one or more hormones from one or more hormone precursors by one or more EECs.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is selected from an appetite-related disease or disorder, a gut motility disease or disorder, diabetes, a neurodegenerative disease or disorder and depression.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a disease or disorder treatable or preventable by inducing or increasing secretion and/or expression of Somatostatin.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a disease or disorder treatable or preventable by decreasing or ceasing secretion of one or more hormones and/or hormone precursors by one or more EECs and/or decreasing or ceasing production of one or more hormones from one or more hormone precursors by one or more EECs.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is selected from an appetite-related disease or disorder, a gut motility disease or disorder, high acid secretion in the stomach and IBS.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. Somatostatin inhibits secretion of most intestinal hormones, including GLP-1. Thus, in some embodiments, the disease or disorder is a disease or disorder in which GLP-1 is implicated.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. Somatostatin inhibits secretion of most intestinal hormones, including GLP-1. Inhibition of Somatostatin may for example enhance GLP-1 release. Thus, in some embodiments, the disease or disorder is a disease or disorder treatable or preventable by increasing or inducing production of GLP-1.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. Somatostatin inhibits secretion of most intestinal hormones, including GLP-1. Increasing expression or secretion of Somatostatin may for example decrease GLP-1 release. Thus, in some embodiments, the disease or disorder is a disease or disorder treatable or preventable by decreasing or ceasing production of GLP-1.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is an appetite-related disease or disorder requiring appetite inhibition.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is an appetite-related disease or disorder requiring appetite inhibition, wherein the disease or disorder is obesity.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is an appetite-related disease or disorder requiring appetite inhibition, wherein the disease or disorder is Prader-Willi syndrome.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is an appetite-related disease or disorder requiring appetite inhibition, wherein the disease or disorder is diabetes.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is an appetite-related disease or disorder requiring appetite inhibition, wherein the disease or disorder is type II diabetes.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a disease or disorder requiring appetite stimulation.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a disease or disorder requiring appetite stimulation, wherein the disease or disorder is anorexia.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a disease or disorder requiring appetite stimulation, wherein the disease or disorder is cancer cachexia.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is diabetes.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is type II diabetes.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a gut motility disease or disorder.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a bowel movement disorder.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is Parkinson's disease.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is gastroparesis.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is a neurodegenerative disease.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is Parkinson's disease.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is Alzheimer's disease.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is depression.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is high acid secretion in the stomach.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is high acid secretion in the stomach caused by abnormal parietal cell activity in the stomach.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is indigestion caused by high acid secretion in the stomach.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is acid reflux caused by high acid secretion in the stomach.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is IBS.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is IBD.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is ulcerative colitis.

In some embodiments, the target is a target in a D-cell, as described herein. In some embodiments, the target is a receptor selected from GFRA3, CALY, RAMP1, ADGRG1, TGFBR3, PTK7, GPR160, PVR, SEZ6L2 and FCGRT. In some embodiments, the human EEC is a D-cell. In some embodiments, the hormone is somatostatin. In some embodiments, the disease or disorder is Crohn's disease.

In some embodiments of the various methods and uses described herein, the human EEC is an EC-cell. In some embodiments, the compound is a compound which targets a target in an EC cell. In some embodiments, the compound is a compound which targets a cell surface receptor on an EC cell. In some embodiments, the target is a target in an EC-cell as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. The inventors have found that such cell surface receptors are expressed on EC-cells. In some embodiments, the compound is Peptide YY (PYY) or Neuropeptide Y (NPY). PYY and NPY target the NPY1R receptor. In some embodiments, the compound is TSH. TSH targets the TSHR receptor. In some embodiments, the compound is Cocaine-and-amphetamine-regulated transcript protein (CARPT). CARPT targets the GPR68 receptor. In some embodiments, the compound is nonionic acid. Nonionic acid targets the OR51E1 receptor. In some embodiments, the compound is GABA. GABA targets the GABBR2 receptor. In some embodiments, the compound is dopamine. Dopamine targets the DRD2 receptor. EC-cells express the enzyme Tph1, which is involved in the synthesis of Serotonin. Thus, EC-cells secrete Serotonin. In some embodiments, the hormone synthesizing enzyme is TPH1. In some embodiments, the hormone is Serotonin. Serotonin is implicated in gut motility disorders (e.g. bowel movement disorders, Parkinson's disease, gastroparesis) and irritable bowel syndrome. Serotonin is elevated in IBS. Serotonin regulates bowel movement and activation of pain receptors. Accordingly, in some embodiments, the disease or disorder is a gut motility disorder or irritable bowel syndrome. In some embodiments, the gut motility disorder is selected from bowel movement disorders, Parkinson's disease and gastroparesis. For example, in some embodiments, the disease or disorder is select from gut motility disorders, bowel movement disorders, Parkinson's disease, gastroparesis and irritable bowel syndrome. In some embodiments, the disease or disorder is IBD. In some embodiments, the disease or disorder is ulcerative colitis. In some embodiments, the disease or disorder is Crohn's disease. In some embodiments, the disease or disorder is depression. In some embodiments, the disease or disorder is diabetes. Thus, in some embodiments, a method for treating such a disease or disorder, e.g. a gut motility disorder, diabetes, depression, irritable bowel syndrome, or IBD, comprises administering a compound that targets a target in an EC cell. Thus, in some embodiments, a method for treating such a disease or disorder, e.g. a gut motility disorder or irritable bowel syndrome, comprises administering a compound that targets a cell surface receptor on an EC cell. Similarly, in some embodiments, there is provided a compound that targets a target in an EC-cell for use in treating or preventing such a disease or disorder, e.g. a gut motility disorder, irritable bowel syndrome, diabetes, depression or IBD. In some embodiments, there is provided a compound that targets a cell surface receptor on an EC-cell for use in treating or preventing such a disease or disorder, e.g. a gut motility disorder or irritable bowel syndrome. EC-cells also secrete CHGA and CHGB. In some embodiments, the one or more hormones are one or more of (e.g. 2 or more or all 3 of) serotonin, CHGA and CHGB. For example, in some embodiments, the hormone is serotonin. In some embodiments, the disease or disorder is a disease or disorder in which one or more of Serotonin, CHGA and CHGB is implicated. For example, in some embodiments, the disease or disorder is a disease or disorder in which Serotonin is implicated. Examples of such diseases and disorders are described herein.

In some embodiments, the compound induces or increases production and/or secretion of serotonin. For example, in some embodiments, the compound induces or increases production of serotonin. For example, in some embodiments, the compound induces or increases secretion of serotonin. In some embodiments, the compound decreases or ceases production and/or secretion of serotonin. For example, in some embodiments, the compound decreases or ceases production of serotonin. For example, in some embodiments, the compound decreases or ceases secretion of serotonin.

In some embodiments, the compound induces or increases expression of TPH1. In some embodiments, the compound decreases or ceases expression of TPH1.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is a disease or disorder in which Serotonin is implicated.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is selected from IBS, depression, diabetes, a gut motility disorder and IBD.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is a disease or disorder treatable or preventable by inducing or increasing secretion and/or production of Serotonin.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is depression, a gut motility disorder or diabetes.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is depression.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is diabetes.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is type II diabetes.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is a gut motility disorder.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is a bowel movement disorder.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is Parkinson's disease.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is gastroparesis.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is a disease or disorder treatable or preventable by decreasing or ceasing secretion and/or production of Serotonin.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is IBS.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is IBD.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is ulcerative colitis.

In some embodiments, the target is a target in an EC-cell, as described herein. In some embodiments, the target is a receptor selected from OR51E1, MCOLN3, TRPA1, NPY1R, GPR160, FFAR2, ADGRG1, GABRB3, SSTR1, SLC7A1, FLVCR1, ERRFI1, GABARAP, GUCY2C, CNIH2, CD36, ADGRG4, GPR68 and TSHR. In some embodiments, the target is a cell surface receptor which is a GPCR selected from FFAR2, GPBAR1, NPY1R, CASR, GABBR2, GPR68, TSHR, DRD2, ADGRG4 and OR51E1. In some embodiments, the human EEC is an EC-cell. In some embodiments, the hormone is Serotonin. In some embodiments, the disease or disorder is Crohn's disease.

In some embodiments of the various methods and uses described herein, the human EEC is a K/G cell. In some embodiments, the compound is a compound which targets a target in a K/G-cell. In some embodiments, the compound is a compound which targets a cell surface receptor on a K/G-cell. In some embodiments, the target is a target in a K/G-cell as described herein. In some embodiments, the target is a receptor selected from SCTR, SSTR2, GUCY2C, CD44, CNIH2, CHRNA5, CALY, ABCC8, AHCYL1 and GRPR. The inventors have found that such cell surface receptors are expressed on K/G-cells. In some embodiments, the compound is Secretin (SCT). Secretin targets the SCTR receptor. In some embodiments, the compound is Gastrin-releasing-peptide (GRP). GRP targets the GRPR receptor. K/G-cells secrete GIP and GAST. In some embodiments, the one or more hormones are one or both of GIP and Gastrin. In some embodiments, the hormone is GIP. In some embodiments, the hormone is Gastrin. GIP is implicated in insulin secretion. Accordingly, in some embodiments, the disease or disorder is diabetes, e.g. type II diabetes. GIP is also implicated in neurodegenerative diseases and disorders, such as Alzheimer's disease and Parkinson's disease. Thus, in some embodiments, the disease or disorder is a neurodegenerative disease or disorder, e.g. Alzheimer's disease or Parkinson's disease. In some embodiments, the disease or disorder is Alzheimer's disease. In some embodiments, the disease or disorder is Parkinson's disease. Gastrin is implicated in parietal cell activity in the stomach. Accordingly, in some embodiments, the disease or disorder is high acid secretion in the stomach, for example, indigestion or acid reflux. Thus, in some embodiments, a method for treating or preventing diabetes, a neurodegenerative disease or disorder or high acid secretion in the stomach comprises administering a compound that targets a target in a K/G-cell. Thus, in some embodiments, a method for treating or preventing diabetes or high acid secretion in the stomach comprises administering a compound that targets a cell surface receptor on a K/G-cell. Similarly, in some embodiments, there is provided a compound that targets a target in a K/G-cell for use in treating or preventing diabetes, a neurodegenerative disease or disorder or high acid secretion in the stomach. In some embodiments, there is provided a compound that targets a cell surface receptor on a K/G-cell for use in treating or preventing diabetes or high acid secretion in the stomach. In some embodiments, the physiological effect is insulin secretion. In some embodiments, the physiological effect is parietal cell activity in the stomach. In some embodiments, the disease or disorder is a disease or disorder in which one or both of GIP and Gastrin is implicated. In some embodiments, the disease or disorder is a disease or disorder in which GIP is implicated. In some embodiments, the disease or disorder is a disease or disorder in which Gastrin is implicated. Examples of such diseases and disorders are described herein.

In some embodiments, the compound induces or increases expression and/or secretion of GIP. For example, in some embodiments, the compound induces or increases expression of GIP. For example, in some embodiments, the compound induces or increases secretion of GIP. In some embodiments, the compound decreases or ceases expression and/or secretion of GIP. For example, in some embodiments, the compound decreases or ceases expression of GIP. For example, in some embodiments, the compound decreases or ceases secretion of GIP.

In some embodiments, the compound induces or increases expression and/or secretion of Gastrin. For example, in some embodiments, the compound induces or increases expression of Gastrin. For example, in some embodiments, the compound induces or increases secretion of Gastrin. In some embodiments, the compound decreases or ceases expression and/or secretion of Gastrin. For example, in some embodiments, the compound decreases or ceases expression of Gastrin. For example, in some embodiments, the compound decreases or ceases secretion of Gastrin.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is GIP. Thus, in some embodiments, the disease or disorder is a disease or disorder in which GIP is implicated.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is GIP. In some embodiments, the disease or disorder is selected from diabetes and a neurodegenerative disease or disorder.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is GIP. Thus, in some embodiments, the disease or disorder is a disease or disorder treatable or preventable by inducing or increasing secretion and/or expression of GIP.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is GIP. In some embodiments, the disease or disorder is diabetes.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is GIP. In some embodiments, the disease or disorder is type II diabetes.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is GIP. In some embodiments, the disease or disorder is a neurodegenerative disease or disorder.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is GIP. In some embodiments, the disease or disorder is Parkinson's Disease.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is GIP. In some embodiments, the disease or disorder is Alzheimer's Disease.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is GIP. In some embodiments, the disease or disorder is a disease or disorder treatable or preventable by ceasing or decreasing secretion and/or expression of GIP.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is gastrin. In some embodiments, the disease or disorder is a disease or disorder in which gastrin is implicated.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is gastrin. In some embodiments, the disease or disorder is a disease or disorder treatable or preventable by ceasing or decreasing secretion and/or expression of Gastrin.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is gastrin. In some embodiments, the disease or disorder is selected from high acid secretion in the stomach, indigestion caused by high acid secretion in the stomach and acid reflux caused by high acid secretion in the stomach.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is gastrin. In some embodiments, the disease or disorder is selected from high acid secretion in the stomach, indigestion caused by high acid secretion in the stomach and acid reflux caused by high acid secretion in the stomach, wherein the high acid secretion is caused by abnormal parietal cell activity in the stomach.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is gastrin. In some embodiments, the disease or disorder is high acid secretion in the stomach.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is gastrin. In some embodiments, the disease or disorder is high acid secretion in the stomach caused by abnormal parietal cell activity in the stomach.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is gastrin. In some embodiments, the disease or disorder is indigestion caused by high acid secretion in the stomach.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is gastrin. In some embodiments, the disease or disorder is acid reflux caused by high acid secretion in the stomach.

In some embodiments, the target is a receptor selected from SCTR, SSTR2, GUCY2C, CD44, CNIH2, CHRNA5, CALY, ABCC8, AHCYL1 and GRPR. In some embodiments, the target is a target in a K/G-cell, as described herein. In some embodiments, the human EEC is a K/G cell. In some embodiments, the hormone is gastrin. In some embodiments, the disease or disorder is a disease or disorder treatable or preventable by inducing or increasing secretion and/or expression of Gastrin.

In some embodiments of the various methods and uses described herein, the human EEC is an L-cell. In some embodiments, the compound is a compound which targets a target in an L-cell. In some embodiments, the compound is a compound which targets a cell surface receptor on an L-cell. In some embodiments, the target is a target in an L-cell as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. The inventors have found that such cell surface receptors are expressed on L-cells. In some embodiments, the compound is Secretin. Secretin targets the SCTR. In some embodiments, the compound is head activator (HA). HA targets GPR37. In some embodiments, the compound is selected from a melanocortin, ACTH and MSH. These target MC1R. L-cells secrete GCG and NTS. GCG is the hormone precursor of GLP-1. GLP-1 reduces gastric emptying and motility. Thus, in some embodiments, the disease or disorder is gut motility disorder (e.g. bowel movement disorders, Parkinson's disease, gastroparesis). In addition, GLP-1 is implicated in appetite inhibition. Thus, in some embodiments, the disease or disorder is an appetite-related disease or disorder. Thus, in some embodiments, the disease or disorder is obesity, anorexia, cancer cachexia or diabetes, e.g. obesity or diabetes. For example, in some embodiments, the disease or disorder is selected from obesity, diabetes and Prader-Willi syndrome. In some embodiments, the disease or disorder is anorexia or cancer cachexia. GLP-1 is also implicated in insulin secretion. For example, GLP-1 agonists are widely used for treatment of type II diabetes. Thus, in some embodiments, the disease or disorder is diabetes, e.g. type II diabetes. Thus, in some embodiments, the disease or disorder is selected from a gut motility disorder, a bowel movement disorder, Parkinson's disease, gastroparesis, obesity, anorexia and diabetes (e.g. type II diabetes). A number of preclinical studies have demonstrated that GLP-1s are beneficial when tested in animal models of Parkinson's Disease, and it is thought that GLP-1 affects neurological and cognitive functions, as well as its regulatory effect on glucose metabolism. In some embodiments, the disease or disorder is a neurodegenerative disease or disorder such as Parkinson's Disease or Alzheimer's Disease. Thus, in some embodiments, a method for treating or preventing a disease or disorder selected from obesity, anorexia, cancer cachexia, diabetes, Prader-Willi syndrome, a gut motility disorder and a neurodegenerative disease or disorder comprises administering a compound that targets a target in an L-cell. In some embodiments, a method for treating or preventing a disease or disorder selected from obesity, anorexia, cancer cachexia, diabetes, Prader-Willi syndrome, a gut motility disorder and/or a neurodegenerative disease or disorder comprises administering a compound that targets a cell surface receptor on an L-cell. Similarly, in some embodiments, there is provided a compound that targets a target in an L-cell for use in treating or preventing a disease or disorder selected from obesity, anorexia, cancer cachexia, Prader-Willi syndrome, a gut motility disorder, diabetes and a neurodegenerative disorder. In some embodiments, there is provided a compound that targets a cell surface receptor on an L-cell for use in treating or preventing a disease or disorder selected from obesity, anorexia, cancer cachexia, Prader-Willi syndrome, a gut motility disorder, diabetes and a neurodegenerative disorder. In some embodiments, the disease or disorder is selected from obesity, anorexia, cancer cachexia, diabetes, a gut motility disorder and/or a neurodegenerative disease or disorder. In some embodiments which comprise treating or preventing a gut motility disorder, the compound reduces GLP-1 levels, e.g. reduces expression of GCG. In some embodiments, the compound reduces GLP-1 levels, e.g. reduces expression and/or secretion of GCG and/or reduces processing of GCG to GLP-1. In some embodiments, the compound increases GLP-1 levels, e.g. increases expression and/or secretion of GCG and/or increases processing of GCG to GLP-1. L-cells also secrete PYY and PPY. In some embodiments, the one or more hormones are one or more of (e.g. 1, 2, 3, 4 or more or all 5 of) GCG-derived products (GLP-1, GLP-2), Peptide YY (PYY), Pancreatic Polypeptide Y (PPY) and Neurotensin (NTS). For example, in some embodiments, the one or more hormones and/or hormone precursors are GCG and/or NTS. For example, in some embodiments, the one or more hormones and/or hormone precursors are GCG, GLP-1 and/or NTS, e.g. GCG and/or GLP-1. In some embodiments, the hormone precursor is GCG. In some embodiments, the hormone is GLP-1. In some embodiments, the hormone is GLP-2. In some embodiments, the hormone is PYY. In some embodiments, the hormone is PPY. In some embodiments, the hormone is NTS. In some embodiments, the physiological effect is gastric emptying, gut motility, insulin secretion, appetite inhibition, appetite stimulation or neurological and cognitive functioning. In some embodiments, the disease or a disorder is a disease or disorder in which one or more of GCG-derived products, GLP-1, GLP-2, Peptide YY (PYY), Pancreatic Polypeptide Y (PPY) and Neurotensin (NTS) is implicated. For example, in some embodiments, the disease or disorder is a disease or disorder in which GLP-1 is implicated. In some embodiments, the disease or disorder is a disease or disorder in which PYY is implicated. In some embodiments, the disease or disorder is a disease or disorder in which PPY is implicated. In some embodiments, the disease or disorder is a disease or disorder in which GCG is implicated. In some embodiments, the disease or disorder is a disease or disorder in which GLP-2 is implicated. Examples of such diseases and disorders are described herein.

In some embodiments, the compound induces or increases expression and/or secretion of GCG. For example, in some embodiments, the compound induces or increases expression of GCG. For example, in some embodiments, the compound induces or increases secretion of GCG. In some embodiments, the compound decreases or ceases expression and/or secretion of GCG. For example, in some embodiments, the compound decreases or ceases expression of GCG. For example, in some embodiments, the compound decreases or ceases secretion of GCG.

In some embodiments, the compound induces or increases expression and/or secretion of NTS. For example, in some embodiments, the compound induces or increases expression of NTS. For example, in some embodiments, the compound induces or increases secretion of NTS. In some embodiments, the compound decreases or ceases expression and/or secretion of NTS. For example, in some embodiments, the compound decreases or ceases expression of NTS. For example, in some embodiments, the compound decreases or ceases secretion of NTS.

In some embodiments, the compound induces or increases expression and/or secretion of PYY. For example, in some embodiments, the compound induces or increases expression of PYY. For example, in some embodiments, the compound induces or increases secretion of PYY. In some embodiments, the compound decreases or ceases expression and/or secretion of PYY. For example, in some embodiments, the compound decreases or ceases expression of PYY. For example, in some embodiments, the compound decreases or ceases secretion of PYY.

In some embodiments, the compound induces or increases expression and/or secretion of PPY. For example, in some embodiments, the compound induces or increases expression of PPY. For example, in some embodiments, the compound induces or increases secretion of PPY. In some embodiments, the compound decreases or ceases expression and/or secretion of PPY. For example, in some embodiments, the compound decreases or ceases expression of PPY. For example, in some embodiments, the compound decreases or ceases secretion of PPY.

In some embodiments, the compound induces or increases production of GLP-1. In some embodiments, the compound decreases or ceases production of GLP-1.

In some embodiments, the compound induces or increases production of GLP-2. In some embodiments, the compound decreases or ceases production of GLP-2.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is a disease or disorder in which GLP-1 is implicated.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is a disease or disorder treatable or preventable by increasing or inducing production of GLP-1.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is selected from a disease or disorder requiring appetite inhibition, diabetes and a neurodegenerative disease or disorder.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is a disease or disorder requiring appetite inhibition.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162.

In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is obesity.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162.

In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is Prader-Willi syndrome.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is diabetes.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is type II diabetes.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is a neurodegenerative disease or disorder.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is Parkinson's disease.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is Alzheimer's disease.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is a disease or disorder treatable or preventable by decreasing or ceasing production of GLP-1.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162.

In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is selected from a disease or disorder requiring appetite stimulation and a gut motility disorder.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is a disease or disorder requiring appetite stimulation.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is anorexia.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is cancer cachexia.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is a gut motility disorder.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is a bowel movement disorder.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is Parkinson's disease.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-1. Thus it will be understood that in some embodiments, the hormone precursor is GCG. In some embodiments, the disease or disorder is gastroparesis.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-2. Thus it will be understood that in some embodiments, the hormone precursor is GCG. In some embodiments, the disease or disorder is a disease or disorder in which GLP-2 is implicated.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-2. Thus it will be understood that in some embodiments, the hormone precursor is GCG. In some embodiments, the disease or disorder is treatable or preventable by increasing or inducing production of GLP-2.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is GLP-2. Thus it will be understood that in some embodiments, the hormone precursor is GCG. In some embodiments, the disease or disorder is treatable or preventable by decreasing or ceasing production of GLP-2.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is NTS. In some embodiments, the disease or disorder is a disease or disorder in which NTS is implicated.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is NTS. In some embodiments, the disease or disorder is treatable or preventable by increasing or inducing expression and/or secretion of NTS.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is NTS. In some embodiments, the disease or disorder is treatable or preventable by decreasing or ceasing expression and/or secretion of NTS.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is a disease or disorder in which PPY is implicated.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is a disease or disorder requiring appetite inhibition or a disease or disorder requiring appetite stimulation.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is treatable or preventable by increasing or inducing expression and/or secretion of PPY.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is a disease or disorder requiring appetite inhibition.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is obesity.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is diabetes.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is type II diabetes.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is Prader-Willi Syndrome.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is treatable or preventable by decreasing or ceasing expression and/or secretion of PPY.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is a disease or disorder requiring appetite stimulation.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is anorexia.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PPY. In some embodiments, the disease or disorder is cancer cachexia.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is a disease or disorder in which PYY is implicated.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is a disease or disorder requiring appetite inhibition or a disease or disorder requiring appetite stimulation.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is treatable or preventable by increasing or inducing expression and/or secretion of PYY.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is a disease or disorder requiring appetite inhibition.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is obesity.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is diabetes.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is type II diabetes.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is Prader-Willi Syndrome.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is treatable or preventable by decreasing or ceasing expression and/or secretion of PYY.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is a disease or disorder requiring appetite stimulation.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is anorexia.

In some embodiments, the target is a target in an L-cell, as described herein. In some embodiments, the target is a receptor selected from GPBAR1, ABCC8, GUCY2C, CALY, SCTR, FGFR1, SSTR2, CD44, IL17RB, CASR, PTPRN2, ADGRG1, KIT, MAGED1, GPR35, TGFBR3, PVR, CNIH2, PKD1, INSR, TMEM97, CLDN3, GABARAP, SEZ6L2, ITGB1, SSTR5 and GPR162. In some embodiments, the target is a cell surface receptor which is a GPCR selected from FFAR2, GPBAR1, GPR162, GPR37, ADGRD1, ADGRA2, MC1R, OR7E47P, AGTR1, CASR, SCTR, SSTR5 and SSTR2. In some embodiments, the human EEC is an L-cell. In some embodiments, the hormone is PYY. In some embodiments, the disease or disorder is cancer cachexia.

In some embodiments of the various methods and uses described herein, the human EEC is an M/X-cell. In some embodiments, the compound is a compound which targets a target in an M/X-cell. In some embodiments, the compound is a compound which targets a cell surface receptor on an M/X-cell. In some embodiments, the target is a target in an M/X-cell as described herein. In some embodiments, the target is a receptor selected from ASGR1, EPHA4, FFAR4, GFRA3, NTRK2, SSTR2, TNFRSF21, ABCC8, CD44, CALY, PVR and IL20RA. The inventors have found that such cell surface receptors are expressed on M/X-cells. In some embodiments, the compound is selected from one or more of IL-10, IL-19, IL-20, IL-24 and IL-26. In some embodiments, a composition comprising two or more (e.g. 2, 3, 4, or more or all 5) of IL-10, IL-19, IL-20, IL-24 and IL-26 is used. These target the IL20RA. M/X-cells secrete MLN and GHRL. Ghrelin is implicated in appetite stimulation. For example, Ghrelin receptor agonists are currently being tested in clinical trials and show very positive results in terms of increasing appetite. Thus, in some embodiments, the disease or disorder is anorexia or cancer cachexia. In some embodiments, the disease or disorder is selected from obesity, diabetes and/or Prader-Willi syndrome. In some embodiments, the disease or disorder is obesity. Motilin is implicated in gut motility disorders. For example, the Motilin agonist erythromycin is clinically used to stimulate peristalsis. Thus, in some embodiments, the disease or disorder is a gut motility disorder (e.g. bowel movement disorders, Parkinson's disease, gastroparesis). Accordingly, in some embodiments, the disease or disorder is selected from an appetite-related disease or disorder (e.g. anorexia, cancer cachexia, obesity, diabetes, Prader-Willi syndrome) and a gut motility disorder (e.g. a bowel movement disorder, Parkinson's disease or gastroparesis). In some embodiments, the disease or disorder is selected from anorexia, cancer cachexia, obesity, gut motility disorders, bowel movement disorders, Parkinson's disease and gastroparesis. Thus, in some embodiments, a method for treating or preventing anorexia, cancer cachexia, obesity, diabetes, Prader-Willi syndrome or a gut motility disorder comprises administering a compound that targets a target in an M/X-cell. In some embodiments, a method for treating or preventing anorexia, cancer cachexia, obesity, diabetes, Prader-Willi syndrome or a gut motility disorder comprises administering a compound that targets a cell surface receptor on an M/X-cell. Similarly, in some embodiments, there is provided a compound that targets a target in an M/X-cell for use in treating or preventing anorexia, cancer cachexia, obesity, diabetes, Prader-Willi syndrome or a gut motility disorder. In some embodiments, there is provided a compound that targets a cell surface receptor on an M/X-cell for use in treating or preventing anorexia, cancer cachexia, obesity or a gut motility disorder. In some embodiments, the disease or disorder is selected from anorexia, cancer cachexia, obesity, or a gut motility disorder. Motilin is also associated with hunger. Thus, in some embodiments, the disease or disorder is a disease or disorder requiring appetite stimulation. In some embodiments, the disease or disorder is anorexia. In some embodiments, the disease or disorder is cancer cachexia. In addition, Motilin is associated with diabetes, in particular type II diabetes. Thus, in some embodiments, the disease or disorder is diabetes. In some embodiments, the diabetes is type II diabetes. Motilin is also associated with increased gallbladder emptying. Thus, in some embodiments, the disease or disorder is a biliary movement disorder. In some embodiments, the disease or disorder is biliary diskinesia. In some embodiments, the disease or disorder is a disease or disorder requiring appetite inhibition. In some embodiments, the disease or disorder is obesity. In some embodiments, the disease or disorder is diabetes. In some embodiments, the disease or disorder is type II diabetes. In some embodiments, the disease or disorder is Prader-Willi syndrome. M/X cells also secrete angiotensin and cerebellin 1. In some embodiments, the one or more hormones are one or more of (e.g. 1, 2, 3, or more or all 4 of) ghrelin, motilin, angiotensin and cerebellin 1. In some embodiments, the one or more hormones are one or both of ghrelin and motilin. In some embodiments, the hormone is ghrelin. In some embodiments, the hormone is motilin. In some embodiments, the hormone is angiotensin. In some embodiments, the hormone is cerebellin 1. In some embodiments, the physiological effect is appetite stimulation, appetite inhibition or gut motility (e.g. peristalsis). In some embodiments, the disease or disorder is a disease or disorder in which one or more of ghrelin, motilin, angiotensin and cerebellin 1 is implicated. In some embodiments, the disease or disorder is a disease or disorder in which ghrelin is implicated. In some embodiments, the disease or disorder is a disease or disorder in which motilin is implicated. In some embodiments, the disease or disorder is a disease or disorder in which angiotensin is implicated. In some embodiments, the disease or disorder is a disease or disorder in which cerebellin 1 is implicated. Examples of such diseases and disorders are described herein. In some embodiments, the disease or disorder is anorexia. In some embodiments, the disease or disorder is cancer cachexia. In some embodiments, the disease or disorder is obesity. In some embodiments, the disease or disorder is diabetes. In some embodiments, the disease or disorder is type II diabetes. In some embodiments, the disease or disorder is Prader-Willi syndrome. In some embodiments, the disease or disorder is a gut motility disorder. In some embodiments, the disease or disorder is a bowel movement disorder. In some embodiments, the disease or disorder is Parkinson's disease. In some embodiments, the disease or disorder is gastroparesis. In some embodiments, the disease or disorder is a biliary movement disorder. In some embodiments, the disease or disorder is biliary diskinesia.

In some embodiments, the compound induces or increases expression and/or secretion of ghrelin. For example, in some embodiments, the compound induces or increases expression of ghrelin. For example, in some embodiments, the compound induces or increases secretion of ghrelin. In some embodiments, the compound decreases or ceases expression and/or secretion of ghrelin. For example, in some embodiments, the compound decreases or ceases expression of ghrelin. For example, in some embodiments, the compound decreases or ceases secretion of ghrelin.

In some embodiments, the compound induces or increases expression and/or secretion of motilin. For example, in some embodiments, the compound induces or increases expression of motilin. For example, in some embodiments, the compound induces or increases secretion of motilin. In some embodiments, the compound decreases or ceases expression and/or secretion of motilin. For example, in some embodiments, the compound decreases or ceases expression of motilin. For example, in some embodiments, the compound decreases or ceases secretion of motilin.

In some embodiments, the compound induces or increases expression and/or secretion of angiotensin. For example, in some embodiments, the compound induces or increases expression of angiotensin. For example, in some embodiments, the compound induces or increases secretion of angiotensin. In some embodiments, the compound decreases or ceases expression and/or secretion of angiotensin. For example, in some embodiments, the compound decreases or ceases expression of angiotensin. For example, in some embodiments, the compound decreases or ceases secretion of angiotensin.

In some embodiments, the compound induces or increases expression and/or secretion of cerebellin 1. For example, in some embodiments, the compound induces or increases expression of cerebellin 1. For example, in some embodiments, the compound induces or increases secretion of cerebellin 1. In some embodiments, the compound decreases or ceases expression and/or secretion of cerebellin 1. For example, in some embodiments, the compound decreases or ceases expression of cerebellin 1. For example, in some embodiments, the compound decreases or ceases secretion of cerebellin 1.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is a disease or disorder in which Motilin is implicated.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is a disease or disorder which is treatable or preventable by increasing or inducing expression and/or secretion of Motilin.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is a gut motility disorder.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is a bowel movement disorder.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is Parkinson's disease.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is gastroparesis.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is a disease or disorder requiring appetite stimulation.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is anorexia.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is cancer cachexia.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is diabetes.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is type II diabetes.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is a biliary movement disorder.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is biliary diskinesia.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is a disease or disorder which is treatable or preventable by decreasing or ceasing expression and/or secretion of Motilin.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is a disease or disorder requiring appetite inhibition.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is obesity.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is diabetes.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is type II diabetes.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Motilin. In some embodiments, the disease or disorder is Prader-Willi syndrome.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Angiotensin. In some embodiments, the disease or disorder is a disease or disorder in which Angiotensin is implicated.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Angiotensin. In some embodiments, the disease or disorder is a disease or disorder which is treatable or preventable by increasing or inducing expression and/or secretion of Angiotensin.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Angiotensin. In some embodiments, the disease or disorder is a gut motility disorder.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Angiotensin. In some embodiments, the disease or disorder is a bowel movement disorder.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Angiotensin. In some embodiments, the disease or disorder is Parkinson's disease. In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Angiotensin. In some embodiments, the disease or disorder is gastroparesis.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Angiotensin. In some embodiments, the disease or disorder is a disease or disorder which is treatable or preventable by decreasing or ceasing expression and/or secretion of Angiotensin.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is a disease or disorder in which Ghrelin is implicated.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is a disease or disorder which is treatable or preventable by increasing or inducing expression and/or secretion of Ghrelin.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is a disease or disorder requiring appetite stimulation.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the human EEC is an M/X-cell. In some embodiments, the hormone is Ghrelin. In some embodiments, the disease or disorder is anorexia.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is cancer cachexia.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is a disease or disorder which is treatable or preventable by decreasing or ceasing expression and/or secretion of Ghrelin.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is a disease or disorder requiring appetite inhibition.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is obesity.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is diabetes.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is type II diabetes.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Ghrelin. In some embodiments, the disease or disorder is Prader-Willi syndrome.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is a disease or disorder in which Cerebellin 1 is implicated.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is a disease or disorder which is treatable or preventable by increasing or inducing expression and/or secretion of Cerebellin 1.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is a disease or disorder requiring appetite stimulation.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is anorexia.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is cancer cachexia.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is a disease or disorder which is treatable or preventable by decreasing or ceasing expression and/or secretion of Cerebellin 1.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is a disease or disorder requiring appetite inhibition.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is obesity.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is diabetes.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is type II diabetes.

In some embodiments, the target is a receptor selected from ASGR1, EPHA4, FFAR4, GFRA3, NTRK2, SSTR2, TNFRSF21, ABCC8, CD44, CALY, PVR and IL20RA. In some embodiments, the target is a target in an M/X-cell, as described herein. In some embodiments, the human EEC is an M/X cell. In some embodiments, the hormone is Cerebellin 1. In some embodiments, the disease or disorder is Prader-Willi syndrome.

In some embodiments, the human EECs are human ECs and/or the one or more targets are Dopa decarboxylase (DDC) and/or SLC18A1. Dopa decarboxylase is involved in serotonin biosynthesis and SLC18A1 is involved in serotonin transport (Lohoff et al., 2006). Accordingly, by way of non-limiting example, the invention provides an isolated population of human ECs which express Dopa decarboxylase (DDC) and/or SLC18A1. The invention likewise provides one or more isolated human ECs which express Dopa decarboxylase (DDC) and/or SLC18A1. In addition, the invention provides a pharmaceutical composition comprising an isolated population of human ECs which express Dopa decarboxylase (DDC) and/or SLC18A1. The invention further provides the use of expression of Dopa decarboxylase (DDC) and/or SLC18A1 as a marker for human ECs. Also provided is a method for isolating a human EC or a population of human ECs comprising selecting cells which express Dopa decarboxylase (DDC) and/or SLC18A1. Likewise, the invention provides a method for identifying a human EC or population of human EECs comprising selecting cells which express Dopa decarboxylase (DDC) and/or SLC18A1. Also provided is the use of Dopa decarboxylase (DDC) and/or SLC18A1 as one or more drug targets. For example, the invention provides a method for identifying/validating Dopa decarboxylase (DDC) and/or SLC18A1 as a drug target in EECs, wherein the method comprises (i) contacting one or more intestinal reporter organoids of the invention with a compound specific for Dopa decarboxylase (DDC) and/or SLC18A1; and (ii) determining whether hormone secretion is affected. In some embodiments, the physiological effect of Dopa decarboxylase is serotonin biosynthesis. The invention therefore further provides a method for modulating serotonin biosynthesis comprising modulating expression of Dopa decarboxylase in human ECs. Similarly, in some embodiments, the physiological effect of SLC18A1 is serotonin transport. Accordingly, the method provides a method for modulating serotonin transport comprising modulating expression of SLC18A1 in human ECs. In some embodiments, the disease or disorder is a disease or disorder in which Serotonin is implicated. Serotonin is implicated in IBS. Thus, in some embodiments, the disease or disorder is IBS. Serotonin is also implicated in gut motility diseases and disorders. Thus, in some embodiments, the disease or disorder is a gut motility disorder, for example, a bowel movement disorder, Parkinson's disease or gastroparesis. In some embodiments, the physiological effect is gut motility. In some embodiments, the disease or disorder is depression. In some embodiments, the disease or disorder is diabetes (in particular type II diabetes). In some embodiments, the disease or disorder is IBD. In some embodiments, the disease or disorder is Crohn's disease. In some embodiments, the disease or disorder is ulcerative colitis. In some embodiments, the physiological effect is bowel movement and/or activation of pain receptors. In some embodiments, the hormone is serotonin.

In some embodiments, the human EECs are human ECs and/or the target is Dopa decarboxylase (DDC) and/or SLC18A1. In some embodiments, the hormone is serotonin. In some embodiments, the disease or disorder is a disease or disorder in which Serotonin is implicated.

In some embodiments, the human EECs are human ECs and/or the target is Dopa decarboxylase (DDC) and/or SLC18A1. In some embodiments, the hormone is serotonin. In some embodiments, the disease or disorder is selected from IBS, depression, diabetes, a gut motility disorder and IBD.

In some embodiments, the human EECs are human ECs and/or the target is Dopa decarboxylase (DDC) and/or SLC18A1. In some embodiments, the hormone is Serotonin. In some embodiments, the disease or disorder is a disease or disorder treatable or preventable by inducing or increasing secretion and/or production of Serotonin.

In some embodiments, the human EECs are human ECs and/or the target is Dopa decarboxylase (DDC) and/or SLC18A1. In some embodiments, the hormone is serotonin. In some embodiments, the disease or disorder is depression, a gut motility disorder or diabetes.

In some embodiments, the human EECs are human ECs and/or the target is Dopa decarboxylase (DDC) and/or SLC18A1. In some embodiments, the hormone is serotonin. In some embodiments, the disease or disorder is depression.

In some embodiments, the human EECs are human ECs and/or the target is Dopa decarboxylase (DDC) and/or SLC18A1. In some embodiments, the hormone is serotonin. In some embodiments, the disease or disorder is diabetes.

In some embodiments, the human EECs are human ECs and/or the target is Dopa decarboxylase (DDC) and/or SLC18A1. In some embodiments, the hormone is serotonin. In some embodiments, the disease or disorder is type II diabetes.

In some embodiments, the human EECs are human ECs and/or the target is Dopa decarboxylase (DDC) and/or SLC18A1. In some embodiments, the hormone is serotonin. In some embodiments, the disease or disorder is a gut motility disorder.

In some embodiments, the human EECs are human ECs and/or the target is Dopa decarboxylase (DDC) and/or SLC18A1. In some embodiments, the hormone is serotonin. In some embodiments, the disease or disorder is a bowel movement disorder.

In some embodiments, the human EECs are human ECs and/or the target is Dopa decarboxylase (DDC) and/or SLC18A1. In some embodiments, the hormone is serotonin. In some embodiments, the disease or disorder is Parkinson's disease.

In some embodiments, the human EECs are human ECs and/or the target is Dopa decarboxylase (DDC) and/or SLC18A1. In some embodiments, the hormone is serotonin. In some embodiments, the disease or disorder is gastroparesis.

In some embodiments, the human EECs are human ECs and/or the target is Dopa decarboxylase (DDC) and/or SLC18A1. In some embodiments, the hormone is serotonin. In some embodiments, the disease or disorder is a disease or disorder treatable or preventable by decreasing or ceasing secretion and/or production of Serotonin.

In some embodiments, the human EECs are human ECs and/or the target is Dopa decarboxylase (DDC) and/or SLC18A1. In some embodiments, the hormone is serotonin. In some embodiments, the disease or disorder is IBS.

In some embodiments, the human EECs are human ECs and/or the target is Dopa decarboxylase (DDC) and/or SLC18A1. In some embodiments, the hormone is serotonin. In some embodiments, the disease or disorder is IBD.

In some embodiments, the human EECs are human ECs and/or the target is Dopa decarboxylase (DDC) and/or SLC18A1. In some embodiments, the hormone is serotonin. In some embodiments, the disease or disorder is ulcerative colitis.

In some embodiments, the human EECs are human ECs and/or the target is Dopa decarboxylase (DDC) and/or SLC18A1. In some embodiments, the hormone is serotonin. In some embodiments, the disease or disorder is Crohn's disease.

In some embodiments, the human EECs are human ECs and/or the target is secretogranin (SCG2). The inventors have found that SCG2 expression is higher in human ECs than in other EEC subtypes. In addition, SCG2 expression was higher in proximal gut ECs compared to distal gut ECs. In some embodiments, high expression of SCG2 is used as a marker for human ECs. In some embodiments, high expression of SCG2 is used as a marker for human ECs from the proximal intestine. In some embodiments, a high level of secretogranin SCG2 is expressed or a high level of secretogranin SCG2 expression is selected for. For example, in some embodiments, EECs with the highest level of SCG2 expression are selected in order to select human ECs, for example, in order to select human ECs from the proximal intestine. In some embodiments, the high level of expression of SCG2 is 10 or more SCG2 transcripts for each 3000 total transcripts in the cell, wherein the total transcripts relate to the transcripts of any genes in the cell. In some embodiments, SCG2 is expressed or expression of SCG2 is selected for. In some embodiments, the human ECs are derived from or derivable from proximal small intestine organoids of the invention. In some embodiments, the human ECs are from the proximal small intestine.

In some embodiments, the human EECs are human ECs and/or the target is the olfactory receptor OR51E1. OR51E1 is a marker of serotonin-producing neuroendocrine tumors in man (Cui et al., 2013) and therefore is implicated in the disease. Neuroendocrine tumours derive from EECs. In some embodiments, the human EC is a human neuroendocrine tumour cell. However, in some embodiments, the human EC is not a human neuroendocrine tumour cell. In some embodiments, the disease or disorder is neuroendocrine cancer or a neuroendocrine tumour. Accordingly, by way of non-limiting example, the invention provides a method for identifying/screening/validating a compound for suitability for treating a neuroendocrine tumour, wherein the method comprises (i) contacting a human intestinal organoid of the invention with the compound; and (ii) determining whether the compound modulates expression of, agonises or antagonises in human ECs OR51E1. The invention provides the use of OR51E1 in human ECs as a drug target. Also provided is a method for identifying/screening/validating a compound for suitability for treating a neuroendocrine tumour, wherein the method comprises (i) contacting a human intestinal reporter organoid of the invention with the compound; and (ii) determining whether hormone secretion is affected. In some embodiments, hormone secretion is affected through action of the compound on OR51E1. The invention further provides a method for treating a neuroendocrine tumour comprising administering a compound that targets OR51E1 in human ECs. In some embodiments, the invention provides a compound for use in treating a neuroendocrine tumour, wherein the treating comprises targeting OR51E1 in human ECs with the compound. Also provided is a method for treating a neuroendocrine tumour, comprising targeting human ECs, for example, in order to kill the human ECs. The invention further provides a method for treating a neuroendocrine tumour, comprising administering a compound that targets human ECs, for example, in order to kill the human ECs. Examples of suitable targets on human ECs are provided herein.

In some embodiments, the human EECs are human ECs and/or the one or more targets are one or more of (e.g. 1, 2, 3 or all 4 of) Dopa decarboxylase (DDC), SLC18A1, secretogranin (SCG2) and OR51E1. In some embodiments, the one or more targets additionally comprise the prototypical EC markers CHGB and/or GPR112 and/or TPH1.

In some embodiments, the human EECs are human G cells and/or the one or more targets are gastrin (GAST) and/or the receptor for Gastrin-releasing peptide, GRPR. Cells producing Gastrin (Gast) are largely restricted to the mouse stomach (Engelstoft et al., 2013a), whereas in man expression continues more distally along the GI tract in EECs termed G-cells. In some embodiments, the human EECs are human G/K cells and/or the one or more targets are one or more of (e.g. at least 1, 2 or all 3 of) gastrin (GAST), incretin GIP and/or GPRP. Incretin GIP is the main hormone product of murine K-cells. The inventors have found that GAST-expression often overlapped with high expression of the incretin GIP. Thus, in some embodiments, expression of GAST and high expression of incretin GIP is used as a marker for human G/K cells. In some embodiments, GAST is expressed and a high level of incretin GIP is expressed or GAST expression and a high level of incretin GIP expression is selected for. In some embodiments, the high level of expression of incretin GIP is 10 or more incretin GIP transcripts for each 3000 total transcripts in the cell, wherein the total transcripts relate to the transcripts of any genes in the cell. GIP is implicated in inducing insulin release, which is relevant for type II diabetes. Thus, in some embodiments, the disease or disorder is diabetes, e.g. type II diabetes. In some embodiments, the disease or disorder is a disease or disorder in which gastrin and/or incretin GIP and/or GPRP is implicated. Examples of such diseases and disorders are described herein. For example, in some embodiments, the disease or disorder is high acid secretion in the stomach. In some embodiments, the disease or disorder is indigestion or acid reflux. In some embodiments, the disease or disorder is a neurodegenerative disease or disorder, e.g. Alzheimer's disease or Parkinson's disease. In some embodiments, the physiological effect is insulin release, e.g. inducing insulin release. In some embodiments, the physiological effect is acid secretion in the stomach. In some embodiments, the physiological effect is parietal cell activity in the stomach. In some embodiments, the one or more hormones are gastrin and/or incretin GIP.

In some embodiments, the human EECs are human G cells and/or the target is gastrin (GAST). In some embodiments, the hormone is gastrin. In some embodiments, the disease or disorder is a disease or disorder in which gastrin is implicated. In some embodiments, the disease or disorder is a disease or disorder treatable or preventable by inducing or increasing expression and/or secretion of gastrin. In some embodiments, the disease or disorder is a disease or disorder treatable or preventable by decreasing or ceasing expression and/or secretion of gastrin. In some embodiments, the disease or disorder is high acid secretion in the stomach. In some embodiments, the disease or disorder is indigestion caused by high acid secretion in the stomach. In some embodiments, the disease or disorder is acid reflux caused by high acid secretion in the stomach. In some embodiments, the high acid secretion is caused by abnormal parietal cell activity in the stomach.

In some embodiments, the human EECs are human G cells and/or the target is the receptor for Gastrin-releasing peptide, GRPR. In some embodiments, the hormone is gastrin. In some embodiments, the disease or disorder is a disease or disorder in which gastrin is implicated. In some embodiments, the disease or disorder is a disease or disorder treatable or preventable by inducing or increasing expression and/or secretion of gastrin. In some embodiments, the disease or disorder is a disease or disorder treatable or preventable by decreasing or ceasing expression and/or secretion of gastrin. In some embodiments, the hormone is gastrin. In some embodiments, the disease or disorder is high acid secretion in the stomach. In some embodiments, the disease or disorder is indigestion caused by high acid secretion in the stomach. In some embodiments, the disease or disorder is acid reflux caused by high acid secretion in the stomach. In some embodiments, the high acid secretion is caused by abnormal parietal cell activity in the stomach.

In some embodiments, the human EECs are human G/K cells and/or the target is gastrin (GAST). In some embodiments, the hormone is gastrin. In some embodiments, the disease or disorder is a disease or disorder in which gastrin is implicated. In some embodiments, the disease or disorder is a disease or disorder treatable or preventable by inducing or increasing expression and/or secretion of gastrin. In some embodiments, the disease or disorder is a disease or disorder treatable or preventable by decreasing or ceasing expression and/or secretion of gastrin. In some embodiments, the disease or disorder is high acid secretion in the stomach. In some embodiments, the disease or disorder is indigestion caused by high acid secretion in the stomach. In some embodiments, the disease or disorder is acid reflux caused by high acid secretion in the stomach. In some embodiments, the high acid secretion is caused by abnormal parietal cell activity in the stomach.

In some embodiments, the human EECs are human G/K cells and/or the target is the receptor for Gastrin-releasing peptide, GRPR. In some embodiments, the hormone is gastrin. In some embodiments, the disease or disorder is a disease or disorder in which gastrin is implicated. In some embodiments, the disease or disorder is a disease or disorder treatable or preventable by inducing or increasing expression and/or secretion of gastrin. In some embodiments, the disease or disorder is a disease or disorder treatable or preventable by decreasing or ceasing expression and/or secretion of gastrin. In some embodiments, the hormone is gastrin. In some embodiments, the disease or disorder is high acid secretion in the stomach. In some embodiments, the disease or disorder is indigestion caused by high acid secretion in the stomach. In some embodiments, the disease or disorder is acid reflux caused by high acid secretion in the stomach. In some embodiments, the high acid secretion is caused by abnormal parietal cell activity in the stomach.

In some embodiments, the human EECs are human G/K cells and/or the target is incretin GIP. In some embodiments, the hormone is incretin GIP. In some embodiments, the disease or disorder is a disease or disorder in which incretin GIP is implicated. In some embodiments, the disease or disorder is a disease or disorder treatable or preventable by increasing or inducing expression and/or secretion of incretin GIP. In some embodiments, the disease or disorder is diabetes, in particular type II diabetes. In some embodiments, the disease or disorder is a neurodegenerative disease or disorder, for example, Alzheimer's disease or Parkinson's disease. In some embodiments, the disease or disorder is a disease or disorder treatable or preventable by decreasing or ceasing expression and/or secretion of incretin GIP.

In some embodiments, the human EECs are human L-cells and/or the one or more targets are one or more of (e.g. at least 1, 2, or all 3 of) GCG, NTS and PYY.

In some embodiments, the human EECs are human L-cells and/or the target is GCG. In some embodiments, the disease or disorder is a disease or disorder in which GCG or a hormone produced from GCG is implicated. In some embodiments, the hormone produced from GCG is GLP-1. In some embodiments, the hormone produced from GCG is GLP-2. In some embodiments, the disease or disorder is a disease or disorder treatable or preventable by increasing or inducing expression and/or secretion of GCG and/or increasing or inducing production of a hormone from GCG. In some embodiments, the disease or disorder is a disease or disorder treatable or preventable by decreasing or ceasing expression and/or secretion of GCG and/or decreasing or ceasing production of a hormone from GCG.

In some embodiments, the human EECs are human L-cells and/or the target is PYY. In some embodiments, the disease or disorder is a disease or disorder in which PYY is implicated. In some embodiments, the disease or disorder is a disease or disorder treatable or preventable by increasing or inducing expression and/or secretion of PYY. In some embodiments, the disease or disorder is a disease or disorder treatable or preventable by decreasing or ceasing expression and/or secretion of PYY.

In some embodiments, the human EECs are human L-cells and/or the target is NTS. In some embodiments, the disease or disorder is a disease or disorder in which NTS is implicated. In some embodiments, the disease or disorder is a disease or disorder treatable or preventable by increasing or inducing expression and/or secretion of NTS. In some embodiments, the disease or disorder is a disease or disorder treatable or preventable by decreasing or ceasing expression and/or secretion of NTS.

In some embodiments, where the one or more targets comprises GCG, the target may be an active peptide product derived from GCG, e.g. GLP-1.

In some embodiments, the human EECs are human D-cells and/or the target is the transcription factor HHEX. HHEX has been described in murine pancreatic and intestinal Sst-producing cells (Haber et al., 2017; Zhang et al., 2014). In some embodiments, the human D-cells lack expression of Amylin (IAPP). Amylin is a peptide hormone expressed in mouse D-cells. Thus, it is surprising that it is not also expressed in human D-cells. The inventors have found that when the HHEX transcription factor is knocked out, SST expression is lost as were the cells expressing SST. In addition, a significant increase in GCG expression (and consequently GLP-1 production) was found. Thus, targeting the HHEX transcription factor may be used to treat or prevent a disease or disorder in which SST or GCG (and consequently GLP-1) is implicated. Thus, in some embodiments, the disease or disorder is a disease or disorder in which one or more of SST, GCG and/or GLP-1 is implicated. In some embodiments, the disease or disorder is a disease or disorder treatable or preventable by ceasing or decreasing expression and/or secretion of SST. In some embodiments, the disease or disorder is a disease or disorder treatable or preventable by increasing or inducing secretion and/or expression and/or production of GCG and/or GLP-1. Examples of such diseases and disorders are described herein. For example, in some embodiments, the disease or disorder is selected from an appetite-related disease or disorder (e.g. obesity, anorexia, cancer cachexia, obesity, diabetes (in particular type II diabetes), Prader-Willi syndrome), a gut motility disease or disorder (e.g. a bowel movement disorder, Parkinson's disease or gastroparesis), diabetes (in particular type II diabetes), a neurodegenerative disease or disorder (e.g. Parkinson's disease or Alzheimer's disease), and depression. In some embodiments, the disease or disorder is a biliary movement disorder. In some embodiments, the disease or disorder is biliary dyskinesia. In some embodiments, the disease or disorder is a disease or disorder treatable or preventable by inducing or increasing expression and/or secretion of SST. In some embodiments, the disease or disorder is a disease or disorder treatable or preventable by decreasing or ceasing secretion and/or expression and/or production of GCG and/or GLP-1. Examples of such diseases and disorders are described herein. For example, in some embodiments, the disease or disorder is selected from an appetite-related disease or disorder (e.g. obesity, anorexia, cancer cachexia, obesity, diabetes (in particular type II diabetes), Prader-Willi syndrome), a gut motility disease or disorder (e.g. a bowel movement disorder, Parkinson's disease or gastroparesis), high acid secretion in the stomach (e.g. indigestion and/or acid reflux) and IBS. In some embodiments, the disease or disorder is IBD (e.g. Crohn's disease or ulcerative colitis). Thus, targeting the HHEX transcription factor may be used to treat or prevent a wide variety of diseases and disorders. In some embodiments, the physiological effect is selected from appetite, gut motility, insulin secretion, neurological or cognitive functioning, acid secretion in the stomach and parietal cell activity in the stomach. GCG levels affect glucose homeostasis. Thus, in some embodiments, the physiological effect is glucose homeostasis. Thus, the invention provides a method for affecting glucose homeostasis comprising inhibiting the HHEX transcription factor in one or more EECs. The inhibiting may comprise, for example, knocking out the HHEX transcription factor or targeting it with an inhibitor. Also provided is a construct for knocking out the HHEX transcription factor, or an HHEX inhibitor, for use in modulating glucose homeostasis. Also provided is a construct for knocking out the HHEX transcription factor in one or more EECs, or an HHEX inhibitor, for use in treating or preventing a disease or disorder in which one or more of SST, GCG and/or GLP-1 is implicated. Also provided is a construct for knocking out the HHEX transcription factor in one or more EECs, or an HHEX inhibitor, for use in treating or preventing diabetes. In some embodiments, the disease or disorder is diabetes, e.g. type II diabetes. Similarly, there is provided a method of treating or preventing a disease or disorder in which one or more of SST, GCG and/or GLP-1 is implicated comprising administering an HHEX knockout construct or an HHEX inhibitor to a patient. For example, there is provided a method of treating or preventing diabetes comprising administering an HHEX knockout construct or an HHEX inhibitor to a patient. In some embodiments, the diabetes is type II diabetes. In some embodiments, the treating or preventing comprises increasing GCG levels and consequently increasing GLP-1 production. In some embodiments, the treating or preventing comprises increasing GLP-1 production. Similarly, there is provided a method of modulating glucose homeostasis comprising administering an HHEX knockout construct or an HHEX inhibitor to a patient. Any suitable construct may be used. In some embodiments, the construct is a construct as described herein, for example, a construct described in the examples. Such a construct may be used as a compound in the present invention. For example, in some embodiments, the HHEX knockout construct uses CRISPR/Cas9 technology. In some embodiments, the knock out construct uses an HHEX forward gRNA comprising the sequence of SEQ ID NO:31 and an HHEX reverse gRNA comprising the sequence of SEQ ID NO:32 and optionally an HHEX forward primer comprising the sequence of SEQ ID NO:35 and an HHEX reverse primer comprising the sequence of SEQ ID NO:36. The invention further provides a method for preventing, reducing or ceasing expression of SST by an EEC comprising knocking out or inhibiting the HHEX transcription factor. In some embodiments, the method comprises generating an intestinal organoid in which HHEX is knocked out, e.g. an organoid of the invention. In some embodiments, the HHEX transcription factor is knocked out in an intestinal organoid, e.g. an organoid of the invention. In some embodiments, the one or more hormones are GCG, GLP (e.g. GLP-1) and/or SST.

In some embodiments, the human EECs are human D-cells and/or the target is SST. In some embodiments, the disease or disorder is a disease or disorder in which SST is implicated. In some embodiments, the disease or disorder is a disease or disorder treatable or preventable by increasing or inducing expression and/or secretion of SST. In some embodiments, the disease or disorder is a disease or disorder treatable or preventable by decreasing or ceasing expression and/or secretion of SST.

In some embodiments, the human EECs are human ECs and/or the target is LMX1A. The inventors have found that when the LMX1A transcription factor is knocked out, the serotonin-positive ECs are lost. The invention further provides a method for preventing or ceasing expression of serotonin by an EC comprising knocking out or inhibiting the LMX1A transcription factor. Thus, in some embodiments, the hormone is serotonin. The invention further provides a method for decreasing the number of ECs or eliminating ECs comprising knocking out or inhibiting the LMX1A transcription factor. In some embodiments, the disease or disorder is a disease or disorder in which Serotonin is implicated. Serotonin is elevated in IBS. Serotonin regulates bowel movement and activation of pain receptors. Thus, a method of inhibiting the LMX1A transcription factor would be advantageous in treating IBS because it would reduce the levels of serotonin. Thus, in some embodiments, the disease or disorder is IBS. Consequently, the invention provides a method for treating IBS comprising inhibiting or knocking out the LMX1A transcription factor. In some embodiments, the treating or preventing comprises reducing the levels of serotonin produced by human EECs. Likewise, in some embodiments, the physiological effect is regulation of bowel movement and/or activation of pain receptors. Serotonin is also implicated in gut motility disorders. Thus, in some embodiments, the disease or disorder is a gut motility disorder. Similarly, in some embodiments, the physiological effect is gut motility. In some embodiments, the disease or disorder is depression. In some embodiments, the disease or disorder is IBD. In some embodiments, the disease or disorder is Crohn's disease. In some embodiments, the disease or disorder is ulcerative colitis. In some embodiments, the disease or disorder is diabetes (in particular type II diabetes). Any suitable construct may be used to knock out LMX1A. In some embodiments, the construct is a construct as described in the examples. For example, in some embodiments, the knock out construct uses an LMX1A forward gRNA comprising the sequence of SEQ ID NO:33 and an LMX1A reverse gRNA comprising the sequence of SEQ ID NO:34, and optionally an LMX1A forward primer comprising the sequence of SEQ ID NO:37 and an LMX1A reverse primer comprising the sequence of SEQ ID NO:38. Thus, in some embodiments, the method for treating IBS comprises administering an LMX1A knockout construct or an LMX1A inhibitor to a patient. Such a construct may be used as a compound in accordance with the present invention. For example, the invention provides a construct for knocking out LMX1A or an LMX1A inhibitor for use in treating or preventing IBS or a gut motility disorder.

In some embodiments, the human EECs are human EECs and/or the target is Midkine (MDK). Midkine is a heparin-binding growth factor. Midkine is a cytokine not previously connected to enteroendocrine cells. The inventors have found that all enteroendocrine cell types express Midkine. In some embodiments, Midkine is expressed or expression of Midkine is selected for. In some embodiments, high expression of Midkine is used as a marker for human EECs. For example, in some embodiments a high level of Midkine is expressed or a high level of Midkine expression is selected for. In some embodiments, the high level of expression of Midkine is 10 or more Midkine transcripts for each 3000 total transcripts in the cell, wherein the total transcripts relate to the transcripts of any genes in the cell. In some embodiments, the disease or disorder is a disease or disorder in which Midkine is implicated. Midkine has been associated with obesity and inhibits insulin signalling in adipocytes (Fan et al., 2014). Thus, in some embodiments, the disease or disorder is obesity. In some embodiments, the disease or disorder is diabetes, for example, type II diabetes. In some embodiments, the physiological effect is insulin signalling, e.g. in adipocytes. For example, in some embodiments, an increase in Midkine expression and/or secretion decreases and/or inhibits insulin signalling. In some embodiments, a decrease in Midkine expression and/or secretion increases insulin signalling. While not previously seen in human EECs, Midkine has previously been reported as a biomarker of human intestinal neuroendocrine tumours (Edfeldt et al., 2017). In some embodiments, the human EEC is a human neuroendocrine tumour cell. However, in some embodiments, the human EEC is not a neuroendocrine tumor cell. In some embodiments, the disease or disorder is a human intestinal neuroendocrine tumour or neuroendocrine cancer. The inventors have surprisingly found that Midkine suppresses expression of all EEC hormones. In particular, the inventors have found that treating enteroendocrine cell differentiated organoids with Midkine reduces the expression of all hormones. This suggests it could act as a secreted molecule imposing a negative feedback on the gut endocrine system. Accordingly, in some embodiments, the physiological effect is hormone expression from human EECs, the target is Midkine, and the human EECs are human EECs. Thus, the invention provides a method for modulating hormone expression in human EECs comprising contacting the human EECs with Midkine and/or modulating the endogenous expression of Midkine. In some embodiments, the expression of all or substantially all hormones in human EECs is modulated. In some embodiments, the expression of one or more (e.g. 1, 2, 3, 4, 5, 6, 7, 8 or more) hormones in human EECs is modulated. For example, the invention provides a method for decreasing hormone expression from human EECs comprising increasing the level of Midkine in the human EECs. In some embodiments, increasing the level of Midkine in the human EEC comprises increasing expression of endogenous Midkine in the human EEC. Similarly, the invention provides a method for decreasing hormone expression from human EECs comprising contacting the human EECs with Midkine. The method may be carried out in vitro or in vivo. For example, in some embodiments, increasing the level of Midkine in the human EEC comprises administering Midkine to a patient. In some embodiments, the expression of hormones in EECs is decreased by at least 5% (e.g. at least 10%, 20%, 30%, 40%, 50%, 60%, 70%, 80%, 90% or 99% or 100%) compared to the level of expression of hormones in a control EEC that has not been treated with Midkine. In some embodiments, the expression of each of the hormones in the human EECs is decreased as described herein. In some embodiments, the total expression level of all the hormones in the human EECs is decreased as described herein. In some embodiments, the expression of one or more (e.g. 1, 2, 3, 4, 5, 6, 7, 8 or more) hormones in human EECs is decreased, as described herein. Decreasing hormone expression in human EECs may be advantageous for treating or preventing a disease or disorder in which too much hormone secretion is implicated. Thus, in some embodiments, the disease or disorder is a disease or disorder treatable or preventable by decreasing or ceasing expression and/or secretion and/or production of one or more hormones by EECs. Examples of such diseases and disorders are described herein. For example, in some embodiments, a disease or disorder treatable or preventable by decreasing or ceasing expression and/or secretion and/or production of one or more hormones is selected from an appetite-related disease or disorder (e.g. obesity, anorexia, cancer cachexia, obesity, diabetes (in particular type II diabetes), Prader-Willi syndrome), a gut motility disease or disorder (e.g. a bowel movement disorder, Parkinson's disease or gastroparesis), high acid secretion in the stomach (e.g. indigestion and/or acid reflux) and IBS. Similarly, the invention provides a method for increasing hormone expression from human EECs comprising decreasing the level of Midkine in the human EEC. Inhibiting Midkine reaching human enteroendocrine cells could boost the expression of many gut hormones. This could benefit all metabolic diseases related to intestinal hormones, such as obesity and diabetes. It could also be used in conjunction with other drugs targeting enteroendocrine cells, to amplify their effects. In some embodiments, decreasing the level of Midkine in the EEC comprises decreasing expression of endogenous Midkine. For example, in some embodiments, decreasing the level of Midkine in the human EEC comprises contacting the EEC with a Midkine antagonist. Thus, in some embodiments, the method comprises contacting Midkine with an inhibitor. In some embodiments, the Midkine antagonist is the Midkine inhibitor 3-[2-(4-fluorobenzyl) imidazo [2,1-beta]thiazol-6-yl]-2H-chromen-2-one, also known as iMDK (Masui, M. et al., 2016). Similarly, the invention provides a method for increasing hormone expression from human EECs comprising contacting the human EECs with an antagonist of the Midkine receptor. The method may be carried out in vitro or in vivo. For example, in some embodiments, decreasing the level of Midkine in the cell comprises administering a Midkine antagonist or an antagonist of the Midkine receptor to a patient. Thus, the invention provides a method for treating or preventing a disease or disorder comprising administering a Midkine antagonist to a patient. In some embodiments, the expression of hormones from EECs is increased by at least 5% (e.g. at least 10%, 20%, 30%, 40%, 50%, 60%, 70%, 80%, 90% or 99% or 100%) compared to the level of expression of hormones in a control EEC that has not been treated with Midkine. In some embodiments, the expression of each of the hormones in the human EECs is increased as described herein. In some embodiments, the total expression level of all the hormones in the human EECs is increased as described herein. In some embodiments, the expression of one or more (e.g. 1, 2, 3, 4, 5, 6, 7, 8 or more) hormones in human EECs is increased, as described herein. Increasing hormone expression in human EECs may be advantageous for treating or preventing a disease or disorder in which insufficient hormone secretion is implicated. Thus, in some embodiments, the disease or disorder is a disease or disorder treatable or preventable by increasing or inducing expression and/or secretion and/or production of one or more hormones by EECs. Examples of such diseases and disorders are described herein. For example, in some embodiments, a disease and disorder treatable or preventable by increasing or inducing expression and/or secretion and/or production of one or more hormones is selected from an appetite-related disease or disorder (e.g. obesity, anorexia, cancer cachexia, obesity, diabetes (in particular type II diabetes), Prader-Willi syndrome), a gut motility disease or disorder (e.g. a bowel movement disorder, Parkinson's disease or gastroparesis), diabetes (in particular type II diabetes), a neurodegenerative disease or disorder (e.g. Parkinson's disease or Alzheimer's disease) and depression. In some embodiments, a disease and disorder treatable or preventable by increasing or inducing expression and/or secretion of Motilin is a biliary movement disorder, e.g. biliary dyskinesia. In some embodiments, the disease or disorder is a disease or disorder as described herein. In some embodiments, the hormone is Midkine. In some embodiments, the one or more hormones and/or hormone precursors are one or more hormones and/or hormone precursors expressed by or produced by EECs. In some embodiments, the one or more hormones and/or hormone precursors are selected from one or more of (e.g. 2, 3, 4 or more of) Ghrelin, Cerebellin 1, CCK, GCG, GLP-1, PYY, PPY, Motilin, Angiotensin, Serotonin, GIP, Gastrin and Secretin. In some embodiments, the physiological effect is selected from appetite, gut motility, bowel movement and/or activation of pain receptors, insulin secretion, neurological and/or cognitive functioning, acid secretion in the stomach and parietal cell activity in the stomach. In some embodiments, a variant or fragment of Midkine is used, or a fragment of a variant of Midkine. For example, in some embodiments, the polypeptide sequence of the variant has at least 75% (e.g. at least 80%, 85%, 90%, 95%, 98%, 99%) sequence identity with the Midkine sequence shown in SEQ ID NO:69.

In some embodiments, the human EECs are human EECs with the exception of human ECs and/or the target is carboxypeptidase CPB1. In some embodiments, the human EECs are human M-X cells and/or the target is carboxypeptidase CPB1. In some embodiments, the method for isolating of identifying a human M-X cell or a population of human M-X cells comprises selecting cells which express the highest expression level of CPB1. Carboxypeptidases are typically involved in hormone processing (Sapio and Fricker, 2014). Expression of CPB1 has been observed in the rat pancreas (Yu et al., 2017). Thus, in some embodiments, the physiological effect is hormone processing, for example, hormone processing in the intestine. Thus, in some embodiments, the disease or disorder is a disease or disorder in which a hormone that is processed is implicated. Examples of such diseases and disorders are provided herein. For example, GLP-1 is a hormone that is processed from GCG. Thus, in some embodiments, the disease or disorder is a disease or disorder in which GLP-1 is implicated. Examples of such diseases and disorders are provided herein. For example, in some embodiments, the disease or disorder is selected from a gut motility disorder (e.g. bowel movement disorders, Parkinson's disease, gastroparesis), an appetite-related disorder (e.g. anorexia, cancer cachexia, obesity, diabetes, Prader-Willi syndrome), diabetes (e.g. type II diabetes) and/or a neurodegenerative disease or disorder (e.g. Parkinson's Disease or Alzheimer's Disease). In some embodiments, the hormone is a processed hormone. In some embodiments, the physiological effect is selected from appetite, gut motility, insulin secretion, neurological and/or cognitive functioning.

In some embodiments, the human EECs are human EECs and/or the target is FGF14. FGF14 has very limited expression in murine EECs. FGF14 belongs to a set of intracellular FGFs which play a role in the clustering of ion channels in neurons (Pablo and Pitta, 2017). EECs use many principles of neurons and are also electrically excitable. Accordingly, in some embodiments, the physiological effect is the clustering of ion channels in neurons.

In some embodiments, the human EECs are human PYY+ cells and/or the target is the olfactory receptor OR51E2. In contrast, the mouse homologue Olfr78 is lowly expressed in ECs only, and so it is surprising that OR51E2 is expressed in human PYY+ cells. The inventors have found that the OL51E2 receptor is also sporadically expressed in other EEC subtypes and so stimulation of this receptor could have broader effects. For example, the inventors have shown that OR51E2 is expressed in human L-cells. Thus, in some embodiments, the human EECs are human L-cells. The inventors have validated OR51E2 as a target receptor using the organoid technology provided in the present application. When the OR51E2 receptor was stimulated, a calcium reporter was activated. Calcium activation is a measure of hormone secretion. Thus, the inventors have shown that stimulation of the OR51E2 receptor leads to hormone secretion. Thus, in some embodiments, the disease or disorder is a disease or disorder in which PYY is implicated. Examples of such diseases and disorders are provided herein. For example, in some embodiments, the disease or a disorder is an appetite-related disease or disorder. In some embodiments, the disease or disorder is a disease or disorder requiring appetite inhibition. In some embodiments, the disease or disorder is selected from obesity, diabetes and Prader-Willi syndrome. In some embodiments, the disease or disorder is a disease or disorder requiring appetite stimulation. In view of the broad expression of OR51E2, in some embodiments, the disease or disorder is a disease or disorder in which a hormone expressed and/or secreted and/or produced by or produced from a hormone precursor secreted by an EEC is implicated. Examples of such diseases and disorders are provided herein. Thus, in some embodiments, the disease or disorder is a disease or disorder as described herein. For example, in some embodiments, the disease or disorder is a disease or disorder in which a hormone expressed and/or secreted and/or produced by an L-cell or produced from a hormone precursor secreted by an L-cell is implicated. Thus, in some embodiments, the disease or disorder is a disease or disorder in which GLP-1 is implicated. In some embodiments, the disease or disorder is a disease or disorder treatable or preventable by increasing or inducing production of GLP-1. In some embodiments, the disease or disorder is a disease or disorder treatable or preventable by ceasing or decreasing production of GLP-1. Examples of such diseases and disorders are provided herein. In some embodiments, the hormone is PYY. In some embodiments, the hormone is GLP-1 and/or NTS. In some embodiments, the disease or disorder is a disease or disorder treatable or preventable by increasing or inducing expression and/or secretion of NTS. In some embodiments, the disease or disorder is a disease or disorder treatable or preventable by ceasing or decreasing expression and/or secretion of NTS. In some embodiments, the hormone is GLP-1. In some embodiments, the physiological effect is appetite.

In some embodiments, the human EECs are human duodenal EECs from the proximal intestine and/or the target is the enzyme tryptophan 2,3-dioxygenase (TDO2). TDO2 can metabolize tryptophan through the kynurenine pathway and is one of the primary regulators of availability of this amino acid. Tryptophan is the precursor of serotonin and TDO2 knockout mice experience increased serotonin levels (Too et al., 2016), suggesting TDO2 could be a local regulator of serotonin production in the gut. Accordingly, in some embodiments, the physiological effect is serotonin production in the gut. Thus, the invention provides a method for modulating serotonin production in the gut comprising modulating expression of TDO2 in human duodenal EECs from the proximal intestine. For example, in some embodiments, the invention provides a method for increasing serotonin production in the gut comprising decreasing expression of TDO2 in human duodenal EECs from the proximal intestine. In some embodiments, the physiological effect is tryptophan metabolism, for example, tryptophan metabolism, e.g. in human EECs. Thus, the invention provides a method for modulating tryptophan metabolism comprising modulating expression of the TDO2 in human duodenal EECs from the proximal intestine. For example, in some embodiments, the invention provides a method for increasing the availability of tryptophan comprising decreasing expression of TDO2 in human duodenal EECs from the proximal intestine. In some embodiments, expression of TDO2 is knocked out. In some embodiments, the disease or disorder is a disease or disorder in which serotonin is implicated. In some embodiments, the disease or disorder is a disease or disorder treatable or preventable by increasing or inducing production and/or secretion of serotonin. In some embodiments, the disease or disorder is a disease or disorder treatable or preventable by ceasing or decreasing production and/or secretion of serotonin. Serotonin is implicated in gut motility disorders (e.g. bowel movement disorders, Parkinson's disease, gastroparesis) and irritable bowel syndrome. Serotonin is elevated in IBS. Serotonin regulates bowel movement and activation of pain receptors. Accordingly, in some embodiments, the disease or disorder is a gut motility disorder or irritable bowel syndrome. In some embodiments, the gut motility disorder is selected from bowel movement disorders, Parkinson's disease and gastroparesis. In some embodiments, the disease or disorder is depression. In some embodiments, the disease or disorder is diabetes (in particular type II diabetes). In some embodiments, the disease or disorder is IBD. In some embodiments, the disease or disorder is Crohn's disease. In some embodiments, the disease or disorder is ulcerative colitis. In some embodiments, the hormone is Serotonin. In some embodiments, the physiological effect is bowel movement and/or activation of pain receptors.

In some embodiments, the human EECs are human EECs and/or the target is the tachykinin peptide-coding TAC3. In contrast, the mouse homologue Tac2 is absent in the murine intestine, and so it is surprising that TAC3 is found in human EECs. TAC3 codes for Neurokinin B. Thus, accordingly, in some embodiments, the human EECs are human EECs and the one or more protein of interest is Neurokinin B. TAC3 has been described as a regulator of secretion of gonadotropin-releasing hormone in humans that is produced in the hypothalamus (Sanger, 2004). Accordingly, in some embodiments, the physiological effect is secretion of gonadotropin-releasing hormone, e.g. by human EECs. However, the main receptor for NKB, NK₃ (coded by TACR3), has been implicated in the regulation of gastrointestinal motility (Sanger, 2004). Accordingly, in some embodiments, the physiological effect is gastrointestinal motility. In some embodiments, the disease or disorder is a gut motility disorder.

In some embodiments, the human EECs are human EECs and/or the one or more targets are FGFR1 and/or B-Klotho (KLB). The hepatokine FGF21 has recently received much attention as a regulator of blood glucose. Accordingly, in some embodiments, the physiological effect is blood glucose levels, the one or more targets are FGFR1 and/or B-Klotho and the human EECs are human EECs. Several FGF21 mimetics are currently being tested for the treatment of diabetes (Kuro-o, 2019). While the receptors for FGF21 are described as a complex of FGFR1 and B-Klotho (KLB), the site of action of FGF21 is much disputed. The inventors observed broad expression of FGFR1 and KLB by human EECs, suggesting that the FGF21 effects could be partially mediated through the gut. Fgfr1 is absent in murine EECs, while Klb is expressed at very low levels, and so it is surprising that these are expressed in human EECs. Accordingly, in some embodiments, the disease or disorder is diabetes. The inventors' findings therefore open the possibility of specifically targeting FGF21 and related therapeutic compounds, e.g. FGF21 mimetics to the gut. Accordingly, in some embodiments, the therapeutic compound is FGF21 or a mimetic, derivative or variant thereof, which is targeted to the gut. In some embodiments, the therapeutic compound is specifically targeted to the gut. In some embodiments, the therapeutic compound is FGF21 or a mimetic, derivative or variant thereof and is targeted to human EECs. In some embodiments, the therapeutic compound is specifically targeted to human EECs. Also provided is a pharmaceutical composition comprising FGF21 or a mimetic, derivative or variant, which is formulated to be targeted to the gut. In some embodiments, the pharmaceutical composition is formulated to be targeted specifically to the gut. In some embodiments, the pharmaceutical composition is formulated to be targeted to human EECs. In some embodiments, the pharmaceutical composition is formulated to be targeted specifically to human EECs.

In some embodiments, the human EECs are human EECs and/or the target is C10ORF10 (also known as DEPP1). This gene is negatively regulated by insulin in the liver and adipocyte tissue and its product controls the ratio between metabolic pathways including ketogenesis and gluconeogenesis (Li et al., 2018). In particular, compared with controls, DEPP overexpression reduced food intake, the energy expenditure rate, and the respiratory quotient. DEPP overexpression significantly increased fatty acid oxidation and ketogenesis but suppressed lipid synthesis and gluconeogenesis (Li et al., 2018). Accordingly, in some embodiments, the physiological effect is controlling the ratio between metabolic pathways including ketogenesis and gluconeogenesis. For example, in some embodiments, the physiological effect is increasing fatty acid oxidation and ketogenesis and reducing lipid synthesis and gluconeogenesis. Thus, in some embodiments, the physiological effect is modulating food intake, the energy expenditure rate, and/or the respiratory quotient, for example, increasing all of these by overexpressing DEPP1 in human EECs. Where appropriate, these may be modulated in the intestine. For example, in some embodiments, the disease or disorder is a disease or disorder associated with food intake, e.g. obesity, Prader-Willi syndrome, diabetes, anorexia or cancer cachexia, for example, obesity or anorexia. In some embodiments, the disease or disorder is a disease or disorder associated with food intake, e.g. obesity or anorexia. In some embodiments, the disease or disorder is obesity.

Accordingly, in some embodiments, the human EECs are human EECs and/or the one or more targets are one or more of (e.g. at least 1, 2, 3, 4, 5, 6 or all 7 of) Midkine, FGF14, tachykinin peptide-coding TAC3, FGFR1, B-Klotho (KLB) and C10ORF10. In some embodiments, the one or more targets additionally comprise the enzyme tryptophan 2,3-dioxygenase (TDO2) and/or the carboxypeptidase CPB1.

In some embodiments, the human EECs are human NTS+ cells and/or the target is LCN15. LCN15 is a lipocalin previously identified as one of the strongest glucose-regulated genes in Caco-2 cells (Boztepe and Gulec, 2018). Although some lipocalins have been implicated in the development of insulin resistance, the function of LCN15 is unknown. However, in view of the function of other lipocalins, LCN15 may also be implicated in the development of insulin resistance. Thus, the identification of LCN15 in human NTS+ cells opens the way for targeting this lipocalin protein in the gut in order to modulate insulin resistance. Accordingly, in some embodiments, the disease or disorder is diabetes, e.g. type II diabetes. In some embodiments, the physiological effect is insulin resistance.

In some embodiments, the human EECs are human M-X cells and/or the target is MLN (Motilin). The hormone Motilin is expressed in human EECs but is not expressed in mice. Thus, MLN+ cells do not exist in mice. Motilin is a regulator of gut motility. As explained above, all the relevant practical applications described above can be applied to this embodiment, mutatis mutandis. For example, in some embodiments, the invention provides an isolated population of human M-X cells expressing MLN. For example, in some embodiments, the physiological effect is regulating gut motility. In some embodiments, the disease or disorder is a disease or disorder in which Motilin is implicated. In some embodiments, the disease or disorder is a gut motility disorder. In some embodiments, the disease or disorder is a disease or disorder requiring appetite stimulation. In some embodiments, the disease or disorder is anorexia. In some embodiments, the disease or disorder is cancer cachexia. In some embodiments, the disease or disorder is diabetes. In some embodiments, the diabetes is type II diabetes. In some embodiments, the disease or disorder is a biliary movement disorder. In some embodiments, the disease or disorder is biliary diskinesia. In some embodiments, the disease or disorder is a disease or disorder requiring appetite inhibition. In some embodiments, the disease or disorder is obesity. In some embodiments, the disease or disorder is diabetes. In some embodiments, the disease or disorder is type II diabetes. In some embodiments, the disease or disorder is Prader-Willi syndrome. Examples of such disorders are provided herein. In some embodiments, the hormone is Motilin.

In some embodiments, the human EECs are human M-X cells and/or the target is GHRL. In some embodiments, the disease or disorder is a disease or disorder in which GHRL is implicated.

In one or more embodiments, the human EECs are human M-X cells and/or the one or more targets are MLN and/or GHRL. In some embodiments, expression of MLN is higher than GHRL. In some embodiments, expression of GHRL is higher than MLN. In embodiments in which expression of GHRL is higher than MLN, the cells may optionally be called human X-M cells rather than human M-X cells.

In one or more embodiments, the human EECs are human M-X cells and/or the target is GHRL. Ghrelin is implicated in appetite stimulation. For example, Ghrelin receptor agonists are currently being tested in clinical trials and show very positive results in terms of increasing appetite. In some embodiments, the disease or disorder is a disease or disorder in which Ghrelin is implicated. Thus, in some embodiments, the disease or disorder is anorexia or cancer cachexia. In some embodiments, the disease or disorder is obesity, diabetes or Prader-Willi syndrome. In some embodiments, the disease or disorder is obesity. In some embodiments, the physiological effect is appetite. In some embodiments, the hormone is Ghrelin.

In one or more embodiments, the human EECs are human M-X cells and/or the target is Motilin. In some embodiments, the disease or disorder is a disease or disorder in which Motilin is implicated. Motilin is implicated in gut motility disorders. For example, the Motilin agonist erythromycin is clinically used to stimulate peristalsis. Thus, in some embodiments, the physiological effect is peristalsis. In some embodiments, the physiological effect is gut motility. Thus, in some embodiments, the disease or disorder is a gut motility disorder (e.g. bowel movement disorders, Parkinson's disease, gastroparesis). In some embodiments, the hormone is Motilin.

In some embodiments, the human EECs are human M-X cells and/or the target is ENPP1. ENPP1 is a known regulator of insulin responses and extracellular ATP levels (Di Paola et al., 2011), that is similarly expressed by murine X-cells. In some embodiments, the disease or disorder is diabetes. For example, in some embodiments, the diabetes is type II diabetes, for example, insulin resistant type II diabetes. As explained above, all the relevant practical applications described above can be applied to this embodiment, mutatis mutandis. For example, by way of non-limiting further example, the invention provides a method for identifying/screening/validating a compound for suitability for treating or preventing diabetes, e.g. as described herein. Similarly, the invention provides a method for treating or preventing diabetes comprising overexpressing ENPP1 in M-X cells the patient. In some embodiments, the treating or preventing comprises contacting human M-X cells with the ENPP1 RNA and/or ENPP1 protein. In some embodiments, the physiological effect is extracellular ATP levels, e.g. in the intestine. Accordingly, by way of non-limiting example, the invention further provides a method for modulating extracellular ATP levels comprising modulating expression of ENPP1 in human M-X cells. In some embodiments, the physiological effect is regulation of insulin responses.

In some embodiments, the human EECs are human M-X cells and/or the target is acyl-CoA synthetase Acsl1 enzyme. Ghrelin (GHRL) is a hormone requiring a specific acyl modification, a process shown to be dependent on this enzyme in mouse stomach X-cells (Bando et al., 2016). In some embodiments, the physiological effect is acylation. Thus, by way of non-limiting example, the invention provides a method for modulating acylation, e.g. of the GHRL hormone, comprising modulating expression of the acyl-CoA synthetase Acsl1 enzyme in human M-X cells. In some embodiments, Acsl1 is expressed or expression of Acsl1 is selected for. In some embodiments, high expression of Acsl1 is used as a marker for human M-X cells. For example, in some embodiments a high level of Acsl1 is expressed or a high level of Acsl1 expression is selected for. In some embodiments, the high level of expression of Acsl1 is 1 or more Acsl1 transcripts for each 3000 total transcripts in the cell, wherein the total transcripts relate to the transcripts of any genes in the cell. In some embodiments, the disease or disorder is a disease or disorder in which Ghrelin is implicated. Thus, in some embodiments, the disease or disorder is an appetite-related disease or disorder. For example, in some embodiments, the disease or disorder is a disease or disorder requiring appetite stimulation. For example, in some embodiments, the disease or disorder is anorexia or cancer cachexia. In some embodiments, the disease or disorder is a disease or disorder requiring appetite inhibition. In some embodiments, the disease or disorder is obesity, diabetes or Prader-Willi syndrome. In some embodiments, the physiological effect is appetite.

In some embodiments, the one or more targets are one or more of (e.g. 1, 2, 3, or all 4 of) MLN, GHRL, ENPP1 and the acyl-CoA synthetase Acsl1 enzyme and/or the human EECs are human M-X cells.

In some embodiments, the human EECs are human M-X cells and/or the target is TRNP1. TRNP1 is involved in cortical folding in the brain and is the only transcription factor specific to M/X cells (Stahl et al., 2013). In some embodiments, the physiological effect is enhancing M/X cell differentiation. For example, in some embodiments, TRNP1 may be targeted to enhance M/X cell differentiation. M/X-cells secrete MLN and GHRL. Thus, in some embodiments, the disease or disorder is a disease or disorder in which MLN and/or GHRL is implicated. Examples of such diseases and disorders are provided herein. For example, in some embodiments, the disease or disorder is a gut motility disorder. In some embodiments, the disease or disorder is an appetite-related disease or disorder. In some embodiments, the disease or disorder is a disease or disorder requiring appetite stimulation. In some embodiments, the disease or disorder is a disease or disorder requiring appetite inhibition. In some embodiments, the physiological effect is selected from appetite and gut motility.

In some embodiments, the human EECs are human M-X cells and/or the target is precerebellin 1 (CBLN1). CBLN1 is a putative hormone and is known to stimulate food intake upon intracerebroventricular injection (similar to the function of Ghrelin) (Gardiner et al., 2010). In some embodiments, the disease or disorder is a disease or disorder in which CBLN1 is implicated. In some embodiments, the disease or disorder is therefore a disease or disorder relating to food intake, e.g. an appetite-related disease or disorder (for example, obesity, diabetes, Prader-Willi syndrome, anorexia or cancer cachexia, e.g. obesity or anorexia). In some embodiments, the disease or disorder is a disease or disorder requiring appetite inhibition. In some embodiments, the disease or disorder is selected from obesity, diabetes and Prader-Willi syndrome. In some embodiments, the disease or disorder is a disease or disorder requiring appetite stimulation. In some embodiments, the disease or disorder is selected from anorexia and cancer cachexia. Thus, in some embodiments, the invention provides a method for identifying/screening/validating a compound for suitability for treating or preventing a disease or disorder relating to food intake, wherein the method comprises (i) contacting an intestinal reporter organoid of the invention comprising tagged CBLN1 with the compound; and (ii) determining whether secretion of CBLN1 is affected. The surprising finding that CBLN1 is expressed in the gut opens the possibility of targeting treatments for such diseases or disorders to the gut or to human M-X cells in the gut. Gut targeting would be preferable to intracerebroventricular injection, due to the invasive nature of the latter. For example, in some embodiments, the treatment or prevention of the disease or disorder comprises increasing expression of CBLN1 in the gut or in human M-X cells in the gut. The skilled person would be able to increase expression of CBLN1 in the gut or in human M-X cells using the teaching described herein in combination with the teaching available in the art. Similarly, in some embodiments, the physiological effect is food intake. For example, in some embodiments, modulating the physiological effect comprises stimulating food intake. In some embodiments, the human M-X cells are GHRL+ cells. Gut peptides have been found to be produced in the brain (Gardiner et al. 2010), which suggests that hormone-producing cells in the brain are similar to EECs in terms of the hormone biology, for example, that they have the same or similar receptors to EECs. In addition, a number of studies have found evidence of signalling from the gut to the brain (through vagal neurons) (Bellono et al. 2017; Kaelberer et al 2018; Raybould et al, 2007).

In some embodiments, the human EECs are human M-X cells and/or the target is Angiotensin (AGT). AGT is a peptide hormone and is a regulator of blood pressure. It is also described as a regulator of contraction of the musculature of human intestinal walls (similar to the gut motility-enhancing function of Motilin) (Ewert et al., 2006). In some embodiments, high expression of AGT is used as a marker for human M-X cells. In some embodiments, a high level of AGT is expressed or a high level of AGT expression is selected for. For example, in some embodiments, EECs with the highest level of AGT expression are selected in order to select human M-X cells. In some embodiments, the high level of expression of AGT is 5 or more AGT transcripts for each 3000 total transcripts in the cell, wherein the total transcripts relate to the transcripts of any genes in the cell. In some embodiments, AGT is expressed or expression of AGT is selected for. In some embodiments, the disease or disorder is a disease or disorder in which AGT is implicated. In some embodiments, the disease or disorder is a disease or disorder relating to gut motility. In some embodiments, the gut motility disorder is selected from a bowel movement disorder, Parkinson's disease or gastroparesis. In some embodiments, the physiological effect is gut motility. In some embodiments, there is provided a method for identifying/screening/validating a compound for suitability for treating or preventing a disease or disorder relating to gut motility, wherein the method comprises (i) contacting an intestinal reporter organoid of the invention comprising tagged AGT with the compound; and (ii) determining whether secretion of AGT is affected. In some embodiments, the physiological effect is blood pressure. In some embodiments, the disease or disorder is a disease or disorder relating to abnormal blood pressure. For example, the disease or disorder may be selected from hypertension, hypotension, hypertensive heart disease, kidney disease, atherosclerosis, arteriosclerosis, eye damage, stroke and vascular dementia. In some embodiments, the disease or disorder is hypertension or hypotension. In some embodiments, there is provided a method for identifying/screening/validating a compound for suitability for treating or preventing a disease or disorder relating to abnormal blood pressure, wherein the method comprises (i) contacting an intestinal reporter organoid of the invention comprising tagged AGT with the compound; and (ii) determining whether secretion of AGT is affected. In some embodiments, the hormone is Angiotensin.

In some embodiments, the human EECs are human M-X cells and/or the target is the sulfate transporter SLC26A7. The inventors have found that M/X cells displayed highest expression of all EECs of the sulfate transporter SLC26A7. Accordingly, in some embodiments, the method for isolating or identifying a human M-X cell or a population of human M-X cells comprises selecting cells which express the highest expression level of SLC26A7. For example, in some embodiments, EECs with the highest level of SLC26A7 expression are selected in order to select human M-X cells. In some embodiments, cells in which the expression of SLC26A7 is 1 or more SLC26A7 transcripts for each 3000 total transcripts in the cell, are selected for, wherein the total transcripts relate to the transcripts of any genes in the cell. In some embodiments, SLC26A7 is expressed or expression of SLC26A7 is selected for. In some embodiments, the physiological effect is sulfate transport.

In some embodiments, the human EECs are human M-X cells and/or the target is the T₄- and Retinol-binding Transthyretin (TTR). Although TTR is expressed by many EECs, both in mouse and human, the inventors have found that M-X cells display much higher levels. Thus, in some embodiments, a high level of TTR is expressed or a high level of expression of TTR is selected for. Accordingly, in some embodiments, the method for isolating or identifying a human M-X cell or a population of human M-X cells comprises selecting human EECs which express the highest expression level of TTR. In some embodiments, high expression of TTR is used as a marker for human M-X cells. In some embodiments, the high level of expression of TTR is 5 or more TTR transcripts for each 3000 total transcripts in the cell, wherein the total transcripts relate to the transcripts of any genes in the cell. In some embodiments, TTR is expressed or expression of TTR is selected for. A TTR mutation underlies the most common form of amyloidosis, characterized by cardiomyopathy and cardiac failure, but also destruction of enteric neurons and associated problems in gastrointestinal motility (Ueda and Ando, 2014). Of interest, it is known that gastrointestinal motility problems are partly relieved by Motilin agonists. Although the liver is suspected as the principle source of amyloid TTR, the results presented herein indicate that local, M-X cell produced TTR may play a role in pathogenesis as well. Thus, the present invention identifies mutated TTR produced by the intestine as a potential novel cause of amyloidosis. Accordingly, in some embodiments, the disease or disorder is amyloidosis, for example, amyloidosis characterised by one or more of cardiomyopathy, cardiac failure, destruction of enteric neurons and problems in gastrointestinal motility. For example, in some embodiments, the treating or preventing comprises improving gastrointestinal motility. Also provided a method for treating or preventing amyloidosis comprising reducing or preventing TTR production in M-X cells. In some embodiments, the method comprises reducing or preventing TTR production by M-X cells or correcting the sequence of the TTR mutation after, concurrently or before targeting TTR produced by the liver. In some embodiments, gene silencing or gene modification techniques (e.g. CRISPR) may be used to reduce or prevent TTR production by M-X cells or to correct the sequence of the TTR mutation. In some embodiments, reducing or preventing TTR production in M-X cells comprises resecting the patient's intestine to remove the M-X cells, for example, removing the ileum. In some embodiments, reducing or preventing TTR produced by M-X cells comprises contacting a patient's M-X cells with an antagonist of TTR production. In some embodiments, the TTR is mutated TTR. In some embodiments, the TTR mutation is implicated in amyloidosis. In some embodiments, the TTR mutation is the Val30Met mutation. In some embodiments, treating or preventing amyloidosis comprises increasing the expression of wild type TTR in M-X cells in the patient. In some embodiments, the physiological effect is amyloidosis.

In some embodiments, the human EECs are human M-X cells and/or the one or more targets are one or more of (e.g. at least 1, 2, 3, 4, 5 or more or all 6 of) TRNP1, precerebellin (CBLN1), IL20-RA, Angiotensin (AGT), SLC26A7, and T4- and Retinol-binding Transthyretin (TTR). In some embodiments, the one or more targets additionally comprise MLN. In some embodiments, the one or more targets additionally comprise one or more of (e.g. at least 1, 2 or all 3 of) GHRL, ENPP1 and acyl-CoA synthetase Acsl1.

The invention provides an isolated human M cell expressing MLN. Also provided is an isolated human M/X cell, wherein the cell expresses MLN and GHRL. In some embodiments, the M/X cell additionally expresses one or more of (e.g. 1, 2, 3, 4, 5, 6, 7 or more or all 8 of) ENPP1, acyl-CoA synthetase Acsl1, TRNP1, precerebellin (CBLN1), IL20-RA, Angiotensin (AGT), SLC26A7, and T4- and Retinol-binding Transthyretin (TTR). In some embodiments, the M/X cell additionally expresses CPB1. The terms “M/X” cell and “M-X” cell are used interchangeably herein. An isolated population of such cells is also provided herein. In some embodiments, the isolated population of human M cells or the isolated population of human M/X cells does not comprise human EECs of any other subtype. An isolated cell as provided herein, or a population of such cells, is useful for further study of such cells, for example, using high-throughput screening.

In some embodiments, the isolated population of human M-X cells additionally comprises L-cells producing MLN. In some embodiments, the isolated population of human M-X cells does not comprise L-cells producing MLN. In some embodiments, L-cells are selected against in order to obtain the isolated population of human M-X cells.

In some embodiments, the human EECs are human GCG+ cells and/or the target is Pancreatic Polypeptide (PPY). In some embodiments, the GCG+ cells are L-cells. In some embodiments, Pancreatic Polypeptide (PPY) is highly expressed or a high level of expression of PPY is selected for. PPY is a well-described peptide hormone from the endocrine pancreas involved in appetite regulation (Cox, 2007). PPY is believed to suppress appetite. It is known to be elevated in anorexia and completely absent in Prader-Willi syndrome, which is associated with an urge to eat constantly. This hormone has not previously been described in the context of the human or mouse small intestine. The invention therefore opens the way for looking for therapeutic compounds that target PPY in the gut (or to human GCG+ cells) for medical applications relating to appetite regulation. For example, it opens the way to use compounds that increase or decrease (as appropriate) expression and/or secretion of PPY by human EECs. In some embodiments, the disease or disorder is a disease or disorder in which PPY is implicated. Thus, in some embodiments, the disease or disorder is a disease or disorder relating to food intake, e.g. an appetite-related disease or disorder (for example, obesity, anorexia or Prader-Willi syndrome). In some embodiments, the disease or disorder is obesity, diabetes or Prader-Willi syndrome. In some embodiments, the disease or disorder is anorexia or cancer cachexia. For example, the invention provides a method for identifying/screening/validating a compound for suitability for treating or preventing a disease or disorder relating to food intake, wherein the method comprises (i) contacting an intestinal organoid of the invention with the compound; and (ii) determining whether the compound modulates expression or section of, agonises or antagonises PPY in EECs. In some embodiments, the organoid is a reporter organoid in which PPY is tagged with a detectable marker. In some embodiments, the physiological effect is appetite regulation, the target is PPY and the human EECs are human GCG+ cells. For example, in some embodiments, increasing the level of PPY suppresses appetite. This is useful for treating or preventing a disease or disorder such as obesity or Prader-Willi syndrome or diabetes. In some embodiments, decreasing the level of PPY increases appetite. This is useful for treating or preventing anorexia or cancer cachexia. In some embodiments, the one or more targets additionally comprise GLP-1. In some embodiments, there is provided an isolated GCG+ cell expressing PPY. In some embodiments, there is provided an isolated population of GCG+ cells expressing PPY. In some embodiments, the hormone is PPY. In some embodiments, the disease or disorder is a disease or disorder in which PPY is implicated. In some embodiments, the disease or disorder is a disease or disorder treatable or preventable by increasing or inducing expression and/or secretion of PPY. In some embodiments, the disease or disorder is treatable or preventable by decreasing or ceasing expression and/or secretion of PPY. For example, in some embodiments, the disease or disorder is an appetite-related disease or disorder. In some embodiments, the disease or disorder is a disease or disorder requiring appetite stimulation. For example, in some embodiments, the disease or disorder is anorexia or cancer cachexia. In some embodiments, the disease or disorder is a disease or disorder requiring appetite inhibition. For example, in some embodiments, the disease or disorder is diabetes, obesity or Prader-Willi syndrome.

In some embodiments, the human EECs are human EECs with the exception of human M-X cells and/or the target is the transcription factor ASCL1. Ascl1 has been described in endocrine cells in the murine lung (Borges et al., 1997). Transcription factors are responsible for expression of genes in the cell type in which they are expressed. Thus, in some embodiments, the disease or disorder is a disease or disorder in which a hormone expressed and/or secreted and/or produced by or produced from a hormone precursor secreted by the human EEC in which the ASCL1 is expressed is implicated. Examples of such diseases and disorders are provided herein.

In some embodiments, the human EECs are human ECs and/or the target is the transcription factor MNX1. MNX1 is known to promote neonatal diabetes when mutated (Pan et al., 2015). Accordingly, in some embodiments, the disease or disorder is neonatal diabetes. In some embodiments, the patient is a neonate. In some embodiments, the patient is a fetus in utero. Also provided a method for treating or preventing neonatal diabetes comprising reducing or preventing MNX1 production or correcting the sequence of the MNX1 mutation in human ECs. In some embodiments, gene silencing or gene modification techniques (e.g. CRISPR) may be used to reduce or prevent MNX1 production by human ECs or to correct the sequence of the MNX1 mutation. In some embodiments, reducing or preventing MNX1 produced by ECs comprises contacting a patient's ECs with an antagonist of MNX1 production. In some embodiments, the MNX1 is mutated MNX1. In some embodiments, the MNX1 mutation is implicated in neonatal diabetes. In some embodiments, treating or preventing neonatal diabetes comprises increasing the expression of wild type MNX1 in ECs in the patient. As mentioned above, transcription factors are responsible for expression of genes in the cell type in which they are expressed. Thus, in some embodiments, the disease or disorder is a disease or disorder in which a hormone expressed and/or secreted and/or produced by or produced from a hormone precursor secreted by an EC-cell is implicated. EC-cells secrete Serotonin. Thus, in some embodiments, the disease or disorder is a disease or disorder in which Serotonin is implicated. Examples of such diseases and disorders are provided herein. For example, in some embodiments, the disease or disorder is a gut motility disorder, IBS, IBD, depression or diabetes. In some embodiments, the disease or disorder is depression, a gut motility disorder or diabetes (in particular type II diabetes). In some embodiments, the disease or disorder is IBS. In some embodiments, the disease or disorder is IBD. In some embodiments, the disease or disorder is ulcerative colitis. In some embodiments, the disease or disorder is Crohn's disease. In some embodiments, the physiological effect is gut motility, bowel movement and/or activation of pain receptors.

In some embodiments, the human EECs are human TPH1+ cells and/or the target is the transcription factor IRX3. IRX3 is a member of the Iroquois homeobox family, and has not been described in murine EECs (Haber et al., 2017). IRX3 has gained attention recently as a neuronal regulator of energy balance, and genetic variants in IRX3 associate with obesity in humans (Schneeberger, 2019). In some embodiments, the disease or disorder is an imbalance in energy levels. In some embodiments, the disease or disorder is obesity. The finding that IRX3 is expressed in human TPH1+ cells opens the way for targeting therapeutic compounds which target IRX3 to the gut, or to human TPH1+ cells. Associated methods are provided herein accordingly. In some embodiments, methods for treating or preventing obesity comprise targeting genetic variants of IRX3 associated with obesity in the gut, for example, in human TPH1+ cells. In some embodiments, a method for treating or preventing obesity comprises reducing or preventing production of IRX3 genetic variants associated with obesity or correcting the sequence of the IRX3 variants associated with obesity in human TPH1+ cells. In some embodiments, gene silencing or gene modification techniques (e.g. CRISPR) may be used to reduce or prevent IRX3 genetic variant production by human TPH1+ cells or to correct the sequence of the IRX3 genetic variant to a variant that is not associated with obesity. In some embodiments, the physiological effect is energy balance. As mentioned above, transcription factors are responsible for expression of genes in the cell type in which they are expressed. Thus, in some embodiments, the disease or disorder is a disease or disorder in which a hormone expressed and/or secreted and/or produced by or produced from a hormone precursor secreted by a TPH1+-cell is implicated. TPH1+-cells secrete Serotonin. Thus, in some embodiments, the disease or disorder is a disease or disorder in which Serotonin is implicated. Examples of such diseases and disorders are provided herein. For example, in some embodiments, the disease or disorder is a gut motility disorder, IBS, IBD, diabetes or depression. In some embodiments, the disease or disorder is depression, a gut motility disorder or diabetes (in particular type II diabetes). In some embodiments, the disease or disorder is IBS. In some embodiments, the disease or disorder is IBD. In some embodiments, the disease or disorder is ulcerative colitis. In some embodiments, the disease or disorder is Crohn's disease. In some embodiments, the physiological effect is gut motility, bowel movement and/or activation of pain receptors.

In some embodiments, the human EECs are human L-cells and/or the target is the receptor GPR162. GPR162 is an orphan receptor not found in mice. It is reported to be expressed in parts of the brain regulating food intake, while genetic variants in GPR162 are linked to impairments in glucose homeostasis (Caruso et al., 2016). In some embodiments, the disease or disorder is a disease or disorder relating to food intake, e.g. an appetite-related disease or disorder (for example, obesity, anorexia or Prader-Willi syndrome). For example, in some embodiments, the disease or disorder is selected from obesity, diabetes and Prader-Willi syndrome. In some embodiments, the disease or disorder is anorexia or cancer cachexia. In some embodiments, the treating or preventing the appetite-related disease or disorder may comprise modulating levels of GLP-1, e.g. produced by human L-cells. In some embodiments, the disease or disorder is a disease or disorder in which GLP-1 is implicated. Examples of such diseases and disorders are provided herein. In some embodiments, the physiological effect is glucose homeostasis. In some embodiments, the GPR162 is a genetic variant of GPR162 linked to impairments in glucose homeostasis.

In some embodiments, the human EECs are human ECs and/or the target is the receptor GPR68. GPR68 is an orphan GPCR uniquely produced by ECs. A recent study found that the orphan peptide CART (cocaine- and amphetamine-regulated protein) can activate GPR68 (Foster et al., 2019). Multiple sources are suggested for CART, including the brain and EECs, and the peptide has a role in the regulation of anxiety, reward and feeding behaviours (Shcherbina et al., 2018). Accordingly, in some embodiments, the disease or disorder is selected from a disease or disorder relating to food intake, or a disease or disorder related to anxiety and/or reward. In some embodiments, the disease or disorder is obesity or anorexia. In some embodiments, the disease or disorder is obesity, diabetes or Prader-Willi syndrome. In some embodiments, the disease or disorder is anorexia or cancer cachexia. In some embodiments, the physiological effect is selected from one or more of anxiety, reward and feeding behaviour. In some embodiments, the compound (e.g. for use in treating a disease or disorder) is CART.

In some embodiments, the human EECs are human EECs and/or the target is the subunit of the GABA-B receptor GABBR2. GABA-A receptors have been reported in some murine EECs. The inventors found that the GABBR2 subunit of the GABA-B receptor was broadly expressed among EECs, potentially allowing these cells to respond to GABA (Hyland and Cryan, 2010). In some embodiments, the therapeutic compound is GABA or a GABA analog. The use of GABA analogs for treating various diseases and disorders is well known in the art. For example, in some embodiments, the disease or disorder is selected from fibromyalgia; nerve pain resulting from diabetes, amputation, singles or another cause; restless legs syndrome; seizures; and epilepsy. In some embodiments, the treatment or prevention comprises targeting the therapeutic compound to the gut or to human EECs in the gut. Also provided is the use of GABBR2 as a drug target.

In some embodiments, the human EECs are human EECs and/or the target is the melanocortin receptor MC1R. MC1R is a hormone receptor. MC4R has been described in murine L-cells as a regulator of hormone secretion that can be exploited by enriching the microbiome with MSH-like producing bacteria (Panaro et al., 2014). In some embodiments, stimulation of MC1R leads to GLP1 secretion. Accordingly, in some embodiments, the physiological effect is modulation of blood glucose levels. Similarly, in some embodiments, the disease or disorder is diabetes. In some embodiments, the disease or disorder is a disease or disorder in which GLP-1 is implicated. Examples of such diseases and disorders are provided herein. In some embodiments, the physiological effect is selected from appetite, gut motility, insulin secretion and neurological and/or cognitive functioning.

In some embodiments, the human EECs are human ECs and/or the target is the receptor for the thyroid-stimulating-hormone TSHR. Serotonin is known to regulate the levels of circulating thyroid hormones (Sullo et al., 2011), and expression of TSHR in ECs suggests that this regulation could work bidirectionally. In some embodiments, affecting serotonin levels by targeting the TSHR in human ECs is useful for treating a disease or disorder as described herein, for example, a disease or disorder affected by serotonin, for example, for treating IBD or diabetes. Thus, in some embodiments, the disease or disorder is a disease or disorder affected by serotonin, for example, diabetes or IBD. In some embodiments, the disease or disorder is a gut motility disorder, e.g. as described herein, IBS, IBD, diabetes or depression. In some embodiments, the disease or disorder is ulcerative colitis. In some embodiments, the disease or disorder is Crohn's disease. In some embodiments, the hormone is serotonin. In some embodiments, the physiological effect is gut motility, bowel movement and/or activation of pain receptors.

In some embodiments, the human EECs are human ECs and/or the target is the receptor for the L-cell hormone PYY, NPY1R. NPY1R has been reported in murine enterocytes as a regulator of electrolyte transport (Goldspink et al., 2018). However, the inventors did not confirm expression of NPY1R in the CHGA-mNeon⁻ population, enriched for human non-EECs which includes enterocytes. Instead, the inventors observed exclusive expression of Npy1r in human Serotonin-producing EC cells. This contrasts with previous suggestions that the PYY-receptor Npy1r is an enterocyte marker in mouse (Goldspink et al., 2018). Accordingly, the invention provides the use of expression of Npy1r as a marker for human ECs. In some embodiments, the physiological effect is electrolyte transport, the target is NPY1R and the human EECs are human ECs. The inventors have found that human EECs can sense extracellular PYY using the NPY1R receptor. In some embodiments, the physiological effect is modulation of serotonin production or secretion, e.g. by human ECs. Thus, in some embodiments, the disease or disorder is a disease or disorder affected by serotonin, for example, diabetes or IBD. In some embodiments, the disease or disorder is a gut motility disorder, e.g. as described herein, IBS, IBD, diabetes or depression. In some embodiments, the disease or disorder is ulcerative colitis. In some embodiments, the disease or disorder is Crohn's disease. In some embodiments, the disease or disorder is a disease or disorder in which PYY is implicated. Examples of such diseases and disorders are provided herein. For example, in some embodiments, the disease or disorder is an appetite-related disease or disorder. In some embodiments, the compound (e.g. for use in treating a disease or disorder) is PYY. In some embodiments, the physiological effect is selected from gut motility, bowel movement and/or activation of pain receptors and appetite.

The invention further provides the 20 most significant expressed markers from the RNA sequencing dataset for the different EEC populations. In some embodiments, these are as shown in the Figures.

For CHGA+ cells, in some embodiments, the 20 most significant expressed RNA markers are: Prostate Stem Cell Antigen (PSCA), H19 Imprinted Maternally Expressed Transcript (H19), Carboxylesterase 1 (CES1), Chromogranin B (CHGB), kinesin family member 1A (KIF1A), Olfactory Receptor Family 51 Subfamily E Member 1 (OR51E1), Crystallin Beta A2 (CRYBA2), Calcium Voltage-Gated Channel Auxiliary Subunit Alpha2delta 1 (CACNA2D1), Solute Carrier Family 18 Member A1 (SLC18A1), RAB3C Member RAS Oncogene Family (RAB3C), Paired box gene 4 (PAX4), LIM Homeobox Transcription Factor 1 Alpha (LMX1A), Sodium Voltage-Gated Channel Alpha Subunit 3 (SCN3A), Potassium Inwardly Rectifying Channel Subfamily J Member 6 (KCNJ6), Synaptic Vesicle Glycoprotein 2A (SV2A), Suppression of tumorigenicity 18 protein (ST18), APC Membrane Recruitment Protein 3 (AMER3), Germ Cell Associated 1 (GSG1), Transmembrane Protein With EGF Like And Two Follistatin Like Domains 2 (TMEFF2).

For GCG+ cells, in some embodiments the 20 most significant expressed RNA markers are: Chromogranin A (CHGA), Glucagon (GCG), Peripherin (PRPH), SV2 Related Protein (SVOP), Potassium Inwardly Rectifying Channel Subfamily J Member 11 (KCNJ11), Serine/Threonine Kinase 32A (STK32A), Somatostatin Receptor 5 (SSTR5), Collagen, type II, alpha 1 (COL2A1), Coronin 2B (CORO2B), RNF157 Antisense RNA 1 (RNF157-AS1), Thrombospondin 4 (THBS4), Sodium Voltage-Gated Channel Alpha Subunit 7 (SCN7A), SCTR, GPBAR1, Peptide YY (PYY), ST18, Cholestocystokinin (CCK), Paired Box 6 (PAX6) and Pancreatic polypeptide (PPY).

For TPH1+ cells, in some embodiments the 20 most significant expressed RNA markers are: CHGA, kinesin family member 1A (KIF1A), Claudin 18 (CLDN18), Solute Carrier Family 18 Member A1 (SLC18A1), LIM Homeobox Transcription Factor 1 Alpha (LMX1A), Sodium Voltage-Gated Channel Alpha Subunit 3 (SCN3A), Family With Sequence Similarity 46 Member B (FAM46B), Proprotein Convertase Subtilisin/Kexin Type 2 (PCSK2), RAB3C Member RAS Oncogene Family (RAB3C), paired box 4 (PAX4), Regenerating Family Member 3 Alpha (REG3A), Protocadherin 20 (PCDH20), Potassium Voltage-Gated Channel Interacting Protein 1 (KCNIP1), Cell Adhesion Molecule 2 (CADM2), Iroquois-class homeodomain protein IRX-3 (IRX3), Suppression of tumorigenicity 18 protein (ST18), APC Membrane Recruitment Protein 3 (AMER3), Chromogranin B (CHGB) and Calcium Voltage-Gated Channel Auxiliary Subunit Alpha2delta 1 (CACNA2D1).

For MLN+ cells, in some embodiments the 20 most significant expressed RNA markers are: Insulin gene enhancer protein ISL-1 (ISL1), Paired box 6 (PAX6), Ghrelin (GHRL), Coagulation Factor X (F10), NK2 Homeobox 2 (NKX2-2), ST18, Chromosome 14 Open Reading Frame 132 (C14orf132), kinesin family member 5C (KIF5C), Vitronectin (VTN), Cripto FRL-1 Cryptic Family 1 (CFC1), Placenta-specific protein 9 (PLAC9), phosphodiesterase 8B (PDE8B), Long Intergenic Non-Protein Coding RNA 643 (LINC00643), S100 Calcium Binding Protein A1 (S100A1), Transgelin 3 (TAGLN3), SET Binding Protein 1 (SETBP1), Adenylate Cyclase 1 (ADCY1), potassium voltage-gated channel subfamily J member 11 (KCNJ11), Serine/Threonine Kinase 32A (STK32A) and Carboxypeptidase B1 (CPB1).

The invention further provides the 20 most significant expressed markers at the protein level for the different EEC populations. In some embodiments, these are as shown in the Figures.

For CHGA+ cells, in some embodiments, the 20 most significant expressed protein markers are: Endoplasmic Reticulum Oxidoreductase 1 Beta (ERO1B), Gastric Inhibitory Polypeptide (GIP), Glucagon (GCG), Signal Transducer And Activator Of Transcription 5A (STAT5A), Carboxypeptidase B1 (CPB1), CD74, Prostate Stem Cell Antigen (PSCA), Peptidylglycine Alpha-Amidating Monooxygenase (PAM), Carboxypeptidase E (CPE), Insulin Like Growth Factor Binding Protein 3 (IGFBP3), Proprotein Convertase Subtilisin/Kexin Type 1 Inhibitor (PCSK1 N), Midkine (MDK), Secretogranin V (SGNE1), Von Willebrand Factor A Domain Containing 5B2 (VWA5B2), Potassium Channel Tetramerization Domain Containing 12 (KCTD12), Chromogranin A (CHGA), GC Vitamin D Binding Protein (HEL-S-51), Somatostatin (SST), Tryptophan Hydroxylase 1 (TPH1) and Carboxylesterase 1 (CES1).

For GCG+ cells, in some embodiments, the 20 most significant expressed protein markers are: VGF Nerve Growth Factor Inducible (VGF), MDK, SGNE1, PAM, CPE, Secretogranin Ill (SCG3), CD74, PCSK1N, IGFBP3, PSCA, Potassium Channel Tetramerization Domain Containing 12 (KCTD12), CHGA, Ribosomal Protein Lateral Stalk Subunit P1 (RPLP1), ERO1B, GIP, GCG, Signal Transducer And Activator Of Transcription 5A (STAT5A), CPB1, Coiled-Coil-Helix-Coiled-Coil-Helix Domain Containing 1 (CHCHD1) and Pancreatic Polypeptide (PPY).

For TPH1+ cells, in some embodiments, the 20 most significant expressed protein markers are: Smoothelin Like 2 (SMTNL2), Solute Carrier Family 18 Member A1 (SLC18A1), Carboxylesterase 1 (CES1), Cornulin (CRNN), Chromogranin B (CHGB), Ras-Related Protein Rab-1A (RAB1), HEL-S-51, SST, TPH1, MIA SH3 Domain Containing (MIA), CD74, IGFBP3, PAM, CPE, PCSK1N, MDK, SGNE1, VWA5B2, KCTD12 and CHGA.

For MLN+ cells, in some embodiments, the 20 most significant expressed protein markers are: Dermcidin (DCD), S100 Calcium Binding Protein A1 (S100A1), GHRL, GCG, STAT5A, Gastrin (GAST), Neurotensin (NTS), CPB1, RPLP1, PCSK1N, MDK, PAM, SGNE1, CPE, SCG3, CD74, PSCA, IGFPB3, KCTD12 and CHGA.

In some embodiments, the invention provides a method of identifying a cell as belonging to a particular EEC subtype comprising determining if the cell expresses one or more (e.g. 1, 2, 3, 4, 5, 6, 7, 8, 9, 10, 11, 12, 13, 14, 15, 16, 17, 18, 19 or more or all 20) of the 20 most significant expressed markers listed above for the particular cell subtype. In some embodiments, the markers are the RNA markers. In some embodiments, the markers are the protein markers. In some embodiments, the markers are the RNA and the protein markers and then one or more (up to all 20) of each of the RNA and protein markers may be used. Thus, these markers may be used as one or more targets according to the invention. For example, they may be used as cell markers for isolating and/or identifying cells using methods of the invention. Also provided are isolated human cells and isolated populations of human cells comprising these markers. For example, in some embodiments, there is provided an isolated human CHGA+ cell, GCG+ cell, TPH1+ cell or MLN+ cell comprising at least 5 (e.g. at least 8, 10, 12, 15, 17, 19 or all 20) of the respective markers. An isolated population of such cells is similarly provided. In some embodiments, the disease or disorder is a disease or disorder in which one or more hormones expressed and/or secreted and/or produced by or produced from a hormone precursor secreted by the human EEC in which the target is expressed is implicated. Examples of such diseases and disorders are provided herein.

In some embodiments, the target in human EECs is selected from PSCA, H19, CES1, CHGB, KIF1A, OR51E1, CRYBA2, CACNA2D1, SLC18A1, RAB3C, PAX4, LMX1A, SCN3A, KCNJ6, SV2A, ST18, AMER3, GSG1 and TMEFF2.

In some embodiments, the target in a human L-cell is selected from CHGA, GCG, PRPH, SVOP, KCNJ11, STK32A, SSTR5, COL2A1, CORO2B, RNF157-AS1, THBS4, SCN7A, SCTR, GPBAR1, PYY, ST18, CCK, PAX6 and PPY.

In some embodiments, the target in a human EC-cell is selected from CHGA, KIF1A, CLDN18, SLC18A1, LMX1A, SCN3A, FAM46B, PCSK2, RAB3C, PAX4, REG3A, PCDH20, KCNIP1, CADM2, IRX3, ST18, AMER3, CHGB and CACNA2D1.

In some embodiments, the target in a human M/X-cell is selected from ISL1, PAX6, GHRL, F10, NKX2-2, ST18, C14orf132, KIF5C, VTN, CFC1, PLAC9, PDE8B, LIN000643, S100A1, TAGLN3, SETBP1, ADCY1, KCNJ11, STK32A and Carboxypeptidase B1 (CPB1).

In some embodiments, the target in human EECs is selected from ERO1 B, GIP, GCG, STAT5A, CPB1, CD74, PSCA, PAM, CPE, IGFBP3, PCSK1N, MDK, SGNE1, VWA5B2, KCTD12, CHGA, HEL-S-51, SST, TPH1 and CES1.

In some embodiments, the target in a human L-cell is selected from VGF, MDK, SGNE1, PAM, CPE, SCG3, CD74, PCSK1N, IGFBP3, PSCA, KCTD12, CHGA, RPLP1, ERO1B, GIP, GCG, STAT5A, CPB1, CHCHD1 and PPY.

In some embodiments, the target in a human EC-cell is selected from SMTNL2, SLC18A1, CES1, CRNN, CHGB, RAB1, HEL-S-51, SST, TPH1, MIA, CD74, IGFBP3, PAM, CPE, PCSK1N, MDK, SGNE1, VWA5B2, KCTD12 and CHGA.

In some embodiments, the target in a human M/X-cell is selected from DCD, S100A1, GHRL, GCG, STAT5A, GAST, NTS, CPB1, RPLP1, PCSK1N, MDK, PAM, SGNE1, CPE, SCG3, CD74, PSCA, IGFPB3, KCTD12 and CHGA.

The inventors have, for the first time, established human EEC subtype-specific proteomes. The reporter organoids of the invention allow subpopulations of EECs to be obtained, e.g. using FACS, and processed for intracellular proteomics. The inventors have identified a number of secreted peptides and proteins not attributed to EECs before. These are described below.

In some embodiments, the human EECs are human EC-cells and/or the target is CHGA_2. CHGA codes for a 457-amino acid preproprotein that is cleaved into many different bioactive products. In some embodiments, CHGA_2 is a peptide product of CHGA which spans terminal sequences of exon 5 and exon 7.

In some embodiments, the human EECs are human EECs and/or the target is Neuropeptide W (NPW). NPW has recently been proposed as an EEC hormone based on bulk RNA sequencing (Roberts et al. 2019). The inventors have shown for the first time that NPW is expressed at the protein level and secreted. NPW is expressed in different parts of the brain. In some embodiments, the disease or disorder is a disease or disorder in which NPW is implicated. In some embodiments, the disease or disorder is a disease or disorder treatable or preventable by increasing or inducing expression and/or secretion of NPW. In some embodiments, the disease or disorder is a disease or disorder treatable or preventable by decreasing or ceasing expression and/or secretion of NPW. NPW increases food intake when injected in the hypothalamus (Levine et al., 2005). In some embodiments, the disease or disorder is therefore a disease or disorder relating to food intake, e.g. an appetite-related disease or disorder (for example, obesity or anorexia). For example, in some embodiments, the disease or disorder is selected from obesity, diabetes and Prader-Willi syndrome. In some embodiments, the disease or disorder is anorexia or cancer cachexia. Thus, in some embodiments, the invention provides a method for identifying/screening/validating a compound for suitability for treating or preventing a disease or disorder relating to food intake, wherein the method comprises (i) contacting an intestinal reporter organoid of the invention comprising tagged NPW with the compound; and (ii) determining whether secretion of NPW is affected. Depending on the disease or disorder to be treated, it may be desirable to increase or decrease secretion of NPW. For example, in embodiments in which the disease or disorder is obesity, diabetes or Prader-Willi syndrome, it is desirable to decrease NPW. In embodiments in which the disease or disorder is anorexia or cancer cachexia, it is desirable to increase NPW. The surprising finding that the NPW protein is expressed in the gut and secreted opens the possibility of targeting treatments for such diseases or disorders to the gut or to human EECs in the gut. Gut targeting would be preferable to injection into the hypothalamus, due to the invasive nature of the latter. For example, in some embodiments, the treatment or prevention of the disease or disorder relating to food intake comprises increasing expression of NPW in the gut or in human EECs in the gut. The skilled person would be able to increase expression of NPW in the gut or in human EECs using the teaching described herein in combination with the teaching available in the art. For example, in some embodiments, the treatment or prevention comprises administering NPW to the gut or to human EECs in the gut. Similarly, in some embodiments, the treatment or prevention comprises administering a compound identified as affecting (e.g. increasing) NPW expression and/or secretion by human EECs, e.g. using a method of the invention. In contrast, in some embodiments, the treatment or prevention of the disease or disorder relating to food intake comprises decreasing expression of NPW in the gut or in human EECs in the gut. The skilled person would be able to decrease expression of NPW in the gut or in human EECs using the teaching described herein in combination with the teaching available in the art. Similarly, in some embodiments, the physiological effect is food intake, the target is NPW and the human EECs are human EECs. For example, in some embodiments, modulating the physiological effect comprises increasing or decreasing food intake.

In some embodiments, the human EECs are human EECs and/or the target is VGF. VGF has recently been proposed as an EEC hormone based on bulk RNA sequencing (Roberts et al. 2019). The inventors have shown for the first time that VGF is expressed at the protein level and secreted. In some embodiments, the disease or disorder is a disease or disorder in which VGF is implicated. In some embodiments, the disease or disorder is a disease or disorder treatable or preventable by increasing or inducing expression and/or secretion of VGF. In some embodiments, the disease or disorder is a disease or disorder treatable or preventable by decreasing or ceasing expression and/or secretion of VGF.

In some embodiments, the human EECs are human L-cells and/or the target is PPY. PPY is a hormone. As mentioned elsewhere herein, in some embodiments, the disease or disorder is a disease or disorder in which PPY is implicated. In some embodiments, the disease or disorder is a disease or disorder treatable or preventable by increasing or inducing expression and/or secretion of PPY. In some embodiments, the disease or disorder is a disease or disorder treatable or preventable by decreasing or ceasing expression and/or secretion of PPY.

In some embodiments, the human EECs are human EECs and/or the target is Midkine. As mentioned elsewhere herein, in some embodiments, the disease or disorder is a disease or disorder in which Midkine is implicated. For example, Midkine may be implicated in a disease or disorder by affecting the secretion of one or more other hormones implicated in the disease or disorder. Thus, in some embodiments, the disease or disorder is a disease or disorder as described herein.

In some embodiments, the human EECs are human M-cells and/or the target is the peptidase CPB1. Thus, in some embodiments, the physiological effect is hormone processing, e.g. in the intestine. As mentioned elsewhere herein, in some embodiments, the disease or disorder is a disease or disorder in which a hormone that has been processed is implicated. In some embodiments, the hormone is GLP-1.

In some embodiments, the human EECs are human EECs and/or the target is REG3A. Mouse EECs, in contrast, express REG4. REG3A is an antimicrobial peptide. Accordingly, in some embodiments, the disease or disorder is a microbial infection, e.g. of the intestine. A method of treating or preventing a microbial infection according to the invention may therefore comprise increasing expression and/or secretion of REG4 in human EECs, for example, by administering a compound which increases expression and/or secretion of REG3A. Similarly, a method for treating or preventing a microbial infection according to the invention may comprise administering REG3A to a patient such that it is delivered to the patient's gut.

In some embodiments, the human EECs are human EECs and/or the target is the enzyme PAM (Peptidyl-glycine alpha-amidating monooxygenase). PAM is known to modify endocrine peptides by C-terminal amidation, important for their activation. Coding variants of PAM have been associated with type 2 diabetes risk and can affect insulin secretion (Thomsen et al., 2018). In some embodiments, the disease or disorder is therefore type II diabetes. In some embodiments, the physiological effect is insulin secretion. In some embodiments, the physiological effect is C-terminal amidation of endocrine peptides, e.g. in the intestine. In some embodiments, the physiological effect is activation of endocrine peptides, e.g. by C-terminal amidation of the endocrine peptides. In some embodiments, the disease or disorder is a disease or disorder in which a hormone that requires C-terminal amidation for its activation is implicated.

In some embodiments, the human EECs are human EECs and/or the target is Nucleobindin-2 (NUC2B) precursor. In some embodiments, the human EECs are human EECs and/or the one or more targets are one or more of (e.g. 1, 2, or more or all 3 of) neuropeptides Nesfatin-1, 2 and 3. It is known that the Nucleobindin-2 (NUC2B) precursor is processed to the neuropeptides Nesfatin-1, 2 and 3 (Ramesh et al., 2015). Nesfatin-1 has recently gained attention as an anorexigenic and insulinotropic peptide, produced in the hypothalamus and pancreas. Accordingly, in some embodiments, the disease or disorder is anorexia. For example, in some embodiments, a method of treating or preventing anorexia comprises decreasing expression and/or secretion of Nestafin-1 and/or inhibiting the activity of Nestafin-1 in human EECs. In some embodiments, the disease or disorder is diabetes. In some embodiments, the diabetes is caused by an insufficiency of insulin. In some embodiments, the diabetes is type I diabetes. For example, in some embodiments, the treating or preventing diabetes comprises increasing the expression and/or secretion of Nestafin-1 in human EECs. In some embodiments, the physiological effect is insulin levels. Nesfatin-1 has been shown to regulate GLP-1 and GIP secretion in vitro (Ramesh et al., 2015). In some embodiments, the physiological effect is GLP-1 and/or GIP secretion by human EECs, e.g. in vitro. In some embodiments, the disease or disorder is a disease or disorder in which GLP-1 is implicated. Examples of such diseases and disorders are provided herein. In some embodiments, the disease or disorder is a disease or disorder in which GIP is implicated. Examples of such diseases and disorders are provided herein. In some embodiments, the physiological effect is selected from appetite, gut motility, insulin secretion and neurological and/or cognitive functioning.

In some embodiments, the human EECs are human EECs and/or the target is Nucleobindin-2 (NUC2B) precursor. In some embodiments, the disease or disorder is anorexia. In some embodiments, the disease or disorder is diabetes. In some embodiments, the disease or disorder is type I diabetes. In some embodiments, the disease or disorder is a disease or disorder in which GLP-1 is implicated. In some embodiments, the disease or disorder is a disease or disorder in which GIP is implicated. In some embodiments, the disease or disorder is a disease or disorder treatable or preventable by increasing or inducing expression and/or secretion of GIP. In some embodiments, the disease or disorder is a disease or disorder treatable or preventable by decreasing or ceasing expression and/or secretion of GIP. In some embodiments, the disease or disorder is a disease or disorder treatable or preventable by increasing or inducing production of GLP-1. In some embodiments, the disease or disorder is a disease or disorder treatable or preventable by decreasing or ceasing production of GLP-1. Examples of such diseases and disorders are described herein

In some embodiments, the human EECs are human EECs and/or the target is Nesfatin-1. In some embodiments, the disease or disorder is anorexia. In some embodiments, the disease or disorder is diabetes. In some embodiments, the disease or disorder is type I diabetes. In some embodiments, the disease or disorder is a disease or disorder in which GIP is implicated. In some embodiments, the disease or disorder is a disease or disorder in which GLP-1 is implicated. In some embodiments, the disease or disorder is a disease or disorder treatable or preventable by increasing or inducing expression and/or secretion of GIP. In some embodiments, the disease or disorder is a disease or disorder treatable or preventable by decreasing or ceasing expression and/or secretion of GIP. In some embodiments, the disease or disorder is a disease or disorder treatable or preventable by increasing or inducing production of GLP-1. In some embodiments, the disease or disorder is a disease or disorder treatable or preventable by decreasing or ceasing production of GLP-1. Examples of such diseases and disorders are described herein.

In some embodiments, the human EECs are human EECs and/or the target is Nestafin-2. In some embodiments, the disease or disorder is anorexia. In some embodiments, the disease or disorder is diabetes. In some embodiments, the disease or disorder is type I diabetes. In some embodiments, the disease or disorder is a disease or disorder in which GLP-1 is implicated. In some embodiments, the disease or disorder is a disease or disorder in which GIP is implicated. In some embodiments, the disease or disorder is a disease or disorder treatable or preventable by increasing or inducing expression and/or secretion of GIP. In some embodiments, the disease or disorder is a disease or disorder treatable or preventable by decreasing or ceasing expression and/or secretion of GIP. In some embodiments, the disease or disorder is a disease or disorder treatable or preventable by increasing or inducing production of GLP-1. In some embodiments, the disease or disorder is a disease or disorder treatable or preventable by decreasing or ceasing production of GLP-1. Examples of such diseases and disorders are described herein.

In some embodiments, the human EECs are human EECs and/or the target is Nestafin-3. In some embodiments, the disease or disorder is anorexia. In some embodiments, the disease or disorder is diabetes. In some embodiments, the disease or disorder is type I diabetes. In some embodiments, the disease or disorder is a disease or disorder in which GLP-1 is implicated. In some embodiments, the disease or disorder is a disease or disorder in which GIP is implicated. In some embodiments, the disease or disorder is a disease or disorder treatable or preventable by increasing or inducing expression and/or secretion of GIP. In some embodiments, the disease or disorder is a disease or disorder treatable or preventable by decreasing or ceasing expression and/or secretion of GIP. In some embodiments, the disease or disorder is a disease or disorder treatable or preventable by increasing or inducing production of GLP-1. In some embodiments, the disease or disorder is a disease or disorder treatable or preventable by decreasing or ceasing production of GLP-1. Examples of such diseases and disorders are described herein.

The inventors have identified a number of targets that are homologues of RNAs and proteins known in the mouse. However, the inventors have surprisingly found that stimulation of the human receptor may lead to secretion of a different hormone/hormone profile than would be obtained by stimulation of the mouse receptor. For example, stimulation of GPBAR1 in human EECs would also regulate PPY (on top of GLP-1, PYY, NTS that are known from mouse). Similarly, stimulation of SSTR5 in human EECs it would also regulate PPY (on top of GLP-1, PYY, NTS that are known from mouse). Similarly, stimulation of FFAR4 in human EECs would also regulate PPY (on top of GLP-1, PYY, NTS that are known from mouse). In addition, FFAR4 is also expressed in Motilin/Ghrelin producing cells which do not exist in mice. Thus, simulation of FFAR4 would also regulate Motilin and/or Ghrelin. Thus, targeting human homologues of mouse proteins can provide surprising effects. Thus, in some embodiments, a human homologue of a mouse gene, RNA or protein is used as the target in a method as described herein.

Thus, the invention provides a method for modulating expression and/or secretion of PPY comprising targeting the GPBAR1 receptor, e.g. on L-cells. Similarly, the invention provides a method for modulating expression and/or secretion of PPY comprising targeting the SSTR5 receptor, e.g. on L-cells. Similarly, the invention provides a method for modulating expression and/or secretion of PPY comprising targeting the FFAR4 receptor, e.g. on L-cells, wherein the method optionally further comprises modulating expression and/or secretion of MLN and/or GHRL. Such methods may be used in the methods of treating and preventing a disease or disorder provided herein. In some embodiments, the disease or disorder is a disease or disorder in which PPY is implicated. In some embodiments, the disease or disorder is a disease or disorder in which MLN and/or GHRL is implicated. Examples of such diseases and disorders are provided herein.

Accordingly, in some embodiments, the target is selected from FFAR4 (e.g. and the human EECs are L-cells or M/X-cells), GPBAR1 (e.g. and the human EECs are L-cells), SSTR5 (e.g. and the human EECs are L-cells), FFAR2 (e.g. and the human EECs are EECs), OR51E1 (e.g. and the human EECs are ECs), ADGRG4 (e.g. and the human EECs are ECs), CASR (e.g. and the human EECs are EECs), PAX4 (e.g. and the human EECs are D-cells or EC-cells), (Aristaless Related Homeobox) ARX (e.g. and the human EECs are EECs), HHEX (e.g. and the human EECs are D-cells), LMX1A (e.g. and the human EECs are ECs), NKX2-2 (e.g. and the human EECs are EECs), PAX6 (e.g. and the human EECs are EECs), (SRY-Box Transcription Factor 4) SOX4 (e.g. and the human EECs are EECs), (Regulatory Factor X6) RFX6 (e.g. and the human EECs are EECs), CHGA (e.g. and the human EECs are EECs), CHGB (e.g. and the human EECs are ECs), (G Protein-Coupled Receptor 112) GPR112 (e.g. and the human EECs are ECs), OR51E1 (e.g. and the human EECs are ECs), (Ectonucleotide Pyrophosphatase/Phosphodiesterase 1) ENPP1 (e.g. and the human EECs are M/X-cells) and (Acyl-CoA Synthetase Long Chain Family Member 1) ACSL1 (e.g. and the human EECs are M/X-cells). In some embodiments, the target is a cell surface receptor selected from: FFAR4 (e.g. and the human EECs are L-cells or M/X-cells), GPBAR1 (e.g. and the human EECs are L-cells), SSTR5 (e.g. and the human EECs are L-cells), FFAR2 (e.g. and the human EECs are EECs), OR51E1 (e.g. and the human EECs are ECs), ADGRG4 (e.g. and the human EECs are ECs) and CASR (e.g. and the human EECs are EECs). In some embodiments, the cell surface receptor is a GPCR. In some embodiments, the target is a transcription factor selected from PAX4 (e.g. and the human EECs are D-cells or EC-cells), ARX (e.g. and the human EECs are EECs), HHEX (e.g. and the human EECs are D-cells), LMX1A (e.g. and the human EECs are ECs), NKX2-2 (e.g. and the human EECs are EECs), PAXB (e.g. and the human EECs are EECs), SOX4 (e.g. and the human EECs are EECs) and RFX6 (e.g. and the human EECs are EECs). In some embodiments, the target is selected from FFAR4 (e.g. and the human EECs are L-cells or M/X-cells), GPBAR1 (e.g. and the human EECs are L-cells), SSTR5 (e.g. and the human EECs are L-cells). In some embodiments, the disease or disorder is a disease or disorder in which a hormone expressed and/or secreted and/or produced by or produced from a hormone precursor secreted by the human EEC in which the target is expressed is implicated. Examples of such diseases and disorders are provided herein.

In some embodiments, the target is FFAR4. In some embodiments, the disease or disorder is a disease or disorder in which a hormone expressed and/or secreted and/or produced by or produced from a hormone precursor secreted by a human L-cell is implicated. In some embodiments, the disease or disorder is a disease or disorder treatable or preventable by increasing or inducing expression and/or secretion and/or production of GLP-1. In some embodiments, the disease or disorder is a disease or disorder treatable or preventable by decreasing or ceasing expression and/or secretion and/or production of GLP-1. Examples of such diseases are provided herein. In some embodiments, the disease or disorder is a disease or disorder in which a hormone expressed and/or secreted and/or produced by or produced from a hormone precursor secreted by a human M/X cell is implicated. In some embodiments, the disease or disorder is a disease or disorder treatable or preventable by increasing or inducing expression and/or secretion and/or production of Motilin. In some embodiments, the disease or disorder is a disease or disorder treatable or preventable by decreasing or ceasing expression and/or secretion and/or production of Motilin. In some embodiments, the disease or disorder is a disease or disorder treatable or preventable by increasing or inducing expression and/or secretion and/or production of Ghrelin. In some embodiments, the disease or disorder is a disease or disorder treatable or preventable by decreasing or ceasing expression and/or secretion and/or production of Ghrelin. Examples of such diseases are provided herein.

In some embodiments, the human EECs are L-cells and/or the target is GPBAR1. In some embodiments, the disease or disorder is a disease or disorder in which a hormone expressed and/or secreted and/or produced by or produced from a hormone precursor secreted by a human L-cell is implicated. In some embodiments, the disease or disorder is a disease or disorder treatable or preventable by increasing or inducing expression and/or secretion and/or production of GLP-1. In some embodiments, the disease or disorder is a disease or disorder treatable or preventable by decreasing or ceasing expression and/or secretion and/or production of GLP-1. Examples of such diseases are provided herein.

In some embodiments, the human EECs are L-cells and/or the target is SSTR5. In some embodiments, the disease or disorder is a disease or disorder in which a hormone expressed and/or secreted and/or produced by or produced from a hormone precursor secreted by a human L-cell is implicated. In some embodiments, the disease or disorder is a disease or disorder treatable or preventable by increasing or inducing expression and/or secretion and/or production of GLP-1. In some embodiments, the disease or disorder is a disease or disorder treatable or preventable by decreasing or ceasing expression and/or secretion and/or production of GLP-1. Examples of such diseases are provided herein.

In some embodiments, the human EECs are EECs and/or the target is FFAR2. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the human EECs are ECs-cells and/or the target is OR51E1. In some embodiments, the disease or disorder is a disease or disorder in which a hormone expressed and/or secreted and/or produced by or produced from a hormone precursor secreted by a human EC-cell is implicated. In some embodiments, the disease or disorder is a disease or disorder treatable or preventable by increasing or inducing expression and/or secretion and/or production of serotonin. In some embodiments, the disease or disorder is a disease or disorder treatable or preventable by decreasing or ceasing expression and/or secretion and/or production of serotonin. Examples of such diseases are provided herein.

In some embodiments, the human EECs are EC-cells and/or the target is ADGRG4. In some embodiments, the disease or disorder is a disease or disorder in which a hormone expressed and/or secreted and/or produced by or produced from a hormone precursor secreted by a human EC-cell is implicated. In some embodiments, the disease or disorder is a disease or disorder treatable or preventable by increasing or inducing expression and/or secretion and/or production of serotonin. In some embodiments, the disease or disorder is a disease or disorder treatable or preventable by decreasing or ceasing expression and/or secretion and/or production of serotonin. Examples of such diseases are provided herein.

In some embodiments, the human EECs are EECs and/or the target is CASR. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the target is SRY-Box Transcription Factor 4 (SOX4). In some embodiments, the disease or disorder is a disease or disorder in which a hormone expressed and/or secreted and/or produced by or produced from a hormone precursor secreted by human EECs is implicated.

In some embodiments, the target is Regulatory Factor X6 (RFX6). In some embodiments, the disease or disorder is a disease or disorder in which a hormone expressed and/or secreted and/or produced by or produced from a hormone precursor secreted by human EECs is implicated.

In some embodiments, the target is CHGA. In some embodiments, the disease or disorder is a disease or disorder in which a hormone expressed and/or secreted and/or produced by or produced from a hormone precursor secreted by human EECs is implicated.

In some embodiments, the target is CHGB. In some embodiments, the disease or disorder is a disease or disorder in which a hormone expressed and/or secreted and/or produced by or produced from a hormone precursor secreted by a human EC-cell is implicated.

In some embodiments, the target is G Protein-Coupled Receptor 112 (GPR112). In some embodiments, the disease or disorder is a disease or disorder in which a hormone expressed and/or secreted and/or produced by or produced from a hormone precursor secreted by a human EC-cell is implicated.

In some embodiments, the target is OR51E1. In some embodiments, the disease or disorder is a disease or disorder in which a hormone expressed and/or secreted and/or produced by or produced from a hormone precursor secreted by human EC-cells is implicated.

In some embodiments, the target is Ectonucleotide Pyrophosphatase/Phosphodiesterase 1 (ENPP1). In some embodiments, the disease or disorder is a disease or disorder in which a hormone expressed and/or secreted and/or produced by or produced from a hormone precursor secreted by human M/X-cells is implicated.

In some embodiments, the target is Acyl-CoA Synthetase Long Chain Family Member 1 (ACSL1). In some embodiments, the disease or disorder is a disease or disorder in which a hormone expressed and/or secreted and/or produced by or produced from a hormone precursor secreted by human M/X-cells is implicated.

In some embodiments, the target is FFAR4. In some embodiments, the disease or disorder is a disease or disorder in which a hormone expressed and/or secreted and/or produced by or produced from a hormone precursor secreted by human L-cells or human M/X-cells is implicated.

In some embodiments, the target is GPBAR1. In some embodiments, the disease or disorder is a disease or disorder in which a hormone expressed and/or secreted and/or produced by or produced from a hormone precursor secreted by human L-cells is implicated.

In some embodiments, the target is SSTR5. In some embodiments, the disease or disorder is a disease or disorder in which a hormone expressed and/or secreted and/or produced by or produced from a hormone precursor secreted by human L-cells is implicated.

In some embodiments, the target is FFAR2. In some embodiments, the disease or disorder is a disease or disorder in which a hormone expressed and/or secreted and/or produced by or produced from a hormone precursor secreted by human EECs is implicated.

In some embodiments, the target is OR51E1. In some embodiments, the disease or disorder is a disease or disorder in which a hormone expressed and/or secreted and/or produced by or produced from a hormone precursor secreted by human EC-cells is implicated.

In some embodiments, the target is ADGRG4. In some embodiments, the disease or disorder is a disease or disorder in which a hormone expressed and/or secreted and/or produced by or produced from a hormone precursor secreted by human EC-cells is implicated.

In some embodiments, the target is CASR. In some embodiments, the disease or disorder is a disease or disorder in which a hormone expressed and/or secreted and/or produced by or produced from a hormone precursor secreted by human EECs is implicated.

In some embodiments, the target is PAX4 (e.g. and the human EECs are D-cells or EC-cells). In some embodiments, the disease or disorder is a disease or disorder in which a hormone expressed and/or secreted and/or produced by or produced from a hormone precursor secreted by human D-cells or EC-cells is implicated.

In some embodiments, the target is ARX. In some embodiments, the disease or disorder is a disease or disorder in which a hormone expressed and/or secreted and/or produced by or produced from a hormone precursor secreted by human EECs is implicated. In some embodiments, the human EECs are all EECs except D-cells and EC-cells.

In some embodiments, the target is HHEX. In some embodiments, the disease or disorder is a disease or disorder in which a hormone expressed and/or secreted and/or produced by or produced from a hormone precursor secreted by human D-cells is implicated.

In some embodiments, the target is LMX1A. In some embodiments, the disease or disorder is a disease or disorder in which a hormone expressed and/or secreted and/or produced by or produced from a hormone precursor secreted by human EC-cells or L-cells is implicated. In some embodiments, the disease or disorder is a disease or disorder in which a hormone expressed and/or secreted and/or produced by or produced from a hormone precursor secreted by human EC-cells is implicated.

In some embodiments, the target is NKX2-2. In some embodiments, the disease or disorder is a disease or disorder in which a hormone expressed and/or secreted and/or produced by or produced from a hormone precursor secreted by human EECs is implicated.

In some embodiments, the target is PAX6. In some embodiments, the disease or disorder is a disease or disorder in which a hormone expressed and/or secreted and/or produced by or produced from a hormone precursor secreted by human EECs is implicated.

In some embodiments, the human EECs are EECs and/or the target is CHGA. In some embodiments, the disease or disorder is a disease or disorder in which a hormone expressed and/or secreted and/or produced by or produced from a hormone precursor secreted by human EECs is implicated. In some embodiments, the disease or disorder is a disease or disorder treatable or preventable by increasing or inducing expression and/or secretion and/or production of a hormone. In some embodiments, the disease or disorder is a disease or disorder treatable or preventable by decreasing or ceasing expression and/or secretion and/or production of a hormone. Examples of such diseases are provided herein.

In some embodiments, the human EECs are EC-cells and/or the target is CHGB. In some embodiments, the disease or disorder is a disease or disorder in which a hormone expressed and/or secreted and/or produced by or produced from a hormone precursor secreted by a human EC-cell is implicated. In some embodiments, the disease or disorder is a disease or disorder treatable or preventable by increasing or inducing expression and/or secretion and/or production of serotonin. In some embodiments, the disease or disorder is a disease or disorder treatable or preventable by decreasing or ceasing expression and/or secretion and/or production of serotonin. Examples of such diseases are provided herein.

In some embodiments, the human EECs are EC-cells and/or the target is G Protein-Coupled Receptor 112 (GPR112). In some embodiments, the disease or disorder is a disease or disorder in which a hormone expressed and/or secreted and/or produced by or produced from a hormone precursor secreted by a human EC-cell is implicated. In some embodiments, the disease or disorder is a disease or disorder treatable or preventable by increasing or inducing expression and/or secretion and/or production of serotonin. In some embodiments, the disease or disorder is a disease or disorder treatable or preventable by decreasing or ceasing expression and/or secretion and/or production of serotonin. Examples of such diseases are provided herein.

In some embodiments, the human EECs are EC-cells and/or the target is OR51E1. In some embodiments, the disease or disorder is a disease or disorder in which a hormone expressed and/or secreted and/or produced by or produced from a hormone precursor secreted by human EC-cells is implicated. In some embodiments, the disease or disorder is a disease or disorder treatable or preventable by increasing or inducing expression and/or secretion and/or production of serotonin. In some embodiments, the disease or disorder is a disease or disorder treatable or preventable by decreasing or ceasing expression and/or secretion and/or production of serotonin. Examples of such diseases are provided herein.

In some embodiments, the human EECs are M/X cells and/or the target is Ectonucleotide Pyrophosphatase/Phosphodiesterase 1 (ENPP1). In some embodiments, the disease or disorder is a disease or disorder in which a hormone expressed and/or secreted and/or produced by or produced from a hormone precursor secreted by human M/X-cells is implicated. In some embodiments, the disease or disorder is a disease or disorder treatable or preventable by increasing or inducing expression and/or secretion and/or production of Motilin. In some embodiments, the disease or disorder is a disease or disorder treatable or preventable by decreasing or ceasing expression and/or secretion and/or production of Motilin. In some embodiments, the disease or disorder is a disease or disorder treatable or preventable by increasing or inducing expression and/or secretion and/or production of Ghrelin. In some embodiments, the disease or disorder is a disease or disorder treatable or preventable by decreasing or ceasing expression and/or secretion and/or production of Ghrelin. Examples of such diseases are provided herein.

In some embodiments, the human EECs are M/X cells and/or the target is Acyl-CoA Synthetase Long Chain Family Member 1 (ACSL1). In some embodiments, the disease or disorder is a disease or disorder in which a hormone expressed and/or secreted and/or produced by or produced from a hormone precursor secreted by human M/X-cells is implicated. In some embodiments, the disease or disorder is a disease or disorder treatable or preventable by increasing or inducing expression and/or secretion and/or production of Motilin. In some embodiments, the disease or disorder is a disease or disorder treatable or preventable by decreasing or ceasing expression and/or secretion and/or production of Motilin. In some embodiments, the disease or disorder is a disease or disorder treatable or preventable by increasing or inducing expression and/or secretion and/or production of Ghrelin. In some embodiments, the disease or disorder is a disease or disorder treatable or preventable by decreasing or ceasing expression and/or secretion and/or production of Ghrelin. Examples of such diseases are provided herein.

In some embodiments, the target is ASCL1. In some embodiments, the disease or disorder is a disease or disorder in which a hormone expressed and/or secreted and/or produced by or produced from a hormone precursor secreted by human EECs except M/X-cells is implicated.

In some embodiments, the target is MNX1. In some embodiments, the disease or disorder is a disease or disorder in which a hormone expressed and/or secreted and/or produced by or produced from a hormone precursor secreted by human EC-cells is implicated.

In some embodiments, the target is a transcription factor. In some embodiments, the transcription factor is selected from ARX (e.g. and the human EECs are all EECs except D-cells and EC-cells), ASCL1 (e.g. and the human EECs are all EECs except M/X-cells), HHEX (e.g. and the human EECs are D-cells), LMX1A (e.g. and the human EECs are EC-cells or D-cells, preferably EC-cells), MNX1 (e.g. and the human EECs are EC-cells), NKX2-2 (e.g. and the human EECs are EECs), PAX4 (e.g. and the human EECs are D-cells and/or EC-cells), PAX6 (e.g. and the human EECs are EECs), SOX4 (e.g. and the human EECs are EECs) and RFX6 (e.g. and the human EECs are EECs). In some embodiments, the disease or disorder is a disease or disorder in which one or more hormones expressed and/or secreted and/or produced by or produced from a hormone precursor secreted by the human EEC in which the target transcription factor is expressed is implicated. Examples of such diseases and disorders are provided herein.

Methods for Validating Targets

The intestinal organoids described herein, for example, the reporter organoids, are particularly useful for validating that a target of interest can be used to affect the differentiation of EECs and/or the secretion of one or more hormones and/or hormone precursors by one or more EEC subtypes. As explained above, the finding that a target of interest is useful in this way opens up a number of practical applications, such as the ability to identify compounds that target the target for use in treating or preventing diseases or disorders in which the hormones and/or hormone precursors secreted by the EEC in which the target is expressed are implicated. A number of practical applications are described elsewhere herein.

The invention provides a method for determining whether modulation of a target receptor controls differentiation of EECs, wherein the method comprises (i) contacting a reporter organoid of the invention with a compound and differentiating intestinal stem cells and/or cells with stem cell potential in the reporter organoid to EECs, wherein the reporter organoid is contacted with the compound before or during differentiating the intestinal stem cells and/or intestinal cells with stem cell potential to EECs; (ii) determining whether secretion of one or more tagged hormones and/or tagged hormone precursors is affected and/or whether expression of one or more tagged hormones, tagged hormone precursors and/or tagged hormone synthesizing enzymes is affected; and (iii) extrapolating an effect on the secretion and/or expression to a change in the number of cells of one or more EEC subtypes present. For example, in some embodiments, step (iii) extrapolates an effect on the secretion and/or expression to a change in the ratio of one or more EEC subtypes compared to one or more other EEC subtypes. Preferably, step (ii) comprises determining whether expression is affected. In preferred embodiments, the compound is a known ligand for the receptor. In some embodiments, the compound is a predicted ligand for the receptor. In preferred embodiments, step (ii) is conducted using a fluorescence assay, for example, a fluorescent plate reader or a fluorescence microscope. The reporter organoid used in step (i) is preferably a stable reporter organoid line, as described herein.

In embodiments of the methods described herein in which the organoid is contacted with the compound before or during differentiating the intestinal stem cells and/or intestinal cells with stem cell potential to EECs, the organoid may be contacted with the compound before, at the same time as, or after the organoid is contacted with the inducer for the inducible transcription factor. However, in embodiments in which the organoid is contacted with the compound after the organoid is contacted with the inducer for the inducible transcription factor, the organoid is preferably contacted with the compound whilst the intestinal stem cells and/or cells with stem cell potential are still being differentiated to EECs. Contacting with the compound and/or the inducer may comprise culturing the organoid in the presence of the compound and/or the inducer.

In some embodiments, contacting the reporter organoid with one or more compounds during differentiation comprises contacting the organoid with the compound at the same time as contacting the organoid with the inducer for the inducible transcription factor. In some embodiments, the organoid is contacted with the compound after contacting the reporter organoid with the inducer. For example, in some embodiments, the reporter organoid is contacted with the compound about 48 hours after it is contacted with the inducer. The reporter organoid is preferably contacted with the compound for a period of time that is sufficient for the compound to have an effect. For example, in some embodiments, the reporter organoid is contacted with the compound (such as a population of bacteria) for about 72 hours. In some embodiments, the organoid is contacted with the compound until 5 days after induction of differentiation of the stem cells and/or cells with intestinal potential to EECs.

The invention provides a further method for determining whether modulation of a target receptor controls differentiation of EECs, wherein the method comprises (i) contacting an intestinal organoid of the invention with a compound and differentiating intestinal stem cells in the organoid to EECs, wherein the organoid is contacted with the compound before or during differentiating the intestinal stem cells and/or intestinal cells with stem cell potential to EECs; (ii) determining whether the transcript levels of one more hormones, hormone precursors and/or hormone synthesizing enzymes is affected; and (iii) extrapolating an effect on transcript levels to a change in the number of cells of one or more EEC subtypes present. For example, in some embodiments, step (iii) extrapolates an effect on transcript levels to a change in the ratio of one or more EEC subtypes compared to one or more other EEC subtypes. In preferred embodiments, the compound is a known ligand for the receptor. In some embodiments, the compound is a predicted ligand for the receptor. In preferred embodiments, step (ii) is conducted using a qPCR assay. The qPCR assay is conducted after a suitable period of time from induction of differentiation of the stem cells to EECs. For example, in some embodiments, the qPCR assay is conducted 3-7, 4-6 or 5 days after induction of differentiation. In some embodiments, the organoid is an intestinal organoid comprising stem cells and/or cells with stem cell potential which comprise an inducible transcription factor for differentiating intestinal stem cells and/or intestinal cells with stem cell potential to EECs but in which the stem cells have not yet been differentiated to EECs. In some embodiments, the organoid is not a reporter organoid. For example, in some embodiments, the organoid does not comprise any tagged hormones, tagged hormone precursors and/or tagged hormone synthesizing enzymes. In some embodiments, the organoid is a reporter organoid. In some embodiments, the reporter organoid is a stable reporter organoid line, as described herein.

The invention further provides a method for determining whether secretion and/or expression of one or more specific hormones, hormone precursors and/or hormone synthesizing enzymes is affected upon engagement of a target receptor, wherein the method comprises: (i) contacting a reporter organoid of the invention with a compound; and (ii) determining whether secretion of one or more tagged hormones and/or tagged hormone precursors is affected and/or whether expression of one or more tagged hormones, tagged hormone precursors and/or tagged hormone synthesizing enzymes is affected. In preferred embodiments, the compound is a known ligand for the receptor. In some embodiments, the compound is a predicted ligand for the receptor. In preferred embodiments, step (ii) is conducted using a fluorescence assay, for example, a fluorescent plate reader or a fluorescence microscope. The stem cells in the reporter organoid used in step (i) have preferably already been differentiated to EECs.

The invention provides a further method for determining whether secretion of one or more hormones and/or hormone precursors is affected upon engagement of a target receptor, wherein the method comprises: (i) contacting an intestinal organoid of the invention with a compound, wherein the intestinal organoid comprises a calcium reporter linked to a detectable marker (e.g. a fluorescent marker); and (ii) determining whether expression of the calcium reporter is affected by monitoring the detectable marker linked to the calcium reporter. In preferred embodiments, the compound is a known ligand for the receptor. In some embodiments, the compound is a predicted ligand for the receptor. Calcium activation is a measure of hormone secretion and so monitoring calcium levels with a calcium reporter can be used as a proxy for directly monitoring secretion. The stem cells in the organoid used in step (i) have preferably already been differentiated to EECs. In some embodiments, the organoid is an organoid obtained by or obtainable by a method for enriching the population of EECs in an intestinal organoid. In some embodiments, the organoid is an intestinal organoid in which at least 10% of the cells are EECs. In some embodiments, the organoid is an intestinal organoid in which more than 50% of the cells are EECs. In some embodiments, the organoid is not a reporter organoid. For example, in some embodiments, the organoid does not comprise any tagged hormones, tagged hormone precursors and/or tagged hormone synthesizing enzymes. In some embodiments, the organoid is a reporter organoid.

The invention provides a further method for determining whether secretion of one or more specific hormones and/or hormone precursors is affected upon engagement of a target receptor, wherein the method comprises: (i) contacting an intestinal organoid of the invention with a compound; and (ii) determining whether the levels of the one or more specific hormones and/or hormone precursors in the supernatant is affected. In preferred embodiments, the compound is a known ligand for the receptor. In some embodiments, the compound is a predicted ligand for the receptor. In preferred embodiments, step (ii) is conducted using an ELISA assay. The stem cells in the organoid used in step (i) have preferably already been differentiated to EECs. In some embodiments, the organoid is an organoid obtained by or obtainable by a method for enriching the population of EECs in an intestinal organoid. In some embodiments, the organoid is an intestinal organoid in which at least 10% of the cells are EECs. In some embodiments, the organoid is an intestinal organoid in which more than 50% of the cells are EECs. In some embodiments, the organoid is not a reporter organoid. For example, in some embodiments, the organoid does not comprise any tagged hormones, tagged hormone precursors and/or tagged hormone synthesizing enzymes. In some embodiments, the organoid is a reporter organoid.

The invention provides a further method for determining whether secretion of one or more proteins or peptides is affected upon engagement of a target receptor, wherein the method comprises: (i) contacting an intestinal organoid of the invention with a compound; and (ii) determining whether the levels of the one or more specific proteins or peptides in the supernatant is affected. In preferred embodiments, the compound is a known ligand for the receptor. In some embodiments, the compound is a predicted ligand for the receptor. In preferred embodiments, step (ii) is conducted by analysing the supernatant obtained from the organoid culture of step (i) using mass spectrometry. In some embodiments, the one or more secreted proteins or peptides are one or more secreted hormones or hormone precursors. However, they may alternatively or additionally be any other secreted product, for example, one or more of (e.g. 1, 2, 3 or more or all 4 of) CPB1, PCSK1, CPE and DPP4. In some embodiments, the method comprises comparing the results with a control organoid which has not been contacted with the compound. This enables profiling for any proteins or peptides whose secretion has been induced, ceased, increased or decreased in the supernatant. Advantageously, therefore, the method can be used to look at multiple proteins and peptides. The stem cells and/or cells with stem cell potential in the organoid used in step (i) have preferably already been differentiated to EECs. In some embodiments, the organoid is an organoid obtained by or obtainable by a method for enriching the population of EECs in an intestinal organoid. In some embodiments, the organoid is an intestinal organoid in which at least 10% of the cells are EECs. In some embodiments, the organoid is an intestinal organoid in which more than 50% of the cells are EECs. In some embodiments, the organoid is not a reporter organoid. For example, in some embodiments, the organoid does not comprise any tagged hormones, tagged hormone precursors and/or tagged hormone synthesizing enzymes. In some embodiments, the organoid is a reporter organoid.

In some embodiments, the method may comprise determining whether secretion and/or expression of one or more specific hormones, hormone precursors and/or hormone synthesizing enzymes is induced, increased, reduced or ceased.

In some embodiments, the methods further comprise comparing the results with the results from a suitable control, for example, a control as described herein. Thus, in some embodiments, the change in secretion and/or expression may be compared to the control. In some embodiments, the control is a control organoid that has not been contacted with the one or more compounds. In some embodiments, the control is a control organoid in which the stem cells and/or cells with stem cell potential have not been differentiated to EECs. In some embodiments, the control is a control organoid in which the target has been knocked out or inactivated. For example, if the effect seen in the experimental organoid is not obtained in the control, for example, if the effect is not obtained in a knockout/inactivated control, this may indicate that the target is implicated in the effect. For example, it may validate the target (e.g. the cell surface receptor) as being targeted by the compound used in the method. In some embodiments, the target is inactivated by way of mutation. In some embodiments, the target (e.g. a cell surface receptor) is inactivated by way of functional blocking, e.g. by prior binding of an antagonist. In some embodiments, the method comprises comparing the results with the results from two or more different types of control, for example, as described herein.

The invention provides a method for determining whether modulation of a transcription factor of interest controls differentiation of EECs, wherein the method comprises (i) obtaining a stable reporter organoid of the invention in which the transcription factor of interest is knocked out; (ii) differentiating intestinal stem cells in the reporter organoid to EECs; (iii) determining whether secretion of one or more tagged hormones and/or tagged hormone precursors is affected and/or whether expression of one or more tagged hormones, tagged hormone precursors and/or tagged hormone synthesizing enzymes is affected; and (iv) extrapolating an effect on the secretion and/or expression to a change in the number of cells of one or more EEC subtypes present. For example, in some embodiments, step (iv) extrapolates an effect on the secretion and/or expression to a change in the ratio of one or more EEC subtypes compared to one or more other EEC subtypes. In some embodiments, step (i) comprises making a reporter organoid of the invention in which the transcription factor is knocked out. Preferably, step (iii) comprises determining whether expression is affected. In preferred embodiments, step (iii) is conducted using a fluorescence assay, for example, a fluorescent plate reader or a fluorescence microscope. The reporter organoid used in step (i) is preferably a stable reporter organoid line, as described herein. In some embodiments, the method further comprises comparing the results with the results from a suitable control, for example, a control as described herein. In some embodiments, the control is a control organoid in which the transcription factor has not been knocked out.

The invention provides a further method for determining whether modulation of a transcription factor of interest controls differentiation of EECs, wherein the method comprises (i) obtaining a stable intestinal organoid of the invention in which the transcription factor of interest is knocked out (ii) differentiating intestinal stem cells in the reporter organoid to EECs; (iii) determining whether the transcript levels of one more hormones, hormone precursors and/or hormone synthesizing enzymes is affected; and (iv) extrapolating an effect on transcript levels to a change in the number of cells of one or more EEC subtypes present. For example, in some embodiments, step (iv) extrapolates an effect on hormone transcript levels to a change in the ratio of one or more EEC subtypes compared to one or more other EEC subtypes. In preferred embodiments, step (iii) is conducted using a qPCR assay. The qPCR assay is conducted after a suitable period of time from induction of differentiation of the stem cells to EECs. For example, in some embodiments, the qPCR assay is conducted 3-7, 4-6 or 5 days after induction of differentiation. In some embodiments, the organoid used in step (i) is an intestinal organoid comprising stem cells and/or cells with stem cell potential which comprise an inducible transcription factor for differentiating intestinal stem cells and/or intestinal cells with stem cell potential to EECs but in which the stem cells have not yet been differentiated to EECs. In some embodiments, the organoid is not a reporter organoid. For example, in some embodiments, the organoid does not comprise any tagged hormones, tagged hormone precursors and/or tagged hormone synthesizing enzymes. In some embodiments, the organoid is a reporter organoid. In some embodiments, the reporter organoid is a stable reporter organoid line, as described herein. In some embodiments, the method further comprises comparing the results with the results from a suitable control, for example, a control as described herein. In some embodiments, the control is a control organoid in which the transcription factor has not been knocked out.

The invention provides a method for determining whether one or more compounds controls differentiation of EECs, wherein the method comprises (i) contacting a reporter organoid of the invention with the one or more compounds and differentiating intestinal stem cells in the reporter organoid to EECs, wherein the reporter organoid is contacted with the one or more compounds before or during differentiating the intestinal stem cells and/or intestinal cells with stem cell potential to EECs; (ii) determining whether secretion of one or more tagged hormones and/or tagged hormone precursors is affected and/or whether expression of one or more tagged hormones, tagged hormone precursors and/or tagged hormone synthesizing enzymes is affected; and (iii) extrapolating an effect on the secretion and/or expression to a change in the number of cells of one or more EEC subtypes present. For example, in some embodiments, step (iii) extrapolates an effect on the secretion and/or expression to a change in the ratio of one or more EEC subtypes compared to one or more other EEC subtypes. Preferably, step (ii) comprises determining whether expression is affected. In some embodiments, step (ii) is conducted using a fluorescence assay, for example, a fluorescent plate reader or a fluorescence microscope. In some embodiments, step (ii) comprises using qPCR to determine whether expression of one or more tagged hormones, tagged hormone precursors and/or tagged hormone synthesizing enzymes is affected. The reporter organoid used in step (i) is preferably a stable reporter organoid line, as described herein.

In some embodiments, the compound for use in a differentiation screen is a BMP pathway activator (for example, BMP, BMP2 or BMP4) and one or more reporter organoids comprising tagged NTS and tagged GLP1 are used. NTS and GLP1 may be tagged in the same reporter organoid or in different reporter organoids.

The invention further provides a method for determining whether one or more compounds affects secretion and/or expression of one or more specific hormones, hormone precursors and/or hormone synthesizing enzymes, wherein the method comprises: (i) contacting a reporter organoid of the invention with the one or more compounds; and (ii) determining whether secretion of one or more tagged hormones and/or tagged hormone precursors is affected and/or whether expression of one or more tagged hormones, tagged hormone precursors and/or tagged hormone synthesizing enzymes is affected. In preferred embodiments, step (ii) is conducted using a fluorescence assay, for example, a fluorescent plate reader or a fluorescence microscope. In some embodiments, fluorescence decay and/or the location of the tagged hormones is visualised and/or quantified. The stem cells in the reporter organoid used in step (i) have preferably already been differentiated to EECs.

The invention provides a further method for determining whether one or more compounds affects secretion of one or more hormones and/or hormone precursors, wherein the method comprises: (i) contacting an intestinal organoid of the invention with the one or more compounds, wherein the intestinal organoid comprises a calcium reporter linked to a detectable marker (e.g. a fluorescent marker); and (ii) determining whether expression of the calcium reporter is affected by monitoring the detectable marker linked to the calcium reporter. The stem cells in the organoid used in step (i) have preferably already been differentiated to EECs. In some embodiments, the organoid is an organoid obtained by or obtainable by a method for enriching the population of EECs in an intestinal organoid. In some embodiments, the organoid is an intestinal organoid in which at least 10% of the cells are EECs. In some embodiments, the organoid is an intestinal organoid in which more than 50% of the cells are EECs. In some embodiments, the organoid is not a reporter organoid. For example, in some embodiments, the organoid does not comprise any tagged hormones, tagged hormone precursors and/or tagged hormone synthesizing enzymes. In some embodiments, the organoid is a reporter organoid.

The invention provides a further method for determining whether one or more compounds affects secretion of one or more specific hormones and/or hormone precursors, wherein the method comprises: (i) contacting an intestinal organoid of the invention with one or more compounds; and (ii) determining whether the levels of the one or more specific hormones and/or hormone precursors in the supernatant is affected. In preferred embodiments, step (ii) is conducted using an ELISA assay. The stem cells in the organoid used in step (i) have preferably already been differentiated to EECs. In some embodiments, the organoid is an organoid obtained by or obtainable by a method for enriching the population of EECs in an intestinal organoid. In some embodiments, the organoid is an intestinal organoid in which at least 10% of the cells are EECs. In some embodiments, the organoid is an intestinal organoid in which more than 50% of the cells are EECs. In some embodiments, the organoid is not a reporter organoid. For example, in some embodiments, the organoid does not comprise any tagged hormones, tagged hormone precursors and/or tagged hormone synthesizing enzymes. In some embodiments, the organoid is a reporter organoid.

Advantageously, use of a method for determining whether one or more compounds controls differentiation of EECs, or of a method for determining whether one or more compounds affects secretion and/or expression of one or more specific hormones, hormone precursors and/or hormone synthesizing enzymes, allows identification of one or more compounds which control differentiation of EECs, irrespective of their target on the EECs.

In some embodiments, the one or more compounds are a library of compounds, for example, a library as described herein. In some embodiments, the one or more compounds are comprised in supernatant from a cell culture, e.g. from a bacterial cell culture. In some embodiments, the one or more compounds are comprised in supernatant from culture of a sample taken from the microbiome (e.g. from the human microbiome). Thus, in some embodiments, step (i) comprises contacting the organoid (e.g. the reporter organoid) with a supernatant, for example, a supernatant as described herein.

In some embodiments, the methods further comprise comparing the results with the results from a suitable control, for example, a control as described herein. In some embodiments, the control is a control organoid that has not been contacted with the one or more compounds.

In some embodiments, the reporter organoid used in step (i) comprises two or more tagged hormones, tagged hormone synthesizing enzymes and/or tagged hormone precursors.

In some embodiments, step (i) comprises contacting multiple cultures of organoids with the one or more compounds. In some embodiments, different hormones, hormone precursors and/or hormone synthesizing enzymes are tagged in the different organoid cultures. In some embodiments in which libraries of compounds are used, individual members of the library are used to contact different cultures of organoids. In some embodiments, organoids are cultured in a multiplex manner. Thus, a biobank of organoids may be used in a method of the invention. In some embodiments, the biobank is a biobank of reporter organoids.

In some embodiments, qPCR is used to validate that a hormone exists. In some embodiments, secretomics is used to validate that a hormone exists. For example, mass spectrometry of the secretome from an organoid of the invention can be used to show existence of the hormone and its secretion.

Secretrome

The human EEC organoid cultures uniquely allow proteomic analysis of secreted products of EECs. In particular, the human EEC reporter organoid cultures allow to assign secreted products to EECs and EEC subtypes, something that is not possible in vivo. EEC hormones are secreted basolaterally, i.e. towards the outside of the organoids, and so in an organoid culture of the invention, the secreted hormones end up in the supernatant following centrifugation.

The invention therefore provides a method for assigning a secreted product to an EEC or to an EEC subtype comprising making one or more reporter organoids according to the invention comprising one or more subtypes of the EECs and determining which subtypes the secreted product is expressed in.

The invention further provides a method for analysing the secretome of EECs or of one or more EEC subtypes comprising making one or more reporter organoids of the invention and determining which tagged hormones and/or hormone precursors are secreted by the EEC or by the one or more EEC subtypes. In some embodiments, determining which tagged hormones and/or hormone precursors are secreted comprises visualising the one or more detectable markers.

The invention provides a method for analysing the secretome of an EEC or a subtype thereof comprising culturing an organoid as described herein, isolating the supernatant of the organoid, and identifying the proteins or peptides present in the supernatant. In some embodiments, the organoid is stimulated with a compound that induces hormone secretion prior to isolating the supernatant. For example, the compound may increase intracellular levels of cAMP. In some embodiments the compound is Forskolin. Any suitable method may be used to identify the proteins or peptides present in the supernatant. In some embodiments, mass spectrometry is used, e.g. LC-MS. For example, in some embodiments, the supernatant is separated into a fraction larger than 10 kDa (representing, amongst others, unprocessed hormones), and a fraction smaller than 10 kDa (representing processed endogenous hormones). In some embodiments, the fraction larger than 10 kDa is subjected to tryptic digestion prior to LC-MS analysis whereas the fraction smaller than 10 kDa is directly analysed using LC-MS.

In some embodiments, the secretome of an organoid established from the proximal small intestine (duodenum), the distal small intestine (ileum), the ascending colon, the descending colon, the transverse colon, the sigmoid colon, the rectum or the jejunum is analysed. In some embodiments, the secretome of an organoid established from the proximal small intestine is analysed. In some embodiments, the secretome of an organoid established from the distal small intestine is analysed. In some embodiments, the secretome of an organoid established from the colon is analysed.

The invention provides a library of hormones, in which the hormones therein comprise, consist or consist essentially of hormones secreted or produced (e.g. from a hormone precursor) by EECs or by one or more EEC subtypes (for example, by one or more human EECs). Similarly, the invention provides a library of hormones in which the proteins therein comprise, consist or consist essentially of hormones secreted or produced by one or more organoids of the invention. In some embodiments, the hormones comprise, consist or consist essentially of hormones secreted or produced by all EECs. In some embodiments, the hormones comprise, consist or consist essentially of hormones secreted or produced by one or more (e.g. 1, 2, 3, 4, 5, 6, 7, 8, 9, 10 or more) subtypes of EECs. In some embodiments, the hormones comprise, consist or consist essentially of hormones secreted or produced by EECs located in a particular region of the intestine, for example, the proximal small intestine (duodenum), the distal small intestine (ileum), the ascending colon, the descending colon, the transverse colon, the sigmoid colon, the rectum or the jejunum. In some embodiments, the hormones comprise, consist or consist essentially of hormones secreted or produced by EECs located in the proximal small intestine, the distal small intestine or the colon (e.g. the ascending colon). In some embodiments, the hormones comprise, consist or consist essentially of hormones secreted or produced by an organoid established from a particular region of the intestine, for example, the proximal small intestine (duodenum), the distal small intestine (ileum), the ascending colon, the descending colon, the transverse colon, the sigmoid colon, the rectum or the jejunum. For example, in some embodiments, the invention provides a library of hormones in which the hormones therein comprise, consist or consist essentially of hormones secreted or produced by proximal small intestinal organoids of the invention. In some embodiments, the invention provides a library of hormones in which the hormones therein comprise, consist or consist essentially of hormones secreted or produced by distal small intestinal organoids of the invention. In some embodiments, the invention provides a library of hormones in which the hormones therein comprise, consist or consist essentially of hormones secreted or produced by colon organoids (e.g. ascending colon organoids) of the invention. In some embodiments, the hormones are human hormones. For example, in some embodiments, the EECs, and where relevant the organoids comprising the EECs, are human. In some embodiments, hormone precursors are also present in the library, which are secreted by the types of EECs and/or organoids recited above. Thus, the library of hormones may be a library of hormones and/or hormone precursors, and the various embodiments may be adapted accordingly.

In some embodiments, at least 80% (e.g. at least 85%, 90%, 95%, 98% or 99%, or 100%) of the hormones in the library of hormones are hormones secreted by the respective EEC(s) or the respective organoid of the invention.

The library of hormones may optionally be termed the “secretome” of an EEC or an organoid.

Similarly, the invention provides one or more (e.g. 1, 2, 3, 4, 5, 6, 7, 8, 9, 10, 11, 12, 13, 14, 15, 16, 17, 18, 19, 20 or more) isolated hormones from EECs or one or more EEC subtypes Preferably, the EECs are human EECs. In some embodiments, the one or more isolated hormones are hormones which are not present in their in vivo environment. In some embodiments, the one or more hormones have been isolated from the culture medium into which they have been secreted and/or have been isolated from an organoid of the invention, for example, from an EEC.

In some embodiments, one or more hormones and/or hormone precursors (e.g. the library) is present in culture medium, for example, in the culture medium that the organoid was cultured in. Thus, the invention provides a culture medium comprising the one or more hormones and/or hormone precursors or library of hormones and/or hormone precursors. In some embodiments, the culture medium additionally comprises one or more organoids of the invention. Thus, the culture medium may be obtained or obtainable by a method of making an organoid as described herein. In some embodiments, the culture medium is obtained or obtainable from an organoid as described herein which has been targeted with a compound, for example, to induce, increase, cease or reduce expression and/or secretion of one or more hormones, hormone precursors and/or hormone synthesizing enzymes. Any suitable culture medium may be used. In some embodiments, the culture medium is a culture medium as described herein, e.g. an expansion medium, a differentiation medium or a basal medium.

In some embodiments, the one or more hormones and/or hormone precursors or the library is present in supernatant obtained from or obtainable from culturing an organoid of the invention in a culture medium. Accordingly, the invention provides a supernatant comprising one or more hormones and/or hormone precursors or a library of hormones and/or hormone precursors as described herein.

Also provided is a culture medium or supernatant which comprises secreted products from an organoid of the invention. For example, the culture medium or supernatant may advantageously contain secreted products such as hormones and/or hormone precursors which represent the secretome of the organoid. The culture medium may be a culture medium as described herein. For example, the culture medium or supernatant may be obtained by or obtainable by culturing an organoid of the invention and optionally obtaining the supernatant, as appropriate.

Thus, advantageously, a library of hormones and/or hormone precursors of the invention (whether tagged, partially tagged or untagged) can represent the naturally occurring secretome of the intestine (or of a particular region thereof, e.g. the duodenum, ileum or ascending colon). This library of hormones and/or hormone precursors can be used to study the biology of the secretome.

In some embodiments, the organoid of the invention has been stimulated with a compound that induces hormone secretion, e.g. prior to isolating the supernatant. For example, the compound may increase intracellular levels of cAMP. In some embodiments the compound is Forskolin.

In embodiments in which the one or more isolated hormones and/or hormone precursors are two or more isolated hormones and/or hormone precursors, these may be a library of isolated hormones and/or hormone precursors according to the invention. Thus, the invention provides a library of two or more (e.g. 2, 3, 4, 5, 6, 7, 8, 9, 10, 11, 12, 13, 14, 15, 16, 17, 18, 19, 20 or more) isolated hormones and/or hormone precursors from one or more EECs or EEC subtypes.

In some embodiments, the one or more hormones and/or hormone precursors (e.g. in the library) are obtained from or are obtainable from a reporter organoid of the invention and so are tagged with one or more detectable markers. In some embodiments, the hormones and/or hormone precursors in the library are not tagged. In some embodiments, some hormones and/or hormone precursors are tagged and some hormones and/or hormone precursors are not tagged.

In some embodiments, the library of hormones and/or hormone precursors comprises two or more different hormones and/or hormone precursors, e.g. 2, 3, 4, 5, 6, 7, 8, 9, 10, 11, 12, 13, 14, 15, 16, 17, 18, 19, 20 or more different hormones and/or hormone precursors.

In some embodiments, the library of hormones comprises or consists of two or more of (e.g. 2, 3, 4, 5, 6, 7, 8, 9 or more or all 10 of) CCK, CHGA, CHGB, GAST, MLN, SCG3, SST, MDK, PAM and REG3A. In some embodiments, the library of hormones comprises or consists of two or more of (e.g. 2, 3, 4, 5, 6 or more or all 7 of) CCK, CHGA, CHGB, GAST, MLN, SCG3 and SST. In some embodiments, the library of hormones comprises or consists of two or more of (e.g. 2 or more or all 3 of) MDK, PAM and REG3A. In some embodiments, the library of hormones comprises or consists of two or more of (e.g. 2, 3, 4, 5, 6, 7, 8, 9, 10, 11, 12, 13, 14, 15 or more or all 16 of) CCK, CHGA, CHGB, GAST, GHRL, GIP, MLN, NTS, SCG2, SCG3, SST, NPW, NUCB2, PAM, REG3A and VGF processed peptides. In some embodiments, the library of hormones comprises or consists of two or more of (e.g. 2, 3, 4, 5, 6, 7, 8, 9, 10 or more or all 11 of) CCK, CHGA, CHGB, GAST, GHRL, GIP, MLN, NTS, SCG2, SCG3 and SST processed peptides. In some embodiments, the library of hormones comprises or consists of two or more of (e.g. 2, 3, 4 or more or all 5 of) NPW, NUCB2, PAM, REG3A, and VGF processed peptides. In some embodiments, the library of hormones comprises or consists of two or more of (e.g. 2, 3, 4, 5, 6, 7, 8, 9, 10, 11, 12, 13, 14, 15, 16 or more or all 17 of) CCK, CHGA, CHGB, GAST, GHRL, GIP, MLN, NTS, SCG2, SCG3, SST, MDK, PAM, REG3A, NPW, NUCB2, VGF hormones and/or processed peptides. In some embodiments, the library of hormones is obtained from or is obtainable from a proximal small intestinal organoid of the invention.

In some embodiments, the library of hormones comprises or consists of two or more of (e.g. 2, 3, 4, 5, 6, 7, 8, 9, 10, 11, 12, 13 or more or all 14 of) CHGA, CHGB, GCG, MLN, PYY, REG4, SCG2, SCG3, SCGN, SST, MDK, NUCB2, PAM and VGF. In some embodiments, the library of hormones comprises or consists of two or more of (e.g. 2, 3, 4, 5, 6, 7, 8, 9 or more or all 10 of) CHGA, CHGB, GCG, MLN, PYY, REG4, SCG2, SCG3, SCGN and SST. In some embodiments, the library of hormones comprises or consists of two or more of (e.g. 2, 3 or more or all 4 of) MDK, NUCB2, PAM and VGF. In some embodiments, the library of hormones comprises or consists of two or more of (e.g. 2, 3, 4, 5, 6, 7, 8, 9, 10, 11, 12, 13 or more or all 14 of) CHGA, GAST, GCG, GHRL, GIP, MLN, NTS, PYY, SCG2, SCG3, SST, UCN3, NPW and VGF processed peptides. In some embodiments, the library of hormones comprises or consists of two or more of (e.g. 2, 3, 4, 5, 6, 7, 8, 9, 10, 11 or more or all 12 of) CHGA, GAST, GCG, GHRL, GIP, MLN, NTS, PYY, SCG2, SCG3, SST and UCN3 processed peptides. In some embodiments, the library of hormones comprises or consists of NPW and VGF. In some embodiments, the library of hormones comprises or consists of two or more of (e.g. 2, 3, 4, 5, 6, 7, 8, 9, 10, 11, 12, 13, 14, 15, 16, 17, 18, 19 or more or all 20 of) CHGA, CHGB, GAST, GCG, GHRL, GIP, MLN, NTS, PYY, REG4, SCG2, SCG3, SCGN, SST, UCN3, MDK, NUCB2, PAM, VGF and NPW hormones and/or processed peptides. In some embodiments, the library of hormones is obtained from or is obtainable from a distal small intestinal organoid of the invention. In some embodiments, the library of hormones additionally or alternatively comprises PPY. Thus, in some embodiments, PPY is added to the lists described herein.

In some embodiments, the library of hormones comprises or consists of a mixture of unprocessed hormones and processed hormones and/or hormone precursors. For example, fragments of hormones may be present. In some embodiments, such fragments are bioactive fragments. For example, in some embodiments, one or more of the hormones is not a full length protein. For example, one or more of the hormones or processed hormones may be trimmed at its C-terminal end. In some embodiments, one or more processed peptides smaller than 10 kDa display C-terminal trimming, for example, which may be due to the endogenous activity of carboxypeptidases. In preferred embodiments, the N-terminal signal peptide is not present in the hormones and processed peptides. For example, in some preferred embodiments, the N-terminal 20-25 amino acids of the prohormones is not present in one or more (e.g. 1, 2, 3, 4, 5, 6, 7, 8, 9, 10 or more), or all of the hormones in the library. These characteristics may be applied to a single isolated hormone of the invention, mutatis mutandis.

In some embodiments in which one or more of (e.g. 2 or more or all 3 of) MDK, REG4 and SCGN are present, these hormones are present in their unprocessed form. In some embodiments, hormones other than MDK, REG4 and SCGN are present in a processed form.

In some embodiments, the one or more hormones or the library of hormones is or comprises GCG-derived peptides. For example, in some embodiments, the library of hormones comprises fragments of the proglucagon pro-hormone. For example, in some embodiments, the library of hormones comprises one or more of (e.g. 1, 2, 3, 4, 5, 6, 7 or more or all 8 of) Glicentin, Glicentin-related polypeptide, Glucagon, Glucagon-like peptide 1, Glucagon-like peptide 1 (7-36), Glucagon-like peptide 1 (7-37), Glucagon-like peptide 2 and Oxyntomodulin. Preproglucagon is encoded by the GCG gene. In some embodiments, such a hormone or library of hormones is obtained from or is obtainable from a distal small intestinal organoid of the invention.

In some embodiments, the one or more hormones or the library of hormones is or comprises Neuronostatin. Neuronostatin is a fragment of the pro-somatostatin hormone. In some embodiments such a hormone or library of hormones is obtained from or is obtainable from a proximal small intestinal organoid of the invention or a distal small intestinal organoid of the invention.

In some embodiments, the one or more hormones or library of hormones is or comprises a bioactive fragment of GHRL. In some embodiments such a hormone or library of hormones is obtained from or is obtainable from a proximal small intestinal organoid of the invention or a distal small intestinal organoid of the invention.

In some embodiments, one or more hormones or a library of hormones obtained from or obtainable from a duodenal organoid of the invention is or comprises one or more bioactive peptides of MLN. In some embodiments, one or more hormones or a library of hormones obtained from or obtainable from an ileal organoid of the invention is not or does not comprise one or more bioactive peptides of MLN.

In some embodiments, CHGA is the most abundant peptide in the library of hormones. The inventors have surprisingly identified secretion of CHGA_2, NPW, VGF, Midkine and the peptidase CPB1 in the secretome of organoids of the invention. These proteins are previously unknown EEC products. Although VGF has been reported as an EEC product before, the inventors have shown secretion for the first time. CHGA_2 is a peptide product of CHGA which spans terminal sequences of exon 5 and exon 7. In some embodiments, the one or more hormones or library of hormones is or comprises one or more of (e.g. 1, 2 or more or all 3 of) NPW, VGF and Midkine.

Thus, the invention provides a method for hormone-mediated signalling which comprises contacting cells in vitro or ex vivo with at least 1 of (e.g. 1, 2 or more or all 3 of) NPW, VGF and Midkine. In some embodiments, the cells are intestinal cells. In some embodiments, the cells are EECs or one or more subtypes of EECs. For example, in some embodiments, the cells are an EEC-containing organoid of the invention. Similarly, in some embodiments, the invention provides a method for screening for NPW, VGF or Midkine activity comprising knocking out one or more (e.g. all) putative receptors for NPW, VGF or Midkine, respectively, in an intestinal organoid of the invention and determining if the effect of NPW, VGF or Midkine is maintained. For example, in the case of Midkine, the method may comprise determining if the reduction in expression of all EEC hormones is maintained. Such a method would also advantageously validate whether NPW, VGF and/or Midkine acts a hormone.

The invention further provides a method of affecting hormone processing comprising contacting cells in vitro or ex vivo with the peptidase CPB1. In some embodiments, the cells are EECs, for example, an EEC-containing organoid of the invention.

The inventors have also surprisingly identified secretion of REG3A, PAM and NUC2B precursor (that is processed to the neuropeptides Nesfatin-1, 2 and 3) by human EECs. In some embodiments, the one or more hormones or library of hormones is or comprises one or more of (e.g. 1, 2 or more or all 3 of) REG3A, PAM and the NUC2B precursor.

In some embodiments, an isolated hormone and/or hormone precursor or a library of such hormones and/or hormone precursors may be used as a compound in the method of the invention. For example, in some embodiments, a supernatant obtained from or obtainable from an organoid culture, which comprises the secretome of the organoid, is used as a compound in a method of the invention.

### Definitions

As used herein, the verb “to comprise” and its conjugations is used in its non-limiting sense to mean that items following the word are included, but items not specifically mentioned are not excluded. In addition the verb “to consist” may be replaced, if necessary, by “to consist essentially of” meaning that a product as defined herein may comprise additional component(s) than the ones specifically identified, said additional component(s) not altering the unique characteristic of the invention. In addition a method as defined herein may comprise additional step(s) than the ones specifically identified, said additional step(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “about” or “approximately” means that the value presented can be varied by +/−10%. The value can also be read as the exact value and so the term “about” can be omitted. For example, the term “about 100” encompasses 90-110 and also 100.

The term “generating” is used herein interchangeably with “making”.

The term “hormone secretion” is intended to encompass secretion of hormones and/or secretion of hormone precursors. If desired, it can be limited to secretion of hormones. For example, in some embodiments, it does not comprise secretion of hormone precursors.

Hormones and hormone precursors are generally referred to herein by their abbreviated names. Their full names are listed here: Cholestocystokin (CCK), Chromogranin A (CHGA), Chromogranin B (CHGB), Gastrin (GAST), Glucagon (GCG), Ghrelin (GHRL), Gastric inhibitory protein (GIP), Motilin (MLN), Neurotensin (NTS), Peptide YY (PYY), Regenerating family member 4 (REG4), Secretogranin 2 (SCG2), Secretogranin 3 (SCG3), Secretogogin (SCGN), Somatostatin (SST), Urocortin 3 (UCN3), Midkine (MDK), Peptidylglycine Alpha-Amidating Monooxygenase (PAM), Regenerating family member 3a (REG3a), Neuropeptide W (NPW), Nucleobindin 2 (NUCB2), VGF nerve growth factor (VGF), Tachykinin 3 (TAC3), Pancreatic polypeptide (PPY), Neurpeptide W (NPW), Cerebellin 1 (CBLN1).

All patent and literature references cited in the present specification are hereby incorporated by reference in their entirety.

The following examples are offered for illustrative purposes only, and are not intended to limit the scope of the present invention in any way.

### EXAMPLES

### Example 1: Materials and Methods

Cell Culture of Human Intestinal Organoids

Tissues from the human duodenum, ileum and colon were obtained from the UMC Utrecht with informed consent of each patient. The patients were diagnosed with small intestinal or colon adenocarcinoma that was resected. A sample from non-transformed, normal mucosa was taken for this study. The study was approved by the UMC Utrecht (Utrecht, The Netherlands) ethical committee and was in accordance with the Declaration of Helsinki and according to Dutch law. This study is compliant with all relevant ethical regulations regarding research involving human participants. Human small intestinal cells were isolated, processed and cultured as described previously (Beumer et al., 2018; Sato et al., 2011)

For differentiation towards EECs, organoids were treated with 1 μg/ml doxycycline (Sigma) in ‘ENR’ medium (Sato et al., 2009). Secretin (Tocris) was used at a concentration of 10 μg/ml. Beta-ionone (Sigma) was used at 100 mg/ml. BMP activation was achieved by withdrawing Noggin from ‘ENR’ and addition of BMP-2 (Peprotech, 50 ng/ml) and BMP-4 (Peprotech, 50 ng/ml). Notch signaling was inhibited by treatment with the Gamma-secretase inhibitor DAPT (Sigma, 10 μM). Wnt inhibition was performed by treatment with the Porcupine inhibitor IWP-2 (Stemgent, 5 μM)

Constructs for EEC-TAG Reporter and Knockout Generation

The NEUROG3 was cloned in a two insert Gibson reaction into BSKS II vector. Of note, two PCR reactions were done: first, NEUROG3 was amplified from human genomic DNA, since the entire coding region lies in one exon. Second, the BSKS vector was amplified. The forward and reverse primers for Gly linker, FLAG, HA and P2A sequence were annealing to each other (Table 3). All three DNA fragments were then combined in BSKS-NEUROG3-Flag-HA-P2A. In the next step, NEUROG3-P2A sequence was excised using EcoRI enzyme and cloned into previously published pLX-NS2 vector (Sachs et al., 2019). Organoids were lentivirally transduced as described before (Koo et al., 2012).

For generation of the reporter organoid lines using CRISPR-HOT, we utilized a method described in (Artegiani, Hendriks et al., 2020). Briefly, we used a targeting plasmid containing a fluorescent protein (mNEON or tdTomato) which can be linearized at a defined base position by a specific sgRNA and Cas9 provided from a second plasmid, which also encodes mCherry (Schmid-Burgk et al., 2016). These two plasmids are co-electroporated with a plasmid encoding the sgRNA for the respective locus (Table 3).

The HDR donor plasmid allows C-terminal knock-in of the fluorescent reporter mClover3 in the TPH1 locus and was generated using pUC118 as a backbone. First, the endogenous SapI site in PUC118 was inactivated. Then, a selection cassette (PGK promoter driven expression of blasticidin) flanked by LoxP and two SapI sites was cloned into the SapI-inactivated pUC118 using infusion cloning (638910, Takara). Subsequently, a P2A sequence and the fluorescent protein mClover3 was PCR amplified (Phusion High fidelity DNA polymerase, M0530S, NEB) from the Addgene plasmid #74252 and cloned upstream of the selection cassette using infusion cloning (638910, Takara) and NotI (R01 89S, NEB) digestion of the pUC118 selection-cassette containing plasmid. Next, homology arms corresponding to the genomic regions, approximately 1000 bp, upstream and downstream of the TPH1 stop codon were PCR amplified (Phusion High fidelity DNA polymerase, M0530S, NEB) from genomic DNA (extracted and purified from human small intestinal organoid DNA). The PCR primers contained overhangs allowing subsequent Golden Gate cloning (Table 3).

The PCR amplified homology arms were purified (QIAquick PCR Purification Kit, 28104, Qiagen) and finally, the targeting vector was generated by SapI (R0569S, NEB) mediated Golden Gate insertion of the homology arms into the pUC118 selection-cassette containing plasmid.

The sgRNA was selected based on the WTSI website (https://www.sanger.ac.uk/htgt/wge/) and chosen as close to the TPH1 stop codon as possible. The gRNA sequence overlapped with the stop codon, so that the homology vector was not cut. The target sequence was ordered as two complementary oligos (IDT) and cloned in the Cas9-EGFP vector (addgene plasmid #48138) following the protocol described before (Ran et al., 2013).

For the generation of HHEX and LMX1A knockout organoids, gRNAs were selected using the WTSI website and cloned in the Cas9-EGFP vector (addgene plasmid #48138) following the protocol described before (Ran et al., 2013). gRNAs used in this story are presented in Table 3.

Human intestinal organoids were transiently transfected using a NEPA21 electroporator and a previously developed protocol (Fujii et al., 2015). 3-7 days after electroporation, either mCherry (for generation of NHEJ-mediated reporter organoids) or EGFP (for generation of HHEX and LMX1A knockout lines) positive cells were sorted using a FACS-ARIA (BD Biosciences). Wnt-surrogate (0.15 nM, U-Protein Expression) and Rho kinase inhibitor (10 μM, Calbiochem) were added to the culture medium up to 1 week after sorting to enhance single cell outgrowth. All reporter organoids were generated in organoid lines also transduced with NEUROG3-overexpression (with or without dTomato) vector.

To confirm correct integration of mNeon/dTomato reporter into the hormone locus, organoids grown from mCherry-positive cells were differentiated towards EECs using overexpression of NEUROG3. Organoids where fluorescent cells appeared during EEC differentiation were picked, digested using TrypLE (TryplE Express; Life Technologies) and clonally expanded to establish stable knock-in organoid lines.

Organoids grown from Cas9-EGFP transfected cells were genotyped for HHEX and LMX1A to confirm homozygous frameshift mutation (primers in Table 3).

Calcium Sensor

A red calcium probe (pTorPE-R-GECO1, addgene plasmid #32465) was used as a template to engineer a cyan genetically encoded calcium probe. The cpApple was replaced with a circular permuted mTurquoise. The resulting probe was dubbed Tq-Ca-FLITS (Turquoise Calcium Fluorescence Lifetime Indicator for Truthful Sensing). A triple nuclear localization signal (3×nls) was added to the N-terminus of the calcium probe to simplify analysis. Details of the engineering and characterization will be described elsewhere (Van der Linden et al., in preparation).

PCRs were performed on Tq-Ca-FLITS (Fw AAACAAGCGGGAGACGTGGAGGAAAACCCTGGACCTCTCGAGatgggatcagatccaaaaaagaagag, Rev ATGGCACTAGGCTAGTTCTAGAcCTACTTCGCTGTCATCATTTGGAC) as well as H2B-mMaroon (Fw TCGGCGCGCCACGCGT, Rev CGTCTCCCGCTTGTTTCAGTAGACTAAAATTCGTCGCGCCAGATCCGCTAGCattaagtttgtgcccc) and the two PCRs were cloned into a lentiviral vector using InPhusion Cloning (Takara), to produce H2B-mMaroon-P2A-Tq-Ca-FLITS, two simultaneously expressed cistrons separated by a de-optimized P2A (Lo et al., 2015).

Live Cell Imaging of Calcium Reporter Organoids

H2B-mMaroon-P2A-Tq-Ca-FLITS organoids were imaged on a Leica SP8 confocal laser scanning microscope, equipped with Argon laser and White Light Laser, the latter allowing spectral flexibility for optimal visualization of all fluorophores. For cell type identification, cells were first imaged in 5 channels (Tq-Ca-FLITS-mTurquoise2, Clover, TdTomato, H2B-Maroon and transmitted light) and subsequently Tq-Ca-FLITS and H2B-mMaroon were time lapse imaged during administration of beta-ionone in XYZT-mode. Post-acquisitional analysis was done with custom-made Fiji-script.

Transmission Electron Microscopy

Organoids were fixed with 1.5% glutaraldehyde in 0.1 M cacodylate buffer. They were kept in the fixative for 24 h at 4° C. Then, they were washed with 0.1 M cacodylate buffer and potsfixed with 1% osmium tetroxide in the same buffer containing 1.5% potassium ferricyanide for 1 hour (dark) at 4° C. Then the samples were dehydrated in ethanol, infiltrated with Epon resin for 2 days, embedded in the same resin and polymerised at 60° C. for 48 hours. Ultrathin sections were obtained using a Leica Ultracut UCT ultramicrotome (Leica Microsystems, Vienna) and mounted on Formvar-coated copper grids. They were stained with 2% uranyl acetate in water and lead citrate. Then, sections were observed in a Tecnai T12 electron microscope equipped with an Eagle 4 k×4 k CCD camera (Thermo Fisher Scientific, The Netherlands).

Alternatively, organoids were chemically fixed at 4° C. with a mixture of 2% paraformaldehyde and 0.2% glutaraldehyde in PB buffer. After washing with PB containing 50 mM glycine, cells were embedding in 12% gelatine and infused in 2.3 M sucrose. Mounted gelatine blocks were frozen in liquid nitrogen. Thin sections were prepared in an ultracryomicrotome (Leica EM Ultracut UC6/FC6, Leica Microsystems, Vienna, Austria). Ultrathin cryosections were collected with 2% methylcellulose in 2.3 M sucrose. The observations were performed in an Electron Microscope Tecnai T12 as mentioned.

Immunostaining

Organoids were stained as described before (Beumer et al., 2018). Primary antibodies used were goat anti-chromogranin A (1:500; Santa Cruz), goat anti-cholestocystokin (sc-21617,1:100; Santa Cruz), rabbit anti-neurotensin (sc-20806,1:100; Santa Cruz), goat anti-somatostatin (sc-7819, 1:100; Santa Cruz), goat anti-serotonin (ab66047, 1:1,000, Abcam), rabbit anti-gastric inhibitory polypeptide (ab22624-50, 1:500; Abcam), goat anti-GLP1 (sc-7782, 1:100; Santa Cruz), rabbit anti-GLP1 (ab22625, 1:200; Abcam), rabbit anti-MLN (HPA069392, 1:200, Atlas antibodies), mouse anti-Gastrin (60346, 1:200, Proteintech), mouse anti beta-Catenin (610154, 1:100; BD transduction laboratories), goat anti-Ghrelin (sc-10368, 1:200; Santa Cruz), rabbit anti-Neuropeptide W (NBP2-57337, 1:100; Novus), rabbit anti-Precerebellin (ABN304, 1:100; Sigma) and rabbit anti-PPY (HPA032122, 1:200; Atlas antibodies). Organoids were incubated with the corresponding secondary antibodies Alexa488-, 568- and 647-conjugated anti-rabbit and anti-goat (1:1,000; Molecular Probes) in blocking buffer containing 4′,6-diamidino-2-phenylindole (DAPI; 1; 1,000, Invitrogen). Sections were embedded in Vectashield (Vector Labs) and imaged using a Sp8 confocal microscope (Leica). Image analysis was performed using ImageJ software.

Fluorescent In Situ Hybridization

FISH was performed using the RNAScope® Multiplex Fluorescent Reagent Kit v2 (Advanced Cell Diagnostics) according to the manufacturer's protocol (Wang et al., 2012). In brief, paraffin embedded ileal surgical sections were deparaffinized, treated with hydrogen peroxide for 10 minutes and boiled in target retrieval buffer for 15 minutes before a 30-minute protease treatment. Probes directed against CHGA/SCTR, CHGA/GCG and GHRL/IL20RA were multiplexed, respectively, amplified and detected using fluorescent probes based on opal dyes. Slides were counterstained with DAPI for 30 seconds, mounted using ProLong™ Gold Antifade Mountant (Thermo Fisher scientific) and images were obtained using a SP8 confocal fluorescent microscope (Leica).

ELISA

The supernatant from organoids either cultured in ENR for 5 days or differentiated towards EECs were collected after a 24-hour stimulation with 10 μM Forskolin (Tocris). GLP-1 concentration was measured using a GLP-1 EIA kit (Rab0201 from Sigma that detects both full-length and N-terminal cleaved GLP-1) following the manufacturer's protocol,

RNA Isolation and Quantitative PCR

Organoid RNA was isolated using a RNAeasy kit (QIAGEN), following the manufacturer's protocol. Quantitative PCR (qPCR) analysis was performed using biological and technical duplicates as described before (Muñoz et al., 2012). Primers were designed using the NCBI primer design tool, tested using a standard curve, and are presented in Table 3.

Single Cell Sorting for RNA Sequencing from Organoids

Organoids were dissociated to single cells using a 10-minute incubation with TrypLE (TrypLE Express; Life Technologies) and repeated mechanical disruption through pipetting. Cells were sorted using a BD FACS Aria (BD Biosciences) based on fluorescence levels. For single cell RNA sequencing, individual cells were collected in 384-well plates with ERCC spike-ins (Agilent), reverse transcription primers and dNTPs (both Promega). Single cell sequencing was performed according to the Sort-seq method (Muraro et al., 2016). Sequencing libraries were generated with TruSeq small RNA primers (Illumina) and sequenced paired-end at 60 and 26 bp read length, respectively, on the Illumina NextSeq.

For bulk RNA sequencing, cells were sorted into Eppendorf tubes containing RLT buffer (RNeasy kit, QIAGEN). 5,000-30,000 cells were sorted per reporter in duplicates (and triplicates for tdTomato negative cells). RNA was extracted using the RNeasy mini kit (QIAGEN) following the manufacturer's instructions. Sequencing libraries were generated using a modified CELseq2 protocol (Hashimshony et al., 2016). 75 bp paired-end sequencing of libraries was performed on an Illumina NextSeq platform.

Single Cell RNA Sequencing Analysis from Organoids

Reads were mapped to the human GRCh37 genome assembly. Sort-seq read counts were filtered to exclude reads with identical library-, cell- and molecule barcodes. UMI counts were adjusted using Poisson counting statistics (Muraro et al., 2016). Cells with fewer than 2,000 unique transcripts were excluded from further analysis.

Subsequently, RaceID3 was used for k-medoids based clustering (knn=10) of cells and differential gene expression analysis between clusters using the standard settings described at https://github.com/dgrun/RaceID3_StemID2_package.

The dataset was then subsetted to require expression of EEC markers and exclude cells based on expression of markers of other cell types with the following transcript count cutoffs: CHGA>5; MUC2<5; FABP1<15; LYZ<15; OLFM4<10. The resulting set of EECs was again subjected to clustering (knn=5) and differential gene expression as described above.

For reporter analyses, cells sorted by fluorescent reporter positivity were analyzed as one dataset per reporter to gain more detailed insights into single EEC subpopulations. The following deviations from standard settings were made per reporter: GCG:knn=5; outlg=1; probthr=0.00001; perplexity=10; MLN: knn=10; probthr=0.0000001; SST: knn=10; perplexity=20

For mouse validation, the tissue-derived single cell count tables from Gehart el al. (2019) were reanalyzed using the procedure and settings described above. No subsetting for EECs was performed.

Bulk RNA Sequencing Analysis

Reads were mapped to the human GRCh37 genome assembly. The counted reads were filtered to exclude reads with identical library- and molecule barcodes. Differential gene expression analysis was performed using the DESeq2 package (Love et al., 2014). For display in heatmaps, genes were ranked by fold change compared against tdTomato negative cells. After filtering for an adjusted p-value<0.05, the row z-score for the top 20 genes was calculated.

Preparation Protocol for Secreted Peptides and Proteins for LC-MS

Organoids differentiated for 5 days to EECs were washed extensively in PBS and stimulated with 10 μM Forskolin (Tocris). Conditioned media was collected for 24 hours and supplemented with 1× Complete Protease Inhibitor Cocktail on harvest (Roche). Potential cell debris was removed by centrifugation at 10,000×g, for 5 min at 4° C. Conditioned media supernatant was denatured in final 4M Urea, 50 mM ammonium bicarbonate and fractionated by molecular weight with a 10 kDa Vivaspin centrifugal device (Sartorius, Göttingen, Germany), at 12,000×g, for 10 min at 4° C. (i) Endogenously processed peptides recovered from the filtrate were acidified to 5% formic acid, desalted by reversed phase C18 1 cc columns (Waters Corporation, Milford, USA), further purified by home-made strong cation exchange STAGE tip, and dried by vacuum centrifugation. (ii) Longer secreted proteins in the 10 kDa retentate were recovered and diluted to final 2 M Urea, 50 mM ammonium bicarbonate, for reduction with dithiothreitol, alkylation with iodoacetamide, and overnight digestion with trypsin (Promega, Madison, USA) at 37° C. Digested peptides were similarly acidified to 5% formic acid, desalted by reversed phase C18 1 cc columns (Waters), and dried by vacuum centrifugation.

Preparation Protocol for FACS-Sorted EECs for Proteome Analyses

FACS sorted enteroendocrine cells were lysed in 8M Urea, 50 mM Ammonium bicarbonate, 0.5% Sodium deoxycholate, 1× cOmplete protease inhibitor, 50 μg/mL DNAse I, and sonicated with the Biorupter (3 cycles, 20 s on, 20 s off at 4° C.) (Diagenode, Liege, Belgium). Cell debris was pelleted by centrifugation at 14,000×g for 1 hour at 15° C., and supernatant containing extracted proteins were reduced, alkylated, diluted 4 times with 50 mM ammonium bicarbonate, and digested sequentially with Lys-C (Wako) and trypsin (Promega). Peptide digests were quenched to 5% formic acid, and sodium deoxycholate was precipitated and removed by centrifugation at 14,000×g, 4° C. for 10 minutes. Peptides in the supernatant were diluted to final 20% acetonitrile and purified by SCX STAGE tips. Eluted peptides were dried by vacuum centrifugation.

Reagents and Resources

A number of reagents and resources suitable for use in the invention are shown in Table 2. In some embodiments, the reagents and resources used in the invention are selected from this table.

TABLE 2

| | | |
|---|---|---|
| REAGENT or RESOURCE | SOURCE | IDENTIFIER |

Antibodies

| | | |
|---|---|---|
| Anti-Chromogranin A | Santa Cruz | sc-1488, RRID: AB_2276319 |
| Anti-Cholestocystokinin | Santa Cruz | sc-21617, RRID: AB_2072464 |
| Anti-Neurotensin | Santa Cruz | sc-20806, RRID: AB_2155562 |
| Anti-Somatostatin | Santa Cruz | sc-7819, RRID: AB_2302603 |
| Anti-Serotonin | Abcam | ab66047, RRID: AB_1142794 |
| Anti-Gastric inhibitory | Abcam | ab22624-50, RRID: AB_2109683 |
| polypeptide | | |
| Anti-GLP1 | Santa Cruz | sc-7782, RRID: AB_2107325 |
| Anti-Motilin | Atlas antibodies | HPA069392, RRID: AB_2686136 |
| Anti-GLP1 | Abcam | ab22625, RRID: AB_447206 |
| Anti-Gastrin | Proteintech | 60346-1-Ig |
| Anti-Ghrelin | Santa Cruz | sc-10368, RRID: AB_2232479 |
| Anti-beta-catenin | BD transduction | #610154, RRID: AB_397555 |
| | laboratories | |
| Anti-Neuropeptide W | Novus biologicals | NBP2-57337 |
| Anti-Precerebellin | Sigma-Aldrich | ABN304 |
| Anti-PPY | Atlas antibodies | HPA032122, RRID: AB_2674164 |
| Alexa Fluor 488 donkey anti-rabbit | Thermo Fisher scientific | A21206, RRID: AB_2535792 |
| Alexa Fluor 488 donkey anti-goat | Thermo Fisher scientific | A11055, RRID: AB_2534102 |
| Alexa Fluor 568 donkey anti-rabbit | Thermo Fisher scientific | A10042, RRID: AB_2534017 |
| Alexa Fluor 568 donkey anti-goat | Thermo Fisher scientific | A11057, RRID: AB_2534104 |
| Alexa Fluor 647 donkey anti-rabbit | Thermo Fisher scientific | A31573, RRID: AB_2536183 |
| Alexa Fluor 647 donkey anti-goat | Thermo Fisher scientific | A32849, RRID: AB_2762840 |
| Alexa Fluor 647 donkey anti-mouse | Thermo Fisher scientific | A31571, RRID: AB_162542 |
| Envision+ System-HRP polymer | DAKO | K4002 |
| anti-rabbit | | |
| Biological Samples | | |
| Human intestinal tissue for | Utrecht Medical Center | N/A |
| organoids | | |
| Human intestinal biopsies for | Addenbrooke's | Ethics: REC 17/EE/0265 |
| RNA sequencing | Hospital, Cambridge | |

Chemicals, Peptides, and Recombinant Proteins

| | | |
|---|---|---|
| 10 kDa Vivaspin centrifugal | Sartorius, Gottingen, | Catalogue # VS0101 |
| device | Germany | |
| Reversed-phase C18 1 cc | Waters Corporation, | Catalogue # WAT054925 |
| columns | Milford, USA | |
| Trypsin enzyme | Promega, Madison, | Catalogue # T1426 |
| | USA | |
| Lysyl endopeptidase enzyme | Wako Chemicals GmbH | Catalogue # 129-02541 |
| (Lys C) | | |
| DNase I | Sigma-Aldrich, | Catalogue #DN25 |
| | Missouri, USA | |
| RNase A | Sigma-Aldrich, | Catalogue # R-6513 |
| | Missouri, USA | |
| Advanced DMEM/F12 | Thermo Fisher scientific | 12634-010 |
| B-27 Supplement | Thermo Fisher scientific | 17504044 |
| GlutaMAX | Thermo Fisher scientific | 35050061 |
| HEPES | Thermo Fisher scientific | 15630080 |
| Penicillin-Streptomycin | Thermo Fisher scientific | 15140122 |
| Wnt surrogate | U-Protein Express | Custom order |
| Noggin conditioned medium | U-Protein Express | Custom order |
| R-spondin conditioned medium | U-Protein Express | Custom order |
| N-Acetyl-L-cysteine | Sigma-Aldrich | A9165 |
| Nicotinamide | Sigma-Aldrich | N0636 |
| Human EGF | Peprotech | AF-100-15 |
| A83-01 | Tocris | 2939 |
| Prostaglandin E2 | Tocris | 2296 |
| Forskolin | Tocris | 1099 |
| A83-01 | Tocris | 2939 |
| SB 202190 | Sigma-Aldrich | S7076 |
| Y-27632 dihydrochloride | Abmole | M1817 |
| Primocin | Invivogen | ant-pm-2 |
| BMP-2 | Peprotech | 120-02C |
| BMP-4 | Peprotech | 120-05ET |
| FGF-21 | Peprotech | 100-42 |
| Secretin | Tocris | 1918 |
| Basement Membrane Extract | R&D Systems | 3533-001-02 |
| (BME), Growth Factor Reduced, | | |
| Type 2 | | |
| DAPI | Thermo Fisher scientific | D1306 |
| Formaldehyde solution 4% | Sigma-Aldrich | 1.00496 |
| SYBR Green | Bio Rad | 1725270 |
| Donkey serum | Golden Bridge | E27-100 |
| | International | |
| Triton X-100 | Sigma-Aldrich | X100-100 ML |
| SORT-seq reagents | (Muraro et al., 2016) | N/A |
| Beta-ionone | Sigma-Aldrich | I12603 |
| DAPT | Sigma-Aldrich | D5942 |
| IWP-2 | Stemcell Techonologies | 72122 |
| SapI | New England Biolabs | R0569S |
| NotI | New England Biolabs | R0189S |
| Phusion High fidelity DNA | New England Biolabs | M0530S |
| polymerase | | |
| TrypIE | Thermo Fisher scientific | 12605010 |
| Vectashield | Vector Labs | H-1000-10 |
| Hyaluronidase | Merck | #385931-25KU |

Critical Commercial Assays

| | | |
|---|---|---|
| RNeasy Mini Kit | QIAGEN | 74104 |
| GLP-1 ELISA kit | Sigma-Aldrich | RAB0201 |
| In-fusion cloning kit | Takara | 638910 |
| QIAquick PCR Purification Kit | Qiagen | 28104 |
| Thermo Scientific reagents for | (Hashimshony et al., | N/A |
| CEL-Seq2 | 2016) | |
| Reagents for library preparation | (Hashimshony et al., | N/A |
| from CEL-Seq2 | 2016) | |
| Miniprep DNA isolation kit | Thermo Fisher scientific | K210003 |
| Midiprep DNA isolation kit | Thermo Fisher scientific | K210005 |

Deposited Data

| | | |
|---|---|---|
| Raw and analyzed sequencing | This paper | GSE XXXXXXX |

Software and Algorithms

| | | |
|---|---|---|
| CFX manager software | Bio-Rad | N/A |
| RaceID3 | (Herman et al., 2018) | N/A |
| GraphPad PRISM 8 | GraphPad | N/A |
| Las X | Leica | N/A |
| Fiji | NIH, Fiji developers | https://imagej.net/Fiji |
| Rstudio | Rstudio | https://rstudio.com/ |
| Adobe illustrator | Adobe inc. | N/A |
| Cellranger | 10x Genomics | Version 2.1.0 (reference |
| | | transcriptome GRCh38-1.2.0) |

Other

| | | |
|---|---|---|
| EVOS Cell Imaging System | Thermo Fisher scientific | N/A |
| EVOS FL Auto 2 Cell Imaging | Thermo Fisher scientific | N/A |
| System | | |
| SP8 confocal microscope | Leica | N/A |
| DM4000 | Leica | N/A |
| NEPA21 electroporator | Nepagene | N/A |
| FACSAria | BD Bioschiences | N/A |
| FACS BD Influx | BD Bioschiences | N/A |

gRNAs and Primers for Use in the Invention

The gRNAs and primers that were used by the inventors and which may be used in the invention if desired are shown in Table 3 below.

TABLE 3

| SEQ ID | Description | Sequence |
|---|---|---|
| NO | | |
| 7 | GCG forward gRNA | CACCGTTCAGACCAAAATCACTGAC |
| 8 | GCG reverse gRNA | AAACGTCAGTGATTTTGGTCTGAAC |
| 9 | MLN forward gRNA | CACCGTCCCCAGCGTGGCCATCACT |
| 10 | MLN reverse gRNA | AAACAGTGATGGCCACGCTGGGGAC |
| 11 | GIP forward gRNA | CACCGAGACAAACCTCTGCAGGCTC |
| 12 | GIP reverse gRNA | AAACGAGCCTGCAGAGGTTTGTCTC |
| 13 | CHGA forward gRNA | CACCGCAGCTGCAGGCACTACGGCG |
| 14 | CHGA reverse gRNA | AAACCGCCGTAGTGCCTGCAGCTGC |
| 15 | NTS forward gRNA | CACCGTTTGAGTATGTAGGGTCTTC |
| 16 | NTS reverse gRNA | AAACGAAGACCCTACATACTCAAAC |
| 17 | GHRL forward gRNA | CACCGGCTTGTGGGCGATCACTTGT |
| 18 | GHRL reverse gRNA | AAACACAAGTGATCGCCCACAAGCC |
| 19 | CCK forward gRNA | CACCGTGGCGGCTGGGTCCTCTAGG |
| 20 | CCK reverse gRNA | AAACCCTAGAGGACCCAGCCGCCAC |
| 21 | SST forward gRNA | CACCGCTGGCTGCAAGAATTTCTTC |
| 22 | SST reverse gRNA | AAACGAAGAAATTCTTGCAGCCAGC |
| 23 | GAST forward gRNA | CACCGCTTCGGCCGCCGCAGTGCTG |
| 24 | GAST reverse gRNA | AAACCAGCACTGCGGCGGCCGAAGC |
| 25 | TPH1 left homology arm | AACGCTCTTCATAAGGGGAGTTAGGCCATTCATGAAAC |
| | forward primer | |
| 26 | TPH1 left homology arm | CGCGCTCTTCCTGTGATACTCGGCTTCCTGCTGACCT |
| | reverse primer | |
| 27 | TPH1 right homology arm | CGCGCTCTTCGCGCCAGTAGCCAGTCATCCAGGAACA |
| | forward primer | |
| 28 | TPH1 right homology arm | AACGCTCTTCGATTAACTCTTCCTTTGATAACAATGCTT |
| | reverse primer | CCT |
| 29 | TPH1 forward gRNA | CACCGACTGGCTACTGTTAGATACT |
| 30 | TPH1 reverse gRNA | AAACAGTATCTAACAGTAGCCAGTC |
| 31 | HHEX forward gRNA | CACCGAAGGCTGGATGGATCGGCGT |
| 32 | HHEX reverse gRNA | AAACACGCCGATCCATCCAGCCTTC |
| 33 | LMX1A forward gRNA | CACCGCCGAGGTGTCGATCGCGCTT |
| 34 | LMX1A reverse gRNA | AAACAAGCGCGATCGACACCTCGGC |
| 35 | HHEX forward primer | TTTACATCGAGGACATCCTGG |
| 36 | HHEX reverse primer | AAACCAATCTGAGTCACGGTGCG |
| 37 | LMX1A forward primer | ATACCCCAGTAGCTGCGTTC |
| 38 | LMX1A reverse primer | CGAGAAGAGGGGGGCATTTA |
| 39 | BSKS forward primer | GGGCTGCAGGAATTCGATAT |
| 40 | BSKS reverse primer | CACTAGTTCTAGAGCGGCCG |
| 41 | Tag forward primer | GGTGGTGGTGGTTCAGGAGGAGGATCGGGAAGCGGA |
| | | GAGGGCAGAGGAAGTCTGCTAACATGCGGTGACGTCG |
| | | AGGAGAATCCTGGACCTGAATTCGGGCTGCAGGAATT |
| | | CGATAT |
| 42 | Tag reverse primer | ATATCGAATTCCTGCAGCCCGAATTCAGGTCCAGGATT |
| | | CTCCTCGACGTCACCGCATGTTAGCAGACTTCCTCTGC |
| | | CCTCTCCGCTTCCCGATCCTCCTCCTGAACCACCACCA |
| | | CC |
| 43 | NEUROG3 forward primer | CCTCCTGAACCACCACCACCCAGAAAATCTGAGAAAGC |
| | | CA |
| 44 | NEUROG3 reverse primer | CGGCCGCTCTAGAACTAGTGGAATTCGCCACCATGAC |
| | | GCCTCAACCCTCGGG |
| 45 | GAPDH forward primer | GGAGCGAGATCCCTCCAAAAT |
| 46 | GAPDH reverse primer | GGCTGTTGTCATACTTCTCATGG |
| 47 | CHGA forward primer | TAAAGGGGATACCGAGGTGATG |
| 48 | CHGA reverse primer | TCGGAGTGTCTCAAAACATTCC |
| 49 | SST forward primer | ACCCAACCAGACGGAGAATGA |
| 50 | SST reverse primer | GCCGGGTTTGAGTTAGCAGA |
| 51 | LYZ forward primer | CTTGTCCTCCTTTCTGTTACGG |
| 52 | LYZ reverse primer | CCCCTGTAGCCATCCATTCC |
| 53 | GAST forward primer | ATGCAGCGACTATGTGTGTATG |
| 54 | GAST reverse primer | GCCCCTGTACCTAAGGGTG |
| 55 | CCK forward primer | AGCTCCTTCTGGACGAATGTC |
| 56 | CCK reverse primer | TGTAGTCCCGGTCACTTATCC |
| 57 | MLN forward primer | ATGGTATCCCGTAAGGCTGTG |
| 58 | MLN reverse primer | CTGGAGTTCGCCATAGGTGAA |
| 59 | NTS forward primer | TGCTTTAGATGGCTTTAGCTTGG |
| 60 | NTS reverse primer | TTCCTGGATTAACTCCCAGTGT |
| 61 | PYY forward primer | GACGCCTACCCCATCAAACC |
| 62 | PYY reverse primer | CCGTCTCTTTTCCCATACCGC |
| 63 | GCG forward primer | ACATTGCCAAACGTCACGATG |
| 64 | GCG reverse primer | TCTGCGGCCAAGTTCTTCAA |
| 65 | TPH1 forward primer | ACGTCGAAAGTATTTTGCGGA |
| 66 | TPH1 reverse primer | ACGGTTCCCCAGGTCTTAATC |
| 67 | OR51E2 forward primer | ACTTCACACATGCCACCTTTG |
| 68 | OR51E2 reverse primer | GAAGACCACGATGCAGTTTCC |

### Example 2: Production of Region-Specific Human EECs

Previous attempts to create human EECs in vitro have relied on growth-factor based differentiation (Beumer et al., 2018) or overexpression of NEUROG3, the key transcription factor to instruct EEC fate (McCracken et al., 2014; Sinagoga et al., 2018). Both iPSC- (Zhang et al., 2019) and adult stem cell (ASC)-based (Chang-Graham et al., 2019) approaches have proven useful to understand aspects of human EEC biology, such as modeling of inherited NEUROG3 mutations and viral infection-mediated serotonin release (Chang-Graham et al., 2019). However, imperfect differentiation and regional restriction of the donor material have limited these studies to a subset of human EECs.

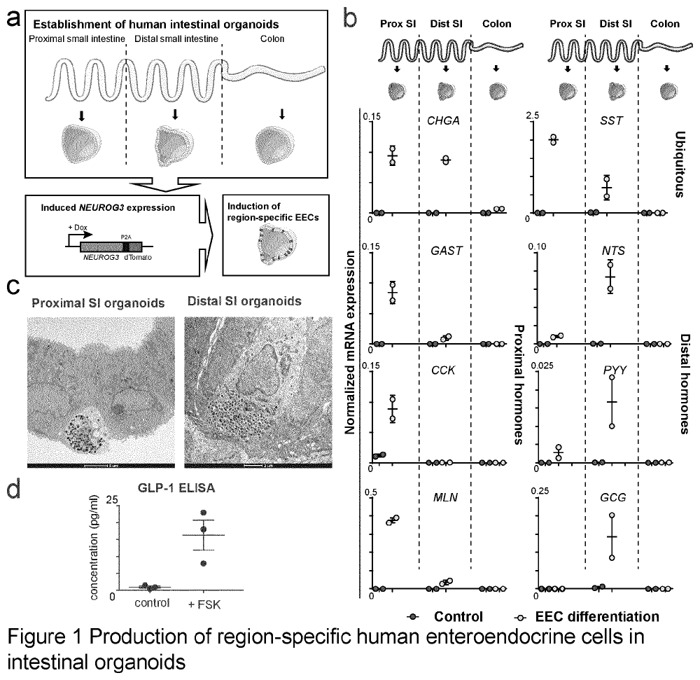

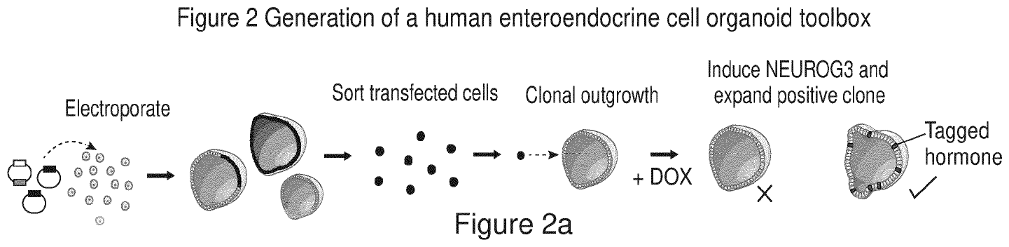

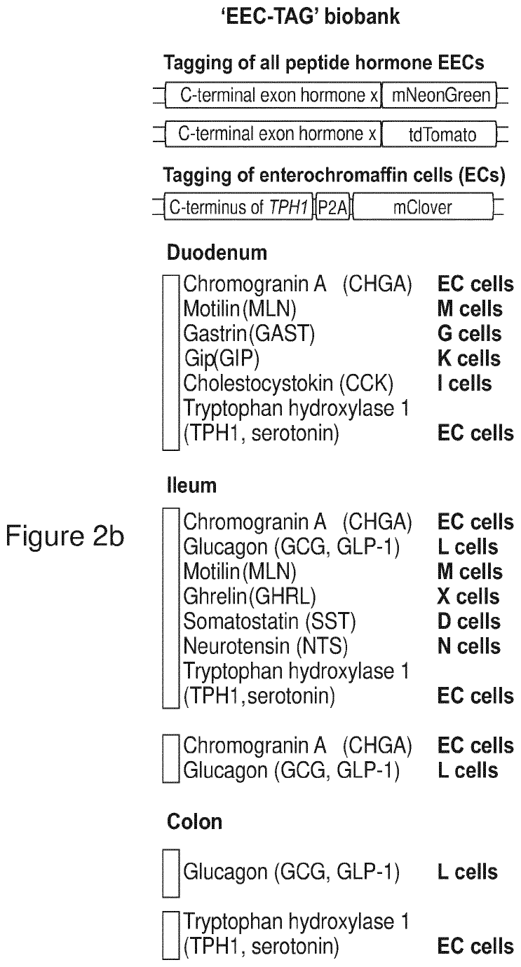

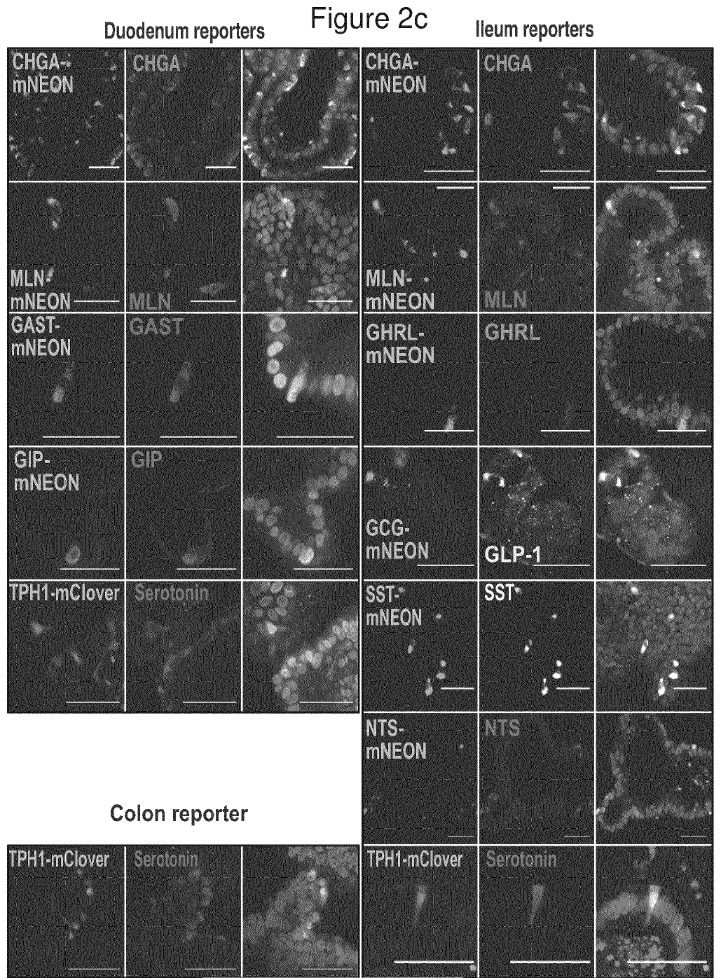

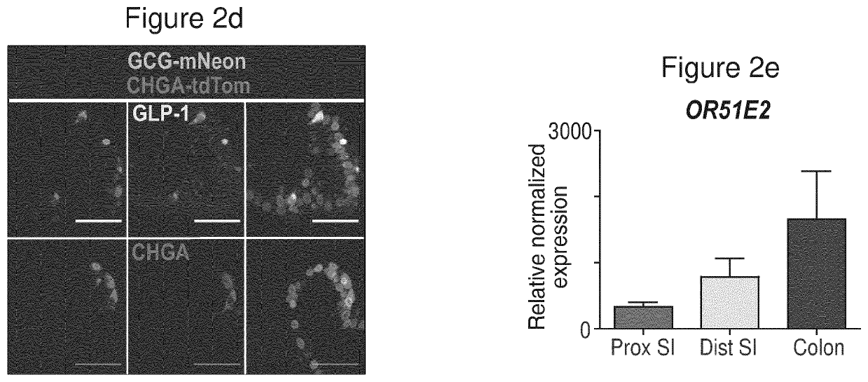

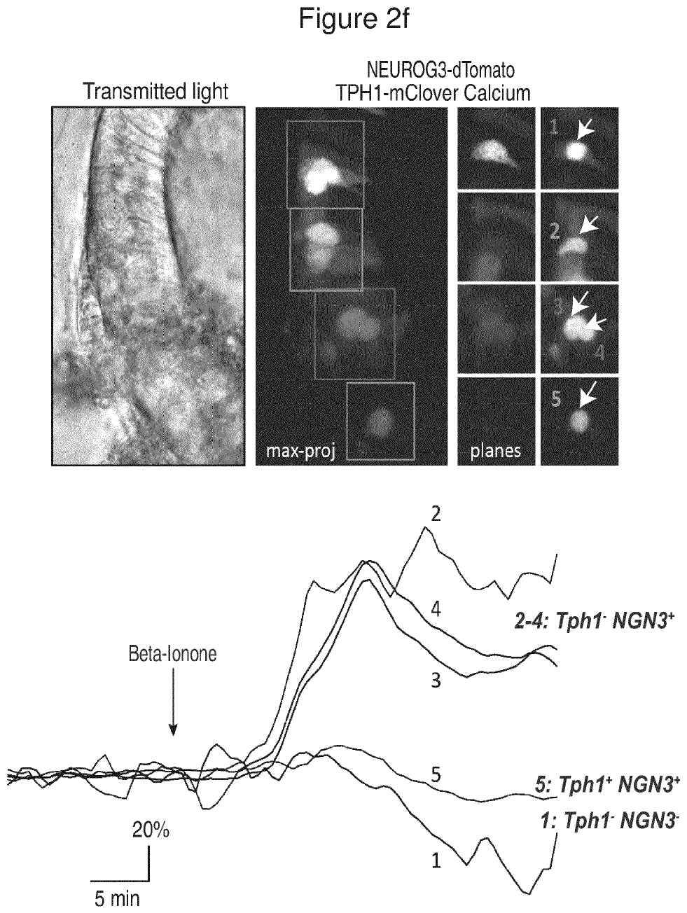

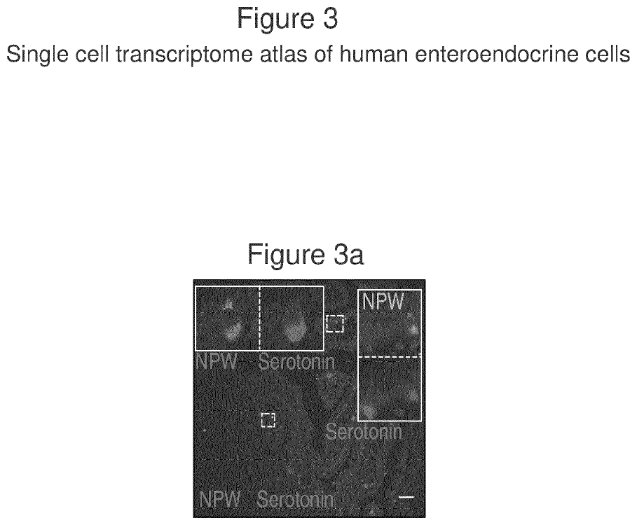

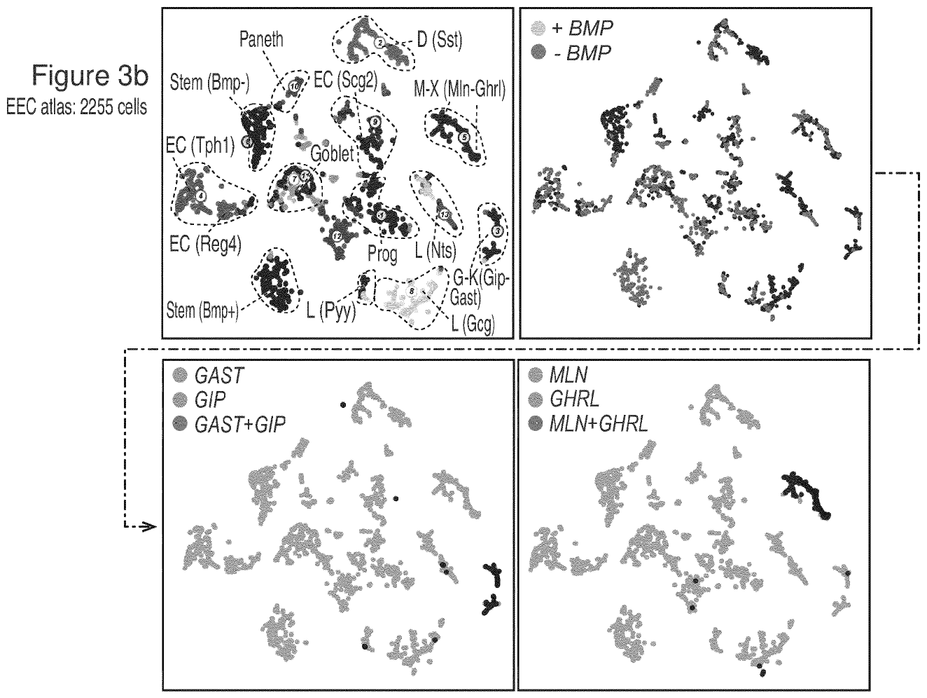

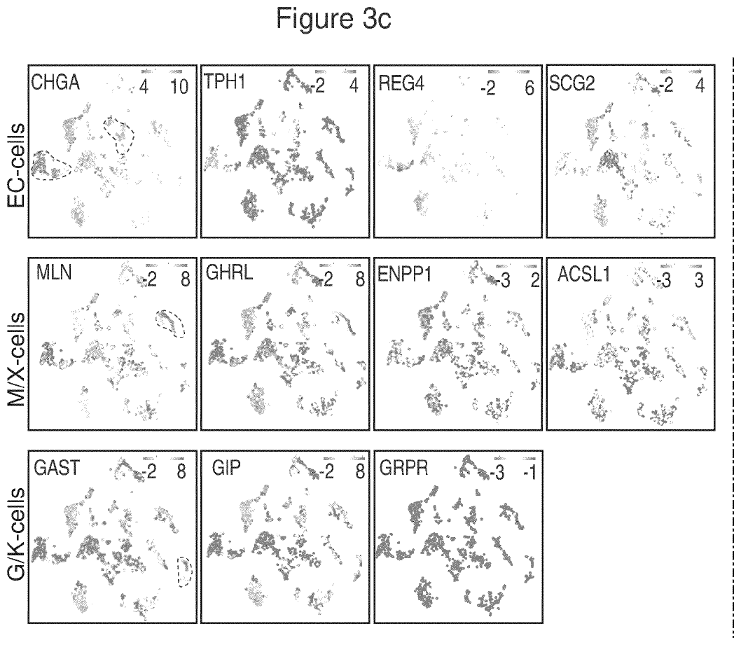

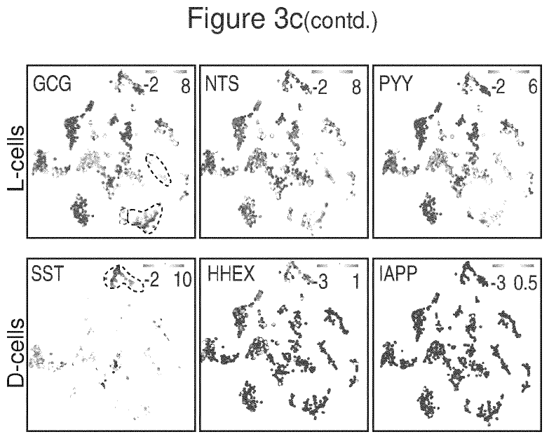

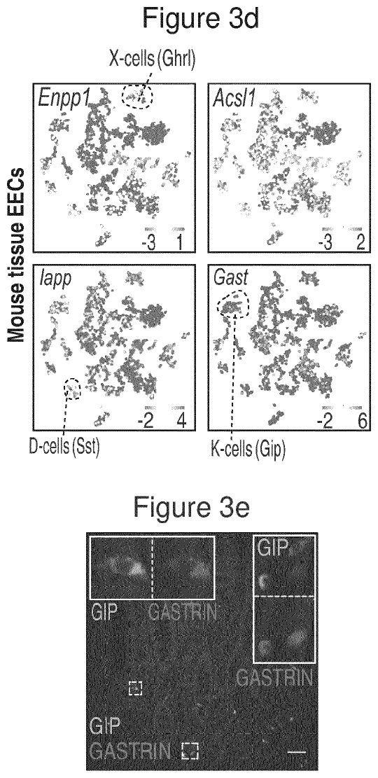

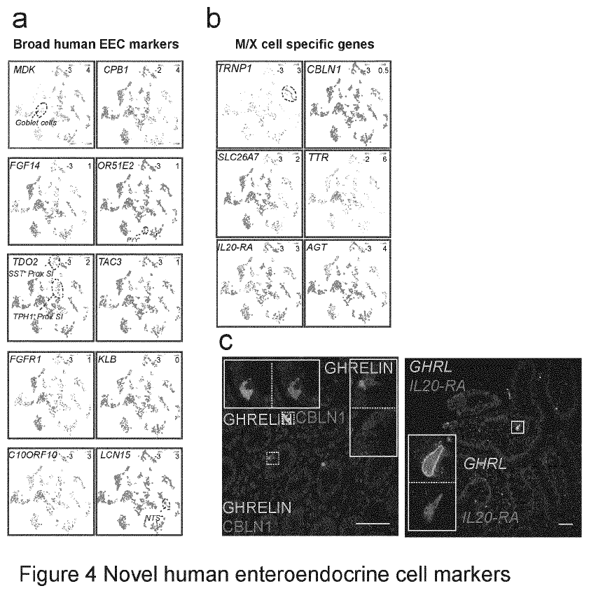

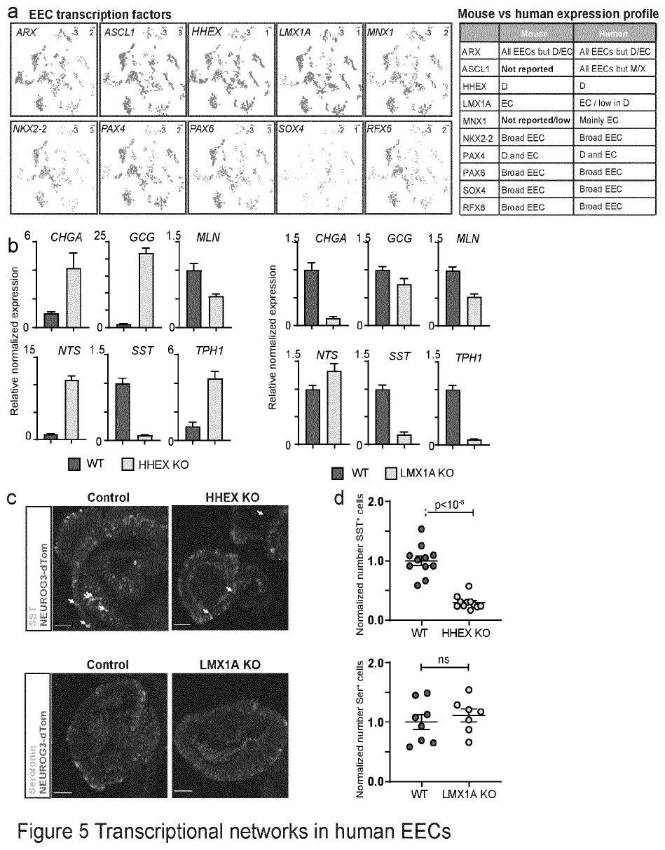

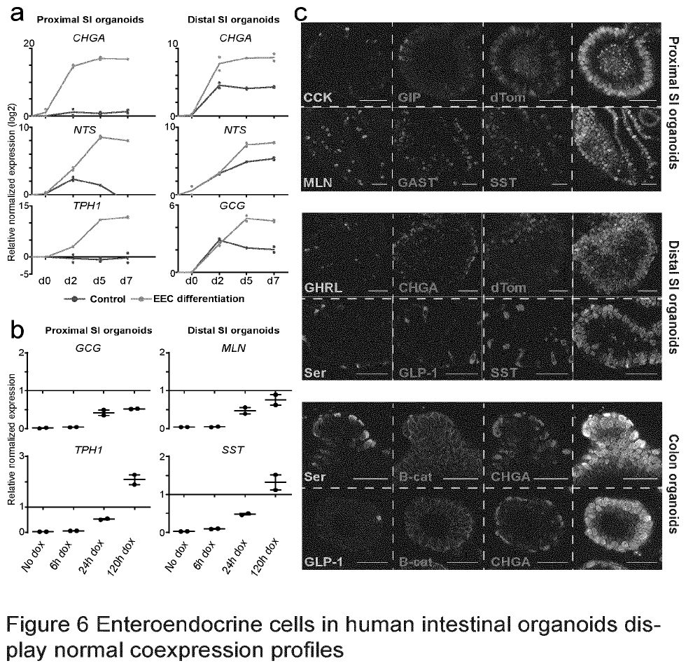

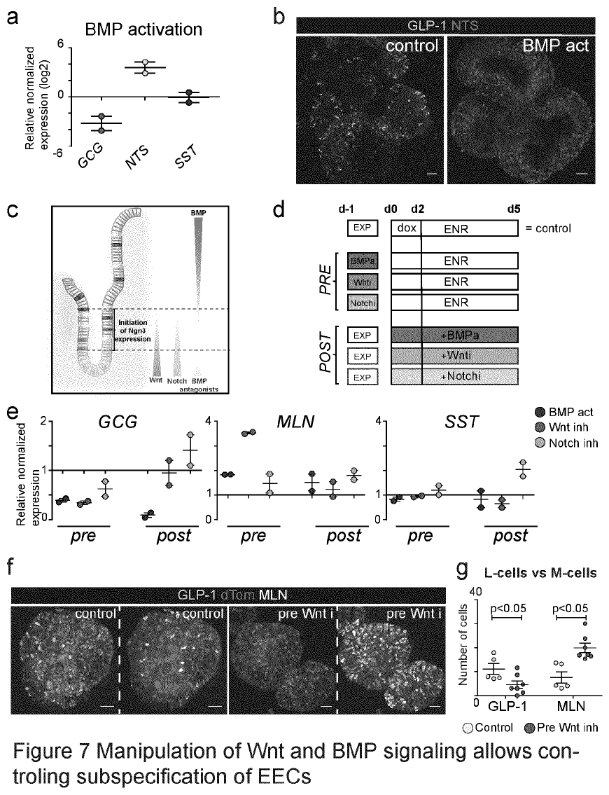

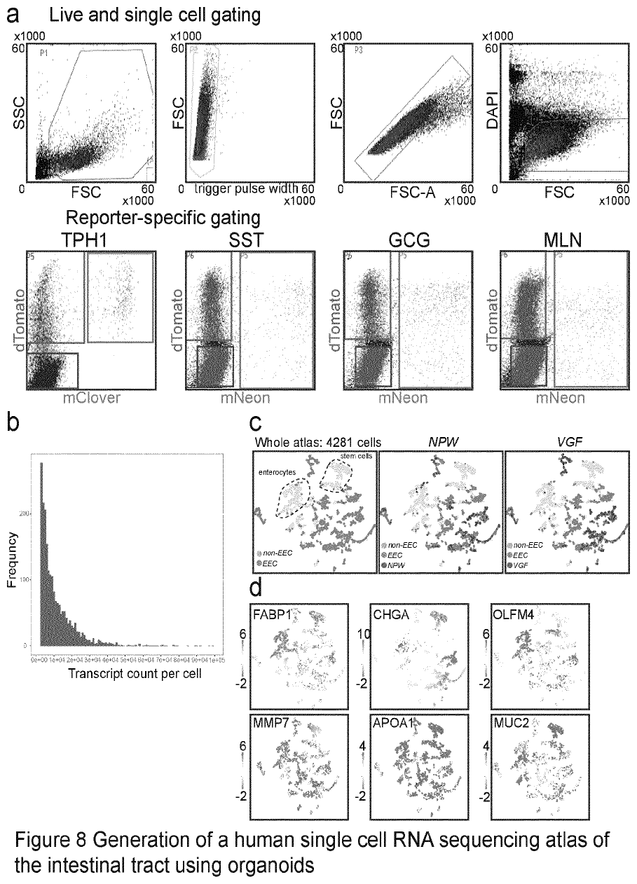

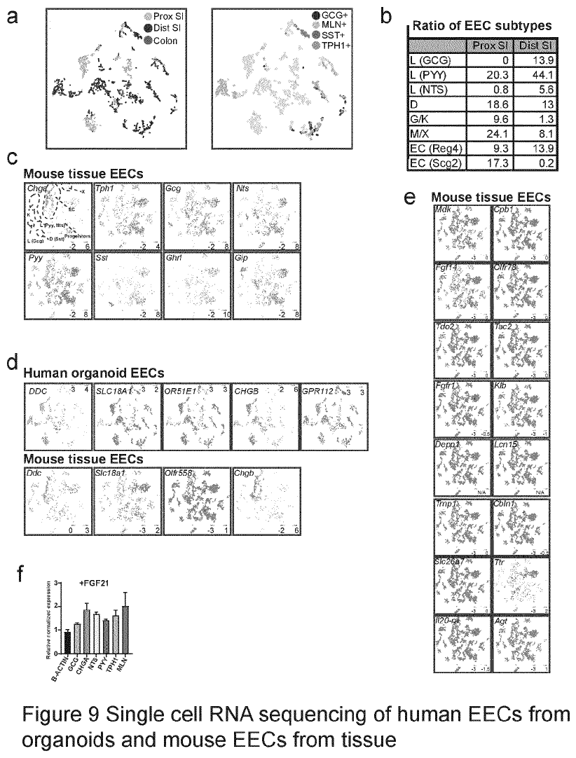

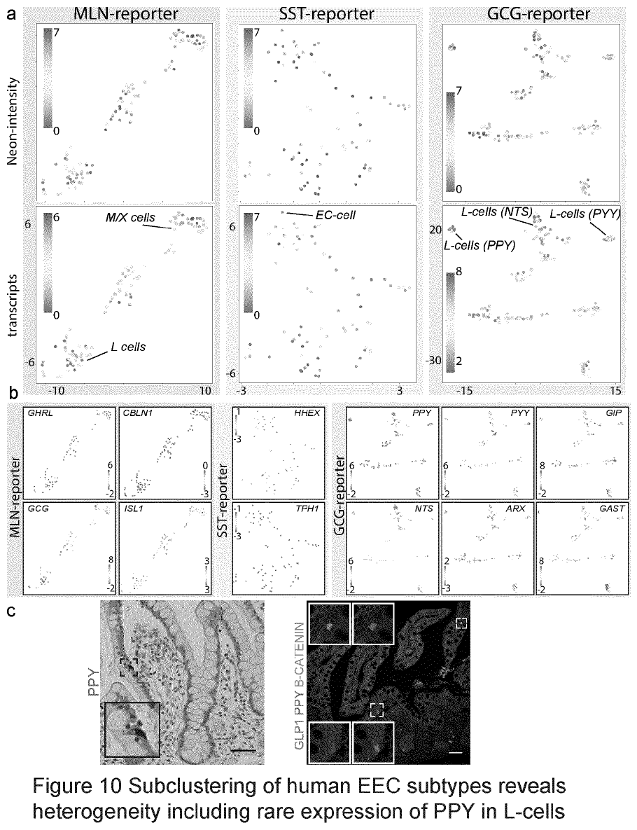

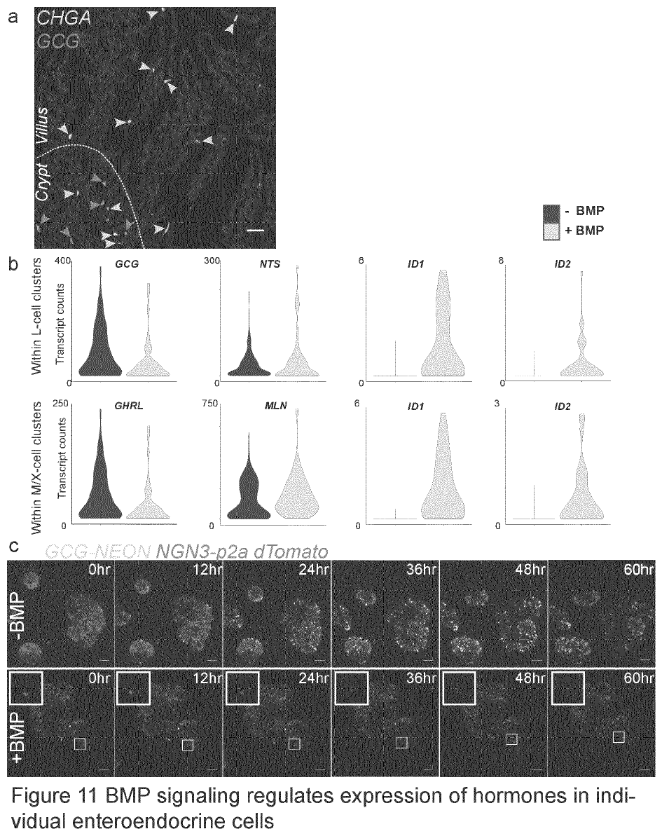

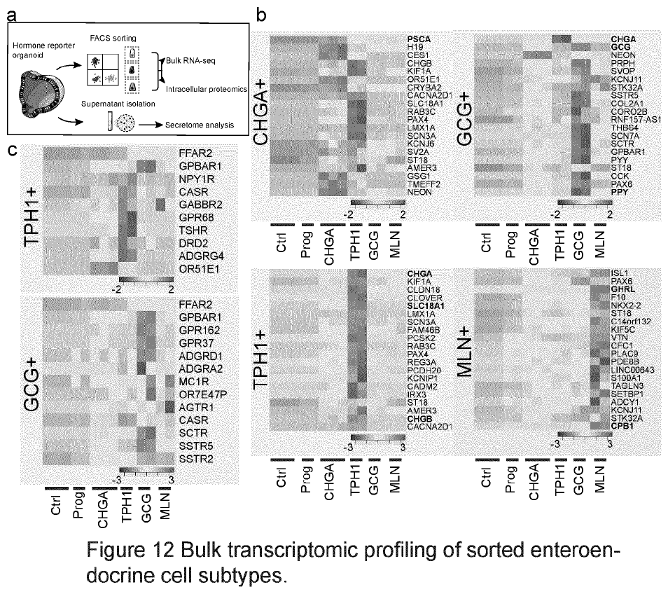

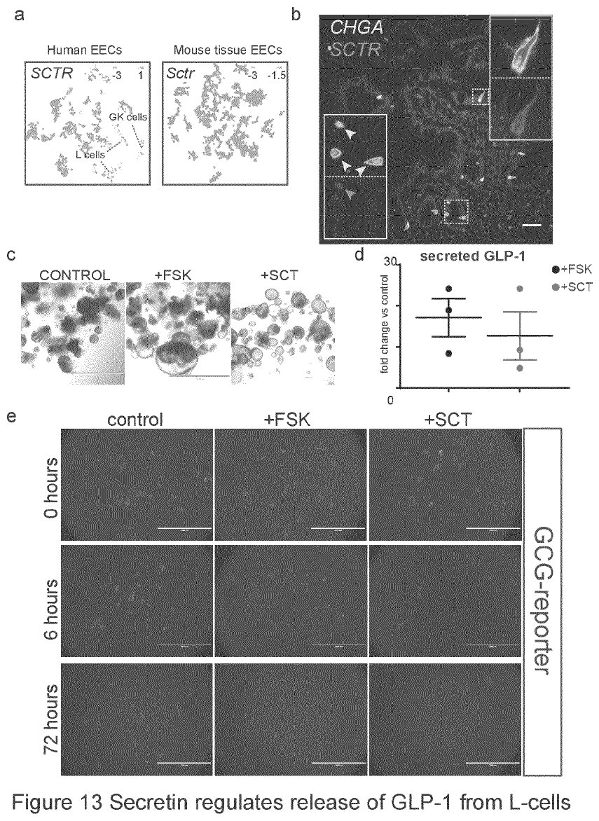

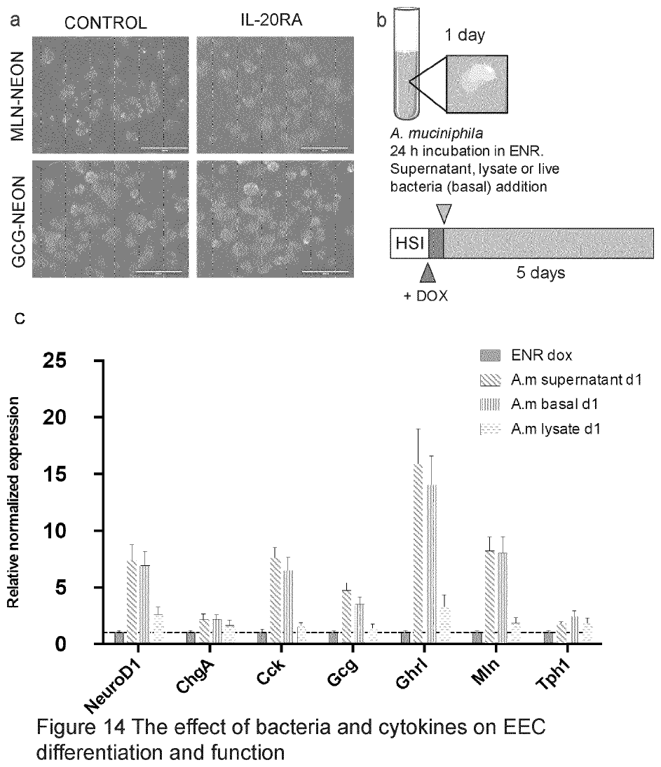

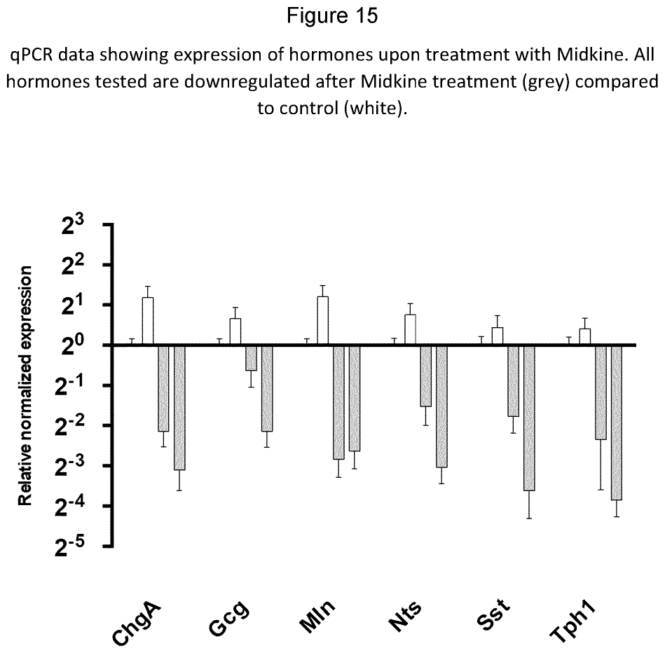

To generate the full spectrum of human EECs, we established organoids from healthy adult proximal small intestine (duodenum), distal small intestine (ileum) and the ascending colon (Sato et al., 2011). These organoids were transduced with a doxycycline-inducible NEUROG3 construct (FIG. 1A). dTomato was inserted 3′ to the NEUROG3 reading frame, separated by a self-cleavable P2A sequence to avoid creating a fusion protein. A 48-hour-pulsed expression of NEUROG3 in the basic medium ‘ENR’ promoted the expression of the broad EEC marker Chromogranin A (CHGA) (FIG. 1B). Proximal SI hormones such as GAST, CCK and MLN were enriched in duodenal organoids, whereas NTS, PYY and GCG were predominantly observed in distal SI organoids. Of note, GCG encodes the preproglucagon prehormone, a protein precursor to a set of hormones including GLP-1 (see below). SST was comparably expressed in proximal and distal organoids, consistent with its profile in the mouse gut. A recent single cell RNA sequencing study of inflammatory bowel disease patients and healthy controls generated the profile of 83 colonic EECs, suggesting that the human colon produces a small repertoire of hormones: serotonin-producing ECs and L-cells positive for GCG and PYY (Parikh et al., 2019). Consistently, induced colon organoids only contained serotonin-producing ECs and GCG-expressing EECs. To establish the optimal window of differentiation towards EECs, we monitored hormone expression over time. We found that hormone expression peaked 5 days after initiation of NEUROG3 expression (FIG. 6A). Therefore, we applied a 5 day-differentiation protocol in ENR throughout the remainder of this study. Under these conditions, EECs in organoids displayed a normal morphology as visualized by transmission electron microscopy. Note the typical basal concentration of hormone vesicles (FIG. 1C).

We tested hormone co-expression by immunofluorescent staining (FIG. 6C). We observed mutually exclusive expression for MLN and GAST, for GHRL and CHGA and for Serotonin and GLP-1, while a subset of GIP-positive cells co-expressed CCK. This closely resembled the co-expression patterns in mice (with the exception of MLN, a pseudogene in mice) (Haber et al., 2017). Virtually all EECs, as identified by the broad marker CHGA, were derived from NEUROG3-overexpressing cells as indicated by dTomato-positivity (FIG. 6C). A definitive hallmark of a mature EEC is its ability to secrete hormones. Indeed, exposure of organoids to Forskolin, a stimulator of adenylate cyclase increasing cAMP levels and thus of general hormone secretion, greatly enhanced GLP-1 levels in the culture supernatant (FIG. 1D).

The ability to enrich for specific EEC subsets in organoids would enhance the applicability of the system. We have previously reported that BMP signaling acts on mature murine EECs in villi, but not in crypts (Beumer et al., 2018). BMP thus controls a switch in hormone repertoire of EECs during their journey from the crypt base to the villus tip. Consistent with our observations in murine EECs, we found that activation of BMP signaling enhances the expression of NTS, while reducing GLP-1 (FIG. 7A,B). These data indicated that human EECs generated in vitro are responsive to BMP. These observations were refined later in this study (see below).

Since the initial expression of NEUROG3 occurs at random positions along the crypt axis in mice (Gehart et al., 2019), we hypothesized that exposure to other crypt differentiation signals (i.e. Notch, Wnt) prior to this expression pulse could potentially influence their eventual fate. We therefore modulated these signals prior to inducing NEUROG3 expression, mimicking the different initiation sites along the intestinal crypt axis (FIG. 7C). As a control, we modulated the same signals after NEUROG3 induction (FIG. 7D). Inhibition of Notch before or after expression of NEUROG3 did not affect EEC differentiation (FIG. 7E). Inhibition of Wnt signaling before (but not after) the NEUROG3 pulse stimulated expression of MLN at the expense of GCG, while having no effect on SST expression (FIG. 7E). Immunofluorescence revealed an increase in the number of MLN-producing cells rather than in the ‘per cell’-expression levels (FIG. 7F), resulting in a strong shift in the ratio between L-cells and M-cells (FIG. 7G).

### Example 3: Generation of a Hormone Reporter Biobank EEC-TAG

Mouse models in which hormone expression is coupled to a fluorescent readout have been generated for several murine EEC hormones: Chga, Gcg, Gip, Cck, Ghrl and Pyy (Engelstoft et al., 2013b, 2015; Gong et al., 2003; Parker et al., 2009; Reimann et al., 2008; Sommer and Mostoslavsky, 2014). These transgenic mouse models have been instrumental to study specific EEC subsets. CRISPR/Cas9 allows the targeting of endogenous loci. Subsequent repair through homology-directed-repair (HDR) or Non-homologous-end-joining (NHEJ) in turn allows for the introduction of exogenous genetic material (Bukhari and Müller, 2019; He et al., 2016; Schmid-Burgk et al., 2016). Enterochromaffin cells (ECs) are characterized by the production of the neurotransmitter serotonin. To mark ECs, we chose to label Tryptophan hydroxylase 1 (TPH1), the rate-limiting enzyme involved in the synthesis of serotonin. Using HDR, we tagged TPH1 with fluorescent mClover separated by a self-cleaving P2A site, and selected targeted organoids using Blasticidin. Our lab has recently optimized a strategy for site-specific introduction of DNA into organoids using NHEJ, a technique termed CRISPR-HOT (Homology-independent Organoid Transgenesis) (Artegiani, Hendriks et al., 2020). We fluorescently labeled a series of secreted hormones as fusion proteins using this method (FIG. 2A). Cells were transfected 1) with a gRNA targeting the hormone locus near its stop codon, 2) a vector encoding mNeon or tdTomato and 3) a vector encoding Cas9, a constitutively produced mCherry fluorescent molecule and a gRNA linearizing the vector encoding the fluorescent molecule. Five days later, transfected cells were sorted for mCherry and plated as single cells. After two weeks, NEUROG3 was induced in the resulting clonal organoids to visualize expression of the fluorescent fusion hormones. Typically, the first fluorescent organoids appeared 2-3 days later and were then clonally expanded.

Applying these approaches to 10 different hormone loci, we generated a small biobank of hormone reporter organoids termed EEC-TAG, consisting of duodenal, ileal and colon organoids in which the major human hormones are marked (FIG. 2A,B). The organoid lines showed complete overlap between fluorescent reporters and the corresponding hormone product (FIG. 2C). The fluorescently tagged hormones localized to cytoplasmic vesicles. Through repeated rounds of fluorescent reporter insertion, reporter lines with more than 1 labelled hormone could be generated (FIG. 2D).

Calcium signaling is one of the mediators of hormone secretion (Goldspink et al., 2018). To measure Ca2+ responses, we stably introduced a Turquoise Ca2+ sensor (Tq-Ca-FLITS) into NEUROG3^{dTomato}TPH1^{mClover} reporter organoids using lentiviral transduction. The resulting genotype of the organoids is NEUROG3^{dTomato}TPH1^{mClover} CaFLITS^{Turquoise}. We chose to stimulate the olfactory receptor OR51E2, of which the mouse homologue (Olfr78) is reported to be expressed in in mouse EECs (Fleischer et al., 2015; Jovancevic et al., 2017). After overexpression of OR51E2, HEK cells elicit a calcium response when stimulated with the selective agonist beta-ionone (Pietraszewska-Bogiel et al., 2019). OR51E2 is most strongly upregulated in distal organoids (FIG. 2E). When reporter organoids were stimulated with beta-ionone, we observed calcium sparking in EECs that were TPH1-negative (FIG. 2F). This provided proof-of-concept that sensors combined with hormone reporters from EEC-TAG can aid the study of functional signaling responses in human EEC subtypes.

### Example 4: Single Cell Transcriptomics of Human EEC Subtypes

Single cell transcriptomics presents a powerful technique to assess heterogeneity among cell populations and identifies genes specific to individual cell types (Haber et al., 2017; Parikh et al., 2019). Due to the paucity of EECs, studies in mice have utilized reporter mice to enrich for hormone-producing cells when performing single cell RNA sequencing. This approach cannot be used for primary human EECs, making the generation of a detailed atlas from small intestinal tissue challenging.

Murine EECs taken from primary tissue and from organoids are essentially identical (Gehart et al., 2019; Grün et al., 2015). We therefore exploited the human NEUROG3-induced organoids to perform single cell RNA sequencing and construct a human EEC atlas. NEUROG3 was induced in duodenal, ileal and colon organoids in the absence or presence of BMP (to generate the crypt- and villus-‘versions’ of EECs, FIG. 7). Data from a total of 8448 cells were subsequently processed by SORT-seq (Muraro et al., 2016) (FIG. 8). We analyzed the transcriptome data using RaceID3, a clustering method based on k-medoids (Herman et al., 2018). After filtering (cut-off: 2000 uniquely expressed transcripts per cell), a broad intestinal cell type atlas was generated from 4281 cells (FIG. 8C). These were derived from the following sources: 1446 cells from duodenum, 2145 cells from ileum, 690 cells from colon. This atlas contained five large clusters: CHGA-positive EECs (2255), and the following well-defined ‘contaminant’ lineages: FABP1-positive enterocytes (585), OLFM4-positive stem cells (113), rare MUC2-positive goblet cells (33), LYZ/MMP7-positive Paneth cells (11), as well as several progenitor populations (FIG. 8C,D).

Neuropeptide W (NPW) and VGF, recently proposed as EEC hormones based on bulk RNA sequencing (Roberts et al., 2019), were broadly expressed among all EEC subtypes (FIG. 8C). While the function of VGF remains elusive, NPW is expressed in different parts of the brain. NPW increases food intake when injected in the hypothalamus (Levine et al., 2005). We confirmed protein expression of NPW by EECs in sections of human intestine by immunofluorescence (FIG. 3A).

We identified all EECs and their progenitors in the dataset by thresholding for expression of the generic EEC marker CHGA and thresholding against MUC2, FABP1, LYZ and OLFM4. After this, we retained 2255 cells (of which 805 cells were BMP-treated) from which we constructed an EEC atlas (FIG. 3B,C, 9A). The major clusters largely overlapped with those described in mouse, and the different EEC subtypes displayed the expected ratios related to their regional identity (FIG. 9B) (Haber et al., 2017). We contrasted our current human EEC atlas with our previously published mouse EEC atlas from tissue (FIG. 9C) (Gehart et al., 2019).

The largest cluster of human EECs was formed by TPH1-expressing Enterochromaffin cells (ECs), expressing the highest levels of CHGA (as in mouse) and representing the most frequent EEC type in vivo (FIG. 3B,C). ECs occurred in three ‘flavors’: REG4high and REG4low cells (in cluster 4) were previously seen also in murine intestine (Haber et al., 2017). A third population of ECs, not separately observed in mice, expressed high levels of the secretogranin SCG2 and occurred mostly in proximal SI organoid cells (cluster 9) (FIG. 3B, C). All human ECs expressed high levels of Dopa decarboxylase (DDC) involved in serotonin biosynthesis, as well as SLC18A1, involved in serotonin transport (FIG. 9D) (Lohoff et al., 2006). The prototypical EC markers CHGB and GPR112 were broadly expressed by human ECs, as was the olfactory receptor OR51E1 (mouse homologue Olfr558), a marker of serotonin-producing neuroendocrine tumors in man (FIG. 9D) (Cui et al., 2013).

Cells producing Gastrin (Gast) are largely restricted to the mouse stomach, whereas in man expression continues more distally along the GI tract in EECs termed G-cells (Engelstoft et al., 2013a). Cells expressing GAST (cluster 3) co-expressed the receptor for Gastrin-releasing peptide, GRPR, a marker of G-cells in the mouse stomach (FIG. 3B,C). GAST-expression often overlapped with high expression of the incretin GIP (same cluster 3), the main hormone product of murine K-cells. We named these cells G/K-cells (FIG. 3B,C). In histological sections of the human intestine, we confirmed largely overlapping expression profiles for these two hormones (FIG. 3E). The L-cell lineage clusters 8 and 13 displayed largely overlapping expression of GCG, NTS and PYY (FIG. 3B, C). Cluster 2 contained SST-positive D-cells, also expressing the transcription factor HHEX. HHEX has been described in murine pancreatic and intestinal Sst-producing cells (Haber et al., 2017; Zhang et al., 2014).

MLN+ cells do not exist in mice. We identified a clearly separated cluster of cells producing MLN and GHRL (cluster 5). A gradient from predominantly MLN- to predominantly GHRL-expressing cells can be observed in t-SNE space (FIG. 3B,C). We termed these M-X or X-M cells (based on the highest expression of either MLN or GHRL, respectively). We hypothesized that these might represent different states of the same cell type. Indeed, BMP treatment reduced levels of GHRL, while MLN levels were slightly increased (FIG. 11B).

M/X cells were further characterized by ENPP1 expression, a known regulator of insulin responses and extracellular ATP levels (Di Paola et al., 2011), that is similarly expressed by murine X-cells (FIG. 3C,D). Ghrelin is a hormone requiring a specific acyl modification, a process shown to be dependent on the acyl-CoA synthetase Acsl1 in mouse stomach X-cells (Bando et al., 2016). Human M/X cells and mouse intestinal X-cells both expressed high levels of this enzyme (FIG. 3C,D).

### Example 5: Genes Uniquely Expressed by Human EEC Subtypes

We next searched for human EEC genes in our organoid data that are not expressed by murine EECs. The heparin-binding growth factor Midkine (MDK) was broadly and highly expressed by all human EEC types, but not or lowly by other cell types such as goblet cells (FIG. 4A). While not previously seen in human EECs, it has been reported as a biomarker of human intestinal neuroendocrine tumors (Edfeldt et al., 2017). Midkine has been associated with obesity and inhibits insulin signaling in adipocytes (Fan et al., 2014). The carboxypeptidase CPB1 was produced by most EECs (highest in M/X cells), with the exception of ECs (FIG. 4A). Carboxypeptidases are typically involved in hormone processing (Sapio and Fricker, 2014). Expression of CPB1 has been observed in the rat pancreas (Yu et al., 2017). FGF14 is a human pan-EEC marker—with very limited expression in murine EECs—and belongs to a set of intracellular FGFs, that play a role in the clustering of ion channels in neurons (FIG. 4A, 11E) (Pablo and Pitta, 2017). The olfactory receptor OR51E2 was sporadically expressed by different EEC subtypes, with highest levels occurring in PYY⁺ cells (FIG. 4A). The mouse homologue Olfr78 on the contrary was lowly expressed in ECs only (FIG. 11E). The enzyme tryptophan 2,3-dioxygenase (TDO2) was found in duodenal EECs from the proximal intestine (FIG. 4A). TDO2can metabolize tryptophan through the kynurenine pathway and is one of the primary regulators of availability of this amino acid. Tryptophan is the precursor of serotonin and TDO2 knockout mice experience increased serotonin levels (Too et al., 2016), suggesting that TDO2 could be a local regulator of serotonin production in the gut. We noted the tachykinin peptide-coding TAC3 as a broadly expressed gene in human EECs, while the mouse homologue Tac2 is not expressed in the murine intestine (FIG. 4A). TAC3 codes for Neurokinin B and has been described as a regulator of secretion of gonadotropin-releasing hormone in humans that is produced in the hypothalamus (Sanger, 2004). However, the main receptor for NKB, NK₃ (coded by TACR3), has been implicated in the regulation of gastrointestinal motility (Sanger, 2004). The hepatokine FGF21 has recently received much attention as a regulator of blood glucose. Several FGF21 mimetics are currently being tested for the treatment of diabetes (Kuro-o, 2019). While the receptors for FGF21 are described as a complex of FGFR1 and B-Klotho (KLB), the site of action of FGF21 is much disputed. We observed broad expression of FGFR1 and KLB by human EECs, suggesting that the FGF21 effects could be partially mediated through the gut (FIG. 4A). Fgfr1 is absent in murine EECs, while Klb is expressed at very low levels (FIG. 11E). C10ORF10 (also known as DEPP1) was widely expressed by human EECs. This gene is negatively regulated by insulin in the liver and adipocyte tissue and its product controls the ratio between metabolic pathways including ketogenesis and gluconeogenesis (Li et al., 2018) (FIG. 4A). Finally, LCN15 was produced particularly by NTS⁺ cells. LCN15 is a lipocalin previously identified as one of the strongest glucose-regulated genes in Caco-2 cells (FIG. 4A) (Boztepe and Gulec, 2018). Although some lipocalins have been implicated in the development of insulin resistance, the function of LCN15 is unknown.

Because of the absence of Motilin-expressing cells in mouse intestine, we then focused on unique genes expressed by these cells but absent in murine X-cells. M/X cells were positive for TRNP1, involved in cortical folding in the brain and the only transcription factor specific to M/X cells (FIG. 4A) (Stahl et al., 2013). A putative hormone, precerebellin 1 (CBLN1), was expressed in all M/X cells (FIG. 4A). CBLN1 is known to stimulate food intake upon intracerebroventricular injection (similar to the function of Ghrelin) (Gardiner et al., 2010). We confirmed CBLN1 expression in human GHRL⁺ cells in vivo using immunofluorescence (FIG. 4B). We noted that M/X cells expressed the receptor for cytokines of the IL10-family (IL20-RA), an observation confirmed in vivo (FIG. 4B). The functionality of this receptor in secretion of motilin is demonstrated in FIG. 14A. We detected high expression of the peptide hormone Angiotensin (AGT). AGT is a regulator of blood pressure, but is also described as a regulator of contraction of the musculature of human intestinal walls (similar to the gut motility-enhancing function of Motilin) (Ewert et al., 2006) (FIG. 4B). Finally, M/X cells displayed highest expression of all EECs of the sulfate transporter SLC26A7 and of T₄- and Retinol-binding Transthyretin (TTR) (FIG. 4B). Although TTR is expressed by many EECs, both in mouse and human, M/X cells display much higher levels (FIG. 4B).

To identify heterogeneity among the different EEC subtypes, we subclustered cells sorted from organoids carrying the individual hormone reporters. Expression of the fluorescent reporters directly correlated with the levels of the pertinent hormone transcripts within the same cell (FIG. 10A). We noted that a substantial number of the cells sorted for MLN-reporter expression were L-cells, due to low level MLN expression in this population (FIG. 10A,B). Surprisingly, we identified a rare subcluster of GCG+-reporter cells that highly expressed Pancreatic Polypeptide (PPY) (Cox, 2007), a well-described peptide hormone from the endocrine pancreas involved in appetite regulation. This hormone has not previously been described in the context of the human or mouse small intestine, but we could confirm its expression and overlap with GLP-1 by staining on human intestinal sections (FIG. 10B,C).

### Example 6: Transcriptional Networks in the Human EEC Lineages

Having established the hierarchy of human EEC subtypes and expression of novel marker genes, we analyzed expression of transcription factors known from mice to specify each of these lineages (FIG. 5A). PAX4 specifies D/EC cells, while expression of ARX promotes all other EEC fates (Beucher et al., 2012). We accordingly detected these transcription factors in human EECs. HHEX and LMX1A defined human D and EC lineages respectively, consistent with their expression profile in mice (FIG. 5A) (Gross et al., 2016). The broad murine EEC transcription factors NKX2-2, PAX6, SOX4 and RFX6 were ubiquitously expressed in human EECs (Gehart et al., 2019). We additionally identify ASCL1 as a broad human EEC transcription factor, absent from M/X-cells and from all mouse EECs (FIG. 5A). Ascl1 has been described in endocrine cells in the murine lung (Borges et al., 1997). MNX1 was highly expressed only by human ECs and is known to promote neonatal diabetes when mutated (FIG. 5A) (Pan et al., 2015). Together these data indicated that human EECs express key transcriptional regulators known from murine EECs, with additional expression of transcription factors described in extra-intestinal endocrine organs. MLN⁺ cells developmentally resembled Ghrelin-producing X-cells from mice based on their transcription factor profile.

To corroborate the role of these transcriptional networks in the generation of different human EEC subtypes, we employed CRISPR/Cas9 for loss-of-function experiments. We chose to knockout the EC-specific LMX1A gene and the D-cell-specific HHEX gene (FIG. 5B). Organoids were transiently transfected with a Cas9-EGFP coding plasmid that included the site-specific gRNA (Ran et al., 2013). Transfected cells were sorted and clonally expanded, after which genotyping was performed to identify homozygous loss-of-function alleles. Lmx1a-null mice die shortly after birth, but display intestinal loss of Tph1 and Chga indicative of reduction in the enterochromaffin lineage (Gross et al., 2016). Organoids circumvent the issue of neonatal lethality for assaying gene function in adult homeostasis. Loss of LMX1A in human organoids led to a strong reduction in TPH1 (FIG. 5B). Although the other EEC lineage marker genes were unaffected, we also observed a milder reduction in SST derived from D-cells. In contrast to mouse EECs, human LMX1A is expressed in D-cells as well (FIG. 5A), pointing to a potential role in their maturation or function in addition to controlling EC development.

HHEX has been linked in genome-wide association studies to the development of type 2 diabetes in humans (Scott et al., 2007). Loss of Hhex in mice impairs the function of Sst-producing cells in pancreatic islets, while the role in the murine intestinal tract was not investigated (Zhang et al., 2014). We found that HHEX gene disruption fully blocked the production of SST, while most other EEC lineages were increased (FIG. 5B). This could be due to progenitors differentiating towards these lineages that otherwise would have become D-cells, or because of a direct (negative) effect of SST on the expression of other EEC hormones. The most striking increase was observed in GCG expression (over 20-fold). In Hhex-knockout mice, pancreatic glucagon similarly increases (Zhang et al., 2014), although the products of intestinal and pancreatic Glucagon-derived peptides act oppositely on glucose homeostasis.

### Example 7: BMP Signaling as Regulator of Hormone Switching in Human EECs

Similar to the mouse intestine, histological analysis revealed crypt-villus gradients of human hormones such as GCG (FIG. 11A). We interrogated BMP dependency of hormone gene expression in the single cell atlas. BMP activation induced NTS in L-cells at the expense of GCG (FIG. 11B). Live-cell imaging of GCG-reporter organoids confirmed that BMP activation decreased reporter expression in individual L-cells (without causing cell death) (FIG. 11C). Additionally, we observed BMP-mediated repression of GHRL in M/X cells, which was accompanied by a mild increase in MLN expression (FIG. 11B). In murine intestine, we have temporally resolved the differentiation process of EEC subtypes and found that expression of Ghrelin diminishes with migration of the X-cell along the crypt-villus axis (Gehart et al., 2019). This expression was not replaced by a second hormone; as said, Motilin does not exist as a coding gene in the mouse genome. Thus, human MLN/GHRL-producing EECs appeared to undergo a BMP-controlled switch in hormone expression as previously described in mouse.

### Example 8: High-Definition Transcriptomic Profiling of EECs

We next utilized our reporter organoid platform to construct ‘deep’ profiles of the EEC subtype transcriptome (FIG. 12A). With current technologies, transcriptomics of pooled cells has a superior sensitivity compared to single cell RNA sequencing. We exploited our reporter organoids to generate a deep transcriptomic signature of EC-, L- and M-cells. In addition, CHGA-mNeon⁺ cells were sorted to generate a broad EEC signature, while CHGA⁻ cells served as non-EEC control. We identified the top 20 uniquely expressed markers from the RNA sequencing dataset for the different EEC populations (FIG. 12B). Due to the higher sensitivity and cell number of bulk RNA sequencing compared to single cell analysis, we uncovered multiple EEC subtype features that went unnoticed in our single cell atlas. For example, the transcription factor IRX3, member of the Iroquois homeobox family, was one of the most defining markers of TPH1+ cells, yet has not been described in murine EECs (Haber et al., 2017) (FIG. 12B). IRX3 has gained attention recently as a neuronal regulator of energy balance, and genetic variants in IRX3 associate with obesity in humans (Schneeberger, 2019).

Sensory receptors of EECs, which are typically GPCRs, tend to be lowly expressed. We analyzed our bulk transcriptomic datasets for receptors enriched in subpopulations of EECs. We noted conserved expression of receptors known from mouse EECs, including FFAR2 (broad EEC), GPBAR1 (L-cell), SSTR5 (L-cell), OR51E1 (mouse homologue Olfr558; EC), ADGRG4 (Gpr112;EC) and the extracellular calcium sensor CASR (broad EEC) (Furness et al., 2013) (FIG. 12C). Newly uncovered receptors included the following: Human EECs expressed multiple orphan receptors, such as GPR162 (L-cells), not found in mice (FIG. 12C) and reported to be expressed in parts of the brain regulating food intake, while genetic variants in GPR162 are linked to impairments in glucose homeostasis (Caruso et al., 2016). GPR68 is an orphan GPCR uniquely produced by ECs (FIG. 12C). A recent study found that the orphan peptide CART (cocaine- and amphetamine-regulated protein) can activate GPR68 (Foster et al., 2019). Multiple sources are suggested for CART, including the brain and EECs, and the peptide has a role in the regulation of anxiety, reward and feeding behaviors (Shcherbina et al., 2018). GABA-A receptors have been reported in some murine EECs, but we find expression of the subunit of the GABA-B receptor GABBR2 broadly among EECs, potentially allowing these cells to respond to GABA (FIG. 12C) (Hyland and Cryan, 2010). We identified production of multiple hormone receptors in EECs, including the melanocortin receptor MC1R (FIG. 12C). MC4R has been described in murine L-cells as a regulator of hormone secretion that can be exploited by enriching the microbiome with MSH-like producing bacteria (Panaro et al., 2014). ECs selectively expressed the receptor for the thyroid-stimulating-hormone, TSHR, not observed before (FIG. 12C). Serotonin is known to regulate the levels of circulating thyroid hormones (Sullo et al., 2011), and expression of TSHR in ECs suggests that this regulation could work bidirectionally. ECs also expressed the receptor for the L-cell hormone PYY, NPY1R (FIG. 12C), reported in murine enterocytes as a regulator of electrolyte transport (Goldspink et al., 2018). We did not confirm expression of NPY1R in the CHGA-mNeon-population, enriched for human non-EECs which includes enterocytes. L-cells highly expressed the Secretin receptor SCTR that we also observed in our single cell atlas, but was not seen in mice (FIG. 12C, 13A). Fluorescent in-situ-hybridization (FISH) confirmed the expression of SCTR in EECs in vivo by overlap with CHGA (FIG. 13B). Since we observed the highest expression of SCTR in L-cells, we measured GLP-1 secretion upon a 24-hour secretin treatment in organoids. Indeed, secretin induced GLP-1 secretion at levels comparable to forskolin as measured by ELISA, or as observed by the loss of intracellular fluorescence of GCG-neon (FIG. 13C-E).

### Example 9: Organoid Assays

Two main types of target have been identified using the cell atlas described herein: receptors, and transcription factors. A number of hormones have also been identified. To validate targets identified from the EEC atlas, two main types of effect may be considered:
1. Differentiation, i.e. biasing EECs towards a subtype which results in more/less production of specific hormones by modulating a target.
2. Secretion, i.e. specific hormones being released upon target engagement.

Thus, receptors and transcription factors identified in the present application using the cell atlas may be validated as a target if they affect differentiation of EECs or if they affect hormone secretion by EECs. In some cases, a hormone itself may be a target if it for example affects differentiation or secretion of a hormone. The methods of the present invention may be used to validate such targets. For example, the inventors have validated a number of targets using the assays described below. These assays may likewise be used to validate the other targets identified in the present application and thereby show that they may be used for the applications provided herein.

Receptors:

Differentiation Screens:
1. Fluorescent reporter hormone levels are assessed using a fluorescent plate reader or fluorescence microscope. Receptors are engaged with their (predicted) ligands at any stage before or during the differentiation process. Fluorescence levels of hormones are measured to assess bias towards EEC subtypes as a result of the treatment. For example, validation of the BMP receptor as a target is shown in FIG. 7 which provides data on GCG/NTS levels (imaging) after BMP stimulation.
2. qPCR to assess hormone transcript levels. Receptors are engaged with their (predicted) ligands at any stage before or during the differentiation process. 5 days after differentiation induction, bias towards EEC subtypes as a result of the treatment is assessed. For example, validation of the Wnt receptor, the Notch receptor and the BMP receptor as targets is shown in FIG. 7 in terms of their regulation of hormone expression by measuring levels of hormone transcripts using qPCR.

Secretion Screens:
1. Fluorescent reporter hormone secretion is assessed using a fluorescent plate reader or fluorescence microscope. The decay/location change of tagged hormones is visualized and quantified. Upon stimulation with a (predicted) ligand, peptide secretion is monitored by fluorescence changes. For example, validation of SCT as an inducer for the secretin receptor as a target is shown in FIG. 13, which provides microscopy time lapses for FSK and SCT stimulation. This shows that intracellular fluorescence decreases as the hormone is secreted. FSK was used as a positive control.
2. Calcium reporters are used as a proxy for induction of secretion. Fluorescence levels of the calcium reporter are monitored in subtypes of EECs upon stimulation. Upon stimulation with a (predicted) ligand, the calcium reporter fluorescence is monitored. Validation of OR51E2 as a receptor target is shown in FIG. 2 which shows activation of the calcium reporter by stimulation of the receptor. Calcium activation is a measure of hormone secretion.
3. ELISA to measure secreted hormones in the organoid supernatant (using commercially available ELISA kits). Can be single hormone or multiplexed. Hormone levels are measured after stimulation with a (predicted) ligand. Validation of SCT as an inducer of the secretin receptor target is shown in FIG. 13 which shows an increase in GLP-1 levels in the supernatant after SCT stimulation. FSK was used as a positive control.
4. Secretomics to discover and quantify secreted products upon stimulation. Profile the multitude of secreted products upon stimulation with candidate molecules. FSK stimulation can be used as a proof of principle for secretion induction by ligands. The supernatant can be measured by mass spectrometry after contacting the organoid with an inducer and compared with a control which has not been contacted with an inducer. This enables profiling for anything that is induced rather than looking specifically at one hormone.

Transcription Factors:

Differentiation Screens:
1. Fluorescent reporter hormone levels are assessed using a fluorescent plate reader or fluorescence microscope. Organoid lines with a stable TF knockout are generated. Upon differentiation induction with doxycycline, fluorescence levels of different hormones can be measured over time or after 5 days to assess bias towards EEC subtypes as a result of the knockout.
2. qPCR to assess hormone transcript levels. Organoid lines with a stable TF knockout are generated. 5 days after differentiation induction, bias towards EEC subtypes as a result of the knockout is assessed. Validation of HHEX and LMX1A as target transcription factors is shown by the knockout experiments in FIG. 5. When the HHEX transcription factor is knocked out, SST expression is lost as were the cells expressing SST. When LMX1A is knocked out, the serotonin-positive ECs are lost.

Unbiased Screens for Libraries:

The inventors have also used a number of screens to identify useful compounds wherein their target is unknown. For example:

Differentiation Screens:
1. Fluorescent reporter hormone levels are assessed using a fluorescent plate reader or fluorescence microscope. Unbiased screens: Libraries of compounds can be screened on organoid lines with (multiple) tagged hormones. Fluorescence levels of hormones are measured to assess bias towards EEC subtypes as a result of the treatment.

Secretion Screens:
1. Fluorescent reporter hormone secretion is assessed using a fluorescent plate reader or fluorescence microscope. The decay/location change of tagged hormones is visualized and quantified. Upon stimulation with compound libraries, peptide secretion is monitored by fluorescence changes.
2. Calcium reporters are used as a proxy for induction of secretion. Fluorescence levels of the calcium reporter are monitored in subtypes of EECs upon stimulation. Upon stimulation with compound libraries, peptide secretion is monitored by fluorescence changes.
3. ELISA to measure secreted hormones in the organoid supernatant (using commercially available ELISA kits). Can be single hormone or multiplexed. Measure hormone levels after stimulation with a compound library.

Screens Confirming Existence and Secretion of Hormones:
1. qPCR—the new hormone can be assessed at the transcript level to show that it exists.
2. Secretomics—mass spectrometry of the secretome can be used to show existence of the new hormone and its secretion.

### Example 10

A fluorescent plate reader can be used to confirm that fluorescence levels (especially of the tdTomato present in all induced EECs) can be detected. The inventors have data from such a plate reader which confirms that fluorescence levels (especially of the tdTomato present in all induced EECs) can be detected.

To increase sensitivity and standardise the procedure, the following assays were performed.

For high-throughput screening, organoids were cultured in a 384-well format, in which fluorescence of the hormones can still be detected.

A first screen was performed to identify compounds that alter the ratio of subtypes of EECs that produce different hormones. For this approach, drug libraries were added at the moment EEC differentiation was initiated in organoids. Changes in relative abundance of EECs were measured in real time with a termination after 5 days, when EECs are mature. Measurement of the number of EEC subtypes was performed in organoid lines in which different hormones were fluorescently tagged.

In order to measure the differentiation efficiency of Glp-1 and Neurotensin-producing cells when treated with the signalling molecule BMP2/4, organoids containing mNeon reporters for either Glp-1 or Neurotensin were differentiated in 5 days towards EECs. EEC differentiation was induced by treating with doxycycline for 48 hours, which induces an overexpression pulse of the EEC lineage transcription factor Neurogenin-3. Mature EECs appeared 3 days later. All Neurogenin-3 overexpressed cells (that are all EEC subtypes) were also tdTomato positive: the mNeongreen hormone (Glp1 or Neurotensin) can thus be compared to the total EEC population.

The growth factor BMP regulates fate choices between Neurotensin- and Glp1-producing cells. BMP was therefore added together with doxycycline at day 0 and kept on the culture for all 5 days of differentiation. After this period, an end point measurement of fluorescence was performed. Organoids not induced with doxycycline served as a negative control for both tdTomato and Neon fluorescence. A well without organoids but with culture media served as a subtraction background for media autofluorescence.

### Example 11: EEC Receptors

The inventors have elucidated a number of cell receptor targets using the methodology described herein. Table 4 shows broad and specific receptors which have been derived from the human EEC atlas. These lists are bioinformatically generated in the following way:
1. Using GeneCards, a list of genes whose proteins are localized in the cell membrane and contain the word “receptor” in their description has been assembled.
2. For broad EEC receptors, genes upregulated in all EECs versus non-EECs in the atlas are considered. Requirements are a corrected p-value of <0.05 (Benjamini Hochberg corrected FDR), a higher expression in EECs compared to non-EECs and presence in the GeneCards list of receptors. Exclusion criterion is the presence in a specific subpopulation list (see 3.), as they are no longer “broad” in that case.
3. For EEC subtype-specific receptors, genes upregulated in one cluster of EECs versus all other EECs are considered. Requirements are analogous to (2.). Receptors can be present in more than 1 subtype lists, as they may be specific to several populations of EECs. If more than 1 cluster contributes to an EEC subtype (which was the case for ECs), we consider both genes specific to each cluster individually and genes upregulated in both clusters combined versus all other EECs. Ghrelin was falsely filtered as a receptor with this approach and removed manually.
4. Genes marked with an asterisk have been missed using this automated approach and added after deeper analysis. All of them are justified as broad/specific EEC receptors.

TABLE 4

| | D-cells | EC-cells | KG-cells | L-cells | MX-cells |
|---|---|---|---|---|---|
| Broad EEC | (SST) | (Tph1) | (GIP/GAST) | (GCG/NTS) | (MLN/GHRL) |
| PLXNB1 | GFRA3 | OR51E1 | SCTR | GPBAR1 | ASGR1 |
| SLC22A17 | CALY | MCOLN3 | SSTR2 | ABCC8 | EPHA4 |
| FZD3 | RAMP1 | TRPA1 | GUCY2C | GUCY2C | FFAR4 |
| ROR1 | ADGRG1 | NPY1R | CD44 | CALY | GFRA3 |
| LEPR | TGFBR3 | GPR160 | CNIH2 | SCTR | NTRK2 |
| NISCH | PTK7 | FFAR2 | CHRNA5 | FGFR1 | SSTR2 |
| EPHB3 | GPR160 | ADGRG1 | CALY | SSTR2 | TNFRSF21 |
| DSTYK | PVR | GAβRB3 | ABCC8 | CD44 | ABCC8 |
| EPOR | SEZ6L2 | SSTR1 | AHCYL1 | IL17RB | CD44 |
| CHRM3 | FCGRT | SLC7A1 | GRPR* | CASR | CALY |
| ITLN1 | | FLVCR1 | | PTPRN2 | PVR |
| ITPR2 | | ERRFI1 | | ADGRG1 | IL20RA* |
| ABL2 | | GABARAP | | KIT | |
| ADGRL1 | | GUCY2C | | MAGED1 | |
| PKD2 | | CNIH2 | | GPR35 | |
| GFRA1 | | CD36 | | TGFBR3 | |
| RGMB | | ADGRG4* | | PVR | |
| KREMEN1 | | GPR68* | | CNIH2 | |
| ABL1 | | TSHR* | | PKD1 | |
| EEF1A1 | | | | INSR | |
| GABBR2* | | | | TMEM97 | |
| MC1R* | | | | CLDN3 | |
| | | | | GABARAP | |
| | | | | SEZ6L2 | |
| | | | | ITGB1 | |
| | | | | SSTR5* | |
| | | | | GPR162* | |

### Example 12: Akkermansia muciniphila

The inventors assessed supernatant from the bacterium Akkermansia muciniphila (DSM 22959) regarding its effect on EEC differentiation (FIG. 141B,C). For this, organoids were induced for 24h with doxycycline and then expose then for 96 h to bacterial supernatant. The bacterial supernatant was generated by culturing bacteria for 24 h in organoid media and removing any bacterial cells by filtration before treatment of the organoids.

EEC differentiation was assessed by qPCR after the treatment. In the case of Akkermansia, this shows specific upregulation of individual hormones (most strikingly GHRL; FIG. 14 B,C). This may be indicative of skewing the differentiation preference towards MX-cells, or (slightly less likely) of increased production per cell in the MX-cell population.

### Example 13: Discussion

Human EECs are rare and have been largely inaccessible for in vitro studies. Yet, they hold great promise as central players in the treatment of metabolic diseases. Our study provides an organoid-based toolbox to address a diversity of questions on human EEC biology. We have generated a high-resolution transcriptomic and proteomic profile of human EECs from three locations along the gastrointestinal tract, including a first assessment of their secreted products. This dataset yields new hormones, transcription factors and receptors, and can be mined for novel therapeutic targets. The expression atlas highlights key differences with murine counterparts, stressing the importance of performing studies on EEC function in a human model system. Since fusions of hormones and fluorescent proteins retain the ability to be secreted, we envision EEC-TAG organoids to be used in studies aimed at identifying specific inducers of hormone secretion, or of controlling differentiation of individual EEC subtypes.

The transcriptional networks generating the different EEC subtypes have been well worked out in mice (Beucher et al., 2012; Gehart et al., 2019; Gross et al., 2016; Piccand et al., 2019). These networks could result from a stochastically acting system that generates fixed ratios of different EECs. This would explain why organoids generate rather conserved ratios of individual subtypes when compared to their tissue of origin, even in the absence of mesenchymal and luminal factors (Beumer et al., 2018). We have shown previously that BMP signaling regulates differential hormone expression within the same EEC subtype, an observation that is extended here for the hormone pair Ghrelin/Motilin (FIG. 11). Inhibition of Wnt signaling before the induced expression of NEUROG3 caused a dramatic shift in EEC distribution from GLP1⁺ L-cells to MLN⁺ M-X-cells (FIG. 7), demonstrating that the ratio of EEC subtypes can be influenced by stem cell niche factors. It remains unknown why L-cells would preferentially be specified within the Wnt^{high} stem cell zone; evidence suggest that their secreted GLP-2 is involved in the regulation of intestinal proliferation and crypt fission (Drucker et al., 1996).

A recent study has surveyed broad human EEC population using antibody-based sorting approaches and bulk RNA sequencing on human patient material (Roberts et al., 2019). This study identified some novel EEC features, such as the expression of Neuropeptide W, confirmed by the current human EEC atlas built from organoids.

We present the first transcriptomic and proteomic profiling of Motilin-producing cells. Motilin is a regulator of gut motility that displayed intriguing evolutionary dynamics, being inactivated independently in lineages leading to the mouse and rat, and guinea pigs (He et al., 2010). The Motilin receptor underwent a similar fate (He et al., 2010). This raises questions as to how the cell type (the X-cell) that produces Motilin diverged from that point. For example, the production of a certain hormone is likely to be accompanied by the expression of receptors that are sensing stimuli associated with this product. We found many similarities between mouse X-cells and the human counterparts, that we termed M-X cells. The developmental transcription factors are similar to mice (FIG. 5A), while the expression of genes required for Ghrelin modifications such as Acsl1 are conserved (FIG. 3C-D). We noted important differences also; among these the expression of putative hormones including CBLN1 and AGT. The latter has been proposed—apart from controlling blood pressure—as motility regulator similar to Motilin (FIG. 4). We also identify a cytokine receptor in M-X cells, IL-20RA, which could link a sensory mechanism for pathogens to a motility response expelling such infection. Of note, irritable bowel syndrome (IBS), characterized by alterations in gut motility, is associated with a reduction in the IL20RA-ligand IL-10, potentially mediated through M-X cells (Gonsalkorale et al., 2003).

While transthyretin (TTR) has been identified in multiple EEC populations, we identify very high levels in M/X cells (FIG. 4A). A TTR mutation underlies the most common form of amyloidosis, characterized by cardiomyopathy and cardiac failure, but also destruction of enteric neurons and associated problems in gastrointestinal motility (Ueda and Ando, 2014). Of interest, gastrointestinal motility problems are partly relieved by Motilin agonists. Although the liver is suspected as the principle source of amyloid TTR, our results indicate that local, M/X-cell produced TTR may play a role in pathogenesis.

The expression of receptors for some EEC hormones by EECs has been reported in mouse, particularly for Somatostatin (e.g. Sstr5 in L-cells) (Chisholm and Greenberg, 2002). We now find that human EECs can also sense extracellular PYY (NPY1R) and Secretin (SCTR). The PYY-receptor Npy1r has been suggested as an enterocyte marker in mouse, a finding we do not confirm (Goldspink et al., 2018). Rather, we observe exclusive expression in human Serotonin-producing ECs. SCTR expression is low in ECs and enriched in EECs producing GCG and GAST/GIP. We show that Secretin can stimulate L-cells to secrete GLP-1. Importantly, a Secretin stimulation test is commonly used in diagnostics of Zollinger-Ellison syndrome patients that suffer from gastrin-producing tumors (Berna et al., 2006). Secretin normally represses blood gastrin by inhibiting the secretion of gastrin from the stomach G-cells (the major site of Gastrin production), likely through modulating the luminal pH. Patients suffering from small intestinal gastrinoma however show sharp increases in serum gastrin upon secretin administration. Our data suggest this to occur through the broad SCTR-expression among small intestinal gastrin-producing G-cells. More broadly, our data indicate that human EECs have an extensive capacity to cross-communicate through their hormone products. Taken together, the EEC atlas and EEC-TAG biobank represent rich resources to identify regulators of human EEC development and function.

### REFERENCES

Adriaenssens, A., Lam, B. Y. H., Billing, L., Skeffington, K., Sewing, S., Reimann, F., and Gribble, F. (2015). A transcriptome-led exploration of molecular mechanisms regulating somatostatin-producing D-cells in the gastric epithelium. Endocrinology 156, 3924-3936.
Altabas, V. and Zjac̆ić-Rotkvić, V. 2015. Anti-ghrelin antibodies in appetite suppression: recent advances in obesity pharmacotherapy. Immunotargets Ther. 4: 123-130.
Amidon, S. et al., (2015). Colon-Targeted Oral Drug Delivery Systems: Design Trends and Approaches. AAPS PharmSciTech, 16(4): 731-741.
Artegiani, B., Hendriks, D., Beumer, J., Kok, R., Zheng, X., Joore, I., Chuva de Sousa Lopes, S., van Zon, J., Tans, S. and Clevers, H. (2020). Fast and efficient generation of knock-in human organoids using homology-independent CRISPR-Cas9 precision genome editing. Nature Cell Biology volume 22, pages 321-331.
Athauda, D. and Foltynie, T. 2016. The glucagon-like peptide 1 (GLP) receptor as a therapeutic target in Parkinson's disease: mechanisms of action. Drug Discovery Today. Vol. 21, 5, 802-818.
Bartfeld, S. et al. (2015). In vitro expansion of human gastric epithelial stem cells and their responses to bacterial infection. Gastroenterology 148, 126-136.e6.
Bando, M., Iwakura, H., Koyama, H., Hosoda, H., Shigematsu, Y., Ariyasu, H., Akamizu, T., Kangawa, K., and Nakao, K. (2016). High incorporation of long-chain fatty acids contributes to the efficient production of acylated ghrelin in ghrelin-producing cells. FEBS Lett. 590, 992-1001.
Barker, N., van Es, J. H., Kuipers, J., Kujala, P., van den Born, M., Cozijnsen, M., Haegebarth, A., Korving, J., Begthel, H., Peters, P. J., et al. (2007). Identification of stem cells in small intestine and colon by marker gene Lgr5. Nature 449, 1003-1007.
Bellono, N. W., Bayrer, J. R., Leitch, D. B., Castro, J., Zhang, C., O'Donnell, T. A., Brierley, S. M., Ingraham, H. A., and Julius, D. (2017). Enterochromaffin Cells Are Gut Chemosensors that Couple to Sensory Neural Pathways. Cell 170, 185-198.e16.
Berna, M. J., Hoffmann, K. M., Long, S. H., Serrano, J., Gibril, F., and Jensen, R. T. (2006). Serum gastrin in Zollinger-Ellison syndrome: II. Prospective study of gastrin provocative testing in 293 patients from the national institutes of health and comparison with 537 cases from the literature. Evaluation of diagnostic criteria, proposal of new. Medicine (Baltimore). 85, 331-364.
Beucher, A., Gjernes, E., Collin, C., Courtney, M., Meunier, A., Collombat, P., and Gradwohl, G. (2012). The homeodomain-containing transcription factors Arx and Pax4 control enteroendocrine subtype specification in mice. PLoS One.
Beumer, J., Artegiani, B., Post, Y., Reimann, F., Gribble, F., Nguyen, T. N., Zeng, H., Van den Born, M., Van Es, J. H., and Clevers, H. (2018). Enteroendocrine cells switch hormone expression along the crypt-to-villus BMP signaling gradient. Nat Cell Biol 20.
Blier, P. and Mansari, M. E. 2013. Serotonin and beyond: therapeutics for major depression. Philos Trans R Soc Lond B Biol Sci. 2013 Apr. 5; 368(1615): 20120536.
Borges, M., Linnoila, R. I., Van De Velde, H. J. K., Chen, H., Nelkin, B. D., Mabry, M., Baylin, S. B., and Ball, D. W. (1997). An achaete-scute homologue essential for neuroendocrine differentiation in the lung. Nature.
Borges-Pereira, L. et al., 2014. The GCaMP3—A GFP-based calcium sensor for imaging calcium dynamics in the human malaria parasite Plasmodium falciparum. MethodsX, 2014, 1, 151-154.
Boztepe, T., and Gulec, S. (2018). Investigation of the influence of high glucose on molecular and genetic responses: An in vitro study using a human intestine model. Genes Nutr. 13.
Bukhari, H., and Müller, T. (2019). Endogenous Fluorescence Tagging by CRISPR. Trends Cell Biol.
Cao, H., Wang, K., Ye, Y.-L., and Shen, J.-H. (2017). Orally Administrated Small Molecule Drugs with Intestine Targeted Profile: Recent Development and Prospects. Curr. Med. Chem. 24, 3921-3937.
Caruso, V., Sreedharan, S., Carlini, V. P., Jacobsson, J. A., Haitina, T., Hammer, J., Stephansson, O., Crona, F., Sommer, W. H., Risérus, U., et al. (2016). mRNA GPR162 changes are associated with decreased food intake in rat, and its human genetic variants with impairments in glucose homeostasis in two Swedish cohorts. Gene 581, 139-145.
Cataldo Bascunan, L. R. et al. 2019. Serotonergic regulation of insulin secretion. Acta Physiol (Oxf). 2019 January; 225(1):e13101.
Chang-Graham, A. L., Danhof, H. A., Engevik, M. A., Tomaro-Duchesneau, C., Karandikar, U. C., Estes, M. K., Versalovic, J., Britton, R. A., and Hyser, J. M. (2019). Human Intestinal Enteroids With Inducible Neurogenin-3 Expression as a Novel Model of Gut Hormone Secretion. Cell. Mol. Gastroenterol. Hepatol.
Charmot, D. (2012). Non-Systemic Drugs: A Critical Review. Curr Pharm Des. 18(10): 1434-1445.
Chisholm, C., and Greenberg, G. R. (2002). Somatostatin-28 regulates GLP-1 secretion via somatostatin receptor subtype 5 in rat intestinal cultures. Am. J. Physiol.—Endocrinol. Metab. 283.
Christensen M. B. (2016). Glucose-dependent insulinotropic polypeptide: effects on insulin and glucagon secretion in humans. Dan Med J. April; 63(4).
Christofides, N. D. Et al. 1979, Effect of motilin on the rate of gastric emptying and gut hormone release during breakfast. Gastroenterology, 76: 903-907.
Coates, M. D. et al. 2017. Review article: the many potential roles of intestinal serotonin (5-hydroxytryptamine, 5-HT) signalling in inflammatory bowel disease. Aliment Pharmacol. Ther. 2017, September 46(6), 569-680.
Couzens, M., Liu, M., Tüchler, C., Kofler, B., Nessler-Menardi, C., Parker, R. M. C., Klocker, H., and Herzog, H. (2000). Peptide YY-2 (PYY2) and pancreatic polypeptide-2 (PPY2): Species-specific evolution of novel members of the neuropeptide Y gene family. Genomics 64, 318-323.
Cox, H. M. (2007). Neuropeptide Y receptors; antisecretory control of intestinal epithelial function. Auton. Neurosci. Basic Clin. 133, 76-85.
Crowell, M. D. (2004). Role of serotonin in the pathophysiology of the irritable bowel syndrome. Br J Pharmacol. April; 141(8): 1285-1293.
Cui, T., Tsolakis, A. V., Li, S. C., Cunningham, J. L., Lind, T., Oberg, K., and Giandomenico, V. (2013). Olfactory receptor 51E1 protein as a potential novel tissue biomarker for small intestine neuroendocrine carcinomas. Eur. J. Endocrinol.
Deloose, E. et al. 2019. Motilin: from gastric motility stimulation to hunger signalling. Nature Reviews Endocrinology, 15, 238-250.
Deloose, E. et al. 2016. The motilin receptor agonist erythromycin stimulates hunger and food intake through a cholinergic pathway. The American Journal of Clinical Nutrition. Vol. 103. Issue 3. 730-737.
Drucker, D. J., Ehrlich, P., Asa, S. L., and Brubaker, P. L. (1996). Induction of intestinal epithelial proliferation by glucagon-like peptide 2. Proc. Natl. Acad. Sci. U.S.A. 93, 7911-7916.
Edfeldt, K., Daskalakis, K., Bäcklin, C., Norlén, O., Tiensuu Janson, E., Westin, G., Hellman, P., and Stålberg, P. (2017). DcR3, TFF3, and Midkine Are Novel Serum Biomarkers in Small Intestinal Neuroendocrine Tumors. Neuroendocrinology.
Eiel, J. N. et al. 2018. Effects of GLP-1 and GIP on Insulin Secretion in Glucose Intolerant, Pancreatic Insufficient Cystic Fibrosis. Diabetes, 2018, 67 (Supplement 1).
Engelstoft, M. S., Egerod, K. L., Lund, M. L., and Schwartz, T. W. (2013a). Enteroendocrine cell types revisited. Curr. Opin. Pharmacol.
Engelstoft, M. S., Park, W. mee, Sakata, I., Kristensen, L. V., Husted, A. S., Osborne-Lawrence, S., Piper, P. K., Walker, A. K., Pedersen, M. H., Nøhr, M. K., et al. (2013b). Seven transmembrane G protein-coupled receptor repertoire of gastric ghrelin cells. Mol. Metab.
Engelstoft, M. S., Lund, M. L., Grunddal, K. V., Egerod, K. L., Osborne-Lawrence, S., Poulsen, S. S., Zigman, J. M., and Schwartz, T. W. (2015). Research Resource: A Chromogranin A Reporter for Serotonin and Histamine Secreting Enteroendocrine Cells. Mol. Endocrinol. 29, 1658-1671.
Ewert, S., Spak, E., Olbers, T., Johnsson, E., Edebo, A., and Fändriks, L. (2006). Angiotensin II induced contraction of rat and human small intestinal wall musculature in vitro. Acta Physiol.
Faivre, E. and Holscher, C. 2013. Neuroprotective effects of D-Ala(2)GIP on Alzheimer's disease biomarkers in an APP/PS1 mouse model. Alzheimers Res. Ther. April 19; 5(2): 20.
Fan, N., Sun, H., Wang, Y., Zhang, L., Xia, Z., Peng, L., Hou, Y., Shen, W., Liu, R., and Peng, Y. (2014). Midkine, a potential link between obesity and insulin resistance. PLoS ONE. 9.
Fleischer, J., Bumbalo, R., Bautze, V., Strotmann, J., and Breer, H. (2015). Expression of odorant receptor Olfr78 in enteroendocrine cells of the colon. Cell Tissue Res. 361, 697-710.
Foster, S. R., Hauser, A. S., Vedel, L., Strachan, R. T., Huang, X. P., Gavin, A. C., Shah, S. D., Nayak, A. P., Haugaard-Kedström, L. M., Penn, R. B., et al. (2019). Discovery of Human Signaling Systems: Pairing Peptides to G Protein-Coupled Receptors. Cell 179, 895-908.e21.
Fujii, M., Matano, M., Nanki, K., and Sato, T. (2015). Efficient genetic engineering of human intestinal organoids using electroporation. Nat. Protoc. 10, 1474-1485.
Furness, J. B., Rivera, L. R., Cho, H.-J., Bravo, D. M., and Callaghan, B. (2013). The gut as a sensory organ. Nat. Rev. Gastroenterol. Hepatol. 1010, 729-740.
Gardiner, J. V., Beale, K. E., Roy, D., Boughton, C. K., Bataveljic, A., Campbell, D. C., Bewick, G. A., Patel, N. A., Patterson, M., Leavy, E. M., et al. (2010). Cerebellin1 is a novel orexigenic peptide. Diabetes, Obes. Metab.
Gavhane, Y. N. and Yadav, A. V. (2012). Loss of orally administered drugs in GI tract. Saudi Pharm J. 2012, October, 20(4): 331-344.
Gehart, H., van Es, J. H., Hamer, K., Beumer, J., Kretzschmar, K., Dekkers, J. F., Rios, A., and Clevers, H. (2019). Identification of Enteroendocrine Regulators by Real-Time Single-Cell Differentiation Mapping. Cell.
Goldspink, D. A., Reimann, F., and Gribble, F. M. (2018). Models and Tools for Studying Enteroendocrine Cells. Endocrinology.
Gong, S., Zheng, C., Doughty, M. L., Losos, K., Didkovsky, N., Schambra, U. B., Nowak, N. J., Joyner, A., Leblanc, G., Hatten, M. E., et al. (2003). A gene expression atlas of the central nervous system based on bacterial artificial chromosomes. Nature.
Gonsalkorale, W. M., Perrey, C., Pravica, V., Whorwell, P. J., and Hutchinson, I. V. (2003). Interleukin 10 genotypes in irritable bowel syndrome: Evidence for an inflammatory component? Gut 52, 91-93.
Gribble, F. M., and Reimann, F. (2017). Signalling in the gut endocrine axis. Physiol. Behav.
Grieco, M. et al. (2019). Glucagon-Like Peptide-1: A Focus on Neurodegenerative Diseases. Front. Neurosci., Volume 13. Article 1112.
Gross, S., Garofalo, D. C., Balderes, D. A., Mastracci, T. L., Dias, J. M., Perlmann, T., Ericson, J., and Sussel, L. (2016). The novel enterochromaffin marker Lmx1a regulates serotonin biosynthesis in enteroendocrine cell lineages downstream of Nkx2.2. Development 143, 2616-2628.
Grün, D., Lyubimova, A., Kester, L., Wiebrands, K., Basak, O., Sasaki, N., Clevers, H., and van Oudenaarden, A. (2015). Single-cell messenger RNA sequencing reveals rare intestinal cell types. Nature 525, 251-255.
Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339.
Hashimshony, T., Senderovich, N., Avital, G., Klochendler, A., de Leeuw, Y., Anavy, L., Gennert, D., Li, S., Livak, K. J., Rozenblatt-Rosen, O., et al. (2016). CEL-Sea2: sensitive highly-multiplexed single-cell RNA-Seq. Genome Biol. 17, 77.
He, J., Irwin, D. M., Chen, R., and Zhang, Y. P. (2010). Stepwise loss of motilin and its specific receptor genes in rodents. J. Mol. Endocrinol. 44, 37-44.
He, X., Tan, C., Wang, F., Wang, Y., Zhou, R., Cui, D., You, W., Zhao, H., Ren, J., and Feng, B. (2016). Knock-in of large reporter genes in human cells via CRISPR/Cas9-induced homology-dependent and independent DNA repair. Nucleic Acids Res.
Hellstrom, P. M. et al. (2008). GLP-1 suppresses gastrointestinal motility and inhibits the migrating motor complex in healthy subjects and patients with irritable bowel syndrome. Neurogastroenterol Motil. June; 20(6):649-59.
Herman, J. S., Sagar, and Grün, D. (2018). FateID infers cell fate bias in multipotent progenitors from single-cell RNA-seq data. Nat. Methods.
Howick, K. et al. (2017). From Belly to Brain: Targeting the Ghrelin Receptor in Appetite and Food Intake Regulation. Int. J. Mol. Sci., February 18(2): 273.
Huang, D. W., Sherman, B. T., and Lempicki, R. A. (2009). Systematic and integrative analysis of large gene lists using DAVID bioinformatics resources. Nat. Protoc. 4, 44-57.
Hyland, N. P., and Cryan, J. F. (2010). A gut feeling about GABA: Focus on GABAB receptors. Front. Pharmacol. OCT.
Israelyan N et al. 2019. Effects of Serotonin and Slow-Release 5-Hydroxytryptophan on Gastrointestinal Motility in a Mouse Model of Depression. Gastroenterology. 2019 August; 157(2):507-521.
Ji C. et al. (2016). Neuroprotective effects of glucose-dependent insulinotropic polypeptide in Alzheimer's disease. Rev Neurosci. January; 27(1):61-70.
Jones, B. et al. (2018). Control of insulin secretion by GLP-1. Peptides. February; 100:75-84.
Jost, W. H. 1997. Gastrointestinal motility problems in patients with Parkinson's disease. Effects of antiparkinsonian treatment and guidelines for management. Drugs. Aging. April; 10(4): 249-258.
Jovancevic, N., Khalfaoui, S., Weinrich, M., Weidinger, D., Simon, A., Kalbe, B., Kernt, M., Kampik, A., Gisselmann, G., Gelis, L., et al. (2017). Odorant receptor 51E2 agonist β-ionone regulates RPE cell migration and proliferation. Front. Physiol. 8.
Kaelberer, M. M., Buchanan, K. L., Klein, M. E., Barth, B. B., Montoya, M. M., Shen, X., and Bohórquez, D. V (2018). A gut-brain neural circuit for nutrient sensory transduction. Science (80-.). 361, eaat5236.
Kim, G. W. et al. (2011). Regulation of Appetite to Treat Obesity. Expert Rev Clin Pharmacol.; 4(2):243-259.
Kitazawa, T. and Kaya, H. (2019). Regulation of Gastrointestinal Motility by Motilin and Ghrelin in Vertebrates. Front. Endocrinol., Vol 10. Article 278.
Kolik, J. et al. 2014. Insulin Secretion Induced by Glucose-dependent Insulinotropic Polypeptide Requires Phosphatidylinositol 3-Kinase y in Rodent and Human β-Cells. J. Biol. Chem. 289, 32109-32120.
Koo, B. K., Stange, D. E., Sato, T., Karthaus, W., Farin, H. F., Huch, M., Van Es, J. H., and Clevers, H. (2012). Controlled gene expression in primary Lgr5 organoid cultures. Nat. Methods 9, 81-83.
Kuro-o, M. (2019). The Klotho proteins in health and disease. Nat. Rev. Nephrol.
Levin, A. D. and van den Brink, G. R. 2014. Selective inhibition of mucosal serotonin as treatment for IBD? Gut, 63: 866-867.
Levine, A. S., Winsky-Sommerer, R., Huitron-Resendiz, S., Grace, M. K., and De Lecea, L. (2005). Injection of neuropeptide W into paraventricular nucleus of hypothalamus increases food intake. Am. J. Physiol.—Regul. Integr. Comp. Physiol.
Li, H. J., Johnston, B., Aiello, D., Caffrey, D. R., Giel-Moloney, M., Rindi, G., and Leiter, A. B. (2014). Distinct cellular origins for serotonin-expressing and enterochromaffin-like cells in the gastric corpus. Gastroenterology 146.
Li, W., Ji, M., Lin, Y., Miao, Y., Chen, S., and Li, H. (2018). DEPP/DEPP1/C10ORF10 regulates hepatic glucose andm fat metabolism partly via ROS-induced FGF21. FASEB J. 32, 5459-5469.
Lindstrom, E. et al. 2001; Control of gastric acid secretion:the gastrin-ECL cell-parietal cell axis. Comp Biochem Physiol A Mol Integr Physiol; 128(3): 505-514.
Lo, C. A., Kays, I., Emran, F., Lin, T. J., Cvetkovska, V., and Chen, B. E. (2015). Quantification of Protein Levels in Single Living Cells. Cell Rep. 13, 2634-2644.
Loh, Y. P., Cheng, Y., Mahata, S. K., Corti, A., and Tota, B. (2012). Chromogranin A and derived peptides in health and disease. In Journal of Molecular Neuroscience, pp. 347-356.
Lohoff, F. W., Dahl, J. P., Ferraro, T. N., Arnold, S. E., Gallinat, J., Sander, T., and Berrettini, W. H. (2006). Variations in the vesicular monoamine transporter 1 gene (VMAT1/SLC18A1) are associated with bipolar I disorder. Neuropsychopharmacology.
Love, M. I., Huber, W., and Anders, S. (2014). Moderated estimation of fold change and dispersion for RNA-seq data with DESeq2. Genome Biol. 15, 550.
Luiking, Y. et al. 1998. Motilin induces gall bladder emptying and antral contractions in the fasted state in humans. Gut. 42(6), 830-835.
MacDonald, P. E. et al. 2002. The Multiple Actions of GLP-1 on the Process of Glucose-Stimulated Insulin Secretion. Diabetes, 2002, 51 (suppl 3): S434-S442.
Marathe, C. S. et al. 2011. Effects of GLP-1 and Incretin-Based Therapies on Gastrointestinal Motor Function. Exp. Diabetes. Res. 2011, 279530.
Masui, M. et al. 2016. Novel Midkine Inhibitor iMDK Inhibits Tumor Growth and Angiogenesis in Oral Squamous Cell Carcinoma. Anticancer Res. June, 36(6): 2775-2781.
McCracken, K. W., Catá, E. M., Crawford, C. M., Sinagoga, K. L., Schumacher, M., Rockich, B. E., Tsai, Y. H., Mayhew, C. N., Spence, J. R., Zavros, Y., et al. (2014). Modelling human development and disease in pluripotent stem-cell-derived gastric organoids. Nature.
McInnes, L., Healy, J., Saul, N., and Großberger, L. (2018). UMAP: Uniform Manifold Approximation and Projection. J. Open Source Softw. 3, 861.
Muñoz, J., Stange, D. E., Schepers, A. G., van de Wetering, M., Koo, B.-K., Itzkovitz, S., Volckmann, R., Kung, K. S., Koster, J., Radulescu, S., et al. (2012). The Lgr5 intestinal stem cell signature: robust expression of proposed quiescent “+4” cell markers. EMBO J. 31, 3079-3091.
Muraro, M. J., Dharmadhikari, G., Grün, D., Groen, N., Dielen, T., Jansen, E., van Gurp, L., Engelse, M. A., Carlotti, F., de Koning, E. J. P., et al. (2016). A Single-Cell Transcriptome Atlas of the Human Pancreas. Cell Syst. 3, 385-394.
Nguyen, T. L. A., Vieira-Silva, S., Liston, A., and Raes, J. (2015). How informative is the mouse for human gut microbiota research? DMM Dis. Model. Mech.
Pablo, J. L., and Pitta, G. S. (2017). FGF14 is a regulator of KCNQ2/3 channels. Proc. Natl. Acad. Sci. U.S.A.
Pan, F. C., Brissova, M., Powers, A. C., Pfaff, S., and Wright, C. V. E. (2015). Inactivating the permanent neonatal diabetes gene Mnx1 switches insulin-producing β-cells to a δ-like fate and reveals a facultative proliferative capacity in aged β-cells. Dev.
Panaro, B. L., Tough, I. R., Engelstoft, M. S., Matthews, R. T., Digby, G. J., Møller, C. L., Svendsen, B., Gribble, F., Reimann, F., Holst, J. J., et al. (2014). The melanocortin-4 receptor is expressed in enteroendocrine I cells and regulates the release of peptide YY and glucagon-like peptide 1 in vivo. Cell Metab. 20, 1018-1029.
Di Paola, R., Caporarello, N., Marucci, A., Dimatteo, C., Iadicicco, C., Del Guerra, S., Prudente, S., Sudano, D., Miele, C., Parrino, C., et al. (2011). ENPP1 affects insulin action and secretion: Evidences from in vitro studies. PLoS One.
Parikh, K., Antanaviciute, A., Fawkner-Corbett, D., Jagielowicz, M., Aulicino, A., Lagerholm, C., Davis, S., Kinchen, J., Chen, H. H., Alham, N. K., et al. (2019). Colonic epithelial cell diversity in health and inflammatory bowel disease. Nature.
Parker, H. E., Habib, A. M., Rogers, G. J., Gribble, F. M., and Reimann, F. (2009). Nutrient-dependent secretion of glucose-dependent insulinotropic polypeptide from primary murine K cells. Diabetologia.
Patten, G. S. and Abeywardena, M. Y. 2017. Effects of Antihypertensive Agents on Intestinal Contractility in the Spontaneously Hypertensive Rat: Angiotensin Receptor System Downregulation by Losartan. J. Pharmacol. Exp. Ther. February, 360(2): 260-266.
Paulmann, N. et al. 2009. Intracellular Serotonin Modulates Insulin Secretion from Pancreatic β-Cells by Protein Serotonylation. PLOS Biology 7(10): e1000229.
Piccand, J., Vagne, C., Blot, F., Meunier, A., Beucher, A., Strasser, P., Lund, M. L., Ghimire, S., Nivlet, L., Lapp, C., et al. (2019). Rfx6 promotes the differentiation of peptide-secreting enteroendocrine cells while repressing genetic programs controlling serotonin production. Mol. Metab. 29, 24-39.
Pietraszewska-Bogiel, A., van Weeren, L., and Goedhart, J. (2019). Seeing cells smell: Dynamic optical measurements of Ca²⁺ and cAMP signaling from Olfactory Receptors transiently expressed in HEK293TN cells. bioRxiv 771261.
Ramesh, N., Mortazavi, S., and Unniappan, S. (2015). Nesfatin-1 stimulates glucagon-like peptide-1 and glucose-dependent insulinotropic polypeptide secretion from STC-1 cells in vitro. Biochem. Biophys. Res. Commun. 462, 124-130.
Ran, F. A., Hsu, P. D., Wright, J., Agarwala, V., Scott, D. A., and Zhang, F. (2013). Genome engineering using the CRISPR-Cas9 system. Nat Protoc. 8.
Raybould, H. E. (2007). Mechanisms of CCK signaling from gut to brain. Curr. Opin. Pharmacol.
Reimann, F., Habib, A. M., Tolhurst, G., Parker, H. E., Rogers, G. J., and Gribble, F. M. (2008). Glucose Sensing in L Cells: A Primary Cell Study. Cell Metab.
Roberts, G. P., Larraufie, P., Richards, P., Kay, R. G., Galvin, S. G., Miedzybrodzka, E. L., Leiter, A., Li, H. J., Glass, L. L., Ma, M. K. L., et al. (2019). Comparison of human and murine enteroendocrine cells by transcriptomic and peptidomic profiling. In Diabetes, p.
Sachs, N., Papaspyropoulos, A., Zomer-van Ommen, D. D., Heo, I., Böttinger, L., Klay, D., Weeber, F., Huelsz-Prince, G., Iakobachvili, N., Amatngalim, G. D., et al. (2019). Long-term expanding human airway organoids for disease modeling. EMBO J. 38.
Sanger, G. J. (2004). Neurokinin NK 1 and NK 3 receptors as targets for drugs to treat gastrointestinal motility disorders and pain. Br. J. Pharmacol. 141, 1303-1312.
Sapio, M. R., and Fricker, L. D. (2014). Carboxypeptidases in disease: Insights from peptidomic studies. Proteomics—Clin. Appl. 8, 327-337.
Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265.
Sato, T., Stange, D. E., Ferrante, M., Vries, R. G. J., Van Es, J. H., Van Den Brink, S., Van Houdt, W. J., Pronk, A., Van Gorp, J., Siersema, P. D., et al. (2011). Long-term expansion of epithelial organoids from human colon, adenoma, adenocarcinoma, and Barrett's epithelium. Gastroenterology 141, 1762-1772.
Schmid-Burgk, J. L., Höning, K., Ebert, T. S., and Hornung, V. (2016). CRISPaint allows modular base-specific gene tagging using a ligase-4-dependent mechanism. Nat. Commun.
Schneeberger, M. (2019). Irx3, a new leader on obesity genetics. EBioMedicine 39, 19-20.
Scott, L. J., Mohlke, K. L., Bonnycastle, L. L., Willer, C. J., Li, Y., Duren, W. L., Erdos, M. R., Stringham, H. M., Chines, P. S., Jackson, A. U., et al. (2007). A genome-wide association study of type 2 diabetes in finns detects multiple susceptibility variants. Science (80-.). 316, 1341-1345.
Shah, M. and Vella, A. (2014). Effects of GLP-1 on appetite and weight. Rev Endocr Metab Disord. September; 15(3): 181-187.
Sharma, D., Verma, S., Vaidya, S., Kalia, K., and Tiwari, V. (2018). Recent updates on GLP-1 agonists: Current advancements & challenges. Biomed. Pharmacother.
Shcherbina, L., Lindqvist, A., Thorén Fischer, A. H., Ahlqvist, E., Zhang, E., Falkmer, S. E., Renström, E., Koffert, J., Honka, H., and Wierup, N. (2018). Intestinal CART is a regulator of GIP and GLP-1 secretion and expression. Mol. Cell. Endocrinol. 476, 8-16.
Shimizu, K. et al. 1995. The mechanism of inhibitory action of secretin on gastric acid secretion in conscious rats. J. Physiol. October 15. 488 (Pt 2): 501-508.
Silva, A. D. and Bloom, S. R. (2012). Gut Hormones and Appetite Control: A Focus on PYY and GLP-1 as Therapeutic Targets in Obesity. Gut Liver. January 6(1): 10-20.
Sinagoga, K. L., McCauley, H. A., Múnera, J. O., Reynolds, N. A., Enriquez, J. R., Watson, C., Yang, H. C., Helmrath, M. A., and Wells, J. M. (2018). Deriving functional human enteroendocrine cells from pluripotent stem cells. Development.
Soll, A H and Walsh, J H. (1979). Regulation of gastric acid secretion. Annu Rev Physiol. 41:35-53.
Sommer, C. A., and Mostoslavsky, G. (2014). Rna-seq analysis of enteroendocrine cells reveals a role for fabp5 in the control of gip secretion. Mol. Endocrinol.
Stahl, R., Walcher, T., De Juan Romero, C., Pilz, G. A., Cappello, S., Irmler, M., Sanz-Aquela, J. M., Beckers, J., Blum, R., Borrell, V., et al. (2013). Trnp1 regulates expansion and folding of the mammalian cerebral cortex by control of radial glial fate. Cell.
Sullo, A., Brizzi, G., and Maffulli, N. (2011). Chronic peripheral administration of serotonin inhibits thyroid function in the rat. Muscles. Ligaments Tendons J. 1, 48-50.
Suzuki, H. et al. 1998. Motilin controls cyclic release of insulin through vagal cholinergic muscarinic pathways in fasted dogs. Am. J. Phsiol. 274(1); G87-95.
Svendsen, B., Pais, R., Engelstoft, M. S., Milev, N. B., Richards, P., Christiansen, C. B., Egerod, K. L., Jensen, S. M., Habib, A. M., Gribble, F. M., et al. (2016). GLP1- and GIP-producing cells rarely overlap and differ by bombesin receptor-2 expression and responsiveness. J. Endocrinol. 228, 39-48.
Tack, J. et al. 2011. Targeting Serotonin Synthesis to Treat Irritable Bowel Syndrome. Gastroenterology. Vol. 141, 2, 420-422.
Thomsen, S. K., Raimondo, A., Hastoy, B., Sengupta, S., Dai, X. Q., Bautista, A., Censin, J., Payne, A. J., Umapathysivam, M. M., Spigelman, A. F., et al. (2018). Type 2 diabetes risk alleles in PAM impact insulin release from human pancreatic β-cells. Nat. Genet. 50, 1122-1131.
Too, L. K., Li, K. M., Suarna, C., Maghzal, G. J., Stocker, R., McGregor, I. S., and Hunt, N. H. (2016). Deletion of TDO2, IDO-1 and IDO-2 differentially affects mouse behavior and cognitive function. Behav. Brain Res. 312, 102-117.
Ueda, M., and Ando, Y. (2014). Recent advances in transthyretin amyloidosis therapy. Transl. Neurodegener.
Vainio, L., Perjes, A., Ryti, N., Magga, J., Alakoski, T., Serpi, R., Kaikkonen, L., Piuhola, J., Szokodi, I., Ruskoaho, H., et al. (2012). Neuronostatin, a novel peptide encoded by somatostatin gene, regulates cardiac contractile function and cardiomyocyte survival. J. Biol. Chem. 287, 4572-4580.
Wang, F., Flanagan, J., Su, N., Wang, L. C., Bui, S., Nielson, A., Wu, X., Vo, H. T., Ma, X. J., and Luo, Y. (2012). RNAscope: A novel in situ RNA analysis platform for formalin-fixed, paraffin-embedded tissues. J. Mol. Diagnostics 14, 22-29.
Worthington, J. J., Reimann, F., and Gribble, F. M. (2018). Enteroendocrine cells-sensory sentinels of the intestinal environment and orchestrators of mucosal immunity. Mucosal Immunol.
Yu, S. L., Han, S., Kim, H. R., Park, J. W., Jin, D. II, and Kang, J. (2017). Phosphorylation of carboxypeptidase B1 protein regulates β-cell proliferation. Int. J. Mol. Med.
Zhang, J., McKenna, L. B., Bogue, C. W., and Kaestner, K. H. (2014). The diabetes gene Hhex maintains δ-cell differentiation and islet function. Genes Dev. 28, 829-834.
Zhang, X., McGrath, P. S., Salomone, J., Rahal, M., McCauley, H. A., Schweitzer, J., Kovall, R., Gebelein, B., and Wells, J. M. (2019). A Comprehensive Structure-Function Study of Neurogenin3 Disease-Causing Alleles during Human Pancreas and Intestinal Organoid Development. Dev. Cell.
Zhang, Z. Q. and Holscher, C. 2020. GIP has neuroprotective effects in Alzheimer and Parkinson's disease models. Peptides, volume 125, 170184.
Zhao, D. et al. 2018. The motilin agonist erythromycin increases hunger by modulating homeostatic and hedonic brain circuits in healthy women: a randomized, placebo-controlled study. Scientific Reports. 8. Article 1819.

1.-268. (canceled)

269. A method for studying secretion, expression, and/or production of one or more EEC-specific proteins, wherein the method comprises detecting the secretion, expression, and/or production, respectively, of the one or more EEC-specific proteins by a biobank of human intestinal organoids, wherein the biobank comprises two or more human intestinal organoids that invention which are each established from different regions of the intestine, and wherein the human intestinal organoid comprises an inducible transcription factor for differentiating intestinal stem cells and/or intestinal cells with stem cell potential to EECs and further comprises one or more EEC-specific genes tagged at its endogenous locus with a detectable marker.

270. The method according to claim 269 wherein the one or more tagged EEC-specific genes are hormones, hormone precursors, and/or hormone synthesizing enzymes.

271. The method according to claim 269, wherein the regions of the intestine are selected from the proximal small intestine (duodenum), the distal small intestine (ileum), the ascending colon, the descending colon, the transverse colon, the sigmoid colon, the rectum, and the jejunum.

272. The method according to claim 269, wherein the biobank comprises human intestinal organoids which together comprise organoids established from all of the regions of the intestinal tract.

273. The method according to claim 269, wherein the biobank comprises organoids established from the proximal small intestine, the distal small intestine, and/or the ascending colon.

274. The method according to claim 270, wherein the tagged hormones and/or tagged hormone precursors comprise one or more of CHGA, MLN, GAST, GIP, CCK, GCG, GHRL, SST, NTS, and TPH1, wherein each hormone and/or hormone precursor is tagged with a detectable marker.

275. The method according to claim 270, wherein the tagged hormones and/or tagged hormone precursors comprise one or more of CHGA, MLN, GAST, GIP, CCK, GCG, GHRL, SST, and NTS.

276. The method according to claim 270, wherein the tagged hormones and/or tagged hormone precursors comprise one or more of:
(a) CCK, CHGA, CHGB, GAST, GCG, GHRL, GIP, MLN, NTS, PYY, REG4, SCG2, SCG3, SCGN, SST, UCN3, MDK, PAM, REG3A, NPW, NUCB2 and VGF;
(b) CCK, CHGA, CHGB, GAST, GCG, GHRL, GIP, MLN, NTS, PYY, REG4, SCG2, SCG3, SCGN, SST, UCN3, MDK, PAM, REG3A, NPW, NUCB2, and VGF, and one or more of TAC3, PPY, NPW and/or CBLN1;
(c) CCK, CHGA, CHGB, GAST, GCG, GHRL, GIP, MLN, NTS, PYY, REG4, SCG2, SCG3, SCGN, SST, and UCN3; or
(d) MDK, PAM, REG3A, NPW, NUCB2, and VGF.

277. The method according to claim 270, wherein the hormone synthesizing enzyme tagged with a detectable marker is TPH1 or DDC.

278. The method according to claim 270, wherein the hormone precursor tagged with a detectable marker is GCG.

279. The method according to claim 270, wherein the biobank comprises one or more of CHGA, MLN, GAST, GIP, CCK, GCG, GHRL, SST, NTS, and TPH1, each of which are tagged with a detectable marker.

280. The method according to claim 279, wherein the biobank comprises CHGA, MLN, GAST, GIP, CCK, GCG, GHRL, SST, NTS, and TPH1, each of which are tagged with a detectable marker.

281. A method for identifying, screening, or validating a compound for suitability for modulating secretion or production of one or more hormones and/or hormone precursors, and/or modulating expression of one or more EEC-specific proteins, wherein the method comprises: (i) contacting the biobank of human intestinal organoids according to claim 269 with the compound; and (ii) determining whether secretion or production of one or more hormones and/or hormone precursors is affected and/or whether expression of one or more EEC-specific genes is affected.

282. A method for identifying, screening, or validating a compound for suitability for modulating secretion or production of one or more hormones and/or hormone precursors, and/or modulating expression of one or more EEC-specific proteins, wherein the method comprises: (i) contacting the biobank of human intestinal organoids according to claim 269; and (ii) determining whether expression of one or more hormones, hormone precursors, and/or hormone synthesizing enzymes is affected.


SEQUENCE LISTING (87 sequences)

SEQ ID NO: 1 - DNA, 644 bp, Homo sapiens
  misc_feature (1)..(644): NEUROG3
tgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccaccttttccgcctgcttgagcccaggcagtctggctttctcagattttctgtga

SEQ ID NO: 2 - protein, 214 aa, Homo sapiens
  misc_feature (1)..(214): NEUROG3
MTPQPSGAPTVQVTRETERSFPRASEDEVTCPTSAPPSPTRTRGNCAEAEEGGCRGAPRKLRARRGGRSRPKSELALSKQRRSRRKKANDRERNRMHNLNSALDALRGVLPTFPDDAKLTKIETLRFAHNYIWALTQTLRIADHSLYALEPPAPHCGELGSPGGSPGDWGSLYSPVSQAGSLSPAASLEERPGLLGATFSACLSPGSLAFSDFL

SEQ ID NO: 3 - DNA, 1051 bp, Homo sapiens
  misc_feature (1)..(1051): NEUROD1
atgaccaaatcgtacagcgagagtgggctgatgggcgagcctcagccccaaggtcctccaagctggacagacgagtgtctcagttctcaggacgaggagcacgaggcagacaagaaggaggacgacctcgaaaccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctca

SEQ ID NO: 4 - protein, 356 aa, Homo sapiens
  misc_feature (1)..(356): NEUROD1
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 5 - DNA, 1064 bp, Homo sapiens
  misc_feature (1)..(1064): ATOH1
tgcaacgcctgctgcatgcagaagagtgggctgaagtgaaggagttgggagaccaccatcgccagccccagccgcatcatctcccgcaaccgccgccgccgccgcagccacctgcaactttgcaggcgagagagcatcccgtctacccgcctgagctgtccctcctggacagcaccgacccacgcgcctggctggctcccactttgcagggcatctgcacggcacgcgccgcccagtatttgctacattccccggagctgggtgcctcagaggccgctgcgccccgggacgaggtggacggccggggggagctggtaaggaggagcagcggcggtgccagcagcagcaagagccccgggccggtgaaagtgcgggaacagctgtgcaagctgaaaggcggggtggtggtagacgagctgggctgcagccgccaacgggccccttccagcaaacaggtgaatggggtgcagaagcagagacggctagcagccaacgccagggagcggcgcaggatgcatgggctgaaccacgccttcgaccagctgcgcaatgttatcccgtcgttcaacaacgacaagaagctgtccaaatatgagaccctgcagatggcccaaatctacatcaacgccttgtccgagctgctacaaacgcccagcggaggggaacagccaccgccgcctccagcctcctgcaaaagcgaccaccaccaccttcgcaccgcggcctcctatgaagggggcgcgggcaacgcgaccgcagctggggctcagcaggcttccggagggagccagcggccgaccccgcccgggagttgccggactcgcttctcagccccagcttctgcgggagggtactcggtgcagctggacgctctgcacttctcgactttcgaggacagcgccctgacagcgatgatggcgcaaaagaatttgtctccttctctccccgggagcatcttgcagccagtgcaggaggaaaacagcaaaacttcgcctcggtcccacagaagcgacggggaattttccccccattcccattacagtgactcggatgaggcaagttag

SEQ ID NO: 6 - protein, 354 aa, Homo sapiens
  misc_feature (1)..(354): ATOH1
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRSHRSDGEFSPHSHYSDSDEAS

SEQ ID NO: 7 - DNA, 25 bp, Artificial Sequence
  GCG forward gRNA
caccgttcagaccaaaatcactgac

SEQ ID NO: 8 - DNA, 25 bp, Artificial Sequence
  GCG reverse gRNA
aaacgtcagtgattttggtctgaac

SEQ ID NO: 9 - DNA, 25 bp, Artificial Sequence
  MLN forward gRNA
caccgtccccagcgtggccatcact

SEQ ID NO: 10 - DNA, 25 bp, Artificial Sequence
  MLN reverse gRNA
aaacagtgatggccacgctggggac

SEQ ID NO: 11 - DNA, 25 bp, Artificial Sequence
  GIP forward gRNA
caccgagacaaacctctgcaggctc

SEQ ID NO: 12 - DNA, 25 bp, Artificial Sequence
  GIP reverse gRNA
aaacgagcctgcagaggtttgtctc

SEQ ID NO: 13 - DNA, 25 bp, Artificial Sequence
  CHGA forward gRNA
caccgcagctgcaggcactacggcg

SEQ ID NO: 14 - DNA, 25 bp, Artificial Sequence
  CHGA reverse gRNA
aaaccgccgtagtgcctgcagctgc

SEQ ID NO: 15 - DNA, 25 bp, Artificial Sequence
  NTS forward gRNA
caccgtttgagtatgtagggtcttc

SEQ ID NO: 16 - DNA, 25 bp, Artificial Sequence
  NTS reverse gRNA
aaacgaagaccctacatactcaaac

SEQ ID NO: 17 - DNA, 25 bp, Artificial Sequence
  GHRL forward gRNA
caccggcttgtgggcgatcacttgt

SEQ ID NO: 18 - DNA, 25 bp, Artificial Sequence
  GHRL reverse gRNA
aaacacaagtgatcgcccacaagcc

SEQ ID NO: 19 - DNA, 25 bp, Artificial Sequence
  CCK forward gRNA
caccgtggcggctgggtcctctagg

SEQ ID NO: 20 - DNA, 25 bp, Artificial Sequence
  CCK reverse gRNA
aaaccctagaggacccagccgccac

SEQ ID NO: 21 - DNA, 25 bp, Artificial Sequence
  SST forward gRNA
caccgctggctgcaagaatttcttc

SEQ ID NO: 22 - DNA, 25 bp, Artificial Sequence
  SST reverse gRNA
aaacgaagaaattcttgcagccagc

SEQ ID NO: 23 - DNA, 25 bp, Artificial Sequence
  GAST forward gRNA
caccgcttcggccgccgcagtgctg

SEQ ID NO: 24 - DNA, 25 bp, Artificial Sequence
  GAST reverse gRNA
aaaccagcactgcggcggccgaagc

SEQ ID NO: 25 - DNA, 38 bp, Artificial Sequence
  TPH1 left homology arm forward primer
aacgctcttcataaggggagttaggccattcatgaaac

SEQ ID NO: 26 - DNA, 37 bp, Artificial Sequence
  TPH1 left homology arm reverse primer
cgcgctcttcctgtgatactcggcttcctgctgacct

SEQ ID NO: 27 - DNA, 37 bp, Artificial Sequence
  TPH1 right homology arm forward primer
cgcgctcttcgcgccagtagccagtcatccaggaaca

SEQ ID NO: 28 - DNA, 42 bp, Artificial Sequence
  TPH1 right homology arm reverse primer
aacgctcttcgattaactcttcctttgataacaatgcttcct

SEQ ID NO: 29 - DNA, 25 bp, Artificial Sequence
  TPH1 forward gRNA
caccgactggctactgttagatact

SEQ ID NO: 30 - DNA, 25 bp, Artificial Sequence
  TPH1 reverse gRNA
aaacagtatctaacagtagccagtc

SEQ ID NO: 31 - DNA, 25 bp, Artificial Sequence
  HHEX forward gRNA
caccgaaggctggatggatcggcgt

SEQ ID NO: 32 - DNA, 25 bp, Artificial Sequence
  HHEX reverse gRNA
aaacacgccgatccatccagccttc

SEQ ID NO: 33 - DNA, 25 bp, Artificial Sequence
  LMX1A forward gRNA
caccgccgaggtgtcgatcgcgctt

SEQ ID NO: 34 - DNA, 25 bp, Artificial Sequence
  LMX1A reverse gRNA
aaacaagcgcgatcgacacctcggc

SEQ ID NO: 35 - DNA, 21 bp, Artificial Sequence
  HHEX forward primer
tttacatcgaggacatcctgg

SEQ ID NO: 36 - DNA, 23 bp, Artificial Sequence
  HHEX reverse primer
aaaccaatctgagtcacggtgcg

SEQ ID NO: 37 - DNA, 20 bp, Artificial Sequence
  LMX1A forward primer
ataccccagtagctgcgttc

SEQ ID NO: 38 - DNA, 20 bp, Artificial Sequence
  LMX1A reverse primer
cgagaagagggcggcattta

SEQ ID NO: 39 - DNA, 20 bp, Artificial Sequence
  BSKS forward primer
gggctgcaggaattcgatat

SEQ ID NO: 40 - DNA, 20 bp, Artificial Sequence
  BSKS reverse primer
cactagttctagagcggccg

SEQ ID NO: 41 - DNA, 116 bp, Artificial Sequence
  Tag forward primer
ggtggtggtggttcaggaggaggatcgggaagcggagagggcagaggaagtctgctaacatgcggtgacgtcgaggagaatcctggacctgaattcgggctgcaggaattcgatat

SEQ ID NO: 42 - DNA, 116 bp, Artificial Sequence
  Tag reverse primer
atatcgaattcctgcagcccgaattcaggtccaggattctcctcgacgtcaccgcatgttagcagacttcctctgccctctccgcttcccgatcctcctcctgaaccaccaccacc

SEQ ID NO: 43 - DNA, 40 bp, Artificial Sequence
  NEUROG3 forward primer
cctcctgaaccaccaccacccagaaaatctgagaaagcca

SEQ ID NO: 44 - DNA, 52 bp, Artificial Sequence
  NEUROG3 reverse primer
cggccgctctagaactagtggaattcgccaccatgacgcctcaaccctcggg

SEQ ID NO: 45 - DNA, 21 bp, Artificial Sequence
  GAPDH forward primer
ggagcgagatccctccaaaat

SEQ ID NO: 46 - DNA, 23 bp, Artificial Sequence
  GAPDH reverse primer
ggctgttgtcatacttctcatgg

SEQ ID NO: 47 - DNA, 22 bp, Artificial Sequence
  CHGA forward primer
taaaggggataccgaggtgatg

SEQ ID NO: 48 - DNA, 22 bp, Artificial Sequence
  CHGA reverse primer
tcggagtgtctcaaaacattcc

SEQ ID NO: 49 - DNA, 21 bp, Artificial Sequence
  SST forward primer
acccaaccagacggagaatga

SEQ ID NO: 50 - DNA, 20 bp, Artificial Sequence
  SST reverse primer
gccgggtttgagttagcaga

SEQ ID NO: 51 - DNA, 22 bp, Artificial Sequence
  LYZ forward primer
cttgtcctcctttctgttacgg

SEQ ID NO: 52 - DNA, 20 bp, Artificial Sequence
  LYZ reverse primer
cccctgtagccatccattcc

SEQ ID NO: 53 - DNA, 22 bp, Artificial Sequence
  GAST forward primer
atgcagcgactatgtgtgtatg

SEQ ID NO: 54 - DNA, 19 bp, Artificial Sequence
  GAST reverse primer
gcccctgtacctaagggtg

SEQ ID NO: 55 - DNA, 21 bp, Artificial Sequence
  CCK forward primer
agctccttctggacgaatgtc

SEQ ID NO: 56 - DNA, 21 bp, Artificial Sequence
  CCK reverse primer
tgtagtcccggtcacttatcc

SEQ ID NO: 57 - DNA, 21 bp, Artificial Sequence
  MLN forward primer
atggtatcccgtaaggctgtg

SEQ ID NO: 58 - DNA, 21 bp, Artificial Sequence
  MLN reverse primer
ctggagttcgccataggtgaa

SEQ ID NO: 59 - DNA, 23 bp, Artificial Sequence
  NTS forward primer
tgctttagatggctttagcttgg

SEQ ID NO: 60 - DNA, 22 bp, Artificial Sequence
  NTS reverse primer
ttcctggattaactcccagtgt

SEQ ID NO: 61 - DNA, 20 bp, Artificial Sequence
  PYY forward primer
gacgcctaccccatcaaacc

SEQ ID NO: 62 - DNA, 21 bp, Artificial Sequence
  PYY reverse primer
ccgtctcttttcccataccgc

SEQ ID NO: 63 - DNA, 21 bp, Artificial Sequence
  GCG forward primer
acattgccaaacgtcacgatg

SEQ ID NO: 64 - DNA, 20 bp, Artificial Sequence
  GCG reverse primer
tctgcggccaagttcttcaa

SEQ ID NO: 65 - DNA, 21 bp, Artificial Sequence
  TPH1 forward primer
acgtcgaaagtattttgcgga

SEQ ID NO: 66 - DNA, 21 bp, Artificial Sequence
  TPH1 reverse primer
acggttccccaggtcttaatc

SEQ ID NO: 67 - DNA, 21 bp, Artificial Sequence
  OR51E2 forward primer
acttcacacatgccacctttg

SEQ ID NO: 68 - DNA, 21 bp, Artificial Sequence
  OR51E2 reverse primer
gaagaccacgatgcagtttcc

SEQ ID NO: 69 - protein, 123 aa, Homo sapiens
VAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 70 - protein, 27 aa, Homo sapiens
HSDGTFTSELSRLREGARLQRLLQGLV

SEQ ID NO: 71 - protein, 97 aa, Homo sapiens
MVFVRRPWPALTTVLLALLVCLGALVDAYPIKPEAPREDASPEELNRYYASLRHYLNLVTRQRYGKRDGPDTLLSKTFFPDGEDRPVRSRSEGPDLW

SEQ ID NO: 72 - protein, 36 aa, Homo sapiens
YPSKPDNPGEDAPAEDMARYYSALRHYINLITRQRY

SEQ ID NO: 73 - protein, 39 aa, Homo sapiens
SYSMEHFRWGKPVGKKRRPVKVYPNGAEDESAEAFPLEF

SEQ ID NO: 74 - protein, 13 aa, Homo sapiens
  MOD_RES (1)..(1): ACETYLATION
  MOD_RES (13)..(13): AMIDATION
SYSMEHFRWGKPV

SEQ ID NO: 75 - protein, 22 aa, Homo sapiens
AEKKDEGPYRMEHFRWGSPPKD

SEQ ID NO: 76 - protein, 12 aa, Homo sapiens
YVMGHFRWDRFG

SEQ ID NO: 77 - protein, 161 aa, Homo sapiens
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 78 - protein, 154 aa, Homo sapiens
MLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMSSA

SEQ ID NO: 79 - protein, 153 aa, Homo sapiens
MLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 80 - protein, 155 aa, Homo sapiens
QEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 81 - protein, 150 aa, Homo sapiens
KHKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCISCASSAREMKSITRMKRIFYRIGNKGIYKAISELDILLSWIKKLLESSQ

SEQ ID NO: 82 - protein, 27 aa, Homo sapiens
  MOD_RES (27)..(27): AMIDATION
VPLPAGGGTVLTKMYPRGNHWAVGHLM

SEQ ID NO: 83 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 84 - protein, 118 aa, Homo sapiens
FCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 85 - protein, 48 aa, Homo sapiens
VPIYEKKYGQVPMCDAGEQCAVRKGARIGKLCDCPRGTSCNSFLLKCL

SEQ ID NO: 86 - protein, 41 aa, Homo sapiens
YGQVPMCDAGEQCAVRKGARIGKLCDCPRGTSCNSFLLKCL

SEQ ID NO: 87 - protein, 11 aa, Homo sapiens [sequence omitted]